(19) United States
(12) Patent Application Publication
(10) Pub. No.: US 2024/0109905 A1
(43) Pub. Date: Apr. 4, 2024

(54) 4-ALKOXY-6-OXO-PYRIDAZINE DERIVATIVES MODULATING NLRP3

(71) Applicant: JANSSEN PHARMACEUTICA NV, Beerse (BE)

(72) Inventors: Daniel OEHLRICH, Mol (BE); Nina VAN OPDENBOSCH, Scheldewindeke (BE); Mohamed LAMKANFI, Berlare (BE); Michael Eric MURATORE, Mauerbach (AT); Dries VAN ROMPAEY, Schoten (BE); Maria LINARES DE LA MORENA, Bargas (Toledo) (ES); Manuel Jesús ALCÁZAR VACA, Toledo (ES); Michiel Luc Maria VAN GOOL, Madrid (ES)

(21) Appl. No.: 18/548,739

(22) PCT Filed: Mar. 3, 2022
(86) PCT No.: PCT/EP2022/055432

§ 371 Date: Sep. 1, 2023

(30) Foreign Application Priority Data
Mar. 4, 2021 (EP) 21160668.6

(51) Int. Cl. C07D 487/04; A61K 45/06; A61P 37/04; C07D 237/16; C07D 401/12; C07D 403/12; C07D 405/12; C07D 471/04

(52) U.S. Cl. CPC C07D 487/04; A61K 45/06; A61P 37/04; C07D 237/16; C07D 401/12; C07D 403/12; C07D 405/12; C07D 471/04

(57) ABSTRACT

The invention relates to novel compounds for use as inhibitors of NLRP3 inflammasome production, wherein such compounds are as defined by compounds of formula (I) and wherein the integers R¹, R², R³ and R⁴ are defined in the description, and where the compounds may be useful as medicaments, for instance for use in the treatment of a disease or disorder that is associated with NLRP3 inflammasome activity.

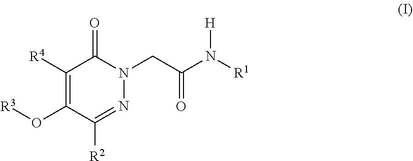

### FIELD OF THE INVENTION

The present invention relates to novel triazinones that are useful as inhibitors of NOD-like receptor protein 3 (NLRP3) inflammasome pathway. The present invention also relates to processes for the preparation of said compounds, pharmaceutical compositions comprising said compounds, methods of using said compounds in the treatment of various diseases and disorders, and medicaments containing them, and their use in diseases and disorders mediated by NLRP3.

### BACKGROUND OF THE INVENTION

Inflammasomes, considered as central signaling hubs of the innate immune system, are multi-protein complexes that are assembled upon activation of a specific set of intracellular pattern recognition receptors (PRRs) by a wide variety of pathogen- or danger-associated molecular patterns (PAMPs or DAMPs). To date, it was shown that inflammasomes can be formed by nucleotide-binding oligomerization domain (NOD)-like receptors (NLRs) and Pyrin- and HIN200-domain-containing proteins (Van Opdenbosch N and Lamkanfi M. Immunity, 2019 Jun. 18; 50(6):1352-1364). The NLRP3 inflammasome is assembled upon detection of environmental crystals, pollutants, host-derived DAMPs and protein aggregates (Tartey S and Kanneganti T D. Immunology, 2019 April; 156(4):329-338). Clinically relevant DAMPs that engage NLRP3 include uric acid and cholesterol crystals that cause gout and atherosclerosis, amyloid-0 fibrils that are neurotoxic in Alzheimer's disease and asbestos particles that cause mesothelioma (Kelley et al., Int J Mol Sci, 2019 Jul. 6; 20 (13)). Additionally, NLRP3 is activated by infectious agents such as Vibrio cholerae; fungal pathogens such as Aspergillus fumigatus and Candida albicans; adenoviruses, influenza A virus and SARS-CoV-2 (Tartey and Kanneganti, 2019 (see above); Fung et al. Emerg Microbes Infect, 2020 Mar. 14; 9(1):558-570).

Although the precise NLRP3 activation mechanism remains unclear, for human monocytes, it has been suggested that a one-step activation is sufficient while in mice a two-step mechanism is in place. Given the multitude in triggers, the NLRP3 inflammasome requires add-on regulation at both transcriptional and post-transcriptional level (Yang Y et al., Cell Death Dis, 2019 Feb. 12; 10(2):128).

The NLRP3 protein consists of an N-terminal pyrin domain, followed by a nucleotide-binding site domain (NBD) and a leucine-rich repeat (LRR) motif on C-terminal end (Sharif et al., Nature, 2019 June; 570(7761):338-343). Upon recognition of PAMP or DAMP, NLRP3 aggregates with the adaptor protein, apoptosis-associated speck-like protein (ASC), and with the protease caspase-1 to form a functional inflammasome. Upon activation, procaspase-1 undergoes autoproteolysis and consequently cleaves gasdermin D (Gsdmd) to produce the N-terminal Gsdmd molecule that will ultimately lead to pore-formation in the plasma membrane and a lytic form of cell death called pyroptosis. Alternatively, caspase-1 cleaves the pro-inflammatory cytokines pro-IL-10 and pro-IL-18 to allow release of its biological active form by pyroptosis (Kelley et al., 2019—see above).

Dysregulation of the NLRP3 inflammasome or its downstream mediators are associated with numerous pathologies ranging from immune/inflammatory diseases, auto-immune/auto-inflammatory diseases (Cryopyrin-associated Periodic Syndrome (Miyamae T. Paediatr Drugs, 2012 Apr. 1; 14(2):109-17); sickle cell disease; systemic lupus erythematosus (SLE)) to hepatic disorders (e.g. non-alcoholic steatohepatitis (NASH), chronic liver disease, viral hepatitis, alcoholic steatohepatitis, and alcoholic liver disease) (Szabo G and Petrasek J. Nat Rev Gastroenterol Hepatol, 2015 July; 12(7):387-400) and inflammatory bowel diseases (e.g. Crohn's disease, ulcerative colitis) (Zhen Y and Zhang H. Front Immunol, 2019 Feb. 28; 10:276). Also, inflammatory joint disorders (e.g. gout, pseudogout (chondrocalcinosis), arthropathy, osteoarthritis, and rheumatoid arthritis (Vande Walle L et al., Nature, 2014 Aug. 7; 512(7512):69-73) were linked to NLRP3. Additionally, kidney related diseases (hyperoxaluria (Knauf et al., Kidney Int, 2013 November; 84(5):895-901), lupus nephritis, hypertensive nephropathy (Krishnan et al., Br J Pharmacol, 2016 February; 173(4):752-65), hemodialysis related inflammation and diabetic nephropathy which is a kidney-related complication of diabetes (Type 1, Type 2 and mellitus diabetes), also called diabetic kidney disease (Shahzad et al., Kidney Int, 2015 January; 87(1):74-84) are associated to NLRP3 inflammasome activation. Reports link onset and progression of neuroinflammation-related disorders (e.g. brain infection, acute injury, multiple sclerosis, Alzheimer's disease) and neurodegenerative diseases (Parkinson's disease) to NLRP3 inflammasome activation (Sarkar et al., NPJ Parkinson's Dis, 2017 Oct. 17; 3:30). In addition, cardiovascular or metabolic disorders (e.g. cardiovascular risk reduction (CvRR), atherosclerosis, type I and type II diabetes and related complications (e.g. nephropathy, retinopathy), peripheral artery disease (PAD), acute heart failure and hypertension (Ridker et al., CANTOS Trial Group. N Engl J Med, 2017 Sep. 21; 377(12):1119-1131; and Toldo S and Abbate A. Nat Rev Cardiol, 2018 April; 15(4):203-214) have recently been associated to NLRP3. Also, skin associated diseases were described (e.g. wound healing and scar formation; inflammatory skin diseases, e.g. acne, hidradenitis suppurativa (Kelly et al., Br J Dermatol, 2015 December; 173(6)). In addition, respiratory conditions have been associated with NLRP3 inflammasome activity (e.g. asthma, sarcoidosis, Severe Acute Respiratory Syndrome (SARS) (Nieto-Torres et al., Virology, 2015 November; 485:330-9)) but also age-related macular degeneration (Doyle et al., Nat Med, 2012 May; 18(5):791-8). Several cancer related diseases/disorders were described linked to NLRP3 (e.g. myeloproliferative neoplasms, leukemias, myelodysplastic syndromes (MOS), myelofibrosis, lung cancer, colon cancer (Ridker et al., Lancet, 2017 Oct. 21; 390(10105):1833-1842; Derangere et al., Cell Death Differ. 2014 December; 21(12):1914-24; Basiorka et al., Lancet Haematol, 2018 September; 5(9): e393-e402, Zhang et al., Hum Immunol, 2018 January; 79(1):57-62).

Several patent applications describe NLRP3 inhibitors, with recent ones including for instance international patent application WO 2020/234715, WO 2020/018975, WO 2020/037116, WO 2020/021447, WO 2020/010143, WO 2019/079119, WO 2019/0166621 and WO 2019/121691, which disclose a range of specific compounds.

There is a need for inhibitors of the NLRP3 inflammasome pathway to provide new and/or alternative treatments for the diseases/disorders mentioned herein.

### SUMMARY OF THE INVENTION

The invention provides compounds which inhibit the NLRP3 inflammasome pathway.

Thus, in an aspect of the invention, there is provided a compound of formula (I),

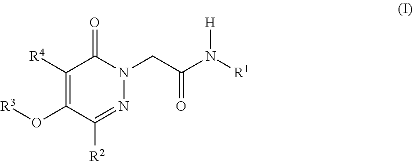

or a pharmaceutically acceptable salt thereof, wherein:
R¹ represents:
   (i) C₃₋₈ cycloalkyl optionally substituted with one or more substituents independently selected from halo; cyano; C₁₋₃ alkyl; haloC₁₋₃ alkyl; —OH; —O—C₁₋₃ alkyl; —O—C₃₋₆ cycloalkyl; —NH₂; —NH-t.Boc; —NHC₁₋₃ alkyl; —N(C₁₋₃ alkyl)₂; piperidine; morpholine; hydroxyC₁₋₃ alkyl; C₁₋₃ alkyl substituted with —NH₂, —NH— C₁₋₃ alkyl, —O—C₁₋₃ alkyl or —SO₂—C₁₋₃ alkyl; —COOH; —COOC₁₋₃ alkyl; —CO—NH—NH₂; —CONH₂; —CONHC₁₋₃ alkyl; —CONHC₃alkynyl; —CON(C₁₋₃ alkyl)₂; —SO₂—C₁₋₃ alkyl; —SO₂—C₃₋₆cycloalkyl; heteroaryl or heterocyclyl;
   (ii) aryl or heteroaryl, each of which is optionally substituted with 1 to 3 substituents independently selected from halo, —OH, —O—C₁₋₃ alkyl, —C₁₋₃ alkyl, haloC₁₋₃ alkyl, hydroxyC₁₋₃ alkyl, hydroxyC₁₋₃ alkoxy, haloC₁₋₃alkoxy; or
   (iii) heterocyclyl, optionally substituted with 1 to 3 substituents independently selected from C₁₋₃ alkyl and C₃₋₆ cycloalkyl;
R² represents:
   (i) C₁₋₃ alkyl optionally substituted with one or more substituents independently selected from halo, —OH and —OC₁₋₃ alkyl;
   (ii) C₃₋₆ cycloalkyl; or
   (iii) C₂₋₄ alkenyl optionally substituted with —OC₁₋₃ alkyl; or
   (iv) —N(R^{2a})R^{2b};
R^{2a} and R^{2b} each represent hydrogen or C₁₋₄ alkyl, or R^{2a} and R^{2b} may be linked together to form a 3- to 4-membered ring optionally substituted by one or more fluoro atoms;
R³ represents:
   (i) C₁₋₆ alkyl optionally substituted with one or more substituents independently selected from halo, —OH, —OC₁₋₃ alkyl, —NH₂, —N(H)C₁₋₃ alkyl, —N(C₁₋₃ alkyl)₂ and —C(O)N(C₁₋₃ alkyl)₂;
   (ii) C₂₋₆ alkenyl optionally substituted with one or more substituents independently selected from halo, —OH, —OC₁₋₃ alkyl, —NH₂, —N(H)C₁₋₃ alkyl, —N(C₁₋₃ alkyl)₂ and —C(O)N(C₁₋₃ alkyl)₂;
   (iii) aryl or heteroaryl, each of which is optionally substituted with 1 to 3 substituents independently selected from halo, —OH, —O—C₁₋₃ alkyl, —C₁₋₃ alkyl, haloC₁₋₃ alkyl, hydroxyC₁₋₃ alkyl, hydroxyC₁₋₃ alkoxy, haloC₁₋₃alkoxy;
   (iv) —X^{1a}—Y^{1a}, in which Y^{1a} represents C₃₋₆ cycloalkyl optionally substituted with one or more substituents independently selected from halo, —OH and —C₁₋₃ alkyl; or
   (v) —X^{1b}—Y^{1b}, in which Y^{1b} represents heterocyclyl, optionally substituted with 1 to 3 substituents independently selected from halo, ═O, C₁₋₃ alkyl and —C(O)—C₁₋₆ alkyl;
X^{1a} and X^{1b} independently represent a —CH₂— linker group or a direct bond (i.e. is not present); R⁴ represents:
   (i) hydrogen;
   (ii) halo;
   (iii) C₁₋₄ alkyl optionally substituted with one or more substituents independently selected from halo, —OH and —OC₁₋₃ alkyl;
   (iv) C₃₋₆ cycloalkyl; or
   (v) —OC₁₋₃ alkyl.

In another aspect there is provided a compound of formula (I), wherein R¹ represents C₃₋₆ cycloalkyl optionally substituted with one or more substituents independently selected from —OH and —C₁₋₃ alkyl.

In another aspect, there is provided compounds of the invention for use as a medicament. In another aspect, there is provided a pharmaceutical composition comprising a therapeutically effective amount of a compound of the invention.

In a further aspect, there is provided compounds of the invention (and/or pharmaceutical compositions comprising such compounds) for use: in the treatment of a disease or disorder associated with NLRP3 activity (including inflammasome activity); in the treatment of a disease or disorder in which the NLRP3 signaling contributes to the pathology, and/or symptoms, and/or progression, of said disease/disorder; in inhibiting NLRP3 inflammasome activity (including in a subject in need thereof); and/or as an NLRP3 inhibitor. Specific diseases or disorders may be mentioned herein, and may for instance be selected from inflammasome-related diseases or disorders, immune diseases, inflammatory diseases, auto-immune diseases, or auto-inflammatory diseases.

In another aspect, there is provided a use of compounds of the invention (and/or pharmaceutical compositions comprising such compounds): in the treatment of a disease or disorder associated with NLRP3 activity (including inflammasome activity); in the treatment of a disease or disorder in which the NLRP3 signaling contributes to the pathology, and/or symptoms, and/or progression, of said disease/disorder; in inhibiting NLRP3 inflammasome activity (including in a subject in need thereof); and/or as an NLRP3 inhibitor.

In another aspect, there is provided use of compounds of the invention (and/or pharmaceutical compositions comprising such compounds) in the manufacture of a medicament for: the treatment of a disease or disorder associated with NLRP3 activity (including inflammasome activity); the treatment of a disease or disorder in which the NLRP3 signaling contributes to the pathology, and/or symptoms, and/or progression, of said disease/disorder; and/or inhibiting NLRP3 inflammasome activity (including in a subject in need thereof).

In another aspect, there is provided a method of treating a disease or disorder in which the NLRP3 signaling contributes to the pathology, and/or symptoms, and/or progression, of said disease/disorder, comprising administering a therapeutically effective amount of a compound of the invention, for instance to a subject (in need thereof). In a further aspect there is provided a method of inhibiting the NLRP3 inflammasome activity in a subject (in need thereof), the method comprising administering to the subject in need thereof a therapeutically effective amount of a compound of the invention.

In further aspect, there is a provided a compound of the invention in combination (including a pharmaceutical combination) with one or more therapeutic agents (for instance as described herein). Such combination may also be provided for use as described herein in respect of compounds of the invention, or, a use of such combination as described herein in respect of compounds of the invention. There may also be provided methods as described herein in respect of compounds of the invention, but wherein the method comprises administering a therapeutically effective amount of such combination.

### DETAILED DESCRIPTION OF THE INVENTION

In an aspect of the invention, there is provided a compound of formula (I),

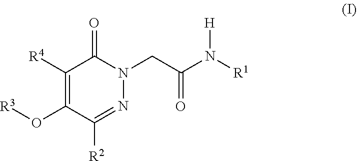

or a pharmaceutically acceptable salt thereof, wherein:
R¹ represents:
   (iv) C₃₋₈ cycloalkyl optionally substituted with one or more substituents independently selected from halo; cyano; C₁₋₃ alkyl; haloC₁₋₃ alkyl; —OH; —O—C₁₋₃ alkyl; —O—C₃₋₆ cycloalkyl; —NH₂; —NH-t.Boc; —NHC₁₋₃ alkyl; —N(C₁₋₃ alkyl)₂; piperidine; morpholine; hydroxyC₁₋₃ alkyl; C₁₋₃ alkyl substituted with —NH₂, —NH— C₁₋₃ alkyl, —O—C₁₋₃ alkyl or —SO₂—C₁₋₃ alkyl; —COOH; —COOC₁₋₃ alkyl; —CO—NH—NH₂; —CONH₂; —CONHC₁₋₃ alkyl; —CONHC₃alkynyl; —CON(C₁₋₃ alkyl)₂; —SO₂—C₁₋₃ alkyl; —SO₂—C₃₋₆cycloalkyl; heteroaryl or heterocyclyl;
   (v) aryl or heteroaryl, each of which is optionally substituted with 1 to 3 substituents independently selected from halo, —OH, —O—C₁₋₃ alkyl, —C₁₋₃ alkyl, haloC₁₋₃ alkyl, hydroxyC₁₋₃ alkyl, hydroxyC₁₋₃ alkoxy, haloC₁₋₃alkoxy; or
   (vi) heterocyclyl, optionally substituted with 1 to 3 substituents independently selected from C₁₋₃ alkyl and C₃₋₆ cycloalkyl;
R² represents:
   (v) C₁₋₃ alkyl optionally substituted with one or more substituents independently selected from halo, —OH and —OC₁₋₃ alkyl;
   (vi) C₃₋₆ cycloalkyl; or
   (vii) C₂₋₄ alkenyl optionally substituted with —OC₁₋₃ alkyl; or
   (viii) —N(R^{2a})R^{2b};
R^{2a} and R^{2b} each represent hydrogen or C₁₋₄ alkyl, or R^{2a} and R^{2b} may be linked together to form a 3- to 4-membered ring optionally substituted by one or more fluoro atoms;
R³ represents:
   (vi) C₁₋₆ alkyl optionally substituted with one or more substituents independently selected from halo, —OH, —OC₁₋₃ alkyl, —NH₂, —N(H)C₁₋₃ alkyl, —N(C₁₋₃ alkyl)₂ and —C(O)N(C₁₋₃ alkyl)₂;
   (vii) C₂₋₆ alkenyl optionally substituted with one or more substituents independently selected from halo, —OH, —OC₁₋₃ alkyl, —NH₂, —N(H)C₁₋₃ alkyl, —N(C₁₋₃ alkyl)₂ and —C(O)N(C₁₋₃ alkyl)₂;
   (viii) aryl or heteroaryl, each of which is optionally substituted with 1 to 3 substituents independently selected from halo, —OH, —O—C₁₋₃ alkyl, —C₁₋₃ alkyl, haloC₁₋₃ alkyl, hydroxyC₁₋₃ alkyl, hydroxyC₁₋₃ alkoxy, haloC₁₋₃alkoxy;
   (ix) —X^{1a}—Y^{1a}, in which Y^{1a} represents C₃₋₆ cycloalkyl optionally substituted with one or more substituents independently selected from halo, —OH and —C₁₋₃ alkyl; or
   (x) —X^{1b}—Y^{1b}, in which Y^{1b} represents heterocyclyl, optionally substituted with 1 to 3 substituents independently selected from halo, ═O, C₁₋₃ alkyl and —C(O)—C₁₋₆ alkyl;
X^{1a} and X^{1b} independently represent a —CH₂— linker group or a direct bond (i.e. is not present);
R⁴ represents:
   (vi) hydrogen;
   (vii) halo;
   (viii) C₁₋₄ alkyl optionally substituted with one or more substituents independently selected from halo, —OH and —OC₁₋₃ alkyl;
   (ix) C₃₋₆ cycloalkyl; or
   (x) —OC₁₋₃ alkyl.

The invention further provides a compound of formula (I),

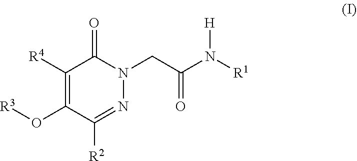

or a pharmaceutically acceptable salt thereof, wherein:
R¹ represents:
   (i) C₃₋₆ cycloalkyl optionally substituted with one or more substituents independently selected from —OH and —C₁₋₃ alkyl;
   (ii) aryl or heteroaryl, each of which is optionally substituted with 1 to 3 substituents independently selected from halo, —OH, —O—C₁₋₃ alkyl, —C₁₋₃ alkyl, haloC₁₋₃ alkyl, hydroxyC₁₋₃ alkyl, hydroxyC₁₋₃ alkoxy, haloC₁₋₃alkoxy; or
   (iii) heterocyclyl, optionally substituted with 1 to 3 substituents independently selected from C₁₋₃ alkyl and C₃₋₆ cycloalkyl;
R² represents:
   (i) C₁₋₃ alkyl optionally substituted with one or more substituents independently selected from halo, —OH and —OC₁₋₃ alkyl;
   (ii) C₃₋₆ cycloalkyl; or
   (iii) C₂₋₄ alkenyl optionally substituted with —OC₁₋₃ alkyl;
   (iv) —N(R^{2a})R^{2b};
R^{2a} and R^{2b} each represent hydrogen or C₁₋₄ alkyl, or R^{2a} and R^{2b} may be linked together to form a 3- to 4-membered ring optionally substituted by one or more fluoro atoms;
R³ represents:
   (i) C₁₋₆ alkyl optionally substituted with one or more substituents independently selected from halo, —OH, —OC₁₋₃ alkyl, —NH₂, —N(H)C₁₋₃ alkyl, —N(C₁₋₃ alkyl)₂ and —C(O)N(C₁₋₃ alkyl)₂;
   (ii) C₂₋₆ alkenyl optionally substituted with one or more substituents independently selected from halo, —OH, —OC₁₋₃ alkyl, —NH₂, —N(H)C₁₋₃ alkyl, —N(C₁₋₃ alkyl)₂ and —C(O)N(C₁₋₃ alkyl)₂;
   (iii) aryl or heteroaryl, each of which is optionally substituted with 1 to 3 substituents independently selected from halo, —OH, —O—C₁₋₃ alkyl, —C₁₋₃ alkyl, haloC₁₋₃ alkyl, hydroxyC₁₋₃ alkyl, hydroxyC₁₋₃ alkoxy, haloC₁₋₃alkoxy;
   (iv) —X^{1a}—Y^{1a}, in which Y^{1a} represents C₃₋₆ cycloalkyl optionally substituted with one or more substituents independently selected from halo, —OH and —C₁₋₃ alkyl; or
   (v) —X^{1b}—Y^{1b}, in which Y^{1b} represents heterocyclyl, optionally substituted with 1 to 3 substituents independently selected from halo, ═O, C₁₋₃ alkyl and —C(O)—C₁₋₆ alkyl;
X^{1a} and X^{1b} independently represent a —CH₂— linker group or a direct bond (i.e. is not present);
R⁴ represents:
   (i) hydrogen;
   (ii) halo;
   (iii) C₁₋₄ alkyl optionally substituted with one or more substituents independently selected from halo, —OH and —OC₁₋₃ alkyl;
   (iv) C₃₋₆ cycloalkyl; or
   (v) —OC₁₋₃ alkyl.

As indicated above, such compounds may be referred to herein as “compounds of the invention”.

Pharmaceutically-acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine

For the purposes of this invention solvates, prodrugs, N-oxides and stereoisomers of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent. Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elsevier, New York-Oxford (1985).

Compounds of the invention may contain double bonds and may thus exist as E (entgegen) and Z (zusammen) geometric isomers about each individual double bond. Positional isomers may also be embraced by the compounds of the invention. All such isomers (e.g. if a compound of the invention incorporates a double bond or a fused ring, the cis- and trans-forms, are embraced) and mixtures thereof are included within the scope of the invention (e.g. single positional isomers and mixtures of positional isomers may be included within the scope of the invention).

Compounds of the invention may also exhibit tautomerism. All tautomeric forms (or tautomers) and mixtures thereof are included within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerisations. Valence tautomers include interconversions by reorganization of some of the bonding electrons.

Compounds of the invention may also contain one or more asymmetric carbon atoms and may therefore exhibit optical and/or diastereoisomerism. Diastereoisomers may be separated using conventional techniques, e.g. chromatography or fractional crystallization. The various stereoisomers may be isolated by separation of a racemic or other mixture of the compounds using conventional, e.g. fractional crystallization or HPLC, techniques. Alternatively the desired optical isomers may be made by reaction of the appropriate optically active starting materials under conditions which will not cause racemization or epimerization (i.e. a ‘chiral pool’ method), by reaction of the appropriate starting material with a ‘chiral auxiliary’ which can subsequently be removed at a suitable stage, by derivatization (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person.

All stereoisomers (including but not limited to diastereoisomers, enantiomers and atropisomers) and mixtures thereof (e.g. racemic mixtures) are included within the scope of the invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

When an absolute configuration is specified, it is according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer.

The compounds of the present invention may exist in unsolvated as well as solvated forms with pharmaceutically acceptable solvents such as water, ethanol, and the like, and it is intended that the invention embrace both solvated and unsolvated forms.

The present invention also embraces isotopically-labelled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ⁴C, ¹³N, ¹⁵O, ¹⁷O, ¹⁸O, ³²P, ³³P, ³⁵S, ¹⁸F, ³⁶Cl, ¹²³I, and ¹²⁵I. Certain isotopically-labelled compounds of the present invention (e.g., those labelled with ³H and ¹⁴C) are useful in compound and for substrate tissue distribution assays. Tritiated (³H) and carbon-14 (¹⁴C) isotopes are useful for their ease of preparation and detectability. Further, substitution with heavier isotopes such as deuterium (i.e., ²H may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labelled compounds of the present invention can generally be prepared by following procedures analogous to those disclosed in the description/Examples hereinbelow, by substituting an isotopically labelled reagent for a non-isotopically labelled reagent.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain. Such a group is attached to the rest of the molecule by a single bond.

C_{2-q} alkenyl when used herein (again where q is the upper limit of the range) refers to an alkyl group that contains unsaturation, i.e. at least one double bond.

C_{3-q} cycloalkyl (where q is the upper limit of the range) refers to an alkyl group that is cyclic, for instance cycloalkyl groups may be monocyclic or, if there are sufficient atoms, bicyclic. In an embodiment, such cycloalkyl groups are monocyclic.

Such cycloalkyl groups are unsaturated. Substituents may be attached at any point on the cycloalkyl group.

The term “halo”, when used herein, preferably includes fluoro, chloro, bromo and iodo.

C_{1-q} alkoxy groups (where q is the upper limit of the range) refers to the radical of formula —OR^{a}, where R^{a} is a C_{1-q} alkyl group as defined herein.

HaloC_{1-q} alkyl (where q is the upper limit of the range) groups refer to C_{1-q} alkyl groups, as defined herein, where such group is substituted by one or more halo. HydroxyC_{1-q} alkyl (where q is the upper limit of the range) refers to C_{1-q} alkyl groups, as defined herein, where such group is substituted by one or more (e.g. one) hydroxy (—OH) groups (or one or more, e.g. one, of the hydrogen atoms is replaced with —OH). Similarly, haloC_{1-q} alkoxy and hydroxyC_{1-q} alkoxy represent corresponding —OC_{1-q} alkyl groups that are substituted by one or more halo, or, substituted by one or more (e.g. one) hydroxy, respectively.

Heterocyclyl groups that may be mentioned include non-aromatic monocyclic and bicyclic heterocyclyl groups in which at least one (e.g. one to four) of the atoms in the ring system is other than carbon (i.e. a heteroatom), and in which the total number of atoms in the ring system is between 3 and 20 (e.g. between three and ten, e.g. between 3 and 8, such as 5- to 8-). Such heterocyclyl groups may also be bridged. Such heterocyclyl groups are saturated. C_{2-q} heterocyclyl groups that may be mentioned include 7-azabicyclo[2.2.1]heptanyl, 6-azabicyclo[3.1.1]heptanyl, 6-azabicyclo[3.2.1]-octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl, oxetanyl, oxiranyl, piperazinyl, piperidinyl, non-aromatic pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocyclyl groups may, where appropriate, be located on any atom in the ring system including a heteroatom. The point of attachment of heterocyclyl groups may be via any atom in the ring system including (where appropriate) a heteroatom (such as a nitrogen atom), or an atom on any fused carbocyclic ring that may be present as part of the ring system. Heterocyclyl groups may also be in the N- or S-oxidised form. In an embodiment, heterocyclyl groups mentioned herein are monocyclic.

Aryl groups that may be mentioned include C₆₋₂₀, such as C₆₋₁₂ (e.g. C₆₋₁₀) aryl groups. Such groups may be monocyclic, bicyclic or tricyclic and have between 6 and 12 (e.g. 6 and 10) ring carbon atoms, in which at least one ring is aromatic. C₆₋₁₀ aryl groups include phenyl, naphthyl and the like, such as 1,2,3,4-tetrahydronaphthyl. The point of attachment of aryl groups may be via any atom of the ring system. For example, when the aryl group is polycyclic the point of attachment may be via atom including an atom of a non-aromatic ring. However, when aryl groups are polycyclic (e.g. bicyclic or tricyclic), they are preferably linked to the rest of the molecule via an aromatic ring. When aryl groups are polycyclic, in an embodiment, each ring is aromatic. In an embodiment, aryl groups mentioned herein are monocyclic or bicyclic. In a further embodiment, aryl groups mentioned herein are monocyclic.

“Heteroaryl” when used herein refers to an aromatic group containing one or more heteroatom(s) (e.g. one to four heteroatoms) preferably selected from N, O and S. Heteroaryl groups include those which have between 5 and 20 members (e.g. between 5 and 10) and may be monocyclic, bicyclic or tricyclic, provided that at least one of the rings is aromatic (so forming, for example, a mono-, bi-, or tricyclic heteroaromatic group). When the heteroaryl group is polycyclic the point of attachment may be via any atom including an atom of a non-aromatic ring. However, when heteroaryl groups are polycyclic (e.g. bicyclic or tricyclic), they are preferably linked to the rest of the molecule via an aromatic ring. In an embodiment, when heteroaryl groups are polycyclic, then each ring is aromatic. Heteroaryl groups that may be mentioned include 3,4-dihydro-1H-isoquinolinyl, 1,3-dihydroisoindolyl, 1,3-dihydroisoindolyl (e.g. 3,4-dihydro-1H-isoquinolin-2-yl, 1,3-dihydroisoindol-2-yl, 1,3-dihydroisoindol-2-yl; i.e. heteroaryl groups that are linked via a non-aromatic ring), or, preferably, acridinyl, benzimidazolyl, benzodioxanyl, benzodioxepinyl, benzodioxolyl (including 1,3-benzodioxolyl), benzofuranyl, benzofurazanyl, benzothiadiazolyl (including 2,1,3-benzothiadiazolyl), benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. When heteroaryl groups are polycyclic in which there is a non-aromatic ring present, then that non-aromatic ring may be substituted by one or more ═O group. In an embodiment, heteroaryl groups mentioned herein may be monocyclic or bicyclic. In a further embodiment, heteroaryl groups mentioned herein are monocyclic.

Heteroatoms that may be mentioned include phosphorus, silicon, boron and, preferably, oxygen, nitrogen and sulfur.

For the avoidance of doubt, where it is stated herein that a group may be substituted by one or more substituents (e.g. selected from C₁₋₆ alkyl), then those substituents (e.g. alkyl groups) are independent of one another. That is, such groups may be substituted with the same substituent (e.g. same alkyl substituent) or different (e.g. alkyl) substituents.

All individual features (e.g. preferred features) mentioned herein may be taken in isolation or in combination with any other feature (including preferred feature) mentioned herein (hence, preferred features may be taken in conjunction with other preferred features, or independently of them).

The skilled person will appreciate that compounds of the invention that are the subject of this invention include those that are stable. That is, compounds of the invention include those that are sufficiently robust to survive isolation from e.g. a reaction mixture to a useful degree of purity.

Various embodiments of the invention will now be described, including embodiments of the compounds of the invention.

In an embodiment, compounds of the invention include those in which R¹ represents: (i) C₃₋₆ cycloalkyl; (ii) aryl or heteroaryl; or (iii) or heterocyclyl, all of which are optionally substituted as herein defined. In a particular embodiment, R¹ represents: (i) C₃₋₆ cycloalkyl; or (ii) aryl or heteroaryl, all of which are optionally substituted as herein defined.

In an embodiment when R¹ represents optionally substituted C₃₋₆ cycloalkyl, then it represents C₃₋₆ cycloalkyl (or, in an embodiment, C₃₋₄ cycloalkyl) optionally substituted by one or two substituents selected from C₁₋₃ alkyl (e.g. methyl) and —OH. In a further embodiment, R¹ represents cyclopropyl (e.g. unsubstituted) or cyclobutyl. In a further embodiment, R¹ represents cyclohexyl. In yet a further embodiment, R¹ represents unsubstituted cyclopropyl or cyclobutyl substituted by —OH and methyl (e.g. at the same carbon atom). In yet a further embodiment, R¹ represents cyclohexyl, for instance substituted by —OH (e.g. by one —OH group). In an embodiment therefore, R¹ represents:

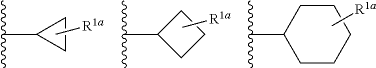

where each R^{1a} represents one or two optional substituents selected from —OH and C₁₋₃ alkyl (e.g. methyl). In a particular embodiment of this aspect, R¹ represents C₃₋₆ cycloalkyl, such as optionally substituted cyclohexyl, optionally substituted cyclobutyl or unsubstituted (or optionally substituted) cyclopropyl, for instance:

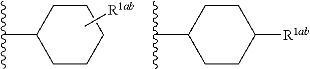

where each R^{1ab} represents one or two optional substituents selected from those defined by R^{1a}, and in an embodiment, represents one optional substituent selected from —OH;

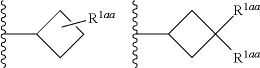

where each R^{1aa} represents one or two optional substituents selected from those defined by R^{1a}, and in an embodiment represents two substituents, methyl and —OH; or

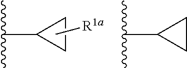

where R^{1a} is as defined above, but where, in a particular embodiment, it is not present.

In an embodiment where R¹ represents aryl or heteroaryl, optionally substituted as defined herein, then it may represent: (i) phenyl; (ii) a 5- or 6-membered mono-cyclic heteroaryl group; or (iii) a 9- or 10-membered bicyclic heteroaryl group, all of which are optionally substituted by one to three substituents as defined herein. In an embodiment, the aforementioned aryl and heteroaryl groups are optionally substituted with one or two (e.g. one) substituent(s) selected from halo (e.g. fluoro), —OH, C₁₋₃ alkyl and —OC₁₋₃ alkyl. In one embodiment, R¹ represents phenyl or a mono-cyclic 6-membered heteroaryl group and in another embodiment it may represent a 9- or 10-membered (e.g. 9-membered) bicyclic heteroaryl group. Hence, in an embodiment, R¹ may represent:

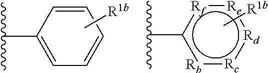

wherein R^{1b} represents one or two optional substituents selected from halo, —CH₃, —OH and —OCH₃ (and in a further embodiment, such optional substituents are selected from fluoro and methoxy), and at least one of R_{b}, R_{c}, R_{d}, Rₑ and R_{f} represents a nitrogen heteroatom (and the others represent CH). In an embodiment, either one or two of R_{b}, R_{c}, R_{d}, Rₑ and R_{f} represent(s) a nitrogen heteroatom, for instance, R_{d} represents nitrogen and, optionally, R_{b} represents nitrogen, or, R_{c} represents nitrogen. In an aspect: (i) R_{b} and R_{d} represent nitrogen; (ii) R_{d} represents nitrogen; or (iii) R_{c} represents nitrogen. Hence, R¹ may represent 3-pyridyl or 4-pyrimidinyl, both of which are optionally substituted as herein defined; however, in an embodiment, such groups are unsubstituted.

In another embodiment, R¹ may represent:

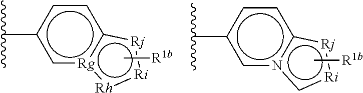

wherein R^{1b} is as defined above (i.e. represents one or two optional substituents as defined above), each ring of the bicyclic system is aromatic, R_{g} represents a N or C atom and any one or two of Rₕ, Rᵢ and Rⱼ (for instance, one or two of Rᵢ and Rⱼ) represents N and the other(s) represent(s) C (provided that, as the skilled person would understand, the rules of valency are adhered to; for instance when one of the atoms of the (hetero)aromatic ring represents C, then it is understood that it may bear a H atom).

In an embodiment R¹ represents:

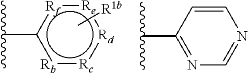

in which R_{b} and R_{d} represent a nitrogen atom, and, in an embodiment, there is no R^{1b} substituent present.

In another embodiment, R¹ represents:

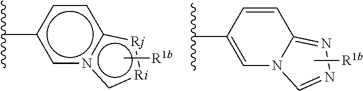

in which one of Rᵢ and Rⱼ represents N and the other represents C, or, both Rᵢ and Rⱼ represent N, and, in an embodiment, there is no R^{1b} substituent present.

In an embodiment R² represents: (i) C₁₋₃ alkyl optionally substituted with one or more substituents independently selected from halo (e.g. fluoro), —OH and —OC₁₋₂ alkyl; (ii) C₃₋₆ cycloalkyl; or (iii) C₂₋₄ alkenyl optionally substituted by —OC₁₋₂ alkyl. In a further embodiment, R² represents C₁₋₃ alkyl optionally substituted with one or more substituents independently selected from halo, —OH and —OC₁₋₂ alkyl, or, R² represents C₃₋₆ cycloalkyl. In yet a further embodiment, R² represents unsubstituted C₁₋₃ alkyl or C₃₋₆ cycloalkyl.

In a particular embodiment R² represents unsubstituted isopropyl or unsubstituted cyclopropyl.

In an embodiment, R³ represents: (i) C₁₋₆ alkyl optionally substituted by one or more substituents independently selected from fluoro, —N(C₁₋₃ alkyl)₂ and —C(O)N(CH₃)₂; (ii) aryl (e.g. phenyl) optionally substituted by one or more substituents selected from halo, —OC₁₋₃ alkyl, —C₁₋₃ alkyl and haloC₁₋₃ alkyl (but is in an embodiment, unsubstituted); (iii) —X^{1a}—Y^{1a}, in which X^{1a} represents —CH₂— or a direct bond, and Y^{1a} represents C₃₋₆ cycloalkyl (e.g. C₃₋₅ cycloalkyl) optionally substituted by one or more (e.g. one or two) halo (e.g. fluoro) atoms; (iv) —X^{1b}—Y^{1b}, in which X^{1b} represents —CH₂— or a direct bond, and Y^{1b} represents heterocyclyl, for instance a 4-6 membered heterocyclyl group, optionally bridged, and containing one or two (e.g. one) heteroatom(s) selected from nitrogen, oxygen and sulfur, and which heterocyclyl group is optionally substituted by one or more substituents selected from halo, ═O, C₁₋₃ alkyl and —C(O)C₁₋₄ alkyl (for instance ═O substituents may be present on a sulfur atom, and —C(O)C₁₋₄ alkyl may be present on a N atom).

In an embodiment, Y^{1a} may represent:

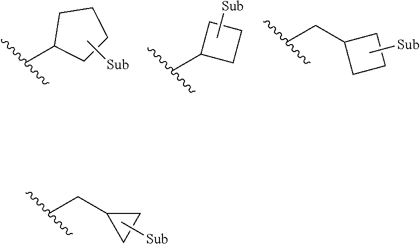

where Sub represents one or more optional substituents that may be present on the cycloalkyl group.

In an embodiment, Y^{1b} may represent:

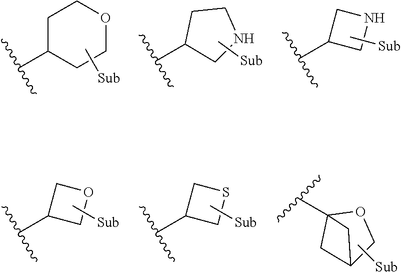

where Sub represents one or more optional substituents that may be present on the heterocyclyl group (including on the heteroatoms, e.g. the sulfur may be substituted with one or two ═O).

In an embodiment, when R³ represents —X^{1a}—Y^{1a}, then it may represent:

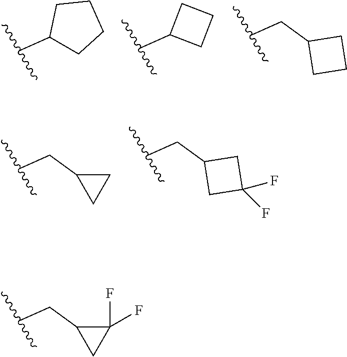

In an embodiment, when R³ represents —X^{1b}—Y^{1b}, then it may represent:

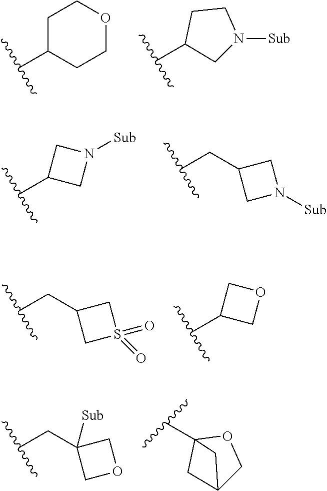

where “Sub” represents an optional substituent as hereinbefore defined.

In an embodiment, R³ represents —CH₃, —CH₂CH₃, —CH₂CH₂CH₃, —CH(CH₃)₂ (isopropyl), —CH(CH₃)—CH₂CH₃, —CH₂—CH(CH₃)₂, —CH₂CF₃, —CH₂CHF₂, —CH₂—C(CH₃)₂—CF₃, —CH₂—C(CH₃)₂F, —CH₂C(CH₃)F2, —C(H)(CH₃)—CF₃, —CH₂CH₂—N(CH₃)₂ or —CH(CH₃)—C(O)N(CH₃)₂. In another embodiment, R³ represents phenyl (e.g. unsubstituted phenyl). In another embodiment, R³ represents -cyclopentyl, -cyclobutyl, —CH₂-cyclopropyl (optionally substituted by two fluoro atoms) or —CH₂-cyclobutyl (optionally substituted by two fluoro atoms). In another embodiment, R³ represents pyrrolidinyl, azetidine, (e.g. 3-pyrrolidinyl or 3-azetidinyl; for instance optionally substituted at the N atom by —C(O)—C₁₋₄ alkyl, such as —C(O)-tert-butyl), —CH₂-azetidine (e.g. —CH₂-(3-azetidine); for instance optionally substituted at the N atom by —C(O)—C₁₋₄ alkyl, such as —C(O)-tert-butyl), —CH₂-thietane (e.g. —CH₂-(3-thietane); for instance where the sulfur atom is substituted with one or two ═O atoms, so forming e.g. a sulfur dione), —CH₂-oxetane (e.g. —CH₂-(3-oxetane); which may be substituted by one or more halo or C₁₋₃ alkyl group e.g. one C₁₋₃ alkyl group that may form a quaternary carbon atom), tetrahydropyran (e.g. 4-tetrahydropyran) or —CH₂-(2-oxabicyclo[2.1.1]hexane).

In an embodiment R⁴ represents hydrogen, halo, C₁₋₃ alkyl or C₃₋₆ cycloalkyl. In a particular embodiment R⁴ represents hydrogen, bromo or cyclopropyl. In a certain embodiment, R⁴ represents hydrogen.

The names of the compounds of the present invention were generated according to the nomenclature rules agreed upon by the Chemical Abstracts Service (CAS) using Advanced Chemical Development, Inc., software (ACD/Name product version 10.01; Build 15494, 1 Dec. 2006) or according to the nomenclature rules agreed upon by the International Union of Pure and Applied Chemistry (IUPAC) using Advanced Chemical Development, Inc., software (ACD/Name product version 10.01.0.14105, October 2006). In case of tautomeric forms, the name of the depicted tautomeric form of the structure was generated. The other non-depicted tautomeric form is also included within the scope of the present invention.

### Preparation of the Compounds

In an aspect of the invention, there is provided a process for the preparation of compounds of the invention, where reference here is made to compounds of formula (I) as defined herein.

Compounds of formula (I) may be prepared by:
(i) reaction of a compound of formula (II),

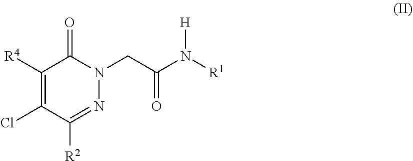

or a derivative thereof, wherein R¹ and R² are as hereinbefore defined, and R⁴ is hydrogen, with a compound of formula (III),

HO—R³ (III)
wherein R³ is as hereinbefore defined, under nucleophilic substitution reaction conditions, for example in the presence of a suitable base (e.g. sodium hydride) in a suitable solvent such as dimethylformamide;
(ii) reaction of a compound of formula (IV),

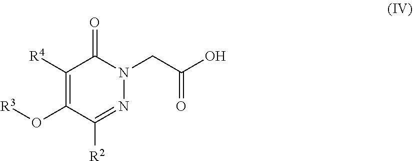

or a derivative thereof (e.g. a salt), wherein R², R³ and R⁴ are as hereinbefore defined, with a compound of formula (V),

H₂N—R¹ (V)
or a derivative thereof, wherein R¹ is as hereinbefore defined, under amide-forming reaction conditions (also referred to as amidation), for example in the presence of a suitable coupling reagent (e.g. 1-[bis(dimethylamino)methylene]-1H-1,2,3-triazolo[4,5-b]pyridinium 3-oxide hexafluorophosphate) in the presence of a suitable base (e.g. diisopropylamine) and an appropriate solvent (e.g. dimethylformamide);
(iii) reaction of a compound of formula (VI),

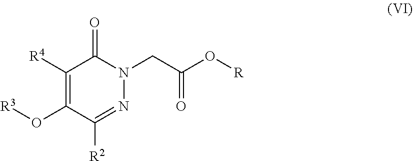

wherein R is C₁₋₄ alkyl and R², R³ and R⁴ are as hereinbefore defined, with a compound of formula (V),

H₂N—R¹ (V)
or a derivative thereof, wherein R¹ is as hereinbefore defined, under amide-forming reaction conditions (also referred to as amidation), for example in the presence of a suitable strong base (e.g. lithium bis(trimethylsilyl)amide) in the presence of an appropriate solvent (e.g. dimethylformamide and tetrahydrofuran);
(iv) by transformation (such transformation step may also take place on intermediates) of a certain compound of formula (I) into another, for example, for compounds of formula (I) in which R has a functional group such as NH₂, such group is optionally protected, deprotection reaction using standard acidic conditions, e.g. with trifluoroacetic acid in a suitable solvent such as dichloromethane.

In general, the compounds of the invention can therefore be made with reference to the procedures above. However, in the interests of versatility, further schemes are provided below in order to provide intermediate compounds of the invention. Further details are provided in the schemes below (as well as in the specific details of the experimental described hereinafter).

In this respect, Scheme 1 outlines a typical synthesis:

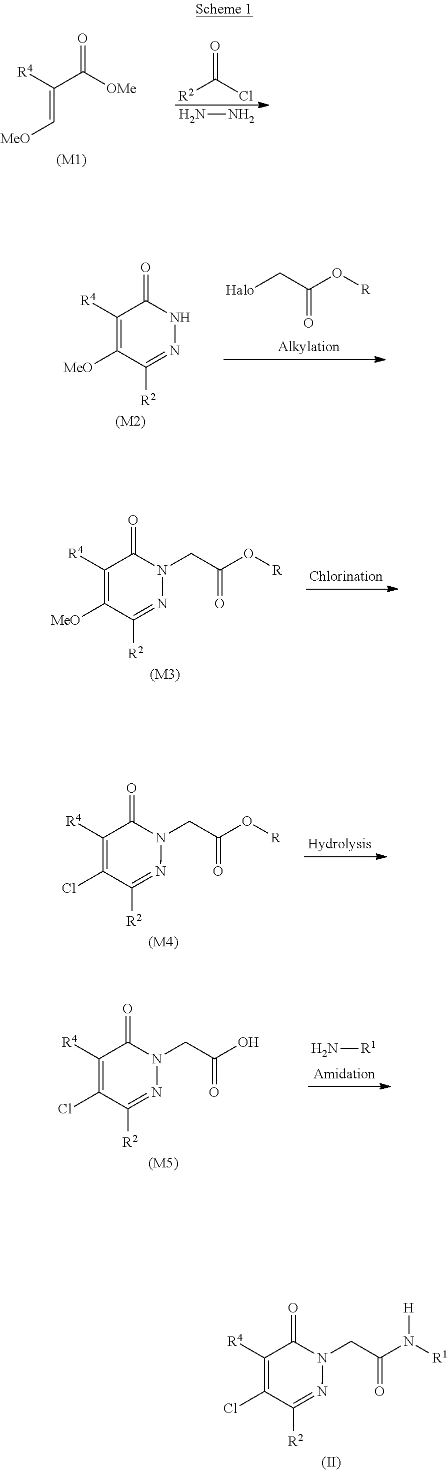

The compound of formula (II), wherein R¹ and R² are as hereinbefore defined, and R⁴ is hydrogen, can be prepared by a reaction sequence shown in Scheme 1 (above), whereby following flow conditions the acrylate ester (M1) is magnesiated by reaction with a strong and non-nucleophilic base, e.g. 2,2,6,6-tetramethylpiperidinylmagnesium chloride lithium chloride, that is quenched with an appropriate acyl chloride, wherein R² is as hereinbefore defined, in the presence of a catalytic amount of copper(I) cyanide and lithium chloride, followed by reaction with hydrazine to give pyridazinone (M2), which is then alkylated with an appropriate alkyl haloacetate, wherein R is C₁₋₄ alkyl, in the presence of a base, e.g. Cs₂CO₃, to provide ester (M3) which is then reacted with a chlorinating reagent, e.g. phosphoryl chloride, to yield intermediate (M4) which is treated under basic conditions, e.g. aqueous LiOH in THF to yield the acid intermediate (M5) followed by amidation with R¹—NH₂ using standard coupling conditions, e.g. HATU and a base, e.g. Hunig's base, to provide a compound of Formula (II).

Alternatively, compounds of the present invention, as described herein, may be prepared by a reaction sequence shown in Scheme 2 (below), whereby an appropriately substituted ester (M3), wherein R⁴ is hydrogen and R is C₁₋₄ alkyl, is treated with an halogenating reagent, e.g. N-bromosuccinimide, to provide a halo-pyridazinone (M6) that is subjected to a Negishi cross-coupling reaction with a zincate, e.g. cyclopropylzinc bromide, using standard conditions, in the presence of a catalyst, e.g. bis(dibenzylideneacetone)palladium and a ligand, e.g. 2-dicyclohexylphosphino-2′,6′-bis(N,N-dimethylamino)biphenyl to give the acid (M7), which is followed by amidation with R¹—NH₂ using standard coupling conditions, e.g. HATU and a base, e.g. Hunig's base, to provide amide (M8).

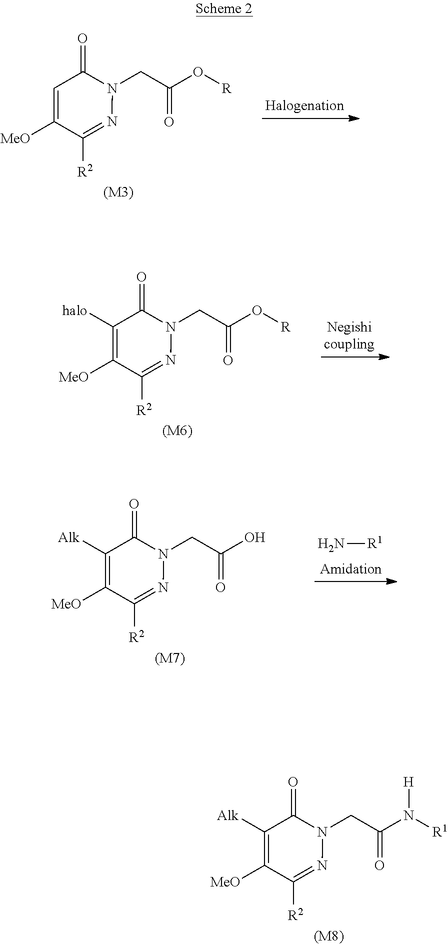

Alternatively, compounds of the present invention, as described herein, may be prepared by a reaction sequence shown in Scheme 3 (below), whereby the dichloropyridazine (M9), is subjected to a Suzuki-type cross-coupling reaction with an appropriate boronate, e.g. cyclopropylboronic acid, using a suitable palladium catalyst, e.g. bis(triphenylphosphine)palladium(II) dichloride and an aqueous base, typically Na₂CO₃, to give alkyl intermediate (M10), which can be treated with acetic acid to yield the pyridazinone (M11), which is then alkylated with an appropriate alkyl haloacetate, wherein R is C₁₋₄ alkyl, in the presence of a base, e.g. Cs₂CO₃, to provide ester (M12) which is then hydrolyzed by reaction of the alkyl ether with a silyl-derivative, e.g. trimethylsilyl iodide, to provide alcohol (M13) that is either reacted with an appropriate substituted alkyl halide R³-halo (Path A), wherein R³ is as hereinbefore defined, using standard conditions in the presence of a base, e.g. Cs₂CO₃, to provide ester M(14), or can be treated with an halogenating reagent (Path B), e.g. N-bromosuccinimide, to provide a halo-pyridazinone (M15) that is then reacted with an appropriate alcohol R³—OH, wherein R³ is as hereinbefore defined, using typical Mitsunobu conditions, e.g. di-tert-butyl azodicarboxylate and triphenylphosphine to give ester M(14).

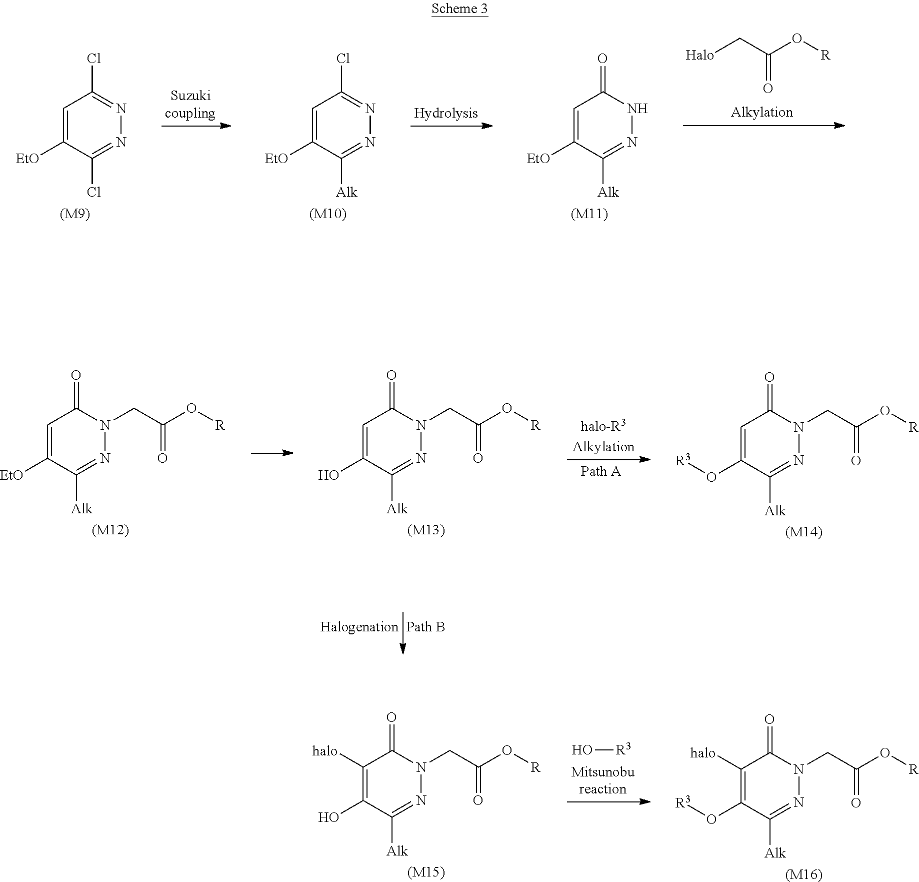

Certain substituents on/in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, etherifications, halogenations, nitrations or couplings.

Compounds of the invention may be isolated from their reaction mixtures using conventional techniques (e.g. recrystallisations, where possible under standard conditions).

It will be appreciated by those skilled in the art that, in the processes described above and hereinafter, the functional groups of intermediate compounds may need to be protected by protecting groups.

The need for such protection will vary depending on the nature of the remote functionality and the conditions of the preparation methods (and the need can be readily determined by one skilled in the art). Suitable amino-protecting groups include acetyl, trifluoroacetyl, t-butoxycarbonyl (BOC), benzyloxycarbonyl (CBz), 9-fluorenyl-methyleneoxycarbonyl (Fmoc) and 2,4,4-trimethylpentan-2-yl (which may be deprotected by reaction in the presence of an acid, e.g. HCl in water/alcohol (e.g. MeOH)) or the like. The need for such protection is readily determined by one skilled in the art. For example the a —C(O)O-tert-butyl ester moiety may serve as a protecting group for a —C(O)OH moiety, and hence the former may be converted to the latter for instance by reaction in the presence of a mild acid (e.g. TFA, or the like).

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P. G. M. Wutz, Wiley-Interscience (1999).

The compounds of the invention as prepared in the hereinabove described processes may be synthesized in the form of racemic mixtures of enantiomers which can be separated from one another following art-known resolution procedures. Those compounds of the invention that are obtained in racemic form may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of the invention involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

### Pharmacology

There is evidence for a role of NLRP3-induced IL-1 and IL-18 in the inflammatory responses occurring in connection with, or as a result of, a multitude of different disorders (Menu et al., Clinical and Experimental Immunology, 2011, 166, 1-15; Strowig et al., Nature, 2012, 481, 278-286). NLRP3 mutations have been found to be responsible for a set of rare autoinflammatory diseases known as CAPS (Ozaki et al., J. Inflammation Research, 2015, 8, 15-27; Schroder et al., Cell, 2010, 140: 821-832; Menu et al., Clinical and Experimental Immunology, 2011, 166, 1-15). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1 beta. NLRP3 has also been implicated in a number of autoinflammatory diseases, including pyogenic arthritis, pyoderma gangrenosum and acne (PAPA), Sweet's syndrome, chronic nonbacterial osteomyelitis (CNO), and acne vulgaris (Cook et al., Eur. J. Immunol., 2010, 40, 595-653).

A number of autoimmune diseases have been shown to involve NLRP3 including, in particular, multiple sclerosis, type-1 diabetes (T1D), psoriasis, rheumatoid arthritis (RA), Behcet's disease, Schnitzler syndrome, macrophage activation syndrome (Braddock et al., Nat. Rev. Drug Disc. 2004, 3, 1-10; Inoue et al., Immunology, 2013, 139, 11-18; Coll et al., Nat. Med. 2015, 21(3), 248-55; Scott et al., Clin. Exp. Rheumatol. 2016, 34(1), 88-93), systemic lupus erythematosus and its complications such as lupus nephritis (Lu et al., J. Immunol., 2017, 198(3), 1119-29), and systemic sclerosis (Artlett et al., Arthritis Rheum. 2011, 63(11), 3563-74). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 2014, 184: 42-54; Kim et al., Am. J. Respir. Crit. Care Med, 2017, 196(3), 283-97). NLRP3 has also been suggested to have a role in a number of central nervous system conditions, including Multiple Sclerosis (MS), Parkinson's disease (PD), Alzheimer's disease (AD), dementia, Huntington's disease, cerebral malaria, brain injury from pneumococcal meningitis (Walsh et al., Nature Reviews, 2014, 15, 84-97; and Dempsey et al., Brain. Behav. Immun. 2017, 61, 306-16), intracranial aneurysms (Zhang et al., J Stroke and Cerebrovascular Dis., 2015, 24, 5, 972-9), and traumatic brain injury (Ismael et al., J Neurotrauma., 2018, 35(11), 1294-1303). NLRP3 activity has also been shown to be involved in various metabolic diseases including type 2 diabetes (T2D) and its organ-specific complications, atherosclerosis, obesity, gout, pseudo-gout, metabolic syndrome (Wen et al., Nature Immunology, 2012, 13, 352-357; Duewell et al., Nature, 2010, 464, 1357-1361; Strowig et al., Nature, 2014, 481, 278-286), and non-alcoholic steatohepatitis (Mridha et al., J. Hepatol. 2017, 66(5), 1037-46). A role for NLRP3 via IL-1 beta has also been suggested in atherosclerosis, myocardial infarction (van Hout et al., Eur. Heart J 2017, 38(11), 828-36), heart failure (Sano et al., J. Am. Coll. Cardiol. 2018, 71(8), 875-66), aortic aneurysm and dissection (Wu et al., Arteriosc/er. Thromb. Vase. Biol., 2017, 37(4), 694-706), and other cardiovascular events (Ridker et al., N. Engl. J. Med., 2017, 377(12), 1119-31).

Other diseases in which NLRP3 has been shown to be involved include: ocular diseases such as both wet and dry age-related macular degeneration (Doyle et al., Nature Medicine, 2012, 18, 791-798; Tarallo et al., Cell 2012, 149(4), 847-59), diabetic retinopathy (Loukovaara et al., Acta Ophthalmol., 2017, 95(8), 803-8), non-infectious uveitis and optic nerve damage (Puyang et al., Sci. Rep. 2016, 6, 20998); liver diseases including non-alcoholic steatohepatitis (NASH) and acute alcoholic hepatitis (Henao-Meija et al., Nature, 2012, 482, 179-185); inflammatory reactions in the lung and skin (Primiano et al., J Immunol. 2016, 197(6), 2421-33) including contact hypersensitivity (such as bullous pemphigoid (Fang et al., J Dermatol Sci. 2016, 83(2), 116-23)), atopic dermatitis (Niebuhr et al., Allergy, 2014, 69(8), 1058-67), Hidradenitis suppurativa (Alikhan et al., J Am. Acad. Dermatol., 2009, 60(4), 539-61), and sarcoidosis (Jager et al., Am. J. Respir. Crit. Care Med., 2015, 191, A5816); inflammatory reactions in the joints (Braddock et al., Nat. Rev. Drug Disc, 2004, 3, 1-10); amyotrophic lateral sclerosis (Gugliandolo et al., Int. J. Mo. Sci., 2018, 19(7), E1992); cystic fibrosis (Iannitti et al., Nat. Commun., 2016, 7, 10791); stroke (Walsh et al., Nature Reviews, 2014, 15, 84-97); chronic kidney disease (Granata et al., PLoS One 2015, 10(3), eoi22272); and inflammatory bowel diseases including ulcerative colitis and Crohn's disease (Braddock et al., Nat. Rev. Drug Disc, 2004, 3, 1-10; Neudecker et a/., J. Exp. Med. 2017, 214(6), 1737-52; Lazaridis et al., Dig. Dis. Sci. 2017, 62(9), 2348-56). The NLRP3 inflammasome has been found to be activated in response to oxidative stress. NLRP3 has also been shown to be involved in inflammatory hyperalgesia (Dolunay et al., Inflammation, 2017, 40, 366-86).

Activation of the NLRP3 inflammasome has been shown to potentiate some pathogenic infections such as influenza and Leishmaniasis (Tate et al., Sci Rep., 2016, 10(6), 27912-20; Novias et al., PLOS Pathogens 2017, 13(2), e1006196).

NLRP3 has also been implicated in the pathogenesis of many cancers (Menu et al., Clinical and Experimental Immunology, 2011, 166, 1-15). For example, several previous studies have suggested a role for IL-1 beta in cancer invasiveness, growth and metastasis, and inhibition of IL-1 beta with canakinumab has been shown to reduce the incidence of lung cancer and total cancer mortality in a randomised, double-blind, placebo-controlled trial (Ridker et al., Lancet., 2017, 390(10105), 1833-42). Inhibition of the NLRP3 inflammasome or IL-1 beta has also been shown to inhibit the proliferation and migration of lung cancer cells in vitro (Wang et al., Onco Rep., 2016, 35(4), 2053-64). A role for the NLRP3 inflammasome has been suggested in myelodysplastic syndromes, myelofibrosis and other myeloproliferative neoplasms, and acute myeloid leukemia (AML) (Basiorka et al., Blood, 2016, 128(25), 2960-75.) and also in the carcinogenesis of various other cancers including glioma (Li et al., Am. J. Cancer Res. 2015, 5(1), 442-9), inflammation-induced tumors (Allen et al., J Exp. Med. 2010, 207(5), 1045-56; Hu et al., PNAS., 2010, 107(50), 21635-40), multiple myeloma (Li et al., Hematology, 2016 21(3), 144-51), and squamous cell carcinoma of the head and neck (Huang et al., J. Exp. Clin. Cancer Res., 2017, 36(1), 116). Activation of the NLRP3 inflammasome has also been shown to mediate chemoresistance of tumor cells to 5-Fluorouracil (Feng et al., J. Exp. Clin. Cancer Res., 2017, 36(1), 81), and activation of NLRP3 inflammasome in peripheral nerve contributes to chemotherapy-induced neuropathic pain (Jia et al., Mol. Pain., 2017, 13, 1-11). NLRP3 has also been shown to be required for the efficient control of viruses, bacteria, and fungi.

The activation of NLRP3 leads to cell pyroptosis and this feature plays an important part in the manifestation of clinical disease (Yan-gang et al., Cell Death and Disease, 2017, 8(2), 2579; Alexander et al., Hepatology, 2014, 59(3), 898-910; Baldwin et al., J. Med. Chem., 2016, 59(5), 1691-1710; Ozaki et al., J. Inflammation Research, 2015, 8, 15-27; Zhen et al., Neuroimmunology Neuroinflammation, 2014, 1(2), 60-65; Mattia et al., J. Med. Chem., 2014, 57(24), 10366-82; Satoh et al., Cell Death and Disease, 2013, 4, 644). Therefore, it is anticipated that inhibitors of NLRP3 will block pyroptosis, as well as the release of pro-inflammatory cytokines (e.g. IL-1 beta) from the cell.

Hence, the compounds of the invention, as described herein (e.g. in any of the embodiments described herein, including by the examples, and/or in any of the forms described herein, e.g. in a salt form or free form, etc) exhibit valuable pharmacological properties, e.g. NLRP3 inhibiting properties on the NLRP3 inflammasome pathway e.g. as indicated in vitro tests as provided herein, and are therefore indicated for therapy or for use as research chemicals, e.g. as tool compounds. Compounds of the invention may be useful in the treatment of an indication selected from: inflammasome-related diseases/disorders, immune diseases, inflammatory diseases, auto-immune diseases, or auto-inflammatory diseases, for example, of diseases, disorders or conditions in which NLRP3 signaling contributes to the pathology, and/or symptoms, and/or progression, and which may be responsive to NLRP3 inhibition and which may be treated or prevented, according to any of the methods/uses described herein, e.g. by use or administration of a compound of the invention, and, hence, in an embodiment, such indications may include:
I. Inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity. Examples of inflammation that may be treated or prevented include inflammatory responses occurring in connection with, or as a result of
   a. a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
   b. a joint condition such as osteoarthritis, systemic juvenile idiopathic arthritis, adult-onset Still's disease, relapsing polychondritis, rheumatoid arthritis, juvenile chronic arthritis, crystal induced arthropathy (e.g. pseudo-gout, gout), or a seronegative spondyloarthropathy (e.g. ankylosing spondylitis, psoriatic arthritis or Reiter's disease);
   c. a muscular condition such as polymyositis or myasthenia gravis;
   d. a gastrointestinal tract condition such as inflammatory bowel disease (including Crohn's disease and ulcerative colitis), gastric ulcer, coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
   e. a respiratory system condition such as chronic obstructive pulmonary disease (COPD), asthma (including bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, adult respiratory distress syndrome, hypersensitivity pneumonitis, or idiopathic interstitial pneumonia;
   f. a vascular condition such as atherosclerosis, Behcet's disease, vasculitides, or Wegener's granulomatosis;
   g. an immune condition, e.g. autoimmune condition, such as systemic lupus erythematosus (SLE), Sjogren's syndrome, systemic sclerosis, Hashimoto's thyroiditis, type I diabetes, idiopathic thrombocytopenia purpura, or Graves disease;
   h. an ocular condition such as uveitis, allergic conjunctivitis, or vernal conjunctivitis;
   i. a nervous condition such as multiple sclerosis or encephalomyelitis;
   j. an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis, Mycobacterium avium intracellulare, Pneumocystis carinii pneumonia, orchitis/epidydimitis, legionella, Lyme disease, influenza A, Epstein Barr virus, viral encephalitis/aseptic meningitis, or pelvic inflammatory disease;
   k. a renal condition such as mesangial proliferative glomerulonephritis, nephrotic syndrome, nephritis, glomerular nephritis, acute renal failure, uremia, or nephritic syndrome;
   l. a lymphatic condition such as Castleman's disease;
   m. a condition of, or involving, the immune system, such as hyper IgE syndrome, lepromatous leprosy, familial hemophagocytic lymphohistiocytosis, or graft versus host disease;
   n. a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH) or primary biliary cirrhosis;
   o. a cancer, including those cancers listed herein below;
   p. a burn, wound, trauma, haemorrhage or stroke;
   q. radiation exposure;
   r. obesity; and/or
   s. pain such as inflammatory hyperalgesia;
II. Inflammatory disease, including inflammation occurring as a result of an inflammatory disorder, e.g. an autoinflammatory disease, such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD);
III. Immune diseases, e.g. auto-immune diseases, such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (T1D), Goodpasture's syndrome, Graves' disease, Guillain-Barre syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjogren's syndrome, systemic sclerosis a systemic connective tissue disorder, Takayasu's arteritis, temporal arteritis, warm autoimmune hemolytic anemia, Wegener's granulomatosis, alopecia universalis, Beliefs disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler syndrome, macrophage activation syndrome, Blau syndrome, giant cell arteritis, vitiligo or vulvodynia;
IV. Cancer including lung cancer, renal cell carcinoma, non-small cell lung carcinoma (NSCLC), Langerhans cell histiocytosis (LCH), myeloproliferative neoplasm (MPN), pancreatic cancer, gastric cancer, myelodysplastic syndrome (MOS), leukaemia including acute lymphocytic leukaemia (ALL) and acute myeloid leukaemia (AML), promyelocytic leukemia (APML, or APL), adrenal cancer, anal cancer, basal and squamous cell skin cancer, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour;
V. Infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr Virus, cytomegalovirus, Varicella-zoster virus, and KSHV), poxviruses (such as vaccinia virus (Modified vaccinia virus Ankara) and Myxoma virus), adenoviruses (such as Adenovirus 5), papillomavirus, or SARS-CoV-2) bacterial infections (e.g. from Staphylococcus aureus, Helicobacter pylori, Bacillus anthracis, Bordetella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes), and prion infections;
VI. Central nervous system diseases such as Parkinson's disease, Alzheimer's disease, dementia, motor neuron disease, Huntington's disease, cerebral malaria, brain injury from pneumococcal meningitis, intracranial aneurysms, traumatic brain injury, multiple sclerosis, and amyotrophic lateral sclerosis;
VII. Metabolic diseases such as type 2 diabetes (T2D), atherosclerosis, obesity, gout, and pseudo-gout;
VIII. Cardiovascular diseases such as hypertension, ischaemia, reperfusion injury including post-M1 ischemic reperfusion injury, stroke including ischemic stroke, transient ischemic attack, myocardial infarction including recurrent myocardial infarction, heart failure including congestive heart failure and heart failure with preserved ejection fraction, embolism, aneurysms including abdominal aortic aneurysm, cardiovascular risk reduction (CvRR), and pericarditis including Dressler's syndrome;
IX. Respiratory diseases including chronic obstructive pulmonary disorder (COPD), asthma such as allergic asthma and steroid-resistant asthma, asbestosis, silicosis, nanoparticle induced inflammation, cystic fibrosis, and idiopathic pulmonary fibrosis;
X. Liver diseases including non-alcoholic fatty liver disease (NAFLD) and nonalcoholic steatohepatitis (NASH) including advanced fibrosis stages F3 and F4, alcoholic fatty liver disease (AFLD), and alcoholic steatohepatitis (ASH);
XI. Renal diseases including acute kidney disease, hyperoxaluria, chronic kidney disease, oxalate nephropathy, nephrocalcinosis, glomerulonephritis, and diabetic nephropathy;
XII. Ocular diseases including those of the ocular epithelium, age-related macular degeneration (AMO) (dry and wet), uveitis, corneal infection, diabetic retinopathy, optic nerve damage, dry eye, and glaucoma;
XIII. Skin diseases including dermatitis such as contact dermatitis and atopic dermatitis, contact hypersensitivity, sunburn, skin lesions, hidradenitis suppurativa (HS), other cyst-causing skin diseases, and acne conglobata;
XIV. Lymphatic conditions such as lymphangitis, and Castleman's disease;
XV. Psychological disorders such as depression, and psychological stress;
XVI. Graft versus host disease;
XVII. Bone diseases including osteoporosis, osteopetrosis;
XVIII. Blood disease including sickle cell disease;
XIX. Allodynia including mechanical allodynia; and
XX. Any disease where an individual has been determined to carry a germline or somatic non-silent mutation in NLRP3.

More specifically the compounds of the invention may be useful in the treatment of an indication selected from: inflammasome-related diseases/disorders, immune diseases, inflammatory diseases, auto-immune diseases, or auto-inflammatory diseases, for example, autoinflammatory fever syndromes (e.g., cryopyrin-associated periodic syndrome), sickle cell disease, systemic lupus erythematosus (SLE), liver related diseases/disorders (e.g. chronic liver disease, viral hepatitis, non-alcoholic steatohepatitis (NASH), alcoholic steatohepatitis, and alcoholic liver disease), inflammatory arthritis related disorders (e.g. gout, pseudogout (chondrocalcinosis), osteoarthritis, rheumatoid arthritis, arthropathy e.g. acute, chronic), kidney related diseases (e.g. hyperoxaluria, lupus nephritis, Type I/Type II diabetes and related complications (e.g. nephropathy, retinopathy), hypertensive nephropathy, hemodialysis related inflammation), neuroinflammation-related diseases (e.g. multiple sclerosis, brain infection, acute injury, neurodegenerative diseases, Alzheimer's disease), cardiovascular/metabolic diseases/disorders (e.g. cardiovascular risk reduction (CvRR), hypertension, atherosclerosis, Type I and Type II diabetes and related complications, peripheral artery disease (PAD), acute heart failure), inflammatory skin diseases (e.g. hidradenitis suppurativa, acne), wound healing and scar formation, asthma, sarcoidosis, age-related macular degeneration, and cancer related diseases/disorders (e.g. colon cancer, lung cancer, myeloproliferative neoplasms, leukemias, myelodysplastic syndromes (MOS), myelofibrosis). In particular, autoinflammatory fever syndromes (e.g. CAPS), sickle cell disease, Type I/Type II diabetes and related complications (e.g. nephropathy, retinopathy), hyperoxaluria, gout, pseudogout (chondrocalcinosis), chronic liver disease, NASH, neuroinflammation-related disorders (e.g. multiple sclerosis, brain infection, acute injury, neurodegenerative diseases, Alzheimer's disease), atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MOS), myelofibrosis).

In particular, compounds of the invention, may be useful in the treatment of a disease or disorder selected from autoinflammatory fever syndromes (e.g. CAPS), sickle cell disease, Type I/Type II diabetes and related complications (e.g. nephropathy, retinopathy), hyperoxaluria, gout, pseudogout (chondrocalcinosis), chronic liver disease, NASH, neuroinflammation-related disorders (e.g. multiple sclerosis, brain infection, acute injury, neurodegenerative diseases, Alzheimer's disease), atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MOS), myelofibrosis). Thus, as a further aspect, the present invention provides the use of a compound of the invention (hence, including a compound as defined by any of the embodiments/forms/examples herein) in therapy. In a further embodiment, the therapy is selected from a disease, which may be treated by inhibition of NLRP3 inflammasome. In another embodiment, the disease is as defined in any of the lists herein. Hence, there is provided any one of the compounds of the invention described herein (including any of the embodiments/forms/examples) for use in the treatment of any of the diseases or disorders described herein (e.g. as described in the aforementioned lists).

### Pharmaceutical Compositions and Combinations

In an embodiment, the invention also relates to a composition comprising a pharmaceutically acceptable carrier and, as active ingredient, a therapeutically effective amount of a compound of the invention. The compounds of the invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations.

In an embodiment, and depending on the mode of administration, the pharmaceutical composition will preferably comprise from 0.05 to 99% by weight, more preferably from 0.1 to 70% by weight, even more preferably from 0.1 to 50% by weight of the active ingredient(s), and, from 1 to 99.95% by weight, more preferably from 30 to 99.9% by weight, even more preferably from 50 to 99.9% by weight of a pharmaceutically acceptable carrier, all percentages being based on the total weight of the composition.

The pharmaceutical composition may additionally contain various other ingredients known in the art, for example, a lubricant, stabilising agent, buffering agent, emulsifying agent, viscosity-regulating agent, surfactant, preservative, flavouring or colorant.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof. The daily dosage of the compound according to the invention will, of course, vary with the compound employed, the mode of administration, the treatment desired and the mycobacterial disease indicated. However, in general, satisfactory results will be obtained when the compound according to the invention is administered at a daily dosage not exceeding 1 gram, e.g. in the range from 10 to 50 mg/kg body weight.

In an embodiment, there is provided a combination comprising a therapeutically effective amount of a compound of the invention, according to any one of the embodiments described herein, and another therapeutic agent (including one or more therapeutic agents). In a further embodiment, there is provided such a combination wherein the other therapeutic agent is selected from (and where there is more than one therapeutic agent, each is independently selected from): farnesoid X receptor (FXR) agonists; anti-steatotics; anti-fibrotics; JAK inhibitors; checkpoint inhibitors including anti-PD1 inhibitors, anti-LAG-3 inhibitors, anti-TIM-3 inhibitors, or anti-POL 1 inhibitors; chemotherapy, radiation therapy and surgical procedures; urate-lowering therapies; anabolics and cartilage regenerative therapy; blockade of IL-17; complement inhibitors; Bruton's tyrosine Kinase inhibitors (BTK inhibitors); Toll Like receptor inhibitors (TLR7/8 inhibitors); CAR-T therapy; anti-hypertensive agents; cholesterol lowering agents; leukotriene A4 hydrolase (LTAH4) inhibitors; SGLT2 inhibitors; 132-agonists; anti-inflammatory agents; nonsteroidal anti-inflammatory drugs (“NSAIDs”); acetylsalicylic acid drugs (ASA) including aspirin; paracetamol; regenerative therapy treatments; cystic fibrosis treatments; or atherosclerotic treatment. In a further embodiment, there is also provided such (a) combination(s) for use as described herein in respect of compounds of the invention, e.g. for use in the treatment of a disease or disorder in which the NLRP3 signaling contributes to the pathology, and/or symptoms, and/or progression, of said disease/disorder, or, a disease or disorder associated with NLRP3 activity (including NLRP3 inflammasome activity), including inhibiting NLRP3 inflammasome activity, and in this respect the specific disease/disorder mentioned herein apply equally here. There may also be provided methods as described herein in respect of compounds of the invention, but wherein the method comprises administering a therapeutically effective amount of such combination (and, in an embodiment, such method may be to treat a disease or disorder mentioned herein in the context of inhibiting NLRP3 inflammasome activity). The combinations mentioned herein may be in a single preparation or they may be formulated in separate preparations so that they can be administered simultaneously, separately or sequentially. Thus, in an embodiment, the present invention also relates to a combination product containing (a) a compound according to the invention, according to any one of the embodiments described herein, and (b) one or more other therapeutic agents (where such therapeutic agents are as described herein), as a combined preparation for simultaneous, separate or sequential use in the treatment of a disease or disorder associated with inhibiting NLRP3 inflammasome activity (and where the disease or disorder may be any one of those described herein), for instance, in an embodiment, the combination may be a kit of parts. Such combinations may be referred to as “pharmaceutical combinations”. The route of administration for a compound of the invention as a component of a combination may be the same or different to the one or more other therapeutic agent(s) with which it is combined. The other therapeutic agent is, for example, a chemical compound, peptide, antibody, antibody fragment or nucleic acid, which is therapeutically active or enhances the therapeutic activity when administered to a patient in combination with a compound of the invention.

The weight ratio of (a) the compound according to the invention and (b) the other therapeutic agent(s) when given as a combination may be determined by the person skilled in the art. Said ratio and the exact dosage and frequency of administration depends on the particular compound according to the invention and the other antibacterial agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present compound of the invention and another antibacterial agent may range from 1/10 to 10/1, more in particular from 1/5 to 5/1, even more in particular from 1/3 to 3/1.

The pharmaceutical composition or combination of the present invention can be in unit dosage of about 1-1000 mg of active ingredient(s) for a subject of about 50-70 kg, or about 1-500 mg, or about 1-250 mg, or about 1-150 mg, or about 1-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

As used herein, term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

The term “subject” as used herein, refers to an animal, preferably a mammal, most preferably a human, for example who is or has been the object of treatment, observation or experiment.

The term “therapeutically effective amount” as used herein, means that amount of compound of the invention (including, where applicable, form, composition, combination comprising such compound of the invention) elicits the biological or medicinal response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by NLRP3, or (ii) associated with NLRP3 activity, or (iii) characterised by activity (normal or abnormal) of NLRP3; or (2) reduce or inhibit the activity of NLRP3; or (3) reduce or inhibit the expression of NLRP3. In another non-limiting embodiment, the term “a therapeutically effective amount” refers to the amount of the compound of the present invention that, when administered to a cell, or a tissue, or a non-cellular biological material, or a medium, is effective to at least partially reduce or inhibit the activity of NLRP3; or at least partially reduce or inhibit the expression of NLRP3.

As used herein, the term “inhibit”, “inhibition” or “inhibiting” refers to the reduction or suppression of a given condition, symptom, or disorder, or disease, or a significant decrease in the baseline activity of a biological activity or process. Specifically, inhibiting NLRP3 or inhibiting NLRP3 inflammasome pathway comprises reducing the ability of NLRP3 or NLRP3 inflammasome pathway to induce the production of IL-1 and/or IL-18. This can be achieved by mechanisms including, but not limited to, inactivating, destabilizing, and/or altering distribution of NLRP3.

As used herein, the term “NLRP3” is meant to include, without limitation, nucleic acids, polynucleotides, oligonucleotides, sense and anti-sense polynucleotide strands, complementary sequences, peptides, polypeptides, proteins, homologous and/or orthologous NLRP molecules, isoforms, precursors, mutants, variants, derivatives, splice variants, alleles, different species, and active fragments thereof.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

“Combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals. The single components may be packaged in a kit or separately. One or both of the components (e.g. powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The term “pharmaceutical combination” as used herein means a product that results from the mixing or combining of more than one therapeutic agent and includes both fixed and non-fixed combinations of the therapeutic agents. The term “pharmaceutical combination” as used herein refers to either a fixed combination in one dosage unit form, or non-fixed combination or a kit of parts for the combined administration where two or more therapeutic agents may be administered independently at the same time or separately within time intervals. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g. tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

### Summary of Pharmacology, Uses, Compositions and Combinations

In an embodiment, there is provided a pharmaceutical composition comprising a therapeutically effective amount of a compound of the invention, according to any one of the embodiments described herein, and a pharmaceutically acceptable carrier (including one or more pharmaceutically acceptable carriers).

In an embodiment, there is provided a compound of the invention, according to any one of the embodiments described herein, for use as a medicament.

In an embodiment, there is provided a compound of the invention, according to any one of the embodiments described herein (and/or pharmaceutical compositions comprising such compound of the invention, according to any one of the embodiment described herein) for use: in the treatment of a disease or disorder associated with NLRP3 activity (including inflammasome activity); in the treatment of a disease or disorder in which the NLRP3 signaling contributes to the pathology, and/or symptoms, and/or progression, of said disease/disorder; in inhibiting NLRP3 inflammasome activity (including in a subject in need thereof); and/or as an NLRP3 inhibitor.

In an embodiment, there is provided a use of compounds of the invention, according to any one of the embodiments described herein (and/or pharmaceutical compositions comprising such compound of the invention, according to any one of the embodiment described herein): in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder; in inhibiting NLRP3 inflammasome activity (including in a subject in need thereof); and/or as an NLRP3 inhibitor.

In an embodiment, there is provided use of compounds of the invention, according to any one of the embodiments described herein (and/or pharmaceutical compositions comprising such compound of the invention, according to any one of the embodiment described herein), in the manufacture of a medicament for: the treatment of a disease or disorder associated with NLRP3 activity (including inflammasome activity); the treatment of a disease or disorder in which the NLRP3 signaling contributes to the pathology, and/or symptoms, and/or progression, of said disease/disorder; and/or inhibiting NLRP3 inflammasome activity (including in a subject in need thereof).

In an embodiment, there is provided a method of treating a disease or disorder in which the NLRP3 signaling contributes to the pathology, and/or symptoms, and/or progression, of said disease/disorder, comprising administering a therapeutically effective amount of a compound of the invention, according to any one of the embodiments described herein (and/or pharmaceutical compositions comprising such compound of the invention, according to any one of the embodiment described herein), for instance to a subject (in need thereof). In a further embodiment, there is provided a method of inhibiting the NLRP3 inflammasome activity in a subject (in need thereof), the method comprising administering to the subject in need thereof a therapeutically effective amount of a compound of the invention, according to any one of the embodiments described herein (and/or pharmaceutical compositions comprising such compound of the invention, according to any one of the embodiment described herein).

In all relevant embodiment of the invention, where a disease or disorder is mentioned (e.g. hereinabove), for instance a disease or disorder in which the NLRP3 signaling contributes to the pathology, and/or symptoms, and/or progression, of said disease/disorder, or, a disease or disorder associated with NLRP3 activity (including NLRP3 inflammasome activity), including inhibiting NLRP3 inflammasome activity, then such disease may include inflammasome-related diseases or disorders, immune diseases, inflammatory diseases, auto-immune diseases, or auto-inflammatory diseases. In a further embodiment, such disease or disorder may include autoinflammatory fever syndromes (e.g. cryopyrin-associated periodic syndrome), liver related diseases/disorders (e.g. chronic liver disease, viral hepatitis, non-alcoholic steatohepatitis (NASH), alcoholic steatohepatitis, and alcoholic liver disease), inflammatory arthritis related disorders (e.g. gout, pseudogout (chondrocalcinosis), osteoarthritis, rheumatoid arthritis, arthropathy e.g. acute, chronic), kidney related diseases (e.g. hyperoxaluria, lupus nephritis, Type I/Type II diabetes and related complications (e.g. nephropathy, retinopathy), hypertensive nephropathy, hemodialysis related inflammation), neuroinflammation-related diseases (e.g. multiple sclerosis, brain infection, acute injury, neurodegenerative diseases, Alzheimer's disease), cardiovascular/metabolic diseases/disorders (e.g. cardiovascular risk reduction (CvRR), hypertension, atherosclerosis, Type I and Type II diabetes and related complications, peripheral artery disease (PAD), acute heart failure), inflammatory skin diseases (e.g. hidradenitis suppurativa, acne), wound healing and scar formation, asthma, sarcoidosis, age-related macular degeneration, and cancer related diseases/disorders (e.g. colon cancer, lung cancer, myeloproliferative neoplasms, leukemia, myelodysplastic syndromes (MOS), myelofibrosis). In a particular aspect, such disease or disorder is selected from autoinflammatory fever syndromes (e.g. CAPS), sickle cell disease, Type I/Type II diabetes and related complications (e.g. nephropathy, retinopathy), hyperoxaluria, gout, pseudogout (chondrocalcinosis), chronic liver disease, NASH, neuroinflammation-related disorders (e.g. multiple sclerosis, brain infection, acute injury, neurodegenerative diseases, Alzheimer's disease), atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MOS), myelofibrosis). In a particular embodiment, the disease or disorder associated with inhibition of NLRP3 inflammasome activity is selected from inflammasome related diseases and disorders, immune diseases, inflammatory diseases, auto-immune diseases, auto-inflammatory fever syndromes, cryopyrin-associated periodic syndrome, chronic liver disease, viral hepatitis, non-alcoholic steatohepatitis, alcoholic steatohepatitis, alcoholic liver disease, inflammatory arthritis related disorders, gout, chondrocalcinosis, osteoarthritis, rheumatoid arthritis, chronic arthropathy, acute arthropathy, kidney related disease, hyperoxaluria, lupus nephritis, Type I and Type II diabetes, nephropathy, retinopathy, hypertensive nephropathy, hemodialysis related inflammation, neuroinflammation-related diseases, multiple sclerosis, brain infection, acute injury, neurodegenerative diseases, Alzheimer's disease, cardiovascular diseases, metabolic diseases, cardiovascular risk reduction, hypertension, atherosclerosis, peripheral artery disease, acute heart failure, inflammatory skin diseases, acne, wound healing and scar formation, asthma, sarcoidosis, age-related macular degeneration, colon cancer, lung cancer, myeloproliferative neoplasms, leukemias, myelodysplastic syndromes and myelofibrosis.

In an embodiment, there is provided a combination comprising a therapeutically effective amount of a compound of the invention, according to any one of the embodiments described herein, and another therapeutic agent (including one or more therapeutic agents). In a further embodiment, there is provided such a combination wherein the other therapeutic agent is selected from (and where there is more than one therapeutic agent, each is independently selected from): farnesoid X receptor (FXR) agonists; anti-steatotics; anti-fibrotics; JAK inhibitors; checkpoint inhibitors including anti-PD1 inhibitors, anti-LAG-3 inhibitors, anti-TIM-3 inhibitors, or anti-POL 1 inhibitors; chemotherapy, radiation therapy and surgical procedures; urate-lowering therapies; anabolics and cartilage regenerative therapy; blockade of IL-17; complement inhibitors; Bruton's tyrosine Kinase inhibitors (BTK inhibitors); Toll Like receptor inhibitors (TLR7/8 inhibitors); CAR-T therapy; anti-hypertensive agents; cholesterol lowering agents; leukotriene A4 hydrolase (LTAH4) inhibitors; SGLT2 inhibitors; 132-agonists; anti-inflammatory agents; nonsteroidal anti-inflammatory drugs (“NSAIDs”); acetylsalicylic acid drugs (ASA) including aspirin; paracetamol; regenerative therapy treatments; cystic fibrosis treatments; or atherosclerotic treatment. In a further embodiment, there is also provided such (a) combination(s) for use as described herein in respect of compounds of the invention, e.g. for use in the treatment of a disease or disorder in which the NLRP3 signaling contributes to the pathology, and/or symptoms, and/or progression, of said disease/disorder, or, a disease or disorder associated with NLRP3 activity (including NLRP3 inflammasome activity), including inhibiting NLRP3 inflammasome activity, and in this respect the specific disease/disorder mentioned herein apply equally here. There may also be provided methods as described herein in respect of compounds of the invention, but wherein the method comprises administering a therapeutically effective amount of such combination (and, in an embodiment, such method may be to treat a disease or disorder mentioned herein in the context of inhibiting NLRP3 inflammasome activity). The combinations mentioned herein may be in a single preparation or they may be formulated in separate preparations so that they can be administered simultaneously, separately or sequentially. Thus, in an embodiment, the present invention also relates to a combination product containing (a) a compound according to the invention, according to any one of the embodiments described herein, and (b) one or more other therapeutic agents (where such therapeutic agents are as described herein), as a combined preparation for simultaneous, separate or sequential use in the treatment of a disease or disorder associated with inhibiting NLRP3 inflammasome activity (and where the disease or disorder may be any one of those described herein).

Compounds of the invention (including forms and compositions/combinations comprising compounds of the invention) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

For instance, compounds of the invention may have the advantage that they have a good or an improved thermodynamic solubility (e.g. compared to compounds known in the prior art; and for instance as determined by a known method and/or a method described herein). Compounds of the invention may have the advantage that they will block pyroptosis, as well as the release of pro-inflammatory cytokines (e.g. IL-1β) from the cell. Compounds of the invention may also have the advantage that they avoid side-effects, for instance as compared to compounds of the prior art, which may be due to selectivity of NLRP3 inhibition. Compounds of the invention may also have the advantage that they have good or improved in vivo pharmacokinetics and oral bioavailability. They may also have the advantage that they have good or improved in vivo efficacy. Specifically, compounds of the invention may also have advantages over prior art compounds when compared in the tests outlined hereinafter (e.g. in Examples C and D).

### General Preparation and Analytical Processes

The compounds according to the invention can generally be prepared by a succession of steps, each of which may be known to the skilled person or described herein.

It is evident that in the foregoing and in the following reactions, the reaction products may be isolated from the reaction medium and, if necessary, further purified according to methodologies generally known in the art, such as extraction, crystallization and chromatography. It is further evident that reaction products that exist in more than one enantiomeric form, may be isolated from their mixture by known techniques, in particular preparative chromatography, such as preparative HPLC, chiral chromatography. Individual diastereoisomers or individual enantiomers can also be obtained by Supercritical Fluid Chromatography (SFC).

The starting materials and the intermediates are compounds that are either commercially available or may be prepared according to conventional reaction procedures generally known in the art.

### Analytical Part

### LC-MS (Liquid Chromatography/Mass Spectrometry)

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.
Table: LCMS Method codes (Flow expressed in mL/min; column temperature (T) in ° C.; Run time in minutes).

| | | | | | Flow | |
|---|---|---|---|---|---|---|
| Method | | | | | Col | Run |
| code | Instrument | column | mobile phase | gradient | T | time |
| Method | Agilent | YMC-pack | A: 0.1% | From 95% A | 2.6 | 6.2 |
| 1 | 1100 | ODS-AQ | HCOOH in | to 5% A in | 35 | |
| | HPLC | C18 (50 × | H₂O | 4.8 min, held | | |
| | DAD | 4.6 mm, 3 | B: CH₃CN | for 1.0 min, | | |
| | LC/MS | μm) | | to 95% A in | | |
| | G1956A | | | 0.2 min. | | |
| Method | Waters: | Waters: | A: 10 mM | From 100% | 0.6 | 3.5 |
| 2 | Acquity ® | BEH | NH₄HCO₃ | A to | 55 | |
| | UPLC ® - | (1.8 μm, | in 95% H₂O + | 5% A in | | |
| | DAD and | 2.1*100 mm) | 5% CH₃CN | 2.10 min, | | |
| | SQD2 | | B: MeOH | to 0% A in | | |
| | | | | 1.4 min | | |
| Method | Waters: | Waters: | A: 10 mM | From 100% | 0.6 | 3.5 |
| 3 | Acquity ® | BEH | CH₃COONH₄ | A to | 55 | |
| | UPLC ® - | (1.7 μm, | in 95% H₂O + | 5% A in | | |
| | DAD and | 2.1*100 mm) | 5% CH₃CN | 2.10 min, | | |
| | SQD | | B: CH₃CN | to 0% A | | |
| | | | | in 0.9 min, | | |
| | | | | to 5% A in | | |
| | | | | 0.5 min | | |
| Method | Waters: | Waters: | A: 10 mM | From 100% | 0.6 | 3.5 |
| 4 | Acquity ® | BEH | NH₄HCO₃ | A to | 55 | |
| | UPLC ® - | (1.7 μm, | in 95% H₂O + | 5% A in | | |
| | DAD and | 2.1*100 mm) | 5% CH₃CN | 2.10 min, | | |
| | SQD | | B: CH₃CN | to 0% A in | | |
| | | | | 0.9 min, to | | |
| | | | | 5% A in 0.5 | | |
| | | | | min | | |
| Method | Waters: | Waters: | A: 95% | From 95% A | 1 | 5 |
| 5 | Acquity ® | BEH C18 | CH₃COONH₄ | to 5% A in | 50 | |
| | IClass | (1.7 μm, | 6.5 mM + 5% | 4.6 min, held | | |
| | UPLC ® - | 2.1 × 50 mm) | CH₃CN, B: | for 0.4 min | | |
| | DAD and | | CH₃CN | | | |
| | Xevo G2-S | | | | | |
| | QTOF | | | | | |
| Method | Waters: | Waters: | A: 95% | From 95% A | 1 | 2 |
| 6 | Acquity ® | BEH C18 | CH₃COONH₄ | to 40% A in | 50 | |
| | IClass | (1.7 μm, | 6.5 mM + 5% | 1.2 min, to | | |
| | UPLC ® - | 2.1 × 50 mm) | CH₃CN, B: | 5% A in | | |
| | DAD and | | CH₃CN | 0.6 min, held | | |
| | Xevo G2-S | | | for 0.2 min | | |
| | QTOF | | | | | |
| Method | Waters: | Waters: | A: 95% | From 95% A | 0.8 | 2.5 |
| 7 | Acquity ® | BEH C18 | CH₃COONH₄ | to 5% A in | 50 | |
| | UPLC ® - | (1.7 μm, | 6.5 mM + 5% | 2.0 min, held | | |
| | DAD and | 2.1 × 50 mm) | CH₃CN, B: | for 0.5 min | | |
| | SQD | | CH₃CN | | | |
| Method | Waters: | Waters: | A: 95% | From 95% A | 1.0 | 5 |
| 8 | Acquity ® | XBridge | CH₃COONH₄ | to 5% A in | 50 | |
| | IClass | C18 (2.5 μm, | 6.5 mM + 5% | 4.6 min, held | | |
| | UPLC ® - | 2.1 × 50 mm) | CH₃CN, B: | for 0.5 min | | |
| | DAD and | | CH₃CN | | | |
| | SQD | | | | | |
| Method | Waters: | Waters: | A: 95% | From 95% A | 0.8 | 5 |
| 9 | Acquity ® | BEH C18 | CH₃COONH₄ | to 5% A in | 50 | |
| | UPLC ® - | (1.7 μm, | 6.5 mM + 5% | 4.5 min, held | | |
| | DAD and | 2.1 × 50 mm) | CH₃CN, B: | for 0.5 min | | |
| | SQD | | CH₃CN | | | |
| Method | Waters: | Waters: | A: 0.1% | From 100% | 0.8 | 2 |
| 10 | Acquity ® | BEH | NH4HCO3 | A to | −55 | |
| | UPLC ® - | (1.7 μm, | in 95% H2O + | 5% A in 1.3 | | |
| | DAD and | 2.1*50 mm) | 5% CH3CN | min, | | |
| | SQD | | B: CH3CN | hold 0.7 min | | |
| Method | Waters: | Waters: | A: 10 mM | From 95% A | 0.8 | 2 |
| 11 | Acquity ® | BEH | CH3COONH4 | to | −55 | |
| | UPLC ® - | (1.7 μm, | in 95% H2O + | 5% A in | | |
| | DAD and | 2.1*50 mm) | 5% CH3CN | 1.3 min, | | |
| | SQD | | B: CH3CN | held for 0.7 | | |
| | | | | min | | |
| Method | Waters: | Waters: | A: 0.1% | From 100% | 0.6 | 3.5 |
| 12 | Acquity ® | BEH | NH4HCO3 | A to | −55 | |
| | UPLC ® - | (1.8 μm, | in 95% H2O + | 5% A in | | |
| | DAD and | 2.1*100 mm) | 5% CH3CN | 2.10 min, | | |
| | SQD | | B: CH3CN | to 0% A in | | |
| | | | | 0.9 min, | | |
| | | | | to 5% A in | | |
| | | | | 0.5 min | | |
| Method | Agilent | Thermo | A: 0.1% | From 90% A | 3 | 3.0 |
| 13 | 1260 | Scientific | HCOOH in | to 10% A in | −30 | |
| | Infinity | Accucore | H2O | 1.5 min, held | | |
| | (Quat. | C18 (50 × | B: CH3CN | for 0.9 min, | | |
| | Pump) | 4.6 mm, 2.6 | | to 95% A in | | |
| | DAD | μm) | | 0.1 min | | |
| | LC/MS | | | | | |
| | G6120 | | | | | |
| | (G1948B) | | | | | |
| Method | Agilent | YMC-pack | A: 0.1% | From 95% A | 2.6 | 6.8 |
| 14 | 1260 | ODS-AQ | HCOOH in | to 5% A in | 35 | |
| | Infinity | C18 (50 × | H2O | 4.8 min, held | | |
| | DAD | 4.6 mm, 3 | B: CH3CN | for 1.0 min, | | |
| | TOF- | μm) | | to 95% A in | | |
| | LC/MS | | | 0.2 min. | | |
| | G6224A | | | | | |
| Method | Waters: | Waters: | A: H2Owith | From 90% A | 1 | 2.5 |
| 15 | Acquity ® | Xbridge | HCO3NH4, | to 0% A in | −25 | |
| | UPLC ® | BEH C18 | 32 mM; B: | 2.0 min, hold | | |
| | H-Class - | (2.1 × | CH3CN | 0.5 min | | |
| | DAD and | 50 mm, | | (equilibration | | |
| | Qda | 2.5 μm) | | step in Pre- | | |
| | (HCLASS- | | | RUN 0.5 | | |
| | PMC) | | | min) | | |
| Method | Agilent: | Waters: | A: HCO3NH4 | From 90% A | 1 | 3 |
| 16 | 1290 | XBridgeC18 | 2.5 g/L (32 | to 0% A in | −25 | |
| | Infinity II - | (2.5 μm, | mM) | 2.0 min, held | | |
| | DAD and | 2.1 × 50 mm) | B: CH3CN | for 0.5 min, | | |
| | MSD/XT | | | to 90% A in | | |
| | | | | 0.2 min, held | | |
| | | | | for 0.3 min | | |
| Method | Waters: | Waters: | A: 95% | From 95% A | 1 | 2.5 |
| 17 | Acquity ® | Xbridge | CH3COONH4 | to 5% A in | −50 | |
| | IClass | BEH C18 | 6.5 mM + 5% | 2.0 min, hold | | |
| | UPLC ® - | (2.1 × | CH3CN, B: | 0.5 min, | | |
| | DAD and | 50 mm, | CH3CN | (equilibration | | |
| | SQD | 2.5 μm) | | step in Pre- | | |
| | (Iclass- | | | RUN 0.5 | | |
| | SQD1) | | | min) | | |
| Method | Waters: | Waters: | A: 95% | From 95% A | 1 | 5 |
| 18 | Acquity ® | BEH C18 | CH3COONH4 | to 5% A in | −50 | |
| | IClass | (1.7 μm, | 6.5 mM + 5% | 4.6 min, held | | |
| | UPLC ® - | 2.1 × 50 mm) | CH3CN, B: | for 0.4 min | | |
| | DAD and | | CH3CN | | | |
| | Xevo G2-S | | | | | |
| | QTOF | | | | | |
| Method | Agilent | YMC-pack | A: 0.1% | From 95% A | 2.6 | 7 |
| 19 | 1290 | ODS-AQ | HCOOH in | to 5% A in 5 | −35 | |
| | Infinity | C18 (50 × | H2O | min, held for | | |
| | DAD | 4.6 mm, 3 | B: CH3CN | 1.0 min, to | | |
| | TOF- | μm) | | 95% A in 0.2 | | |
| | LC/MS | | | min. | | |
| | G6230B | | | | | |
| | ISET | | | | | |
| | emulating | | | | | |
| | G1311C/ | | | | | |
| | G1329B | | | | | |
| Method | Waters: | Waters: | A: 95% | From 95% A | 1.0 | 5 |
| 20 | Acquity ® | XBridge | CH3COONH4 | to 5% A in | 50 | |
| | IClass | C18 (2.5 μm, | 6.5 mM + 5% | 4.6 min, held | | |
| | UPLC ® - | 2.1 × 50 mm) | CH3CN, B: | for 0.5 min | | |
| | DAD and | | CH3CN | | | |
| | SQD | | | | | |

### NMR

For a number of compounds, ¹H NMR spectra were recorded on a Bruker Avance III spectrometer operating at 300 or 400 MHz, on a Bruker Avance 111-HD operating at 400 MHz, on a Bruker Avance NEO spectrometer operating at 400 MHz, on a Bruker Avance Neo spectrometer operating at 500 MHz, or on a Bruker Avance 600 spectrometer operating at 600 MHz, using CHLOROFORM-d (deuterated chloroform, CDCl₃), DMSO-d₆ (deuterated DMSO, dimethyl-d6 sulfoxide), METHANOL-d₄ (deuterated methanol), BENZENE-d₆ (deuterated benzene, C₆D₆) or ACETONE-d₆ (deuterated acetone, (CD₃)₂CO) as solvents. Chemical shifts (δ) are reported in parts per million (ppm) relative to tetramethylsilane (TMS), which was used as internal standard.

### Melting Points

Values are either peak values or melt ranges, and are obtained with experimental uncertainties that are commonly associated with this analytical method.

Method A: For a number of compounds, melting points were determined in open capillary tubes on a Mettler Toledo MP50. Melting points were measured with a temperature gradient of 10° C./minute. Maximum temperature was 300° C. The melting point data was read from a digital display and checked from a video recording system

Method B: For a number of compounds, melting points were determined with a DSC823e (Mettler Toledo) apparatus. Melting points were measured with a temperature gradient of 10° C./minute. Standard maximum temperature was 300° C.

### Experimental Part

Hereinafter, the term “m.p.” means melting point, “aq.” means aqueous, “r.m.” means reaction mixture, “rt” means room temperature, ‘DIPEA’ means N,N-diiso-propylethylamine, “DIPE” means diisopropylether, ‘THF’ means tetrahydrofuran, ‘DMF’ means dimethylformamide, ‘DCM’ means dichloromethane, “EtOH” means ethanol ‘EtOAc’ means ethyl acetate, “AcOH” means acetic acid, “iPrOH” means isopropanol, “iPrNH₂” means isopropylamine, “MeCN” or “ACN” means acetonitrile, “MeOH” means methanol, “Pd(OAc)₂” means palladium(II)diacetate, “rac” means racemic, ‘sat.’ means saturated, ‘SFC’ means supercritical fluid chromatography, ‘SFC-MS’ means supercritical fluid chromatography/mass spectrometry, “LC-MS” means liquid chromatography/mass spectrometry, “GCMS” means gas chromatography/mass spectrometry, “HPLC” means high-performance liquid chromatography, “RP” means reversed phase, “UPLC” means ultra-performance liquid chromatography, “Rₜ” (or “RT”) means retention time (in minutes), “[M+H]⁺” means the protonated mass of the free base of the compound, “DAST” means diethylaminosulfur trifluoride, “DMTMM” means 4-(4,6-dimethoxy-1,3,5-triazin-2-yl)-4-methylmorpholinium chloride, “HATU” means O-(7-azabenzotriazol-1-yl)-N,N,N′,N′-tetramethyluronium hexafluorophosphate (1-[bis(dimethylamino)methylene]-1H-1,2,3-triazolo[4,5-b]pyridinium 3-oxide hexafluorophosphate), “Xantphos” means (9,9-dimethyl-9H-xanthene-4,5-diyl)bis[diphenylphosphine], “TBAT” means tetrabutyl ammonium triphenyldifluorosilicate, “TFA” means trifluoroacetic acid, “Et₂O” means diethylether, “DMSO” means dimethylsulfoxide, “SiO₂” means silica, “XPhos Pd G3” means (2-dicyclohexylphosphino-2′,4′,6′-triisopropyl-1,1′-biphenyl)[2-(2′-amino-1,1′-biphenyl)]palladium(II) ethanesulfonate, “CDCl₃” means deuterated chloroform, “MW” means microwave or molecular weight, “min” means minutes, “h” means hours, “rt” means room temperature, “quant” means quantitative, “n.t.” means not tested, “Cpd” means compound, “POCl₃” means phosphorus(V) oxychloride.

For key intermediates, as well as some final compounds, the absolute configuration of chiral centers (indicated as R and/or S) were established via comparison with samples of known configuration, or the use of analytical techniques suitable for the determination of absolute configuration, such as VCD (vibrational circular dichroism) or X-ray crystallography. When the absolute configuration at a chiral center is unknown, it is arbitrarily designated R*.

### Examples

### Synthesis of 6-isopropyl-5-methoxypyridazin-3(2H)-one 1A

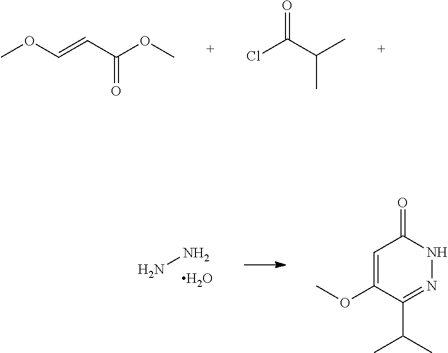

A solution of methyl trans-3-methoxyacrylate [34846-90-7] (20 mL, 1.08 g/mL, 186.02 mmol) in dry THF (245 mL) and 2,2,6,6-Tetramethylpiperidinylmagnesium chloride lithium chloride complex solution [898838-07-8] (265.75 mL, 0.77 M, 204.62 mmol) were pumped through a 10 mL coil at 40° C. (2.5 mL/min each line, 2.5 min residence time). The following out solution collected over a solution of copper cyanide [544-92-3](18.33 g, 204.62 mmol) and lithium chloride [7447-41-8] (17.35 g, 409.25 mmol) in dry THF (200 mL) at 20° C. (water bath). The mixture was stirred for 20 min at RT. A solution of isobutyryl chloride [79-30-1] (23.32 mL, 223.23 mmol) in dry THF (90 mL) was added (drop funnel) at 20° C. and the mixture was stirred for 30 min at RT. Then sat. NaHCO₃ in water (357 mL) and 8% aq. NH₃ (438 mL) were added and the mixture was extracted with Et₂O. The organic layer was separated, dried (MgSO4), filtered and concentrated in vacuo (minimum vacuum: 150 mbar) to afford a solution.

This solution was taken up with EtOH (288 mL) and hydrazinium hydroxide [7803-57-8] (55.64 mL, 744.09 mmol) was added. The reaction mixture was stirred at 120° C. for 1 h. The mixture was concentrated in vacuo and taken up with DCM. Then acidified with 1M HCl (pH=2). The solid was filtered off through a celite pad and the organic layer separated, dried (MgSO₄), filtered and the solvent evaporated. The crude product was purified by flash column chromatography (EtOAc in DCM 0/100 to 100/0). The desired fractions were collected and concentrated. The solid was washed with EtOH and dried to yield 6-isopropyl-5-methoxypyridazin-3(2H)-one 1A (16.37 g, 52%) as a white solid.

¹H NMR (300 MHz, CDCl₃) δ 1.19 (d, J=6.8 Hz, 6H), 3.13 (hept, J=6.8 Hz, 1H), 3.83 (s, 3H), 6.10 (s, 1H), 10.70 (s, 1H).

### Synthesis of ethyl 2-(3-isopropyl-4-methoxy-6-oxopyridazin-1(6H)-yl) acetate 1B

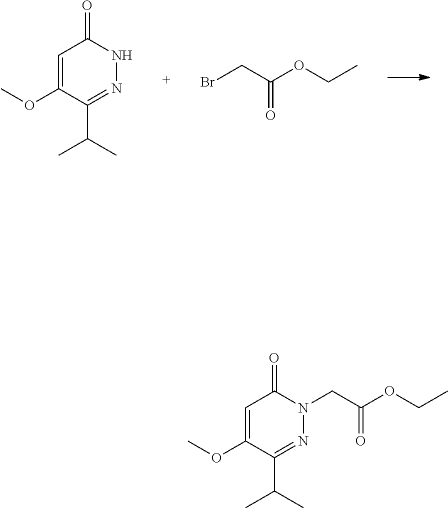

Ethyl bromoacetate [105-36-2] (10.5 mL, 92.80 mmol) was added to a stirred suspension of 6-isopropyl-5-methoxypyridazin-3(2H)-one 1A (14.32 g, 85.14 mmol) and Cs₂CO₃ [534-17-8] (41.61 g, 127.71 mmol) in ACN (116 mL) and DMF (55 mL) at RT. The reaction mixture was stirred in a metallic reactor at 120° C. (preheated oil bath) for 30 min. The crude was filtrated through celite and washed with EtOAc. The filtrate solvents were evaporated and the residue was purified by flash column chromatography (EtOAc in heptane 0/100 to 100/0). The desired fractions were collected and concentrated in vacuo to afford ethyl 2-(3-isopropyl-4-methoxy-6-oxopyridazin-1(6H)-yl) acetate 1B (20.82 g, 95%) as an oil that precipitates as an off-white solid upon standing.

¹H NMR (300 MHz, CDCl₃) δ 1.18 (d, J=6.8 Hz, 6H), 1.28 (t, J=7.1 Hz, 3H), 3.12 (hept, J=6.9 Hz, 1H), 3.82 (s, 3H), 4.23 (q, J=7.1 Hz, 2H), 4.80 (s, 2H), 6.12 (s, 1H) Synthesis of ethyl 2-(4-hydroxy-3-isopropyl-6-oxopyridazin-1(6H)-yl)acetate 2B

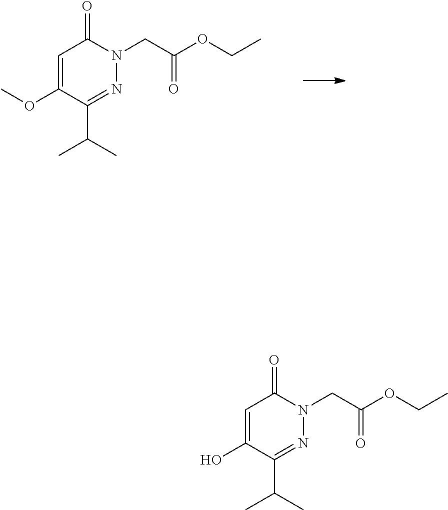 [SMILES: CCOC(=O)Cn1nc(C(C)C)c(OC)cc1=O ; CCOC(=O)Cn1nc(C(C)C)c(O)cc1=O]

Chlorotrimethylsilane [75-77-4] (1.81 mL, 0.86 g/mL, 14.16 mmol) and sodium iodide [7681-82-5] (2.14 g, 14.16 mmol) were added to a stirred solution of ethyl 2-(3-isopropyl-4-methoxy-6-oxopyridazin-1(6H)-yl) acetate 1B (1 g, 3.54 mmol) in acetonitrile anhydrous (20 mL) at rt under nitrogen atmosphere. The mixture was stirred at 130° C. for 20 min under microwave irradiation. The mixture was diluted with sat. aqueous NaHCO3 (32 mL) and 10% aqueous Na2S2O3 (32 mL) and extracted with AcOEt. The organic layer was separated, dried (MgSO4), filtered and the solvents evaporated in vacuo. The crude product was purified by flash column chromatography (silica 25 g; DMM (9:1) in DCM 0/100 to 100/0). The desired fractions were collected and concentrated in vacuo to yield ethyl 2-(4-hydroxy-3-isopropyl-6-oxopyridazin-1(6H)-yl)acetate 2B (600 mg, yield 70%) as a white solid.

### Synthesis of ethyl 2-(5-chloro-4-hydroxy-3-isopropyl-6-oxopyridazin-1(6H)-yl)acetate 3B

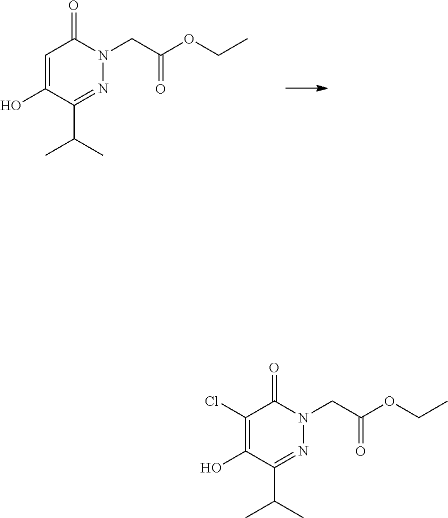 [SMILES: CCOC(=O)Cn1nc(C(C)C)c(O)cc1=O ; CCOC(=O)Cn1nc(C(C)C)c(O)c(Cl)c1=O]

N-Chlorosuccinimide [128-09-6] (6.27 g, 46.99 mmol) was added to a solution of ethyl 2-(4-hydroxy-3-isopropyl-6-oxopyridazin-1(6H)-yl)acetate 2B (5.48 g, 22.81 mmol) in DMF (49 mL) and the mixture was stirred for 16h at rt. The mixture was poured into an ice-cooled 2N HCl solution (10 ml) and extracted with DCM. The organic layer was separated, dried (MgSO4) and evaporated in vacuo. The crude product was purified by flash column chromatography (silica 80 g; AcOEt in heptane 0/100 to 20/80). The desired fractions were collected and concentrated in vacuo to yield ethyl 2-(5-chloro-4-hydroxy-3-isopropyl-6-oxopyridazin-1(6H)-yl)acetate 3B (4.96 g, yield 78%) as a yellow oil.

Additional analogues were synthesized according to the above procedure using the appropriate reagent.

| Reagent | Intermediate | Product |
|---|---|---|
| 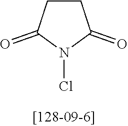 | 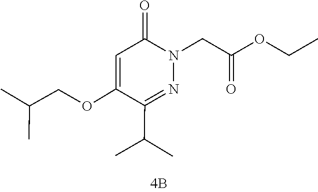 [SMILES: CCOC(=O)Cn1nc(C(C)C)c(OCC(C)C)cc1=O] | 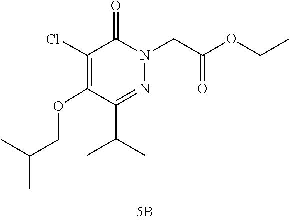 [SMILES: CCOC(=O)Cn1nc(C(C)C)c(OCC(C)C)c(Cl)c1=O] |
| 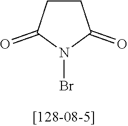 | 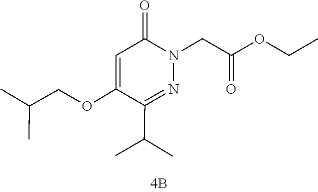 [SMILES: CCOC(=O)Cn1nc(C(C)C)c(OCC(C)C)cc1=O] | 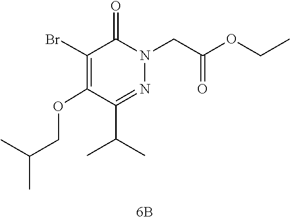 [SMILES: CCOC(=O)Cn1nc(C(C)C)c(OCC(C)C)c(Br)c1=O] |

### Synthesis of ethyl 2-(4-isobutoxy-3-isopropyl-6-oxo-5-(trifluoromethyl)pyridazin-1(6H)-yl)acetate 7B

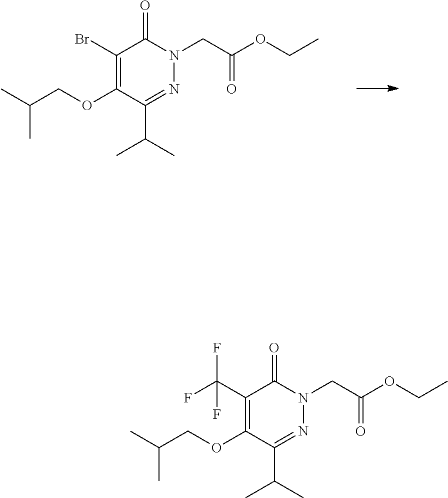

CuI [7681-65-4] (374 mg, 1.96 mmol) was added to a stirred suspension of ethyl 2-(5-bromo-4-isobutoxy-3-isopropyl-6-oxopyridazin-1(6H)-yl)acetate 6B (491 mg, 1.31 mmol) and methyl 2,2-difluoro-2-(fluorosulfonyl)acetate [680-15-9] (250 μl, 1.96 mmol) in N,N-dimethylformamide (6.6 ml). The mixture was stirred at 100° C. for 18h. The crude was filtered through celite. The mixture was diluted with water and extracted with EtOAc. The organic layer was separated and washed with aqueous ammonia, dried (MgSO4), filtered and the solvents evaporated in vacuo. The residue was purified by flash column chromatography (silica 12 g; EtOAc in heptane 0/100 to 20/80). The desired fractions were collected and concentrated in vacuo to yield ethyl 2-(4-isobutoxy-3-isopropyl-6-oxo-5-(trifluoromethyl)pyridazin-1(6H)-yl)acetate 7B (217 mg, yield 45%) as a clear oil.

### Synthesis of ethyl 2-(4-isobutoxy-3-isopropyl-6-oxo-5-vinylpyridazin-1(6H)-yl)acetate 8B

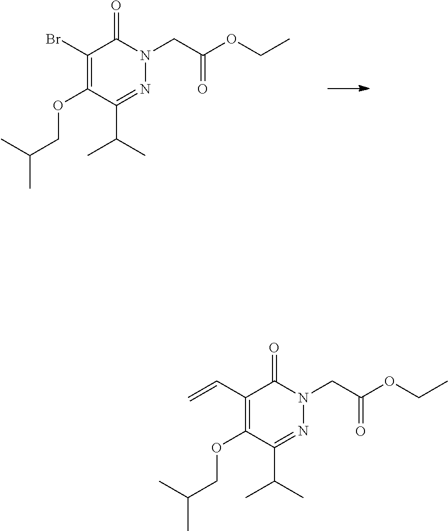

A mixture of ethyl 2-(5-bromo-4-isobutoxy-3-isopropyl-6-oxopyridazin-1(6H)-yl)acetate 6B (1.25 g, 3.33 mmol), vinylboronic acid pinacol ester [75927-49-0] (0.85 mL, 0.91 g/mL, 5 mmol), Pd(PPh3)4 [14221-01-3] (230.95 mg, 0.2 mmol), potassium carbonate [584-08-7] (3.33 mL, 2 M, 6.66 mmol) and DME [110-71-4] (16 mL) was stirred and heated under nitrogen atmosphere for 2 h at 120° C. The mixture was evaporated, taken up in water/saturated bicarbonate solution, extracted with DCM, dried on MgSO4, filtered off and evaporated again. The crude is purified via column chromatography (silica, heptane: EtOAc 100:0 to 70:30) to obtain ethyl 2-(4-isobutoxy-3-isopropyl-6-oxo-5-vinylpyridazin-1(6H)-yl)acetate 8B (640 mg, yield 60%).

### Synthesis of ethyl 2-(5-formyl-4-isobutoxy-3-isopropyl-6-oxopyridazin-1(6H)-yl)acetate 9B

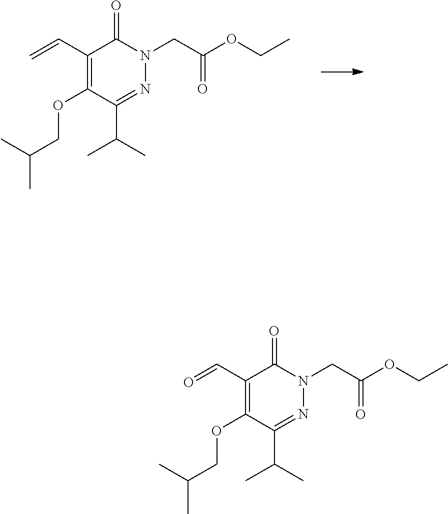

4-Methylmorpholine N-oxide [7529-22-8] (654.04 mg, 5.58 mmol), sodium periodate [7790-28-5] (1592.22 mg, 7.44 mmol) and ethyl 2-(4-isobutoxy-3-isopropyl-6-oxo-5-vinylpyridazin-1(6H)-yl)acetate 8B (600 mg, 1.86 mmol) were placed in a 100 mL RB equipped with a magnetic stir bar. These solids were suspended in 1,4-dioxane (12 mL) and water, distilled (5 mL) and osmium tetroxide [20816-12-0] (756 μL, 1 g/mL, 0.074 mmol) was added. The suspension was stirred vigorously at r.t. for 18h. The mixture was diluted with brine and a saturated solution of NaHCO3 and extracted with DCM. The organic layer is dried over MgSO4 and filtered and the solvent is evaporated under vacuum. The crude is purified via column chromatography (silica, Heptane:EtOAc 100:0 to 70:30). Desired fractions are combined to obtain ethyl 2-(5-formyl-4-isobutoxy-3-isopropyl-6-oxopyridazin-1(6H)-yl)acetate 9B (535 mg, yield 89%) as a yellow oil.

### Synthesis of ethyl 2-(5-(1-ethoxyvinyl)-4-isobutoxy-3-isopropyl-6-oxopyridazin-1(6H)-yl)acetate 11B

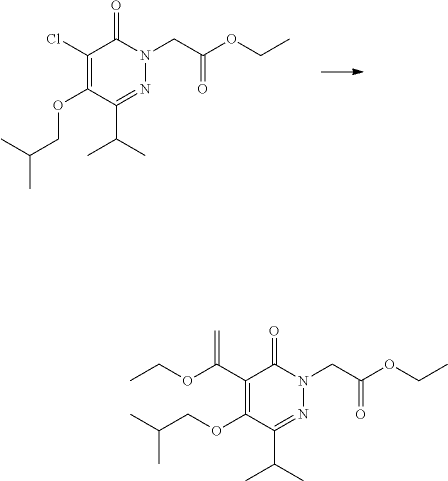

To a mixture of ethyl 2-(5-chloro-4-isobutoxy-3-isopropyl-6-oxopyridazin-1(6H)-yl)acetate 5B (500 mg, 1.51 mmol) in dry THF (18.5 mL), bis(tri-tert-butylphosphine)palladium(0) [53199-31-8] (154.49 mg, 0.3 mmol) was added followed by tributyl(1-ethoxyvinyl)stannane [97674-02-7] (1.02 mL, 1.07 g/mL, 3.02 mmol). The mixture was stirred for overnight at 90° C. Then bis(tri-tert-butylphosphine)palladium(0) [53199-31-8] (154.49 mg, 0.3 mmol) and tributyl(1-ethoxyvinyl)stannane [97674-02-7](0.5 mL, 1.07 g/mL, 1.5 mmol) were added and the reaction mixture is stirred at 90° C. over weekend. The crude was evaporated in vacuo and was purified by column chromatography (Silica; EtOAc in heptane 0/100 to 30/70). The desired fractions were collected and concentrated in vacuo to afford ethyl 2-(5-(1-ethoxyvinyl)-4-isobutoxy-3-isopropyl-6-oxopyridazin-1(6H)-yl)acetate 11B (380 mg, yield 69%) as a yellow oil.

### Synthesis of ethyl 2-(5-acetyl-4-isobutoxy-3-isopropyl-6-oxopyridazin-1(6H)-yl)acetate 12B

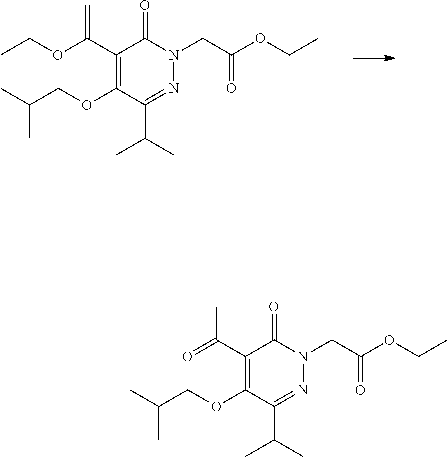

To a mixture of ethyl 2-(5-(1-ethoxyvinyl)-4-isobutoxy-3-isopropyl-6-oxopyridazin-1(6H)-yl)acetate 11B (380 mg, 1.04 mmol) in THF (5 mL), HCl (2M in H2O) [7647-01-0] (1.73 mL, 2 M, 3.46 mmol) was added. The mixture was stirred for 2 h at rt, the crude was extracted twice with DCM, the combined organic layers were evaporated in vacuo to get ethyl 2-(5-acetyl-4-isobutoxy-3-isopropyl-6-oxopyridazin-1(6H)-yl)acetate 12B (329 mg, yield 94%) as a yellow oil.

### Synthesis of methyl 2-(5-(1-hydroxyethyl)-4-isobutoxy-3-isopropyl-6-oxopyridazin-1(6H)-yl)acetate 13B

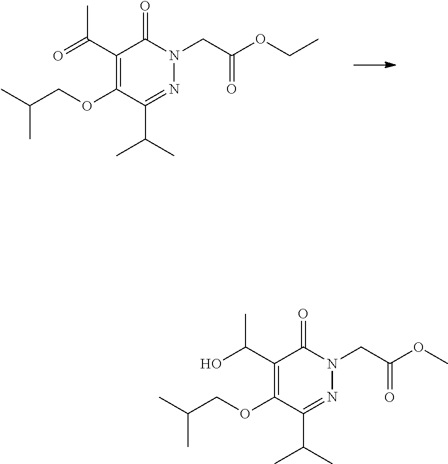

To a mixture of ethyl 2-(5-acetyl-4-isobutoxy-3-isopropyl-6-oxopyridazin-1(6H)-yl)acetate 12B (330 mg, 0.98 mmol) in MeOH (20 mL), NaBH4 [16940-66-2] (45 mg, 1.17 mmol) was added at 0° C. The mixture was stirred for 5 h at rt, then NaBH4 [16940-66-2] (36 mg, 0.98 mmol) was added and the reaction mixture was stirred at rt overnight. The reaction mixture was evaporated in vacuo at 30° C. and treated with saturated solution of NH4Cl and DCM, the mixture was vigorously stirred for 2h, the organic layer (basic pH) was separated, the aqueous layer was extracted with more DCM, the corresponding organic layers were dried and evaporated in vacuo. The crude was purified by column chromatography (silica, heptane:EtOAc 100/0 to 65/35) to obtain methyl 2-(5-(1-hydroxyethyl)-4-isobutoxy-3-isopropyl-6-oxopyridazin-1(6H)-yl)acetate 13B (239 mg, yield 71%) as a transparent oil.

### Synthesis of ethyl 2-(5-chloro-4-(cyclopropylmethoxy)-3-isopropyl-6-oxopyridazin-1(6H)-yl)acetate 14B

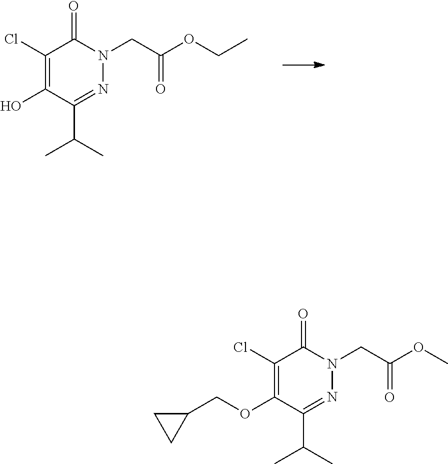 [SMILES: CCOC(=O)Cn1nc(C(C)C)c(O)c(Cl)c1=O ; COC(=O)Cn1nc(C(C)C)c(OCC2CC2)c(Cl)c1=O]

DIPEA [7087-68-5] (0.39 mL, 0.75 g/mL, 2.25 mmol) was added to a stirred solution of ethyl 2-(5-chloro-4-hydroxy-3-isopropyl-6-oxopyridazin-1(6H)-yl)acetate 3B (300 mg, 1.07 mmol) and (bromomethyl)cyclopropane [7051-34-5] (0.21 mL, 1.39 g/mL, 2.14 mmol) in CH3CN (2.1 mL). The mixture was stirred at 150° C. for 15 min under microwave irradiation. Solvents were concentrated in vacuo and purified by flash column chromatography (silica, EtOAc in DCM 0/100 to 100/0). The desired fractions were collected and concentrated in vacuo to yield ethyl 2-(5-chloro-4-(cyclopropylmethoxy)-3-isopropyl-6-oxopyridazin-1(6H)-yl)acetate 14B (287 mg, yield 82%) as a dark brown oil.

Additional analogues were synthesized according to the above conditions, using the appropriate reagents.

| Reagent | Intermediate | Product. |
|---|---|---|
| 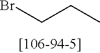 | 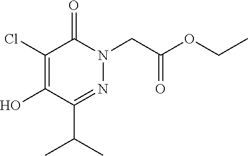 [SMILES: CCOC(=O)Cn1nc(C(C)C)c(O)c(Cl)c1=O] | 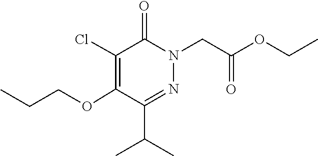 [SMILES: CCCOc1c(C(C)C)nn(CC(=O)OCC)c(=O)c1Cl] |
| 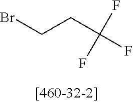 | 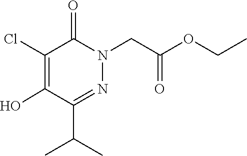 [SMILES: CCOC(=O)Cn1nc(C(C)C)c(O)c(Cl)c1=O] | 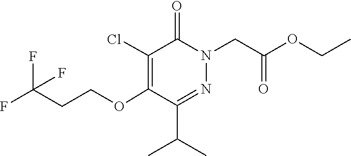 [SMILES: CCOC(=O)Cn1nc(C(C)C)c(OCCC(F)(F)F)c(Cl)c1=O] |
| 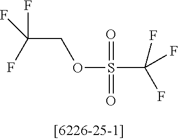 [SMILES: O=S(=O)(OCC(F)(F)F)C(F)(F)F] | 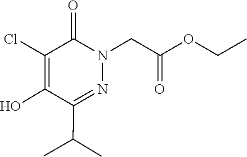 [SMILES: CCOC(=O)Cn1nc(C(C)C)c(O)c(Cl)c1=O] | 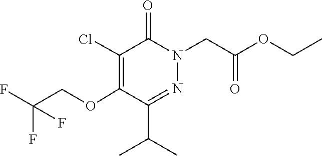 [SMILES: CCOC(=O)Cn1nc(C(C)C)c(OCC(F)(F)F)c(Cl)c1=O] |
| 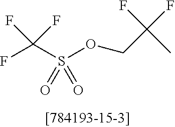 | 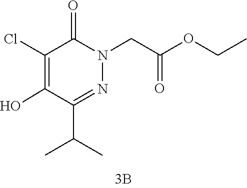 [SMILES: CCOC(=O)Cn1nc(C(C)C)c(O)c(Cl)c1=O] | 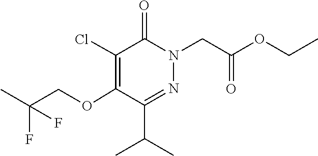 [SMILES: CCOC(=O)Cn1nc(C(C)C)c(OCC(C)(F)F)c(Cl)c1=O] |
| 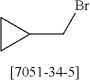 | 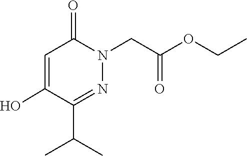 [SMILES: CCOC(=O)Cn1nc(C(C)C)c(O)cc1=O] | 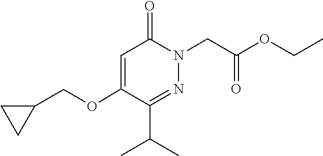 [SMILES: CCOC(=O)Cn1nc(C(C)C)c(OCC2CC2)cc1=O] |
| | 2B | |

### Synthesis of ethyl 2-(4-(benzyloxy)-3-isopropyl-6-oxopyridazin-1(6H)-yl)acetate

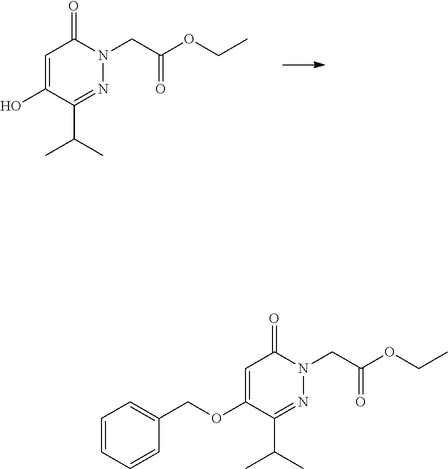 [SMILES: CCOC(=O)Cn1nc(C(C)C)c(O)cc1=O ; CCOC(=O)Cn1nc(C(C)C)c(OCc2ccccc2)cc1=O]

Benzyl bromide [100-39-0] (2.01 mL, 1.44 g/mL, 16.92 mmol) and Cs2CO3 [534-17-8](6.78 g, 20.81 mmol) were added to a stirred solution of ethyl 2-(4-hydroxy-3-isopropyl-6-oxopyridazin-1(6H)-yl)acetate 2B (2.03 g, 8.46 mmol) in DMF (34 mL) at rt under nitrogen atmosphere. The reaction mixture was stirred in a sealed tube at 120° C. (preheated oil bath) for 20 min. The mixture was diluted With sat. aqueous NaHCO3 and extracted with AcOEt. The organic layer was separated, dried (MgSO4), filtered and the solvents evaporated in vacuo. The crude product was purified by flash column chromatography (silica 25 g; DMM (9:1) in DCM 0/100 to 100/0). The desired fractions were collected and concentrated in vacuo to yield ethyl 2-(4-(benzyloxy)-3-isopropyl-6-oxopyridazin-1(6H)-yl)acetate (2.37 g, 84% yield) as a white solid.

Additional analogues were synthesized according to the above procedure using the appropriate reagent.

| Reagent | Intermediate | Product |
|---|---|---|
| 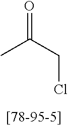 | 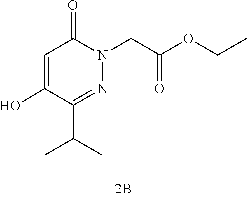 [SMILES: CCOC(=O)Cn1nc(C(C)C)c(O)cc1=O] | 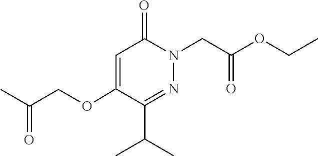 [SMILES: CCOC(=O)Cn1nc(C(C)C)c(OCC(C)=O)cc1=O] |
| 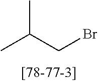 | 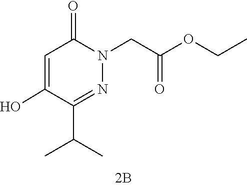 [SMILES: CCOC(=O)Cn1nc(C(C)C)c(O)cc1=O] | 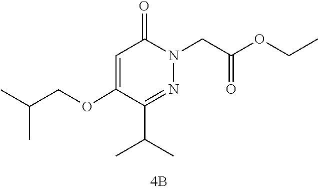 [SMILES: CCOC(=O)Cn1nc(C(C)C)c(OCC(C)C)cc1=O] |
| 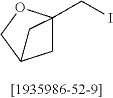 | 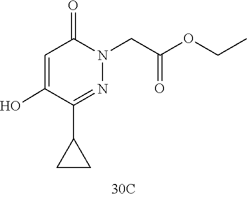 [SMILES: CCOC(=O)Cn1nc(C2CC2)c(O)cc1=O] | 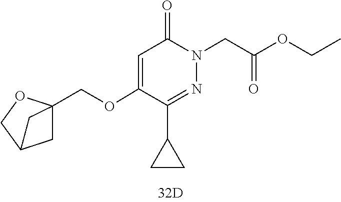 [SMILES: CCOC(=O)Cn1nc(C2CC2)c(OCC23CC(CO2)C3)cc1=O] |

### Synthesis of ethyl 2-(5-fluoro-4-isobutoxy-3-isopropyl-6-oxopyridazin-1(6H)-yl)acetate

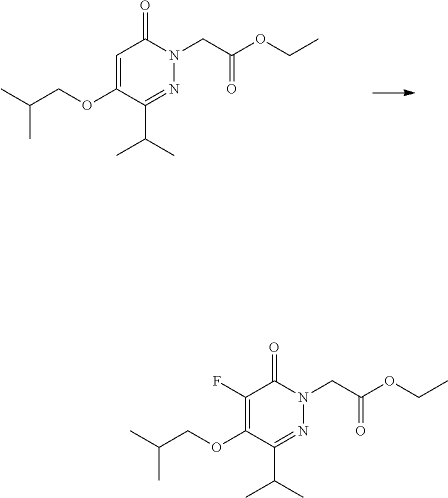

A 20-mL MW vial was charged with ethyl 2-(4-isobutoxy-3-isopropyl-6-oxopyridazin-1(6H)-yl)acetate 4B (400 mg, 1.34 mmol) and 1-chloromethyl-4-fluoro-1,4-diazoniabicyclo[2.2.2]octane bis(tetrafluoroborate) [140681-55-6] (521 mg, 1.47 mmol). The vial was sealed and ACN (11.5 mL) was added. The vial was placed in the microwave and heated at 70° C. for 45 minutes and then ah and 10 minutes at 100° C. The crude mixture was concentrated in vacuo and the obtained residue suspended in DCM (10 mL) and filtered. The filtrate was evaporated under vacuum to obtain a crude (460 mg) which was purified by column chromatography (silica, heptane: EtOAc 100:0 to 65:35) to obtain ethyl 2-(5-fluoro-4-isobutoxy-3-isopropyl-6-oxopyridazin-1(6H)-yl)acetate (144 mg, yield 33%) as a transparent oil.

### Synthesis of 3,3,3-trifluoropropyl 4-methylbenzenesulfonate

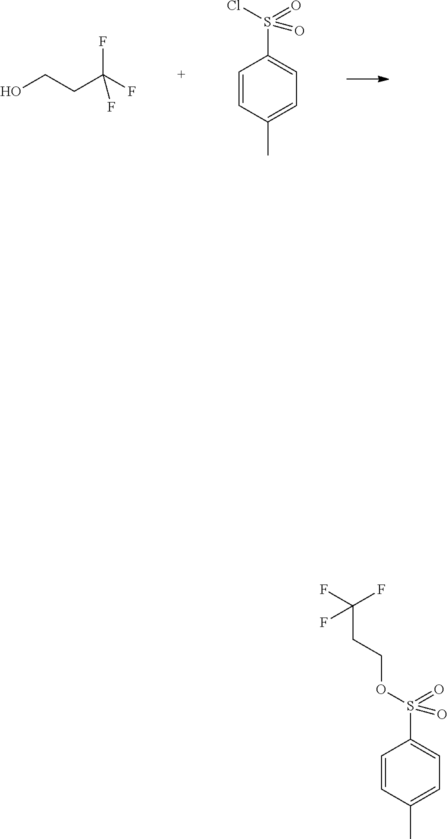 [SMILES: OCCC(F)(F)F ; Cc1ccc(S(=O)(=O)Cl)cc1 ; Cc1ccc(S(=O)(=O)OCCC(F)(F)F)cc1]

Triethylamine [121-44-8] (1.22 mL, 0.73 g/mL, 8.77 mmol) was added to a solution of 3,3,3-trifluoropropan-1-ol [2240-88-2] (500 mg, 4.38 mmol) in DCM (15 mL). Then 4-toluenesulphonyl chloride [98-59-9] (869 mg, 4.56 mmol) was added in portions with stirring under ice-cooling at 5° C. The reaction mixture was stirred at RT overnight. The mixture was diluted with water and extracted with DCM (3×). The combined organic layers were dried (MgSO4), filtered and the solvents evaporated in vacuo to yield 3,3,3-trifluoropropyl 4-methylbenzenesulfonate (898 mg, 74% yield) as a white solid.

Additional analogues were synthesized according to the above procedure using the appropriate reagent.

| Reagent | Product |
|---|---|
| 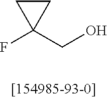 | 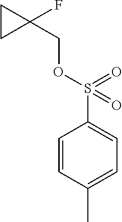 [SMILES: Cc1ccc(S(=O)(=O)OCC2(F)CC2)cc1] |

### Synthesis of ethyl 2-(3-isopropyl-6-oxo-4-(3,3,3-trifluoropropoxy)pyridazin-1(6H)-yl)acetate

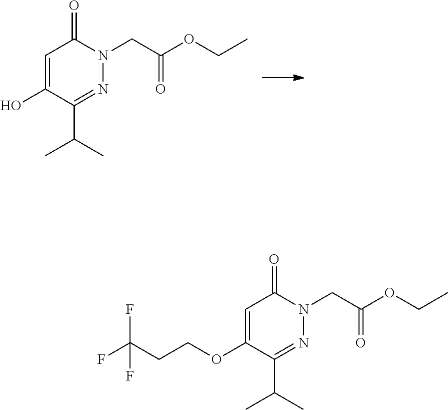 [SMILES: CCOC(=O)Cn1nc(C(C)C)c(O)cc1=O ; CCOC(=O)Cn1nc(C(C)C)c(OCCC(F)(F)F)cc1=O]

3,3,3-Trifluoropropyl 4-methylbenzenesulfonate (124 mg, 0.45 mmol) was added to a stirred suspension of ethyl 2-(4-hydroxy-3-isopropyl-6-oxopyridazin-1(6H)-yl)acetate 2B (100 mg, 0.42 mmol) and cesium carbonate [534-17-8] (203 mg, 0.62 mmol) in acetonitrile (567 μL) and N,N-dimethylformamide (267 μL), at rt. The reaction mixture was stirred in a metallic reactor at 120° C. (preheated oil bath) for 30 min. The crude was filtrated through celite and washed with EtOAc. The filtrate solvents were evaporated and the residue was purified by flash column chromatography (silica 12 g; EtOAc in heptane 0/100 to 100/0). The desired fractions were collected and concentrated in vacuo to yield ethyl 2-(3-isopropyl-6-oxo-4-(3,3,3-trifluoropropoxy)pyridazin-1(6H)-yl)acetate (48 mg, 34% yield) as a yellow oil.

Additional analogues were synthesized according to the above procedure using the appropriate reagent.

| Reagent | Product |
|---|---|
| 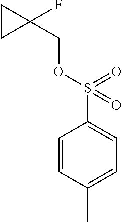 [SMILES: Cc1ccc(S(=O)(=O)OCC2(F)CC2)cc1] | 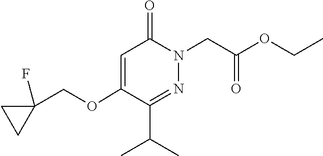 [SMILES: CCOC(=O)Cn1nc(C(C)C)c(OCC2(F)CC2)cc1=O] |

### Synthesis of ethyl 2-(4-(2,2-difluoropropoxy)-3-isopropyl-6-oxopyridazin-1(6H)-yl)acetate 10B

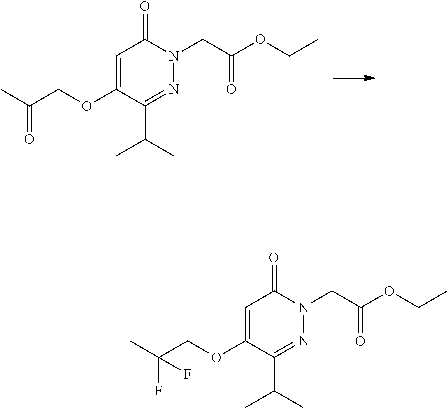

DAST [38078-09-0] (173 μL, 1.22 g/L, 1.31 mmol) was added dropwise to a solution of ethyl 2-(3-isopropyl-6-oxo-4-(2-oxopropoxy)pyridazin-1(6H)-yl)acetate (129 mg, 0.44 mmol) in DCM dry (0.5 mL) at 0° C. under nitrogen. The reaction mixture was stirred at rt for 16 h. The mixture was diluted with NaHCO3 aq. sat. and extracted with DCM. The combined organic layers were washed with brine and dried over MgSO4, filtered and concentrated. The crude product was purified by flash column chromatography (silica 12 g; AcOEt in heptane 0/100 to 40/60). The desired fractions were collected and concentrated to yield ethyl 2-(4-(2,2-difluoropropoxy)-3-isopropyl-6-oxopyridazin-1(6H)-yl)acetate 10B (62 mg, 44% yield) as a white solid.

Additional analogues were synthesized according to the above procedure using the appropriate reagent.

| Intermediate | Product |
|---|---|
| 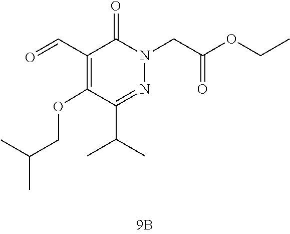 [SMILES: CCOC(=O)Cn1nc(C(C)C)c(OCC(C)C)c(C=O)c1=O] | 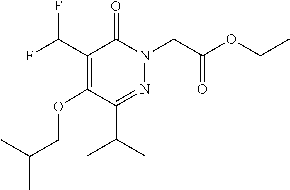 [SMILES: CCOC(=O)Cn1nc(C(C)C)c(OCC(C)C)c(C(F)F)c1=O] |

### Synthesis of ethyl 2-(4-chloro-3-isopropyl-6-oxopyridazin-1(6H)-yl) acetate 1C

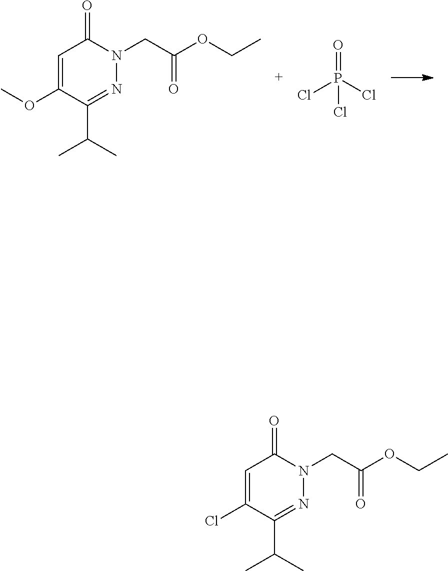 [SMILES: CCOC(=O)Cn1nc(C(C)C)c(OC)cc1=O ; O=P(Cl)(Cl)Cl ; CCOC(=O)Cn1nc(C(C)C)c(Cl)cc1=O]

Ethyl 2-(3-isopropyl-4-methoxy-6-oxopyridazin-1(6H)-yl) acetate 1B (19.2 g, 75.51 mmol) was put into several sealed vials (12×1600 mg) and purged and filled with nitrogen three times. Dry ACN (168 mL, 12×14 mL) was added and the solid was dissolved. Phosphoryl chloride (14.04 mL, 12×1.17 mL, 151.01 mmol) was added and the mixture was heated at 160° C. for 20 min under microwave irradiation. All the different reactions were combined and the excess of phosphoryl chloride was quenched with ice-water and the mixture was extracted with EtOAc. The organic layers were separated, combined, dried (MgSO₄), filtered and the solvents evaporated in vacuo. The residue was purified by flash column chromatography (EtOAc in heptane 0/100 to 10/90). The desired fractions were collected and concentrated in vacuo to afford the ethyl 2-(4-chloro-3-isopropyl-6-oxopyridazin-1(6H)-yl) acetate 1C (14.95 g, 76%) as a clear yellow oil.

¹H NMR (400 MHz, CDCl₃) δ 1.23 (d, J=6.8 Hz, 6H), 1.28 (t, J=7.1 Hz, 3H), 3.25 (hept, J=6.7 Hz, 1H), 4.24 (q, J=7.1 Hz, 2H), 4.83 (s, 2H), 7.01 (s, 1H).

### Synthesis of N-([1,2,4] triazolo[4,3-a] pyridin-6-yl)-2-(4-chloro-3-isopropyl-6-oxopyridazin-1(6H)-yl) acetamide 1E

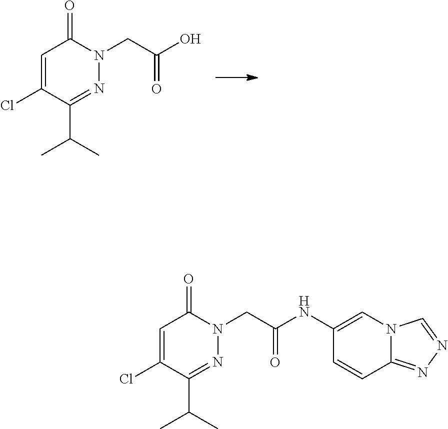 [SMILES: CC(C)c1nn(CC(=O)O)c(=O)cc1Cl ; CC(C)c1nn(CC(=O)Nc2ccc3nncn3c2)c(=O)cc1Cl]

DIPEA [7087-68-5] (11.2 mL, 64.99 mmol) was added to a stirred solution of 2-(4-chloro-3-isopropyl-6-oxopyridazin-1(6H)-yl) acetic acid 1D (2.8 g, 12.14 mmol), [1,2,4]triazolo[4,3-A]pyridin-7-amine [1082448-58-5] (1.79 g, 13.35 mmol) and HATU [148893-10-1] (5.15 g, 13.55 mmol) in DMF (56 mL). The mixture was stirred at RT for 2.5 h. The mixture was diluted with sat. NaHCO₃ in water and extracted with EtOAc (100 mL×4) and then with a mixture EtOAc/THF (7/3, 70 mL×2). The combined organic layers were dried (Na₂SO₄), filtered and the solvents evaporated in vacuo to yield a beige solid.

This solid was triturated with ACN, filtered and washed with additional ACN to yield N-([1,2,4] triazolo[4,3-a] pyridin-6-yl)-2-(4-chloro-3-isopropyl-6-oxopyridazin-1(6H)-yl) acetamide 1E (3.48 g, yield 83%) as an off white solid.

The filtrate was evaporated in vacuo and purified by flash column chromatography (silica; MeOH in DCM 0/100 to 10/90). The desired fractions were collected and concentrated in vacuo to yield additional N-([1,2,4] triazolo[4,3-a] pyridin-6-yl)-2-(4-chloro-3-isopropyl-6-oxopyridazin-1(6H)-yl) acetamide 1E (334 mg, yield 8%) as a beige solid.

LCMS (Rₜ: 0.78, Area %: 100, MW: 346.09, BPM1: 347.10, Method 6)

¹H NMR (400 MHz, DMSO-d₆) δ ppm 1.20 (d, J=6.94 Hz, 6H) 3.18-3.29 (m, 1H) 4.92 (s, 2H) 7.29 (dd, J=9.71, 1.85 Hz, 1H) 7.34 (s, 1H) 7.79 (d, J=9.71 Hz, 1H) 9.20 (dd, J=1.62, 0.92 Hz, 1H) 9.23 (d, J=0.69 Hz, 1H) 10.39-10.83 (m, 1H)

Additional analogues were synthesized according to the above procedure, using the appropriate reagents.

| Reagent | Intermediate | Product |
|---|---|---|
| 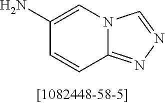 | 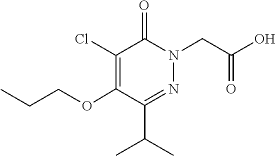 [SMILES: CCCOc1c(C(C)C)nn(CC(=O)O)c(=O)c1Cl] | 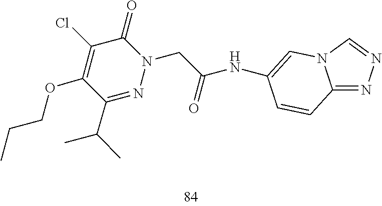 [SMILES: CCCOc1c(C(C)C)nn(CC(=O)Nc2ccc3nncn3c2)c(=O)c1Cl] |
| 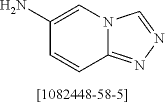 | 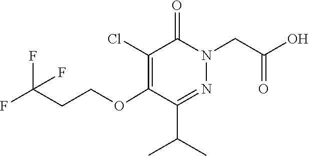 [SMILES: CC(C)c1nn(CC(=O)O)c(=O)c(Cl)c1OCCC(F)(F)F] | 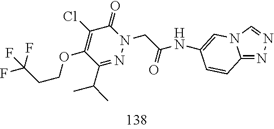 [SMILES: CC(C)c1nn(CC(=O)Nc2ccc3nncn3c2)c(=O)c(Cl)c1OCCC(F)(F)F] |
| 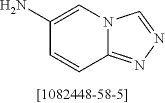 | 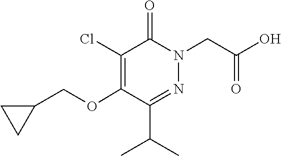 [SMILES: CC(C)c1nn(CC(=O)O)c(=O)c(Cl)c1OCC1CC1] | 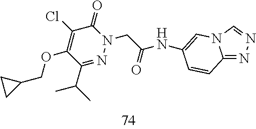 [SMILES: CC(C)c1nn(CC(=O)Nc2ccc3nncn3c2)c(=O)c(Cl)c1OCC1CC1] |
| 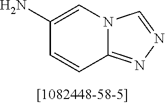 | 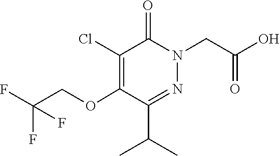 [SMILES: CC(C)c1nn(CC(=O)O)c(=O)c(Cl)c1OCC(F)(F)F] | 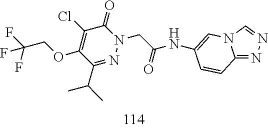 [SMILES: CC(C)c1nn(CC(=O)Nc2ccc3nncn3c2)c(=O)c(Cl)c1OCC(F)(F)F] |
| 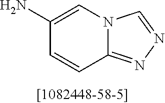 | 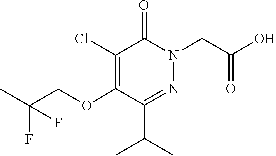 [SMILES: CC(C)c1nn(CC(=O)O)c(=O)c(Cl)c1OCC(C)(F)F] | 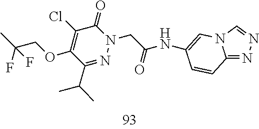 [SMILES: CC(C)c1nn(CC(=O)Nc2ccc3nncn3c2)c(=O)c(Cl)c1OCC(C)(F)F] |
| 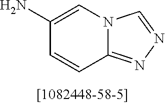 | 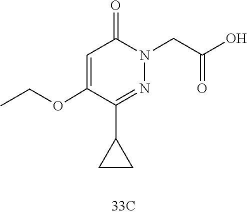 [SMILES: CCOc1cc(=O)n(CC(=O)O)nc1C1CC1] | 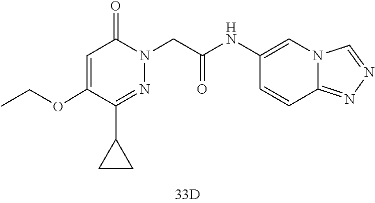 [SMILES: CCOc1cc(=O)n(CC(=O)Nc2ccc3nncn3c2)nc1C1CC1] |
| 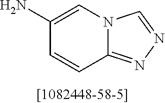 | 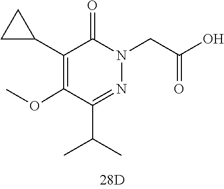 [SMILES: COc1c(C(C)C)nn(CC(=O)O)c(=O)c1C1CC1] | 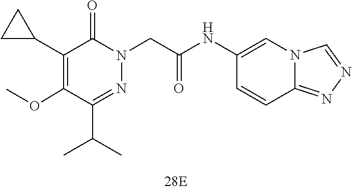 [SMILES: COc1c(C(C)C)nn(CC(=O)Nc2ccc3nncn3c2)c(=O)c1C1CC1] |
| 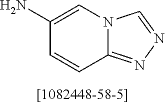 | 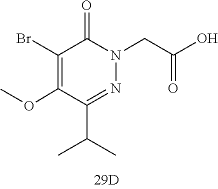 [SMILES: COc1c(C(C)C)nn(CC(=O)O)c(=O)c1Br] | 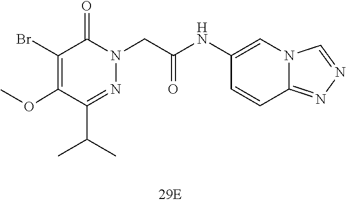 [SMILES: COc1c(C(C)C)nn(CC(=O)Nc2ccc3nncn3c2)c(=O)c1Br] |
| 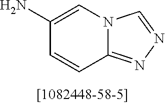 | 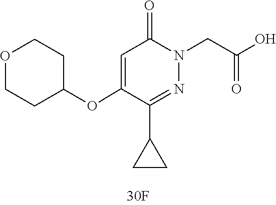 [SMILES: O=C(O)Cn1nc(C2CC2)c(OC2CCOCC2)cc1=O] | 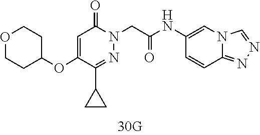 [SMILES: O=C(Cn1nc(C2CC2)c(OC2CCOCC2)cc1=O)Nc1ccc2nncn2c1] |
| 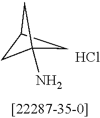 | 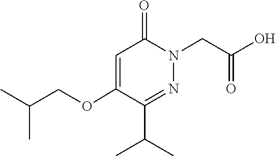 [SMILES: CC(C)COc1cc(=O)n(CC(=O)O)nc1C(C)C] | 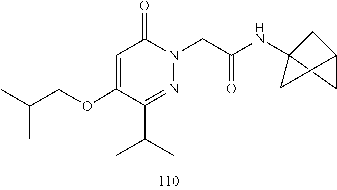 [SMILES: CC(C)COc1cc(=O)n(CC(=O)NC23CC(C2)C3)nc1C(C)C] |
| 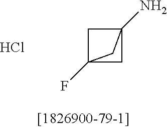 | 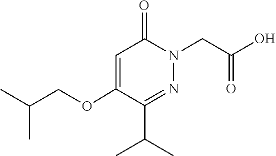 [SMILES: CC(C)COc1cc(=O)n(CC(=O)O)nc1C(C)C] | 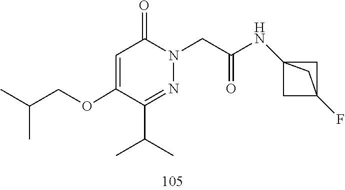 [SMILES: CC(C)COc1cc(=O)n(CC(=O)NC23CC(F)(C2)C3)nc1C(C)C] |
| 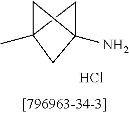 | 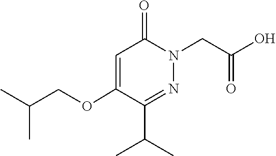 [SMILES: CC(C)COc1cc(=O)n(CC(=O)O)nc1C(C)C] | 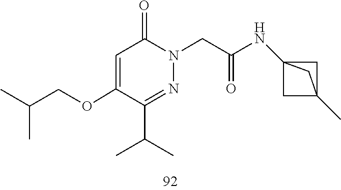 [SMILES: CC(C)COc1cc(=O)n(CC(=O)NC23CC(C)(C2)C3)nc1C(C)C] |
| 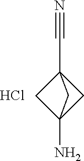 | 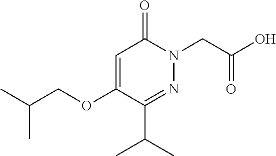 [SMILES: CC(C)COc1cc(=O)n(CC(=O)O)nc1C(C)C] | 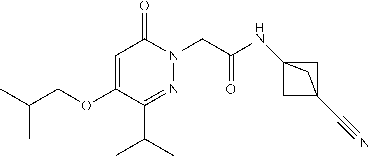 [SMILES: CC(C)COc1cc(=O)n(CC(=O)NC23CC(C#N)(C2)C3)nc1C(C)C] |
| [2170371-90-9] | | 66 |
| 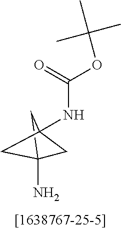 | 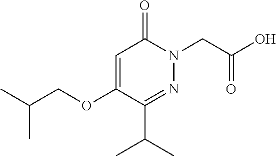 [SMILES: CC(C)COc1cc(=O)n(CC(=O)O)nc1C(C)C] | 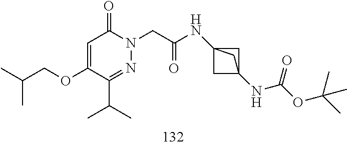 [SMILES: CC(C)COc1cc(=O)n(CC(=O)NC23CC(NC(=O)OC(C)(C)C)(C2)C3)nc1C(C)C] |
| 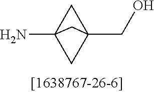 | 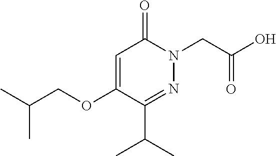 [SMILES: CC(C)COc1cc(=O)n(CC(=O)O)nc1C(C)C] | 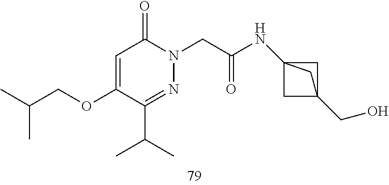 [SMILES: CC(C)COc1cc(=O)n(CC(=O)NC23CC(CO)(C2)C3)nc1C(C)C] |
| 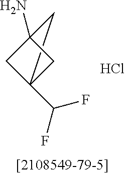 | 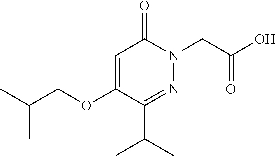 [SMILES: CC(C)COc1cc(=O)n(CC(=O)O)nc1C(C)C] | 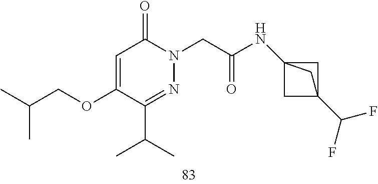 [SMILES: CC(C)COc1cc(=O)n(CC(=O)NC23CC(C(F)F)(C2)C3)nc1C(C)C] |
| 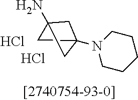 [SMILES: Cl ; Cl ; NC12CC(N3CCCCC3)(C1)C2] | 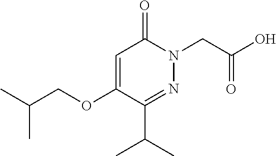 [SMILES: CC(C)COc1cc(=O)n(CC(=O)O)nc1C(C)C] | 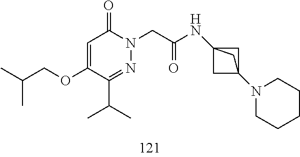 [SMILES: CC(C)COc1cc(=O)n(CC(=O)NC23CC(N4CCCCC4)(C2)C3)nc1C(C)C] |
| 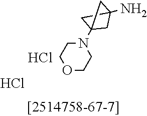 [SMILES: Cl ; NC12CC(N3CCOCC3)(C1)C2 ; Cl] | 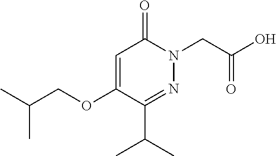 [SMILES: CC(C)COc1cc(=O)n(CC(=O)O)nc1C(C)C] | 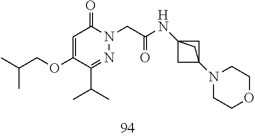 [SMILES: CC(C)COc1cc(=O)n(CC(=O)NC23CC(N4CCOCC4)(C2)C3)nc1C(C)C] |
| 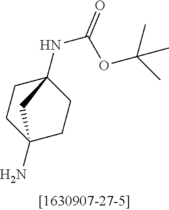 [SMILES: CC(C)(C)OC(=O)N[C@]12CC[C@@](N)(CC1)C2] | 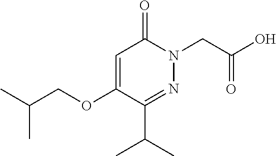 [SMILES: CC(C)COc1cc(=O)n(CC(=O)O)nc1C(C)C] | 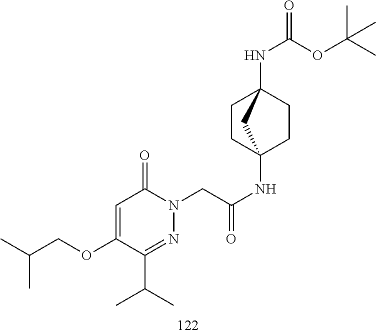 [SMILES: CC(C)COc1cc(=O)n(CC(=O)N[C@]23CC[C@@](NC(=O)OC(C)(C)C)(CC2)C3)nc1C(C)C] |
| 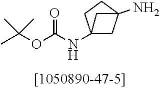 | 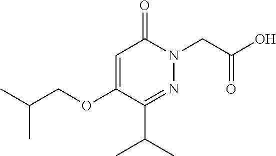 [SMILES: CC(C)COc1cc(=O)n(CC(=O)O)nc1C(C)C] | 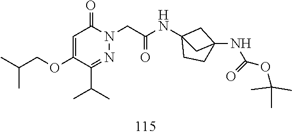 [SMILES: CC(C)COc1cc(=O)n(CC(=O)NC23CCC(NC(=O)OC(C)(C)C)(C2)C3)nc1C(C)C] |
| 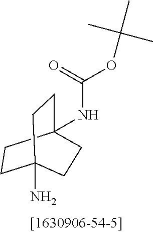 [SMILES: CC(C)(C)OC(=O)NC12CCC(N)(CC1)CC2] | 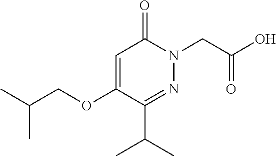 [SMILES: CC(C)COc1cc(=O)n(CC(=O)O)nc1C(C)C] | 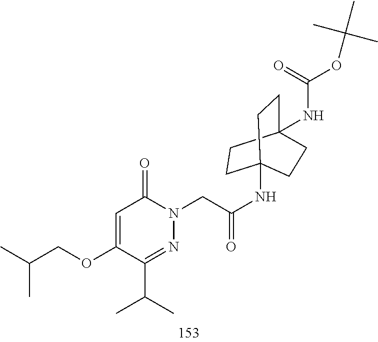 [SMILES: CC(C)COc1cc(=O)n(CC(=O)NC23CCC(NC(=O)OC(C)(C)C)(CC2)CC3)nc1C(C)C] |
| 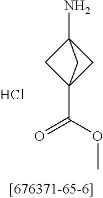 | 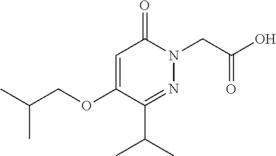 [SMILES: CC(C)COc1cc(=O)n(CC(=O)O)nc1C(C)C] | 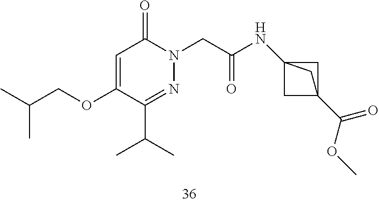 [SMILES: COC(=O)C12CC(NC(=O)Cn3nc(C(C)C)c(OCC(C)C)cc3=O)(C1)C2] |
| 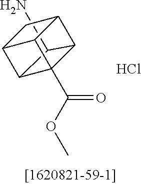 | 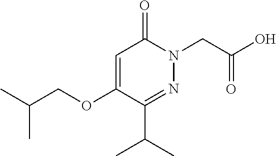 [SMILES: CC(C)COc1cc(=O)n(CC(=O)O)nc1C(C)C] | 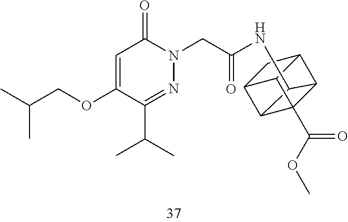 [SMILES: COC(=O)C12C3C4CC3C1C(NC(=O)Cn1nc(C(C)C)c(OCC(C)C)cc1=O)C42] |
| 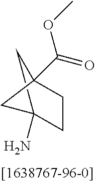 | 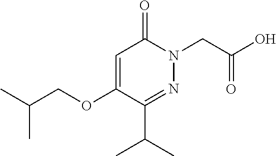 [SMILES: CC(C)COc1cc(=O)n(CC(=O)O)nc1C(C)C] | 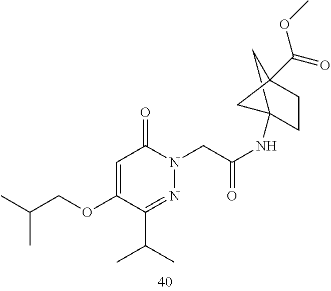 [SMILES: COC(=O)C12CCC(NC(=O)Cn3nc(C(C)C)c(OCC(C)C)cc3=O)(C1)C2] |
| 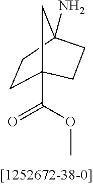 | 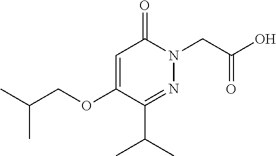 [SMILES: CC(C)COc1cc(=O)n(CC(=O)O)nc1C(C)C] | 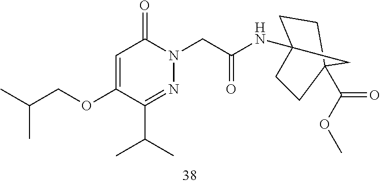 [SMILES: COC(=O)C12CCC(NC(=O)Cn3nc(C(C)C)c(OCC(C)C)cc3=O)(CC1)C2] |
| 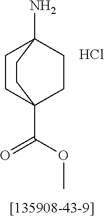 | 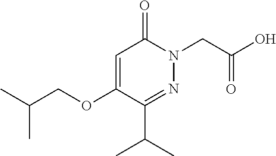 [SMILES: CC(C)COc1cc(=O)n(CC(=O)O)nc1C(C)C] | 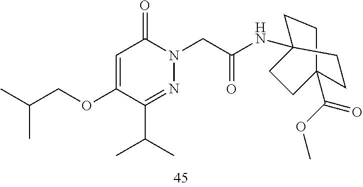 [SMILES: COC(=O)C12CCC(NC(=O)Cn3nc(C(C)C)c(OCC(C)C)cc3=O)(CC1)CC2] |
| 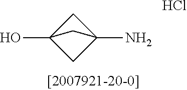 | 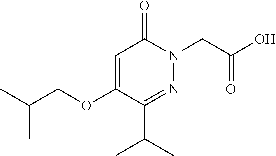 [SMILES: CC(C)COc1cc(=O)n(CC(=O)O)nc1C(C)C] | 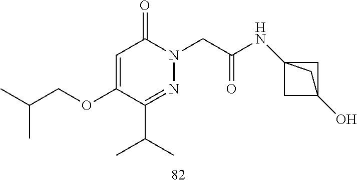 [SMILES: CC(C)COc1cc(=O)n(CC(=O)NC23CC(O)(C2)C3)nc1C(C)C] |
| 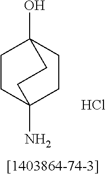 | 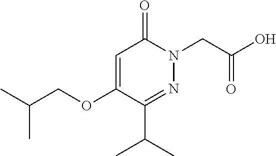 [SMILES: CC(C)COc1cc(=O)n(CC(=O)O)nc1C(C)C] | 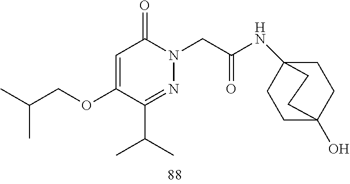 [SMILES: CC(C)COc1cc(=O)n(CC(=O)NC23CCC(O)(CC2)CC3)nc1C(C)C] |
| 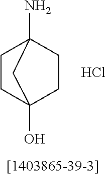 | 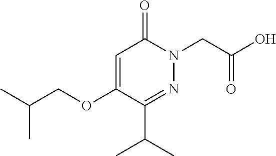 [SMILES: CC(C)COc1cc(=O)n(CC(=O)O)nc1C(C)C] | 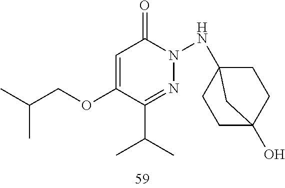 [SMILES: CC(C)COc1cc(=O)n(NC23CCC(O)(CC2)C3)nc1C(C)C] |
| 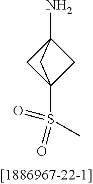 | 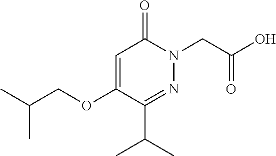 [SMILES: CC(C)COc1cc(=O)n(CC(=O)O)nc1C(C)C] | 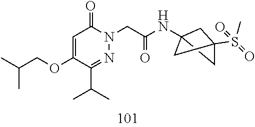 [SMILES: CC(C)COc1cc(=O)n(CC(=O)NC23CC(S(C)(=O)=O)(C2)C3)nc1C(C)C] |
| 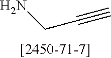 | 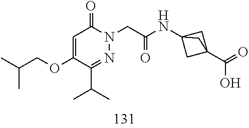 [SMILES: CC(C)COc1cc(=O)n(CC(=O)NC23CC(C(=O)O)(C2)C3)nc1C(C)C] | 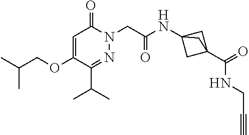 [SMILES: C#CCNC(=O)C12CC(NC(=O)Cn3nc(C(C)C)c(OCC(C)C)cc3=O)(C1)C2] |

### Synthesis of N-(3-(2-hydroxypropan-2-yl)bicyclo[1.1.1]pentan-1-yl)-2-(4-isobutoxy-3-isopropyl-6-oxopyridazin-1(6H)-yl)acetamide 85

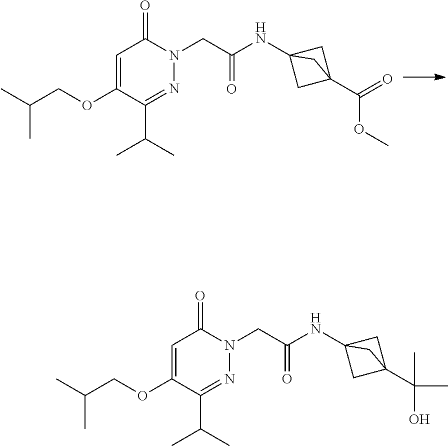

Methylmagnesium bromide solution (3.2M in 2-MeTHF) [75-16-1] (200 μL, 3.2 M, 0.64 mmol) was added dropwise to a stirred solution of methyl 3-(2-(4-isobutoxy-3-isopropyl-6-oxopyridazin-1(6H)-yl)acetamido)bicyclo[1.1.1]pentane-1-carboxylate (55 mg, 0.14 mmol) in 5 mL of anhydrous THF at −78° C. The resulting mixture was allowed to warm to 0° C. and stirred 1 h. Water was carefully added to the mixture, followed by EtOAc. The organic layer was separated, washed with brine (×2), dried (MgSO4), filtered of and evaporated under reduced pressure. The crude was purified via Prep HPLC (Stationary phase: RP)(Bridge Prep C18 OBD-10 μm, 30×150 mm, Mobile phase: 0.25% NH4HCO3 solution in water, CH3CN). The purest fractions were collected, evaporated under reduced pressure and coevaporated with MeOH to afford N-(3-(2-hydroxypropan-2-yl)bicyclo[1.1.1]pentan-1-yl)-2-(4-isobutoxy-3-isopropyl-6-oxopyridazin-1(6H)-yl)acetamide 85 (26.3 mg, yield 48%) as a sticky yellow oil.

### Synthesis of N-(3-(hydrazinecarbonyl)bicyclo[1.1.1]pentan-1-yl)-2-(4-isobutoxy-3-isopropyl-6-oxopyridazin-1(6H)-yl)acetamide

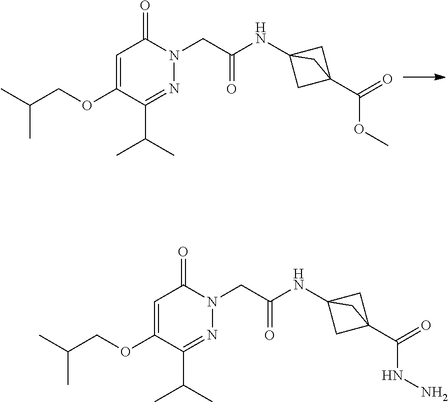

Hydrazine hydrate [7803-57-8] (0.31 mL, 1.03 g/mL, 6.43 mmol) was added to a stirred solution of methyl 3-(2-(4-isobutoxy-3-isopropyl-6-oxopyridazin-1(6H)-yl)acetamido)bicyclo[1.1.1]pentane-1-carboxylate 36 (250 mg, 0.64 mmol) in EtOH (1.9 mL) The mixture was stirred at 80° C. for 16h. The solvent was concentrated in vacuo and dried at 50° C. for 3h to yield N-(3-(hydrazinecarbonyl)bicyclo[1.1.1]pentan-1-yl)-2-(4-isobutoxy-3-isopropyl-6-oxopyridazin-1(6H)-yl)acetamide (260 mg, yield 75%) as a white solid.

### Synthesis of 2-(4-isobutoxy-3-isopropyl-6-oxopyridazin-1(6H)-yl)-N-(3-(5-methyl-1,3,4-oxadiazol-2-yl)bicyclo[1.1.1]pentan-1-yl)acetamide 119

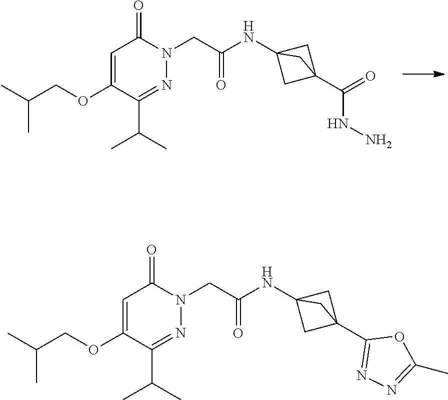 [SMILES: CC(C)COc1cc(=O)n(CC(=O)NC23CC(C(=O)NN)(C2)C3)nc1C(C)C ; Cc1nnc(C23CC(NC(=O)Cn4nc(C(C)C)c(OCC(C)C)cc4=O)(C2)C3)o1]

N-(3-(hydrazinecarbonyl)bicyclo[1.1.1]pentan-1-yl)-2-(4-isobutoxy-3-isopropyl-6-oxopyridazin-1(6H)-yl)acetamide (50 mg, 0.13 mmol) was suspended in THF (0.74 mL) under nitrogen. DIPEA [7087-68-5] (44.02 μL, 0.75 g/mL, 0.26 mmol) was then added followed by acetyl chloride [75-36-5] (10.03 μL, 1.1 g/mL, 0.14 mmol) at 0° C. Resulting slurry was warmed to RT (then solubilized in THF) and stirred at that temperature for 10 min. Then Burgess reagent [29684-56-8] (121.63 mg, 0.51 mmol) was added. Reaction mixture was warmed to 130° C. under microwave irradiation for 30 min. The mixture was diluted with EtOAc (2 mL) and washed with NaHCO3 (2 mL). The organic phase was separated, dried (Na2SO4), filtered and concentrated in vacuo. The residue was sent to RP HPLC. Conditions: Stationary phase: C18 XBridge 30×100 mm 10 μm. Mobile phase: NH4HCO3 0.25% solution in Water and CH3CN, yielding 2-(4-isobutoxy-3-isopropyl-6-oxopyridazin-1(6H)-yl)-N-(3-(5-methyl-1,3,4-oxadiazol-2-yl)bicyclo[1.1.1]pentan-1-yl)acetamide 119 (24.4 mg, yield 46%) as a white solid.

### Synthesis of 2-(4-isobutoxy-3-isopropyl-6-oxopyridazin-1(6H)-yl)-N-(3-(5-methyloxazol-2-yl)bicyclo[1.1.1]pentan-1-yl)acetamide 139

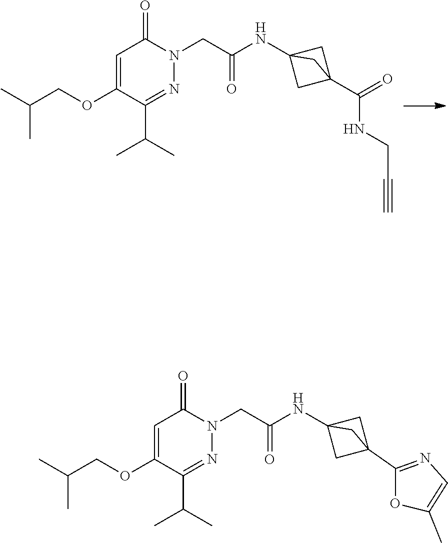

Zinc trifluoromethanesulfonate [54010-75-2] (1.69 mg, 0.0046 mmol) was added to a stirred suspension of 3-(2-(4-isobutoxy-3-isopropyl-6-oxopyridazin-1(6H)-yl)acetamido)-N-(prop-2-yn-1-yl)bicyclo[1.1.1]pentane-1-carboxamide (38.5 mg, 0.093 mmol) in toluene (0.5 mL). The mixture was stirred at 150° C. for 30 min under MW irradiation. Then more zinc trifluoromethanesulfonate [54010-75-2] (1.69 mg, 0.0046 mmol) was added. The mixture was stirred at 150° C. for 30 min under MW irradiation. The mixture was diluted with DCM (2 mL) and water (2 mL). Phases were separated. The aqueous phase was back extracted with DCM (2 ml). The combined organic layers were dried (Na2SO4), filtered and concentrated in vacuo. The residue was sent to RP HPLC. Conditions: Stationary phase: C18 XBridge 30×100 mm 10 μm. Mobile phase: NH4HCO3 0.25% solution in Water and CH3CN, yielding 2-(4-isobutoxy-3-isopropyl-6-oxopyridazin-1(6H)-yl)-N-(3-(5-methyloxazol-2-yl)bicyclo[1.1.1]pentan-1-yl)acetamide 139 (15.4 mg, yield 40%) as a white solid.

### Synthesis of N-hydroxyacetimidamide

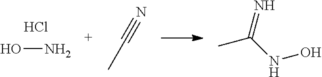

A solution of hydroxylamine hydrochloride [5470-11-1] (1200 mg, 17.27 mmol) and NaOH [1310-73-2] (691.15 mg, 17.28 mmol) in water (6 mL) was added (in about 15 min) to CH3CN (18 mL). The mixture was stirred at room temperature for 64h. The solvent was concentrated in vacuo and the residue treated with ethanol; the resulting suspension was filtered and the solvent removed under reduced pressure yielding N-hydroxyacetimidamide (1200 mg, yield 94%) as a white solid, used in the next step without further purification.

### Synthesis of 2-(4-isobutoxy-3-isopropyl-6-oxopyridazin-1(6H)-yl)-N-(3-(3-methyl-1,2,4-oxadiazol-5-yl)bicyclo[1.1.1]pentan-1-yl)acetamide 90

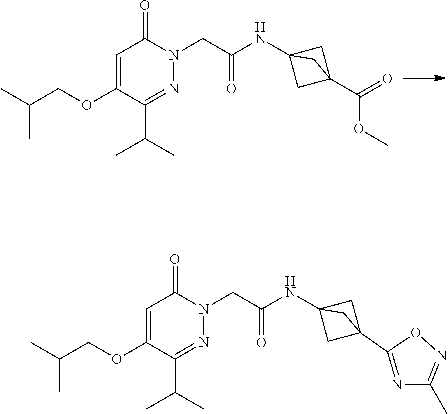

To a solution of methyl 3-(2-(4-isobutoxy-3-isopropyl-6-oxopyridazin-1(6H)-yl)acetamido)bicyclo[1.1.1]pentane-1-carboxylate 36 (50 mg, 0.13 mmol) in toluene (0.1 mL) was added N-hydroxyacetimidamide (10.41 mg, 0.14 mmol) and K2C03 [584-08-7] (19.42 mg, 0.14 mmol). The mixture was stirred at 110° C. for 16h. Then, more N-hydroxyacetimidamide (10.41 mg, 0.14 mmol) and K2CO3 [584-08-7] (19.42 mg, 0.14 mmol) were added. The mixture was stirred at 110° C. for 6h. The reaction mixture was cooled to room temperature, diluted with EtOAc (5 mL) and washed successively with water (2×2.5 mL) and brine (2.5 mL). The organic phase was dried (Na2SO4), filtered and concentrated in vacuo. The residue was sent to RP HPLC. Conditions: Stationary phase: C18 XBridge 30×100 mm 10 μm. Mobile phase: NH4HCO3 0.25% solution in Water and CH3CN, to yield 2-(4-isobutoxy-3-isopropyl-6-oxopyridazin-1(6H)-yl)-N-(3-(3-methyl-1,2,4-oxadiazol-5-yl)bicyclo[1.1.1]pentan-1-yl)acetamide 90 (16.9 mg, yield 32%) as a white solid.

### Synthesis of N-((1r,4r)-4-aminobicyclo[2.2.1]heptan-1-yl)-2-(4-isobutoxy-3-isopropyl-6-oxopyridazin-1(6H)-yl)acetamide 75

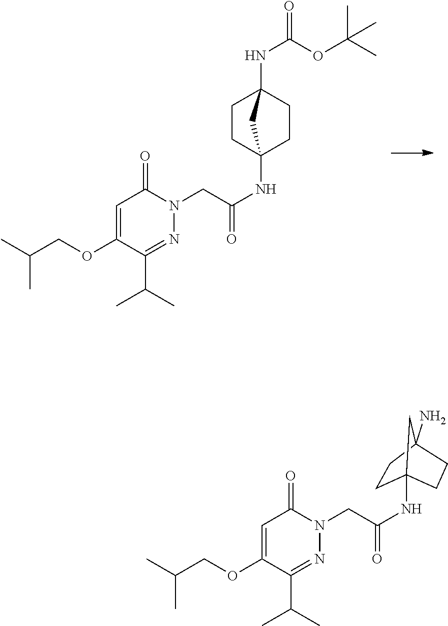

TFA [76-05-1] (270 μL, 1.49 g/mL, 3.53 mmol) was added to a stirred solution of tert-butyl ((1r,4r)-4-(2-(4-isobutoxy-3-isopropyl-6-oxopyridazin-1(6H)-yl)acetamido)bicyclo[2.2.1]heptan-1-yl)carbamate 122 (40 mg, 0.084 mmol) in DCM (0.55 mL). The mixture was stirred at room temperature for 1h. The solvent was concentrated in vacuo. The residue was dissolved in MeOH passed through a SCX-2 cartridge eluting with 7N solution of ammonia in MeOH. The solvent was concentrated in vacuo to yield N-((1r,4r)-4-aminobicyclo[2.2.1]heptan-1-yl)-2-(4-isobutoxy-3-isopropyl-6-oxopyridazin-1(6H)-yl)acetamide 75 (27 mg, yield 85% o) as a white solid.

Additional analogues were synthesised according to the above procedure, using the appropriate reagent.

| Intermediate | Product |
|---|---|
| 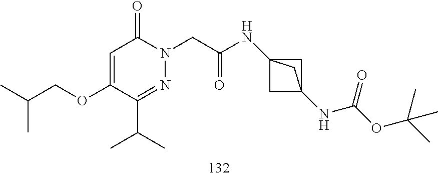 [SMILES: CC(C)COc1cc(=O)n(CC(=O)NC23CC(NC(=O)OC(C)(C)C)(C2)C3)nc1C(C)C] | 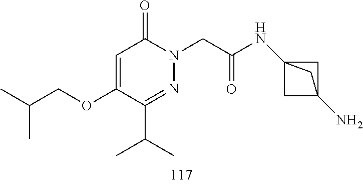 [SMILES: CC(C)COc1cc(=O)n(CC(=O)NC23CC(N)(C2)C3)nc1C(C)C] |
| 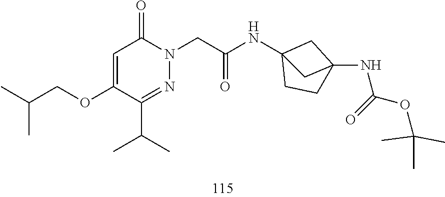 [SMILES: CC(C)COc1cc(=O)n(CC(=O)NC23CCC(NC(=O)OC(C)(C)C)(C2)C3)nc1C(C)C] | 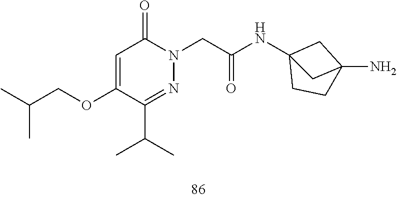 [SMILES: CC(C)COc1cc(=O)n(CC(=O)NC23CCC(N)(C2)C3)nc1C(C)C] |
| 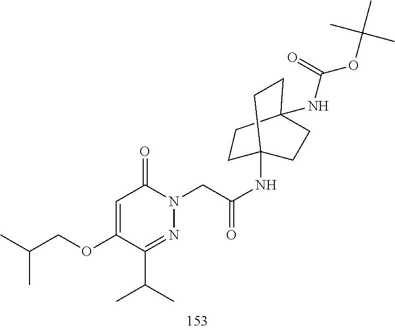 [SMILES: CC(C)COc1cc(=O)n(CC(=O)NC23CCC(NC(=O)OC(C)(C)C)(CC2)CC3)nc1C(C)C] | 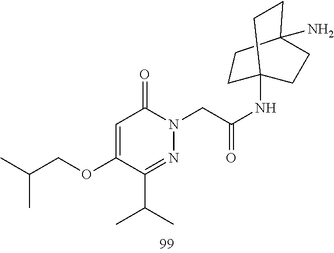 [SMILES: CC(C)COc1cc(=O)n(CC(=O)NC23CCC(N)(CC2)CC3)nc1C(C)C] |
| 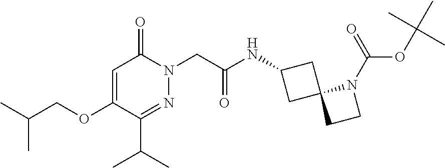 [SMILES: CC(C)COc1cc(=O)n(CC(=O)N[C@H]2C[C@]3(CCN3C(=O)OC(C)(C)C)C2)nc1C(C)C] | 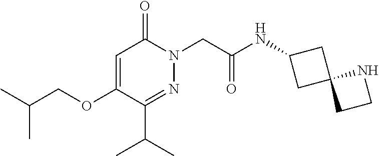 [SMILES: CC(C)COc1cc(=O)n(CC(=O)N[C@H]2C[C@]3(CCN3)C2)nc1C(C)C] |

### Synthesis of 2-(4-isobutoxy-3-isopropyl-6-oxopyridazin-1(6H)-yl)-N-((4s,6r)-1-methyl-1-azaspiro[3.3]heptan-6-yl)acetamide 89

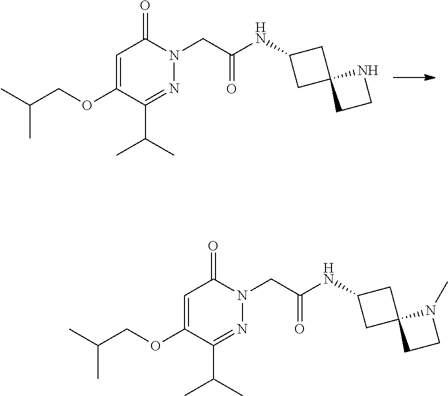 [SMILES: CC(C)COc1cc(=O)n(CC(=O)N[C@H]2C[C@]3(CCN3)C2)nc1C(C)C ; CC(C)COc1cc(=O)n(CC(=O)N[C@H]2C[C@]3(CCN3C)C2)nc1C(C)C]

Formaldehyde solution [50-00-0] (31 μL, 0.41 mmol) was added to a stirred solution of 2-(4-isobutoxy-3-isopropyl-6-oxopyridazin-1(6H)-yl)-N-((4s,6r)-1-azaspiro[3.3]heptan-6-yl)acetamide (131 mg, 0.28 mmol) and triethylamine [121-44-8](76 uL, 0.55 mmol) in MeOH (3.5 mL) at rt. The mixture was stirred for 5 min and then sodium cyanoborohydride [25895-60-7] (26 mg, 0.41 mmol) was added and the mixture was stirred at rt for 16 h. The mixture was diluted with NaHCO3 (saturated in water) and extracted with EtOAc. The organic layer was separated, dried (MgSO4) and filtered. The solvent was evaporated in vacuo. The crude product was purified by flash column chromatography (silica 25 g; NH3 (7M in MeOH)/MeOH/DCM 0/0/100 to 20/1/1). The crude was purified by reverse phase (Phenomenex Gemini C18 30×100 mm 5 μm Column; from 70% [25 mM NH4HCO3]-30% [ACN:MeOH (1:1)] to 27% [25 mM NH4HCO3]-73% [ACN:MeOH (1:1)]). The desired fractions were collected and concentrated in vacuo to yield 2-(4-isobutoxy-3-isopropyl-6-oxopyridazin-1(6H)-yl)-N-((4s,6r)-1-methyl-1-azaspiro[3.3]heptan-6-yl)acetamide 89 (50 mg, yield 48%) as a white solid.

Additional analogues were synthetized according to the above procedure using the appropriate reagents.

| Intermediate | Product |
|---|---|
| 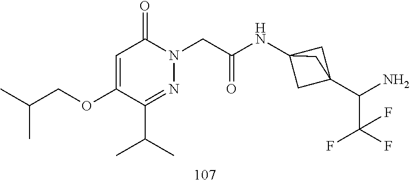 [SMILES: CC(C)COc1cc(=O)n(CC(=O)NC23CC(C(N)C(F)(F)F)(C2)C3)nc1C(C)C] | 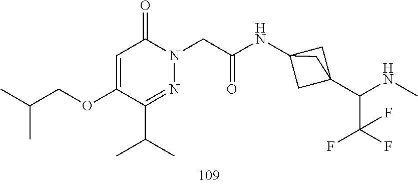 [SMILES: CNC(C(F)(F)F)C12CC(NC(=O)Cn3nc(C(C)C)c(OCC(C)C)cc3=O)(C1)C2] |

### Synthesis of N-((4s,6r)-1-ethyl-1-azaspiro[3.3]heptan-6-yl)-2-(4-isobutoxy-3-isopropyl-6-oxopyridazin-1(6H)-yl)acetamide 81

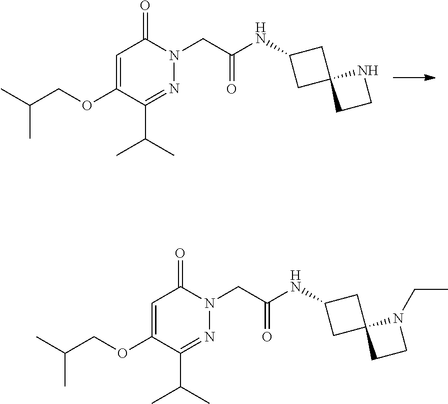 [SMILES: CC(C)COc1cc(=O)n(CC(=O)N[C@H]2C[C@]3(CCN3)C2)nc1C(C)C ; CCN1CC[C@]12C[C@H](NC(=O)Cn1nc(C(C)C)c(OCC(C)C)cc1=O)C2]

Bromoethane [74-96-4] (41 uL, 0.6 mmol) and DIPEA [7087-68-5] (575 uL, 3.3 mmol) were added to a solution of 2-(4-isobutoxy-3-isopropyl-6-oxopyridazin-1(6H)-yl)-N-((4s,6r)-1-azaspiro[3.3]heptan-6-yl)acetamide (131 mg, 0.28 mmol) in ACN [75-05-8] (3.7 mL). The mixture was stirred and heated at 85° C. for 16 h. The reaction mixture was diluted with water and extracted with EtOAc. The organic layer was separated, dried (MgSO4), filtered and the solvents evaporated in vacuo. The crude product was purified by flash column chromatography (silica 25 g; EtOAc in heptane 0/100 to 80/20). The crude was purified by reverse phase (Phenomenex Gemini C18 30×100 mm 5 μm Column; from 70% [25 mM NH4HCO3]-30% [ACN:MeOH (1:1)] to 27% [25 mM NH4HCO3]-73% [ACN:MeOH (1:1)]). The desired fractions were collected and concentrated in vacuo to yield N-((4s,6r)-1-ethyl-1-azaspiro[3.3]heptan-6-yl)-2-(4-isobutoxy-3-isopropyl-6-oxopyridazin-1(6H)-yl)acetamide 81 (42 mg, yield 39%) as a white solid.

### Synthesis of N-([1,2,4]triazolo[4,3-b]pyridazin-6-yl)-2-(5-chloro-4-(cyclopropylmethoxy)-3-isopropyl-6-oxopyridazin-1(6H)-yl)acetamide 44

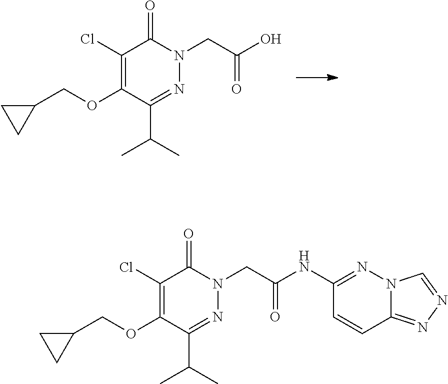 [SMILES: CC(C)c1nn(CC(=O)O)c(=O)c(Cl)c1OCC1CC1 ; CC(C)c1nn(CC(=O)Nc2ccc3nncn3n2)c(=O)c(Cl)c1OCC1CC1]

To a mixture of 2-(5-chloro-4-(cyclopropylmethoxy)-3-isopropyl-6-oxopyridazin-1(6H)-yl)acetic acid (115 mg, 0.34 mmol) in dry pyridine (5.7 ml), [1,2,4]triazolo[4,3-b]pyridazin-6-amine [19195-46-1] (76 mg, 0.52 mmol) was added. The mixture was sonicated for 10 min and then stirred for 40 min at rt. Titanium(IV) chloride [7550-45-0] (1.37 mL, 1 M, 1.37 mmol) was added dropwise at rt. The mixture was stirred for 1h at rt and then heated at 80° C. for 24 h. The solvent was evaporated in vacuo and the crude was treated with HCl (2 N) till acid PH, the crude was extracted with AcOEt (3×5 ml) the combined organic layers were evaporated to afford an oil. The residue was purified by flash column chromatography (silica, EtOAc in DCM 0/100 to 100/0 and then MeOH in EtOAc 0/100 to 15/85). The desired fractions were collected and concentrated in vacuo. The residue was triturated with MeOH (some drops) and DIPE. The solid was stirred at RT for 2h. The solid was filtered off, washed with DIPE and dried under vacuo at 55° C. for 72h to yield N-([1,2,4]triazolo[4,3-b]pyridazin-6-yl)-2-(5-chloro-4-(cyclopropylmethoxy)-3-isopropyl-6-oxopyridazin-1(6H)-yl)acetamide 44 (50 mg, yield 36% o) as a white solid.

Additional analogues were synthesized according to the above procedure, using the appropriate reagents.

| Intermediate | Product |
|---|---|
| 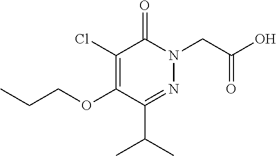 [SMILES: CCCOc1c(C(C)C)nn(CC(=O)O)c(=O)c1Cl] | 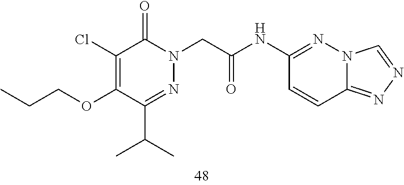 [SMILES: CCCOc1c(C(C)C)nn(CC(=O)Nc2ccc3nncn3n2)c(=O)c1Cl] |
| 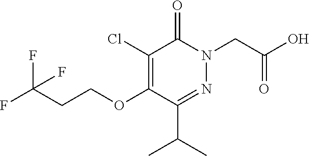 [SMILES: CC(C)c1nn(CC(=O)O)c(=O)c(Cl)c1OCCC(F)(F)F] | 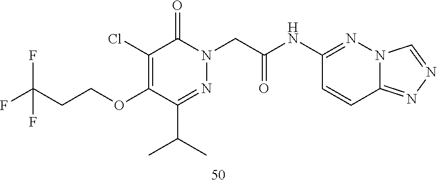 [SMILES: CC(C)c1nn(CC(=O)Nc2ccc3nncn3n2)c(=O)c(Cl)c1OCCC(F)(F)F] |
| 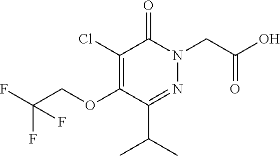 [SMILES: CC(C)c1nn(CC(=O)O)c(=O)c(Cl)c1OCC(F)(F)F] | 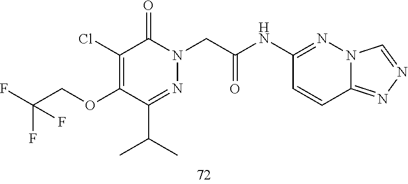 [SMILES: CC(C)c1nn(CC(=O)Nc2ccc3nncn3n2)c(=O)c(Cl)c1OCC(F)(F)F] |
| 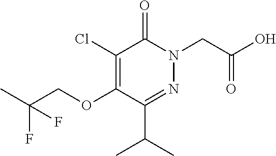 [SMILES: CC(C)c1nn(CC(=O)O)c(=O)c(Cl)c1OCC(C)(F)F] | 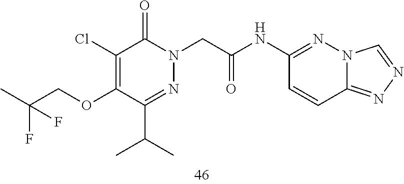 [SMILES: CC(C)c1nn(CC(=O)Nc2ccc3nncn3n2)c(=O)c(Cl)c1OCC(C)(F)F] |

### Synthesis of tert-butyl 3-((1-(2-([1,2,4]triazolo[4,3-a]pyridin-6-ylamino)-2-oxoethyl)-3-isopropyl-6-oxo-1,6-dihydropyridazin-4-yl)oxy)pyrrolidine-1I-carboxylate 1F

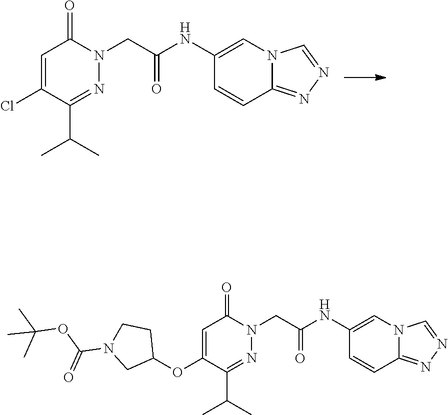 [SMILES: CC(C)c1nn(CC(=O)Nc2ccc3nncn3c2)c(=O)cc1Cl ; CC(C)c1nn(CC(=O)Nc2ccc3nncn3c2)c(=O)cc1OC1CCN(C(=O)OC(C)(C)C)C1]

1-boc-azetidine-3-yl-methanol [142253-56-3] (78 mg, 0.42 mmol) was added to a stirred suspension of NaH (60% dispersion in mineral oil) [7646-69-7] (18 mg, 0.45 mmol) in anhydrous DMF (1 mL) at 0° C. and under N₂. The mixture was stirred at 0° C. for 5 min and at RT for 15 min. Then, a suspension of N-([1,2,4] triazolo[4,3-a] pyridin-6-yl)-2-(4-chloro-3-isopropyl-6-oxopyridazin-1(6H)-yl) acetamide 1E (80 mg, 0.23 mmol) in DMF anhydrous (1.5 mL) was added at 0° C. The resulting mixture was stirred at RT for 5 min and then at 150° C. for 10 min under microwave irradiation.

A drop of water was added to the mixture and stirred for 15 min, the mixture was dried (Na2SO4), filtered and the solvents evaporated in vacuo. The crude was purified by RP HPLC (Stationary phase: C18 XBridge 30×100 mm 5 μm, Mobile phase: Gradient from 90% NH₄HCO₃ 0.25% solution in Water, 10% ACN to 10% NH₄HCO₃ 0.25% solution in Water, 90% ACN). The different product fractions were combined and the solvent was evaporated in vacuo to yield tert-butyl 3-((1-(2-([1,2,4]triazolo[4,3-a]pyridin-6-ylamino)-2-oxoethyl)-3-isopropyl-6-oxo-1,6-dihydropyridazin-4-yl)oxy)pyrrolidine-1-carboxylate 1F (88.5 mg, 77%) as an off white solid.

LCMS (Rₜ: 1.74, Area %: 98.25, MW: 497.24, BPM1: 498.2, Method 5)

¹H NMR (400 MHz, DMSO-d₆) δ ppm 1.15 (d, J=6.70 Hz, 6H) 1.38 (s, 9H) 2.99 (tt, J=8.29, 5.35 Hz, 1H) 3.08 (quin, J=6.88 Hz, 1H) 3.74 (br s, 2H) 3.97 (br s, 2H) 4.16 (d, J=5.09 Hz, 2H) 4.83 (s, 2H) 6.33 (s, 1H) 7.30 (dd, J=9.83, 1.97 Hz, 1H) 7.79 (d, J=9.71 Hz, 1H) 9.20 (dd, J=1.62, 0.92 Hz, 1H) 9.24 (d, J=0.69 Hz, 1H) 10.55 (br s, 1H)

Additional analogs were accessed using similar reaction conditions, using the appropriate reagent.

| Number | Reagent | Final compound | Yield |
|---|---|---|---|
| 2F | 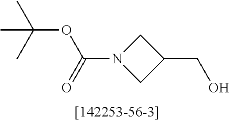 | 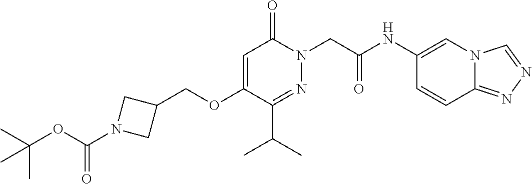 [SMILES: CC(C)c1nn(CC(=O)Nc2ccc3nncn3c2)c(=O)cc1OCC1CN(C(=O)OC(C)(C)C)C1] | 77% |
| 3F | 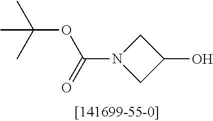 | 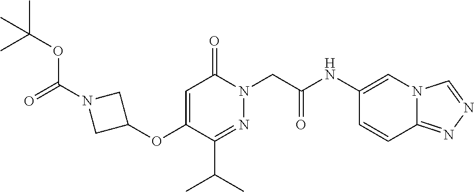 [SMILES: CC(C)c1nn(CC(=O)Nc2ccc3nncn3c2)c(=O)cc1OC1CN(C(=O)OC(C)(C)C)C1] | 82% |
| 4F | 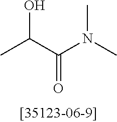 | 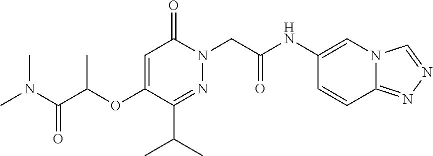 [SMILES: CC(Oc1cc(=O)n(CC(=O)Nc2ccc3nncn3c2)nc1C(C)C)C(=O)N(C)C] | 56% |
| 5F | 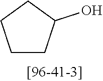 | 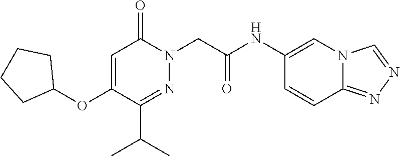 [SMILES: CC(C)c1nn(CC(=O)Nc2ccc3nncn3c2)c(=O)cc1OC1CCCC1] | 12% |
| 6F | 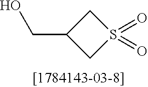 | 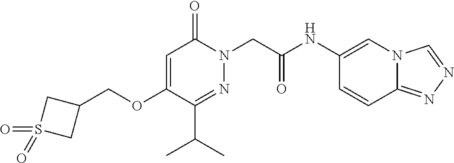 [SMILES: CC(C)c1nn(CC(=O)Nc2ccc3nncn3c2)c(=O)cc1OCC1CS(=O)(=O)C1] | 44% |
| 7F | 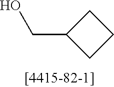 | 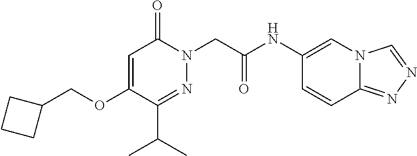 [SMILES: CC(C)c1nn(CC(=O)Nc2ccc3nncn3c2)c(=O)cc1OCC1CCC1] | 24% |
| 8F | 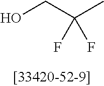 | 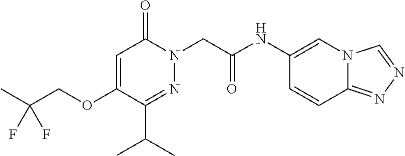 [SMILES: CC(C)c1nn(CC(=O)Nc2ccc3nncn3c2)c(=O)cc1OCC(C)(F)F] | 20% |
| 9F | 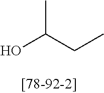 | 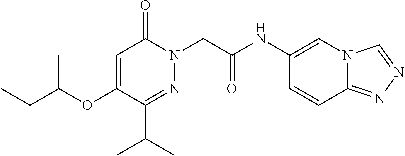 [SMILES: CCC(C)Oc1cc(=O)n(CC(=O)Nc2ccc3nncn3c2)nc1C(C)C] | 27% |
| 162 | 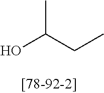 | 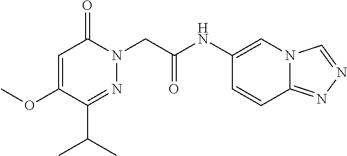 [SMILES: COc1cc(=O)n(CC(=O)Nc2ccc3nncn3c2)nc1C(C)C] | 16% |
| 10F | 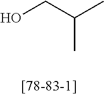 | 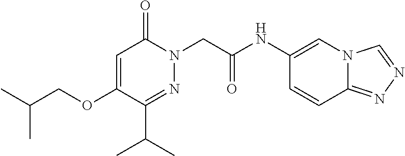 [SMILES: CC(C)COc1cc(=O)n(CC(=O)Nc2ccc3nncn3c2)nc1C(C)C] | 26% |
| 11F | 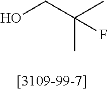 | 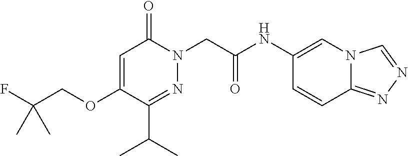 [SMILES: CC(C)c1nn(CC(=O)Nc2ccc3nncn3c2)c(=O)cc1OCC(C)(C)F] | 33% |
| 12F | 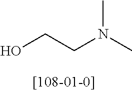 | 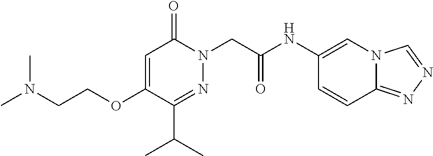 [SMILES: CC(C)c1nn(CC(=O)Nc2ccc3nncn3c2)c(=O)cc1OCCN(C)C] | 26% |
| 13F | 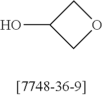 | 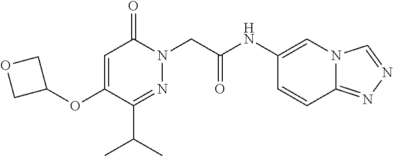 [SMILES: CC(C)c1nn(CC(=O)Nc2ccc3nncn3c2)c(=O)cc1OC1COC1] | 36% |
| 14F | 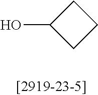 | 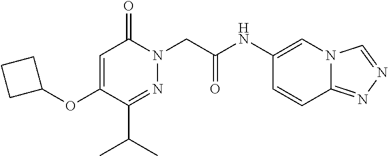 [SMILES: CC(C)c1nn(CC(=O)Nc2ccc3nncn3c2)c(=O)cc1OC1CCC1] | 29% |
| 15F | 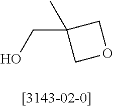 | 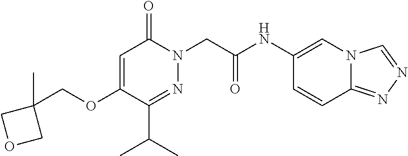 [SMILES: CC(C)c1nn(CC(=O)Nc2ccc3nncn3c2)c(=O)cc1OCC1(C)COC1] | 30% |
| 16F | 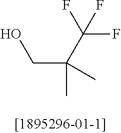 | 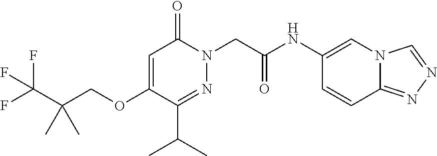 [SMILES: CC(C)c1nn(CC(=O)Nc2ccc3nncn3c2)c(=O)cc1OCC(C)(C)C(F)(F)F] | 30% |
| 17F | 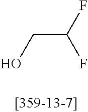 | 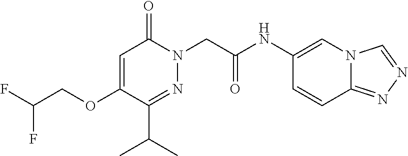 [SMILES: CC(C)c1nn(CC(=O)Nc2ccc3nncn3c2)c(=O)cc1OCC(F)F] | 39% |
| 18F | 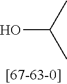 | 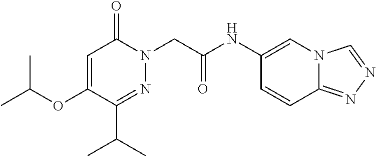 [SMILES: CC(C)Oc1cc(=O)n(CC(=O)Nc2ccc3nncn3c2)nc1C(C)C] | 27% |
| 19F | 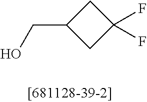 | 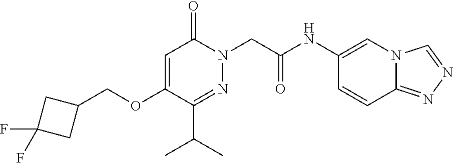 [SMILES: CC(C)c1nn(CC(=O)Nc2ccc3nncn3c2)c(=O)cc1OCC1CC(F)(F)C1] | 34% |
| 20F | 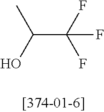 | 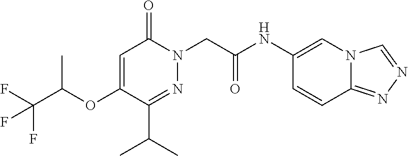 [SMILES: CC(C)c1nn(CC(=O)Nc2ccc3nncn3c2)c(=O)cc1OC(C)C(F)(F)F] | 33% |
| 21F | 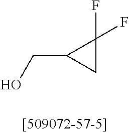 | 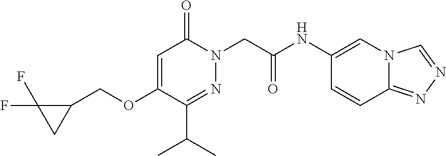 [SMILES: CC(C)c1nn(CC(=O)Nc2ccc3nncn3c2)c(=O)cc1OCC1CC1(F)F] | 22% |
| 22F | 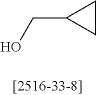 | 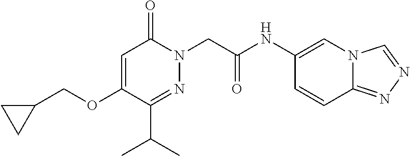 [SMILES: CC(C)c1nn(CC(=O)Nc2ccc3nncn3c2)c(=O)cc1OCC1CC1] | 19% |
| 23F | 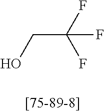 | 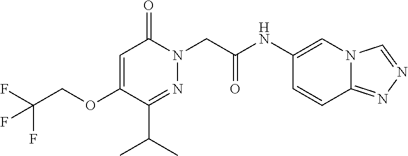 [SMILES: CC(C)c1nn(CC(=O)Nc2ccc3nncn3c2)c(=O)cc1OCC(F)(F)F] | 21% |
| 24F | 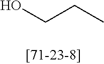 | 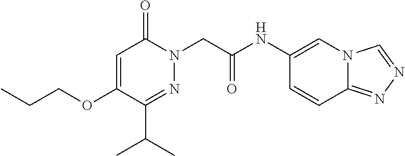 [SMILES: CCCOc1cc(=O)n(CC(=O)Nc2ccc3nncn3c2)nc1C(C)C] | 23% |
| 25F | 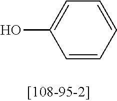 | 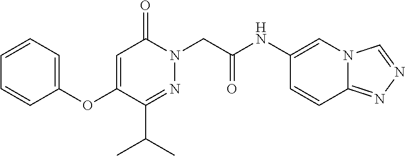 [SMILES: CC(C)c1nn(CC(=O)Nc2ccc3nncn3c2)c(=O)cc1Oc1ccccc1] | 48% |
| 26F | 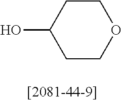 | 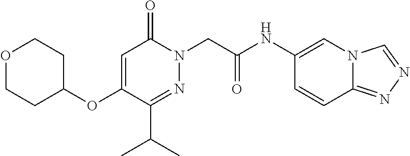 [SMILES: CC(C)c1nn(CC(=O)Nc2ccc3nncn3c2)c(=O)cc1OC1CCOCC1] | 27% |
| 64 | 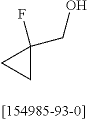 | 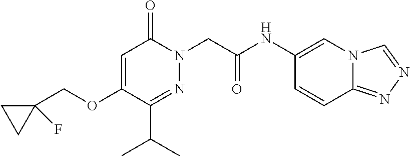 [SMILES: CC(C)c1nn(CC(=O)Nc2ccc3nncn3c2)c(=O)cc1OCC1(F)CC1] | 60% |

### Synthesis of N-([1,2,4]triazolo[4,3-b]pyridazin-6-yl)-2-(3-isopropyl-4-methoxy-6-oxopyridazin-1(6H)-yl)acetamide 27C

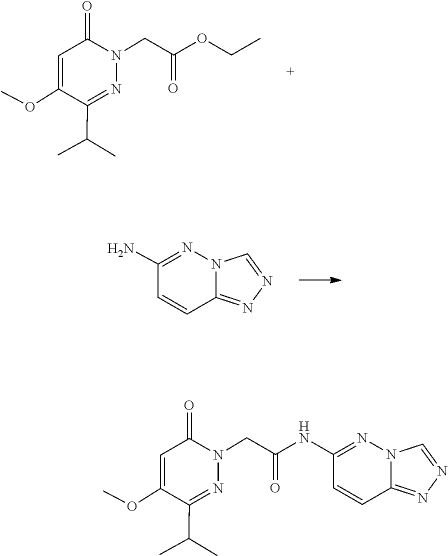 [SMILES: CCOC(=O)Cn1nc(C(C)C)c(OC)cc1=O ; Nc1ccc2nncn2n1 ; COc1cc(=O)n(CC(=O)Nc2ccc3nncn3n2)nc1C(C)C]

Lithium bis(trimethylsilyl)amide [4039-32-1] (0.53 mL, 1 M, 0.53 mmol) was added to a stirred suspension of [1,2,4]triazolo[4,3-B]pyridazin-6-amine [19195-46-1] (39 mg, 0.27 mmol) in DMF (1 mL) at 0° C. under N₂. The mixture was stirred at 0° C. for 10 min and then ethyl 2-(3-isopropyl-4-methoxy-6-oxopyridazin-1(6H)-yl) acetate 1B (60 mg, 0.24 mmol) in THF (1 mL) was added at 0° C. The resulting mixture was stirred at this temperature for 10 min and then at RT for 1.5 h.

The mixture was diluted with NH₄Cl (10% in water) and extracted with EtOAc (×3). The organic layer was separated, dried (Na₂SO₄), filtered and the solvents evaporated in vacuo to yield N-([1,2,4]triazolo[4,3-b]pyridazin-6-yl)-2-(3-isopropyl-4-methoxy-6-oxopyridazin-1(6H)-yl)acetamide 27C (52 mg, yield 64%) as a white solid.

LCMS (Rₜ: 1.13, Area %: 100.00, MW: 343.14, BPM1: 344.14, Method 5)

¹H NMR (500 MHz, DMSO-d₆) δ ppm 1.14 (d, J=6.87 Hz, 6H) 3.05-3.14 (m, 1H) 3.85 (s, 3H) 4.91 (s, 2H) 6.31 (s, 1H) 7.91 (br d, J=10.07 Hz, 1H) 8.34 (dd, J=9.99, 0.69 Hz, 1H) 9.52 (d, J=0.76 Hz, 1H) 11.28-11.54 (m, 1H).

Additional analogues were synthesized according to the above procedure, using the

| Reagent | Intermediate | Product |
|---|---|---|
| 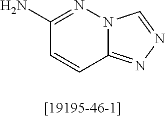 | 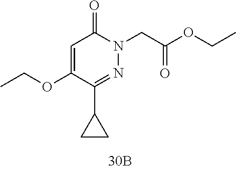 [SMILES: CCOC(=O)Cn1nc(C2CC2)c(OCC)cc1=O] | 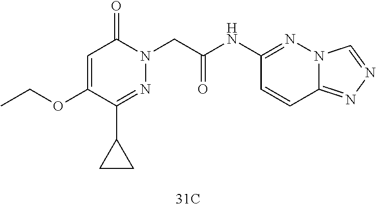 [SMILES: CCOc1cc(=O)n(CC(=O)Nc2ccc3nncn3n2)nc1C1CC1] |
| 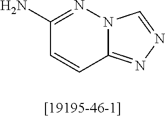 | 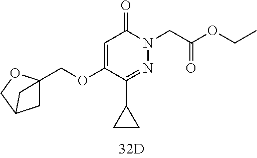 [SMILES: CCOC(=O)Cn1nc(C2CC2)c(OCC23CC(CO2)C3)cc1=O] | 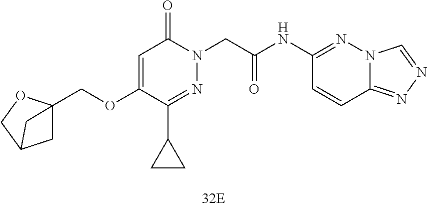 [SMILES: O=C(Cn1nc(C2CC2)c(OCC23CC(CO2)C3)cc1=O)Nc1ccc2nncn2n1] |
| 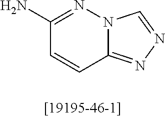 | 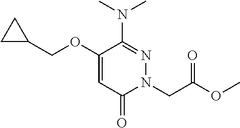 [SMILES: COC(=O)Cn1nc(N(C)C)c(OCC2CC2)cc1=O] | 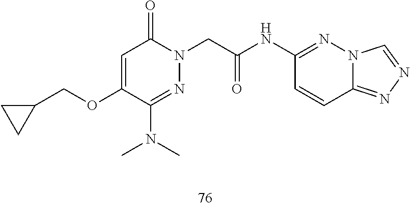 [SMILES: CN(C)c1nn(CC(=O)Nc2ccc3nncn3n2)c(=O)cc1OCC1CC1] |
| 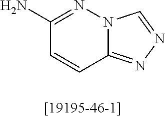 | 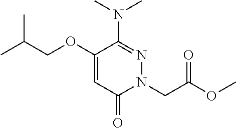 [SMILES: COC(=O)Cn1nc(N(C)C)c(OCC(C)C)cc1=O] | 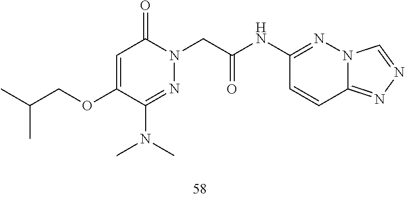 [SMILES: CC(C)COc1cc(=O)n(CC(=O)Nc2ccc3nncn3n2)nc1N(C)C] |
| 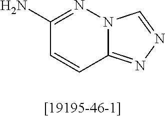 | 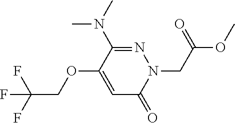 [SMILES: COC(=O)Cn1nc(N(C)C)c(OCC(F)(F)F)cc1=O] | 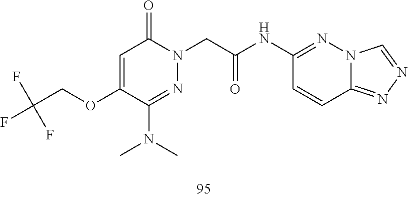 [SMILES: CN(C)c1nn(CC(=O)Nc2ccc3nncn3n2)c(=O)cc1OCC(F)(F)F] |
| 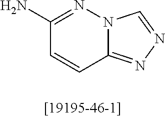 | 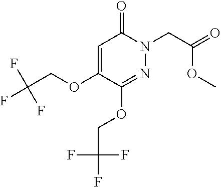 [SMILES: COC(=O)Cn1nc(OCC(F)(F)F)c(OCC(F)(F)F)cc1=O] | 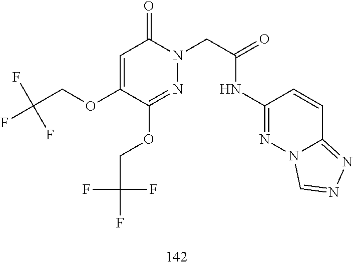 [SMILES: O=C(Cn1nc(OCC(F)(F)F)c(OCC(F)(F)F)cc1=O)Nc1ccc2nncn2n1] |
| 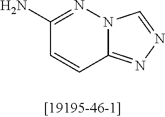 | 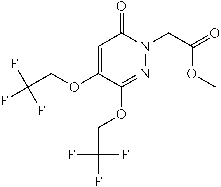 [SMILES: COC(=O)Cn1nc(OCC(F)(F)F)c(OCC(F)(F)F)cc1=O] | 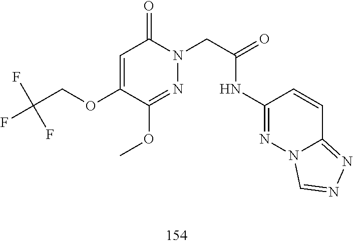 [SMILES: COc1nn(CC(=O)Nc2ccc3nncn3n2)c(=O)cc1OCC(F)(F)F] |
| 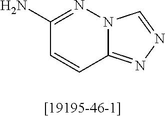 | 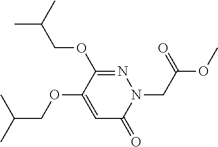 [SMILES: COC(=O)Cn1nc(OCC(C)C)c(OCC(C)C)cc1=O] | 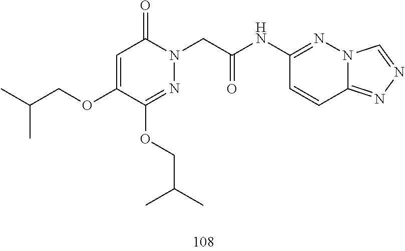 [SMILES: CC(C)COc1cc(=O)n(CC(=O)Nc2ccc3nncn3n2)nc1OCC(C)C] |
| 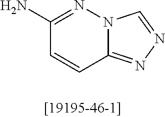 | 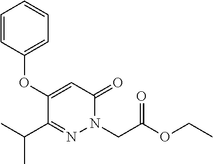 [SMILES: CCOC(=O)Cn1nc(C(C)C)c(Oc2ccccc2)cc1=O] | 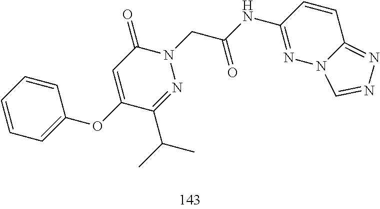 [SMILES: CC(C)c1nn(CC(=O)Nc2ccc3nncn3n2)c(=O)cc1Oc1ccccc1] |
| 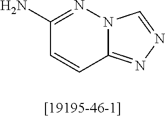 | 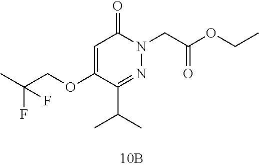 [SMILES: CCOC(=O)Cn1nc(C(C)C)c(OCC(C)(F)F)cc1=O] | 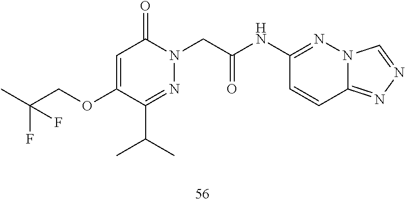 [SMILES: CC(C)c1nn(CC(=O)Nc2ccc3nncn3n2)c(=O)cc1OCC(C)(F)F] |
| 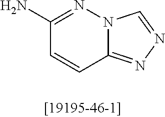 | 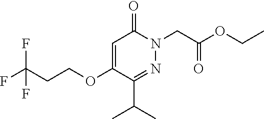 [SMILES: CCOC(=O)Cn1nc(C(C)C)c(OCCC(F)(F)F)cc1=O] | 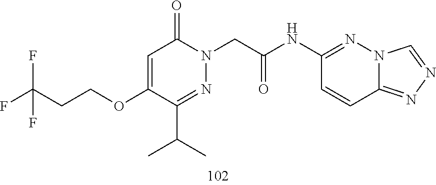 [SMILES: CC(C)c1nn(CC(=O)Nc2ccc3nncn3n2)c(=O)cc1OCCC(F)(F)F] |
| 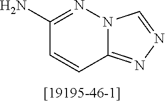 | 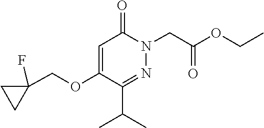 [SMILES: CCOC(=O)Cn1nc(C(C)C)c(OCC2(F)CC2)cc1=O] | 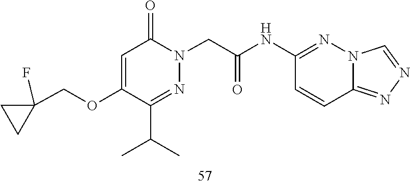 [SMILES: CC(C)c1nn(CC(=O)Nc2ccc3nncn3n2)c(=O)cc1OCC1(F)CC1] |
| 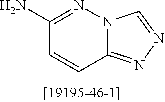 | 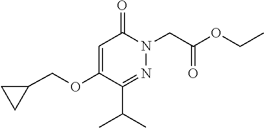 [SMILES: CCOC(=O)Cn1nc(C(C)C)c(OCC2CC2)cc1=O] | 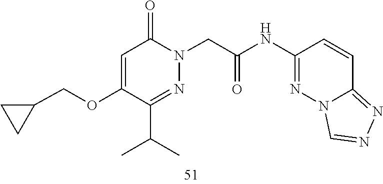 [SMILES: CC(C)c1nn(CC(=O)Nc2ccc3nncn3n2)c(=O)cc1OCC1CC1] |
| 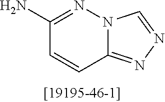 | 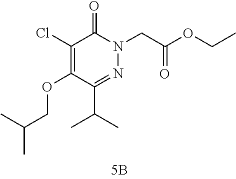 [SMILES: CCOC(=O)Cn1nc(C(C)C)c(OCC(C)C)c(Cl)c1=O] | 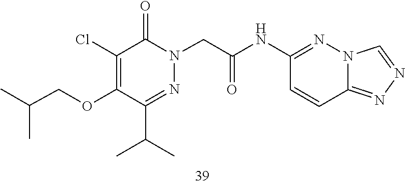 [SMILES: CC(C)COc1c(C(C)C)nn(CC(=O)Nc2ccc3nncn3n2)c(=O)c1Cl] |
| 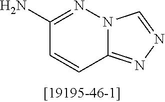 | 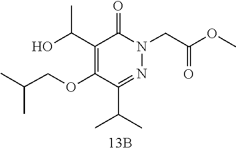 [SMILES: COC(=O)Cn1nc(C(C)C)c(OCC(C)C)c(C(C)O)c1=O] | 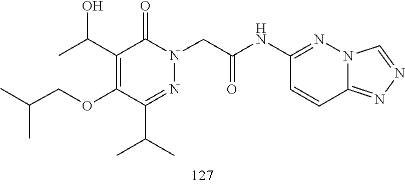 [SMILES: CC(C)COc1c(C(C)C)nn(CC(=O)Nc2ccc3nncn3n2)c(=O)c1C(C)O] |
| 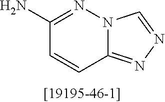 | 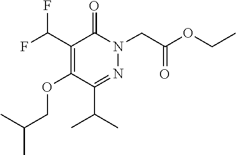 [SMILES: CCOC(=O)Cn1nc(C(C)C)c(OCC(C)C)c(C(F)F)c1=O] | 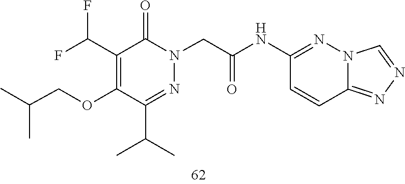 [SMILES: CC(C)COc1c(C(C)C)nn(CC(=O)Nc2ccc3nncn3n2)c(=O)c1C(F)F] |
| 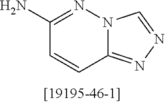 | 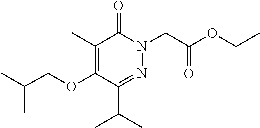 [SMILES: CCOC(=O)Cn1nc(C(C)C)c(OCC(C)C)c(C)c1=O] | 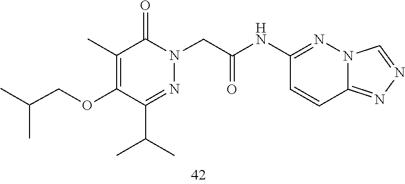 [SMILES: Cc1c(OCC(C)C)c(C(C)C)nn(CC(=O)Nc2ccc3nncn3n2)c1=O] |
| 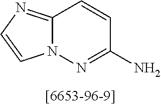 | 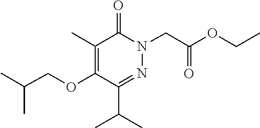 [SMILES: CCOC(=O)Cn1nc(C(C)C)c(OCC(C)C)c(C)c1=O] | 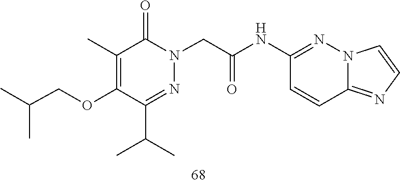 [SMILES: Cc1c(OCC(C)C)c(C(C)C)nn(CC(=O)Nc2ccc3nccn3n2)c1=O] |
| 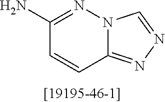 | 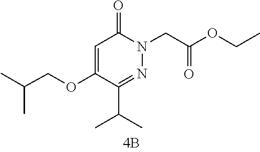 [SMILES: CCOC(=O)Cn1nc(C(C)C)c(OCC(C)C)cc1=O] | 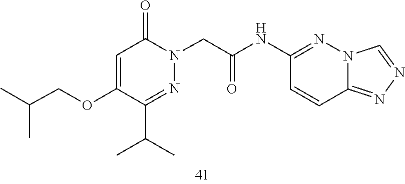 [SMILES: CC(C)COc1cc(=O)n(CC(=O)Nc2ccc3nncn3n2)nc1C(C)C] |
| 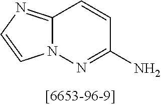 | 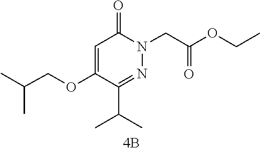 [SMILES: CCOC(=O)Cn1nc(C(C)C)c(OCC(C)C)cc1=O] | 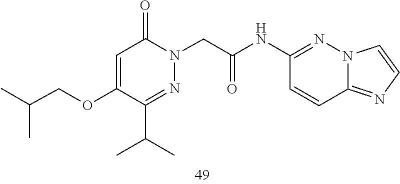 [SMILES: CC(C)COc1cc(=O)n(CC(=O)Nc2ccc3nccn3n2)nc1C(C)C] |
| 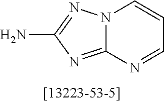 | 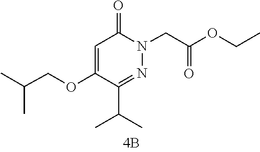 [SMILES: CCOC(=O)Cn1nc(C(C)C)c(OCC(C)C)cc1=O] | 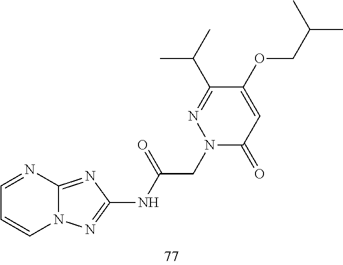 [SMILES: CC(C)COc1cc(=O)n(CC(=O)Nc2nc3ncccn3n2)nc1C(C)C] |

### Synthesis of ethyl 2-(5-bromo-3-isopropyl-4-methoxy-6-oxopyridazin-1(6H)-yl) acetate 28C

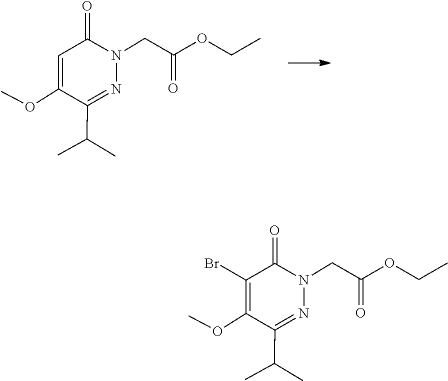 [SMILES: CCOC(=O)Cn1nc(C(C)C)c(OC)cc1=O ; CCOC(=O)Cn1nc(C(C)C)c(OC)c(Br)c1=O]

N-bromosuccinimide (629.95 mg, 3.54 mmol) was added to a stirred solution of ethyl 2-(3-isopropyl-4-methoxy-6-oxopyridazin-1(6H)-yl) acetate 1B (600 mg, 2.36 mmol) in DMF (7 mL) at RT. The mixture was stirred in a sealed tube at 75° C. for 3 h. The mixture was diluted with saturated aq. NaHCO₃ and extracted with EtOAc. The organic layer was separated, washed with brine, dried (MgSO₄), filtered and the solvents evaporated in vacuo. The residue was purified by flash column chromatography (silica; EtOAc in heptane 0/100 to 15/85). The desired fractions were collected and concentrated in vacuo to yield ethyl 2-(5-bromo-3-isopropyl-4-methoxy-6-oxopyridazin-1(6H)-yl) acetate 28C (554 mg, yield 70%) as a clear oil.

¹H NMR (300 MHz, CDCl₃) δ ppm 4.86 (s, 2H), 4.24 (q, J=7.1 Hz, 2H), 4.09 (s, 3H), 3.13 (hept, J=6.9 Hz, 1H), 1.28 (t, J=7.1 Hz, 3H), 1.20 (d, J=6.8 Hz, 6H).

Additional analogues were synthesized according to the above procedure using the

| Intermediate | Product |
|---|---|
| 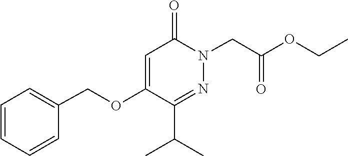 [SMILES: CCOC(=O)Cn1nc(C(C)C)c(OCc2ccccc2)cc1=O] | 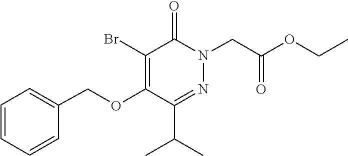 [SMILES: CCOC(=O)Cn1nc(C(C)C)c(OCc2ccccc2)c(Br)c1=O] |

### Synthesis of 2-(4-(benzyloxy)-3-isopropyl-5-methyl-6-oxopyridazin-1(6H)-yl)acetic acid

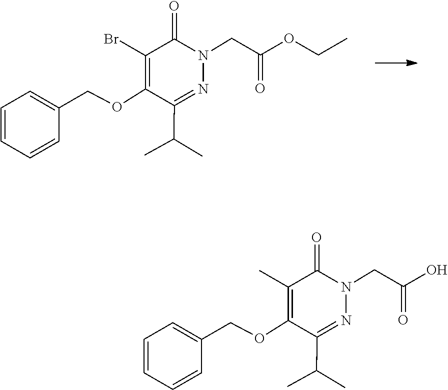 [SMILES: CCOC(=O)Cn1nc(C(C)C)c(OCc2ccccc2)c(Br)c1=O ; Cc1c(OCc2ccccc2)c(C(C)C)nn(CC(=O)O)c1=O]

Ethyl 2-(4-(benzyloxy)-5-bromo-3-isopropyl-6-oxopyridazin-1(6H)-yl)acetate (892 mg, 2.18 mmol) and methylboronic acid [13061-96-6] (333 mg, 5.45 mmol) were added to a stirred solution of sodium carbonate [497-19-8] (693 mg, 6.54 mmol) in dioxane (7 mL) and water (2 mL) under nitrogen. Then, Pd(dppf)Cl2·CH2Cl2 [95464-05-4] (89 mg, 0.11 mmol) was added. The reaction mixture was stirred at 95° C. for 18 h. The mixture was diluted with saturated aq NaHCO3 and extracted with EtOAc (×3). 2 M HCl was added to the aqueous layer until pH=2 and extracted with EtOAc (×2). The organic layer was separated, dried (MgSO4), filtered and the solvents evaporated in vacuo to yield 2-(4-(benzyloxy)-3-isopropyl-5-methyl-6-oxopyridazin-1(6H)-yl)acetic acid (231 mg, 34% yield) as a brown solid. The crude product was used in the next step without a further purification.

### Synthesis of ethyl 2-(4-isobutoxy-3-isopropyl-5-methyl-6-oxopyridazin-1(6H)-yl)acetate

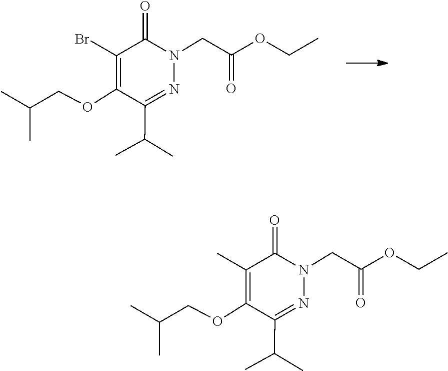

Ethyl 2-(5-bromo-4-isobutoxy-3-isopropyl-6-oxopyridazin-1(6H)-yl)acetate 6B (600 mg, 1.6 mmol) and methylboronic acid [13061-96-6] (244 mg, 4 mmol) were added to a stirred solution of sodium carbonate [497-19-8] (508 mg, 4.8 mmol) in dioxane (4.9 mL) and water (1.2 mL) under nitrogen. Then, Pd(dppf)C12 CH₂Cl2 [95464-05-4] (65 mg, 0.08 mmol) was added. The reaction mixture was stirred at 95° C. for 18 h. The mixture was diluted with saturated aq NaHCO3 and extracted with AcOEt (×3). The organic layer was separated, dried (MgSO4), filtered and the solvents evaporated in vacuo. The crude product was purified by flash column chromatography (silica 12 g; AcOEt in heptane 0/100 to 20/80). The desired fractions were collected and concentrated in vacuo to yield ethyl 2-(4-isobutoxy-3-isopropyl-5-methyl-6-oxopyridazin-1(6H)-yl)acetate (69 mg, yield 14%) as a colourless oil.

### Synthesis of 2-(5-cyclopropyl-3-isopropyl-4-methoxy-6-oxopyridazin-1(6H)-yl)acetic acid 28D

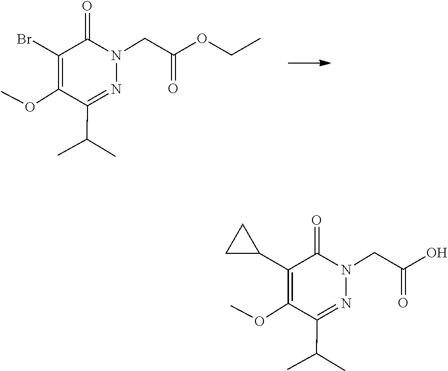 [SMILES: CCOC(=O)Cn1nc(C(C)C)c(OC)c(Br)c1=O ; COc1c(C(C)C)nn(CC(=O)O)c(=O)c1C1CC1]

Cyclopropylzinc bromide [126403-68-7] (1.11 mL, 0.5 M, 0.55 mmol) was added to a stirred solution of ethyl 2-(5-bromo-3-isopropyl-4-methoxy-6-oxopyridazin-1(6H)-yl) acetate 28C (55 mg, 0.14 mmol), bis(dibenzylideneacetone)palladium [32005-36-0] (4.0 mg, 0.0069 mmol) and 2-dicyclohexylphosphino-2′,6′-bis(N,N-dimethylamino)biphenyl (6.05 mg, 0.014 mmol). The mixture was stirred at 60° C. for 16 h. Water (3 mL) and EtOAc (4 mL) were added. Phases were separated. The aqueous phase was back extracted with EtOAc (2×4 mL). The combined organic layers were dried (Na₂SO₄), filtered and concentrated in vacuo to yield 2-(5-cyclopropyl-3-isopropyl-4-methoxy-6-oxopyridazin-1(6H)-yl)acetic acid 28D (55 mg, yield 31%, purity 21%) as a brown oil, used in the next step without further purification.

LCMS (Rₜ: 0.66, Area %: 21.10, MW: 266.00, BPM1: 267.3, Method 7)

### Synthesis of 6-chloro-3-cyclopropyl-4-ethoxy-pyridazine 29F

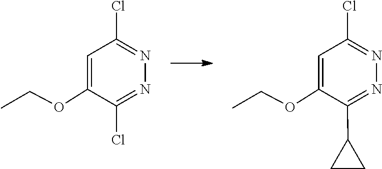

Cyclopropylboronic acid [411235-57-9] (10 g, 116.42 mmol), bis(triphenylphosphine)palladium(II) dichloride [13965-03-2] (1.5 g, 2.14 mmol) and Na₂CO₃ (20 g, 188.7 mmol) were added to a stirred solution of 3,6-dichloro-4-ethoxy-pyridazine [98142-29-1] (15 g, 77.7 mmol) in toluene (200 mL) and water (50 mL) under N₂. The mixture was stirred at 110° C. for 18 h. The mixture was extracted with EtOAc (3×300 mL), the organic layer was separated, dried (Na₂SO₄), filtered and the solvents were concentrated in vacuo. The residue was purified preparative HPLC (gradient elution: 0.1% TFA in ACN/0.1% TFA in H₂O). The desired fractions were collected, basified with NaHCO₃ solution and extracted with DCM (3×300 mL). The combined organic layers were separated, dried (Na₂SO₄), filtered and the solvents evaporated in vacuo to yield 6-chloro-3-cyclopropyl-4-ethoxy-pyridazine 29F (4.7 g, yield 31%).

LCMS (Rₜ: 1.08, Area %: 86.43, MW: 198, BPM1: 199, Method: 7)

¹H NMR (400 MHz, CDCl₃) δ ppm 1.01-1.15 (m, 2H) 1.23-1.38 (m, 2H) 1.53 (t, J=7.05 Hz, 3H) 2.39 (tt, J=8.29, 4.88 Hz, 1H) 4.13 (q, J=7.01 Hz, 2H) 6.73 (s, 1H)

### Synthesis of 6-cyclopropyl-5-ethoxypyridazin-3(2H)-one 30A

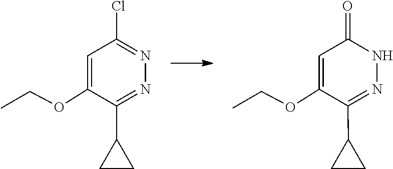

AcOH [64-19-7] (4.6 mL, 80.43 mmol) was added to a stirred solution of 6-chloro-3-cyclopropyl-4-ethoxy-pyridazine 29F (1.57 g, 7.9 mmol) in THF (3.15 mL). The mixture was stirred at 100° C. for 16 h. The solvent was concentrated in vacuo. The residue was triturated with MeOH and DIPE. The solid was dried under vacuo to yield 6-cyclopropyl-5-ethoxypyridazin-3(2H)-one 30A (630 mg, yield 44%) as an off white solid.

LCMS (Rₜ: 0.73, Area %: 100.00, MW: 180.09, BPM1: 181.1, Method 6)

¹H NMR (400 MHz, DMSO-d6) δ ppm 0.74-0.81 (m, 2H) 0.82-0.91 (m, 2H) 1.36 (t, J=7.05 Hz, 3H) 2.05 (tt, J=8.24, 5.06 Hz, 1H) 4.08 (q, J=6.94 Hz, 2H) 6.10 (s, 1H) 12.25 (br s, 1H)

### Synthesis of ethyl 2-(3-cyclopropyl-4-ethoxy-6-oxopyridazin-1(6H)-yl)acetate 30B

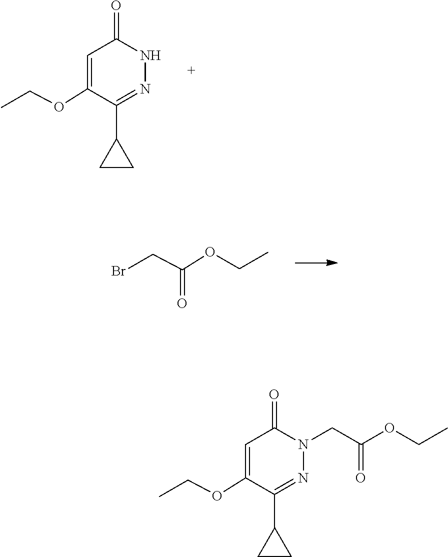

Ethyl bromoacetate [105-36-2] (310 μL, 2.8 mmol) was added to a stirred suspension of 6-cyclopropyl-5-ethoxypyridazin-3(2H)-one 30A (464 mg, 2.57 mmol) and Cs₂CO₃ [534-17-8] (1267.13 mg, 3.89 mmol) in ACN (4.65 mL). The mixture was stirred at 150° C. for 10 min under microwave irradiation. The crude was filtered through celite and washed with EtOAc (20 mL). The filtrate was concentrated in vacuo. The residue was purified by flash column chromatography (silica; EtOAc in DCM 0/100 to 50/50). The desired fractions were collected and concentrated in vacuo to yield ethyl 2-(3-cyclopropyl-4-ethoxy-6-oxopyridazin-1(6H)-yl)acetate 30B (429 mg, yield 62.57%) as a yellow solid.

LCMS (Rₜ: 1.12, Area %: 96.05, MW: 266.13, BPM1: 267.1, Method 6)

¹H NMR (400 MHz, CDCl₃) δ ppm 0.81-1.05 (m, 4H) 1.27 (t, J=7.17 Hz, 3H) 1.48 (t, J=7.05 Hz, 3H) 2.12 (tt, J=8.03, 5.26 Hz, 1H) 4.04 (q, J=6.94 Hz, 2H) 4.21 (q, J=7.17 Hz, 2H) 4.73 (s, 2H) 6.08 (s, 1H)

### Synthesis of ethyl 2-(3-cyclopropyl-4-hydroxy-6-oxopyridazin-1(6H)-yl)acetate 30C

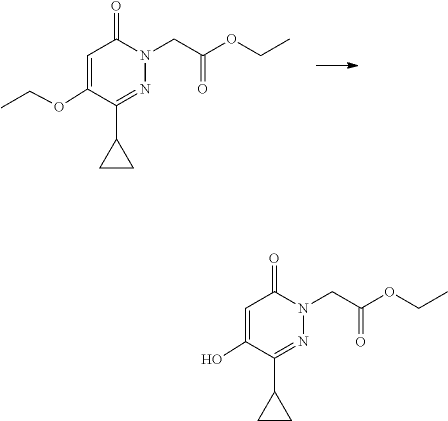 [SMILES: CCOC(=O)Cn1nc(C2CC2)c(OCC)cc1=O ; CCOC(=O)Cn1nc(C2CC2)c(O)cc1=O]

TMSI [16029-98-4] (640 μL, 4.46 mmol) was added to a solution of ethyl 2-(3-cyclopropyl-4-ethoxy-6-oxopyridazin-1(6H)-yl)acetate 30B (288 mg, 1.08 mmol) in ACN (10 mL). The mixture was heated at 130° C. for 20 min under microwave irradiation. Na₂SO₄·10H₂O was added and the mixture was stirred at RT for 1 h. The solid was filtered off and the solvent evaporated in vacuo. The residue was purified by flash column chromatography (silica; MeOH in DCM 0/100 to 20/80). The desired fractions were collected and concentrated in vacuo to yield ethyl 2-(3-cyclopropyl-4-hydroxy-6-oxopyridazin-1(6H)-yl)acetate 30C (210.6 mg, yield 82%) as a greenish solid, used in the next step without further purification.

LCMS (Rₜ: 0.73, Area %: 100.00, MW: 180.09, BPM1: 181.1, Method 6)

¹H NMR (400 MHz, DMSO-d₆) δ ppm 0.72-0.82 (m, 2H) 0.83-0.92 (m, 2H) 1.18 (t, J=7.05 Hz, 3H) 1.98-2.17 (m, 1H) 4.11 (q, J=7.09 Hz, 2H) 4.64 (s, 2H) 5.98 (s, 1H) 11.68 (s, 1H)

### Synthesis of ethyl 2-(5-bromo-3-cyclopropyl-4-hydroxy-6-oxopyridazin-1(6H)-yl)acetate 30D

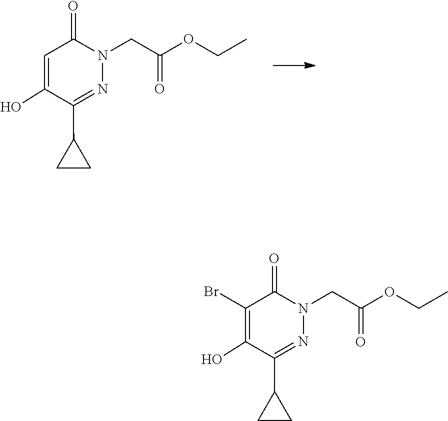 [SMILES: CCOC(=O)Cn1nc(C2CC2)c(O)cc1=O ; CCOC(=O)Cn1nc(C2CC2)c(O)c(Br)c1=O]

N-bromosuccinimide [128-08-5] (100 mg, 0.56 mmol) was added to a stirred suspension of ethyl 2-(3-cyclopropyl-4-hydroxy-6-oxopyridazin-1(6H)-yl)acetate 30C (130 mg, 0.55 mmol)) in ACN (2.6 mL). The mixture was stirred at RT for 2 h. The mixture was quenched with 2N HCl (1.5 mL) and DCM (3 mL) was added. The mixture was stirred at RT for 30 min. Phases were separated. Aqueous phase was back extracted with DCM (3×5 mL). The combined organic layers were dried (Na₂SO₄), filtered and concentrated in vacuo to yield ethyl 2-(5-bromo-3-cyclopropyl-4-hydroxy-6-oxopyridazin-1(6H)-yl)acetate 30D (151.8 mg, yield 88%) as a yellow solid.

LCMS (Rₜ: 0.56, Area %: 80.84, MW: 316.01, BPM1: 317.0, Method 5)

¹H NMR (500 MHz, DMSO-d₆) δ ppm 0.73-0.83 (m, 2H) 0.85-0.97 (m, 2H) 1.15-1.26 (m, 3H) 2.16 (tt, J=8.14, 5.13 Hz, 1H) 4.06-4.21 (m, 2H) 4.70-4.79 (m, 2H)

### Synthesis of acetate ethyl 2-(3-cyclopropyl-6-oxo-4-((tetrahydro-2H-pyran-4-yl)oxy)pyridazin-1(6H)-yl)acetate 30E

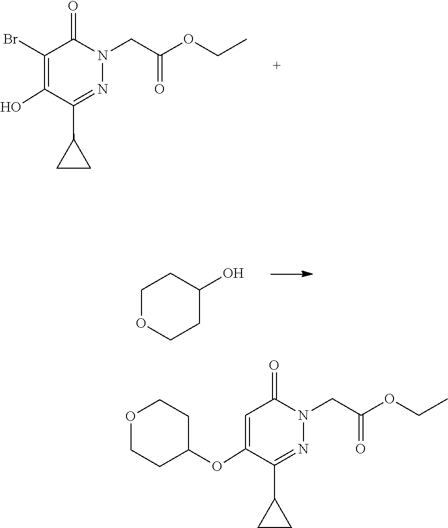 [SMILES: CCOC(=O)Cn1nc(C2CC2)c(O)c(Br)c1=O ; OC1CCOCC1 ; CCOC(=O)Cn1nc(C2CC2)c(OC2CCOCC2)cc1=O]

Di-tert-butyl azodicarboxylate [870-50-8] (103.5 mg, 0.45 mmol) was added to a stirred suspension of ethyl 2-(5-bromo-3-cyclopropyl-4-hydroxy-6-oxopyridazin-1(6H)-yl)acetate 30D (95 mg, 0.3 mmol), tetrahydro-2H-pyran-4-ol [2081-44-9] (35 μL, 0.37 mmol) and PPh₃ [603-35-0] (120 mg, 0.46 mmol) in THF (2.5 mL). The mixture was stirred at 120° C. for 20 min under microwave irradiation and at 150° C. for 20 min under microwave irradiation. The mixture was diluted with EtOAc and washed with a sat. solution of NaHCO₃. The organic layer was separated, dried (Na₂SO₄), filtered and concentrated in vacuo. The residue was purified by flash column chromatography (silica; EtOAc in DCM, 0/100 to 100/0). The desired fractions were collected and concentrated in vacuo to yield ethyl 2-(3-cyclopropyl-6-oxo-4-((tetrahydro-2H-pyran-4-yl)oxy)pyridazin-1(6H)-yl)acetate 30E (31.8 mg, yield 26%, purity 79%) as a yellow oil, used in the next step without further purification.

LCMS (Rₜ: 1.12, Area %: 79.28, MW: 322.11, BPM1: 323.2, Method 7)

### Synthesis of 2-(3-cyclopropyl-6-oxo-4-((tetrahydro-2H-pyran-4-yl)oxy)pyridazin-1(6H)-yl)acetic acid 30F

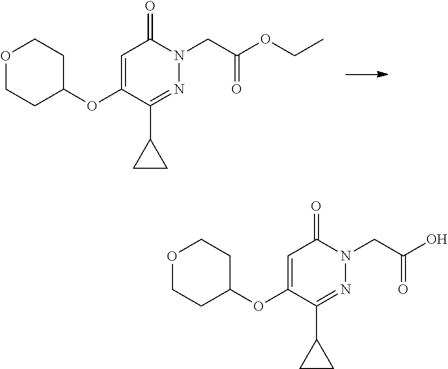 [SMILES: CCOC(=O)Cn1nc(C2CC2)c(OC2CCOCC2)cc1=O ; O=C(O)Cn1nc(C2CC2)c(OC2CCOCC2)cc1=O]

LiOH [1310-65-2] (12 mg, 0.5 mmol) in water (0.11 mL) was added to a stirred solution of ethyl 2-(3-cyclopropyl-6-oxo-4-((tetrahydro-2H-pyran-4-yl)oxy)pyridazin-1(6H)-yl)acetate 30E (31.8 mg, 0.1 mmol) in 1,4-dioxane (0.2 mL). The mixture was stirred at RT for 16 h. The solvent was concentrated in vacuo. The residue was treated with 2 N HCl (2 mL) and extracted with EtOAc (3×2 mL) and DCM/MeOH (9.5/0.5) (2 mL). The combined organic layers were dried (Na₂SO₄), filtered and concentrated in vacuo to yield 2-(3-cyclopropyl-6-oxo-4-((tetrahydro-2H-pyran-4-yl)oxy)pyridazin-1(6H)-yl)acetic acid 30F (31.9 mg, yield 76%, purity 83%) as a yellow oil.

LCMS (Rₜ: 0.47, Area %: 82.62, MW: 294.12, BPM1: 295.1, Method 6)

Additional analogues were synthesized according to the above procedure, using the appropriate reagents.

| Intermediate | Product |
|---|---|
| 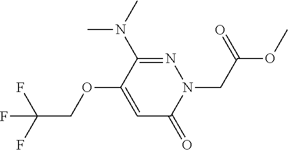 [SMILES: COC(=O)Cn1nc(N(C)C)c(OCC(F)(F)F)cc1=O] | 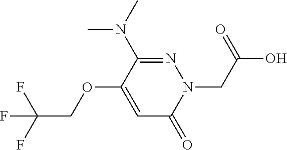 [SMILES: CN(C)c1nn(CC(=O)O)c(=O)cc1OCC(F)(F)F] |
| 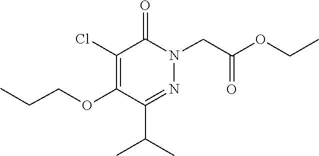 [SMILES: CCCOc1c(C(C)C)nn(CC(=O)OCC)c(=O)c1Cl] | 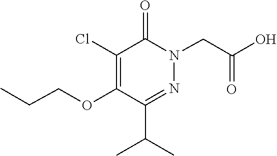 [SMILES: CCCOc1c(C(C)C)nn(CC(=O)O)c(=O)c1Cl] |
| 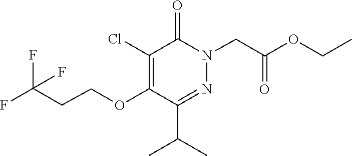 [SMILES: CCOC(=O)Cn1nc(C(C)C)c(OCCC(F)(F)F)c(Cl)c1=O] | 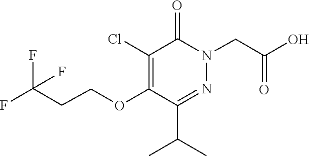 [SMILES: CC(C)c1nn(CC(=O)O)c(=O)c(Cl)c1OCCC(F)(F)F] |
| 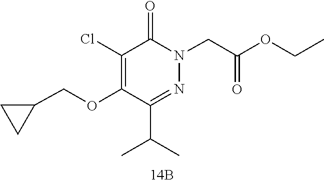 [SMILES: CCOC(=O)Cn1nc(C(C)C)c(OCC2CC2)c(Cl)c1=O] | 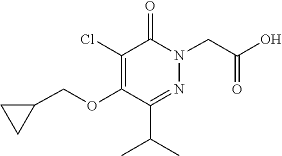 [SMILES: CC(C)c1nn(CC(=O)O)c(=O)c(Cl)c1OCC1CC1] |
| 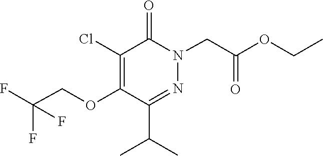 [SMILES: CCOC(=O)Cn1nc(C(C)C)c(OCC(F)(F)F)c(Cl)c1=O] | 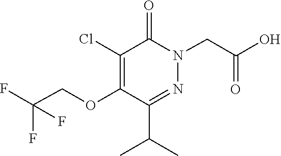 [SMILES: CC(C)c1nn(CC(=O)O)c(=O)c(Cl)c1OCC(F)(F)F] |
| 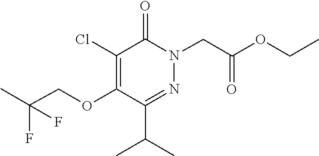 [SMILES: CCOC(=O)Cn1nc(C(C)C)c(OCC(C)(F)F)c(Cl)c1=O] | 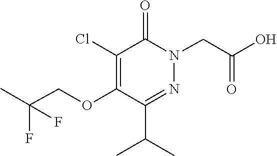 [SMILES: CC(C)c1nn(CC(=O)O)c(=O)c(Cl)c1OCC(C)(F)F] |
| 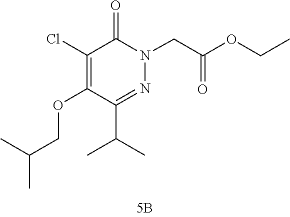 [SMILES: CCOC(=O)Cn1nc(C(C)C)c(OCC(C)C)c(Cl)c1=O] | 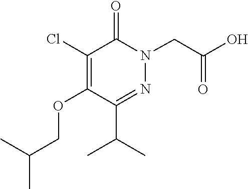 [SMILES: CC(C)COc1c(C(C)C)nn(CC(=O)O)c(=O)c1Cl] |
| 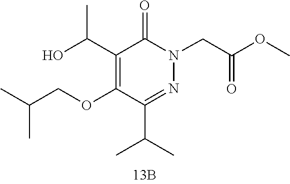 [SMILES: COC(=O)Cn1nc(C(C)C)c(OCC(C)C)c(C(C)O)c1=O] | 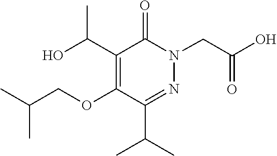 [SMILES: CC(C)COc1c(C(C)C)nn(CC(=O)O)c(=O)c1C(C)O] |
| 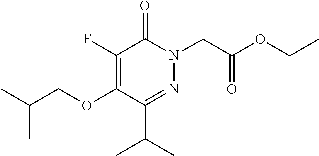 [SMILES: CCOC(=O)Cn1nc(C(C)C)c(OCC(C)C)c(F)c1=O] | 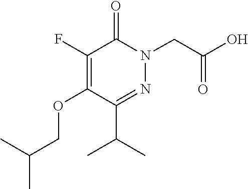 [SMILES: CC(C)COc1c(C(C)C)nn(CC(=O)O)c(=O)c1F] |
| 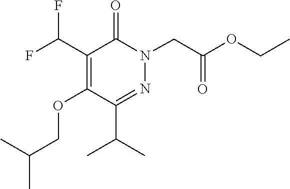 [SMILES: CCOC(=O)Cn1nc(C(C)C)c(OCC(C)C)c(C(F)F)c1=O] | 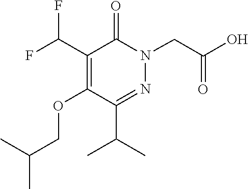 [SMILES: CC(C)COc1c(C(C)C)nn(CC(=O)O)c(=O)c1C(F)F] |
| 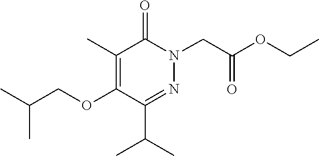 [SMILES: CCOC(=O)Cn1nc(C(C)C)c(OCC(C)C)c(C)c1=O] | 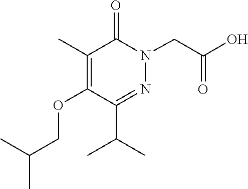 [SMILES: Cc1c(OCC(C)C)c(C(C)C)nn(CC(=O)O)c1=O] |
| 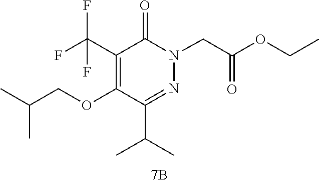 [SMILES: CCOC(=O)Cn1nc(C(C)C)c(OCC(C)C)c(C(F)(F)F)c1=O] | 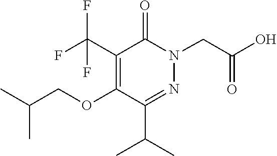 [SMILES: CC(C)COc1c(C(C)C)nn(CC(=O)O)c(=O)c1C(F)(F)F] |
| 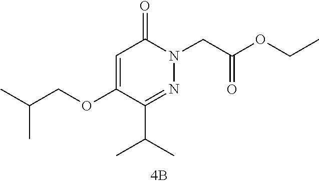 [SMILES: CCOC(=O)Cn1nc(C(C)C)c(OCC(C)C)cc1=O] | 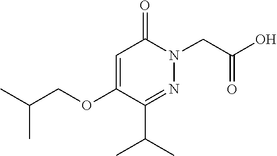 [SMILES: CC(C)COc1cc(=O)n(CC(=O)O)nc1C(C)C] |
| 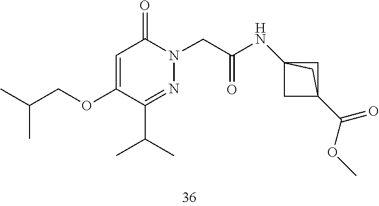 [SMILES: COC(=O)C12CC(NC(=O)Cn3nc(C(C)C)c(OCC(C)C)cc3=O)(C1)C2] | 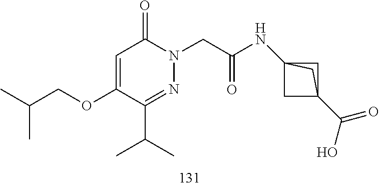 [SMILES: CC(C)COc1cc(=O)n(CC(=O)NC23CC(C(=O)O)(C2)C3)nc1C(C)C] |
| 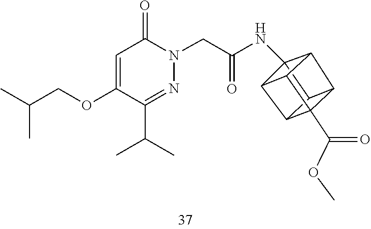 [SMILES: COC(=O)C12C3C4C1C1C2C3C41NC(=O)Cn1nc(C(C)C)c(OCC(C)C)cc1=O] | 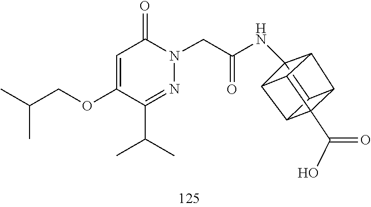 [SMILES: CC(C)COc1cc(=O)n(CC(=O)NC23C4C5C2C2C3C4C52C(=O)O)nc1C(C)C] |
| 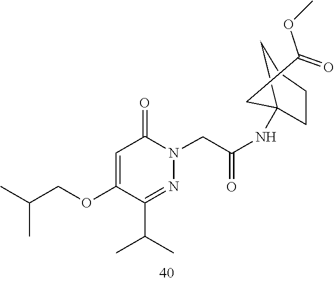 [SMILES: COC(=O)CC1(NC(=O)Cn2nc(C(C)C)c(OCC(C)C)cc2=O)CCCC1] | 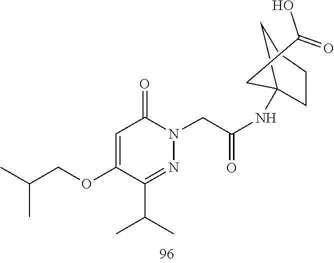 [SMILES: CC(C)COc1cc(=O)n(CC(=O)NC2(CC(=O)O)CCCC2)nc1C(C)C] |
| 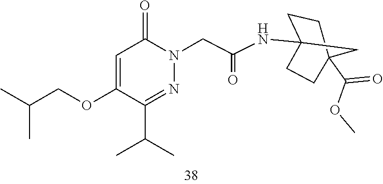 [SMILES: COC(=O)C12CCC(NC(=O)Cn3nc(C(C)C)c(OCC(C)C)cc3=O)(CC1)C2] | 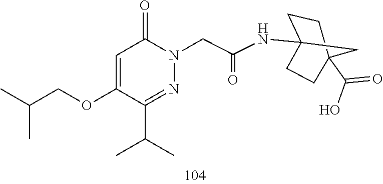 [SMILES: CC(C)COc1cc(=O)n(CC(=O)NC23CCC(C(=O)O)(CC2)C3)nc1C(C)C] |
| 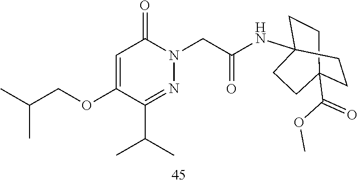 [SMILES: COC(=O)C12CCC(NC(=O)Cn3nc(C(C)C)c(OCC(C)C)cc3=O)(CC1)CC2] | 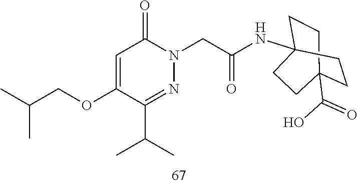 [SMILES: CC(C)COc1cc(=O)n(CC(=O)NC23CCC(C(=O)O)(CC2)CC3)nc1C(C)C] |
| 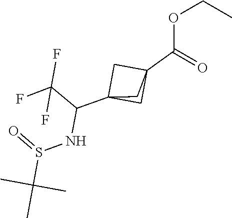 [SMILES: CCOC(=O)C12CC(C(NS(=O)C(C)(C)C)C(F)(F)F)(C1)C2] | 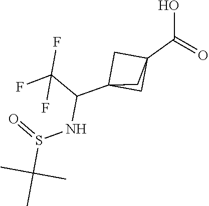 [SMILES: CC(C)(C)S(=O)NC(C(F)(F)F)C12CC(C(=O)O)(C1)C2] |
| 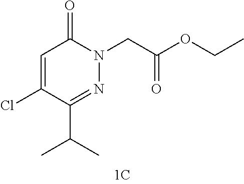 [SMILES: CCOC(=O)Cn1nc(C(C)C)c(Cl)cc1=O] | 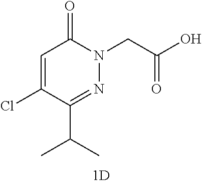 |
| 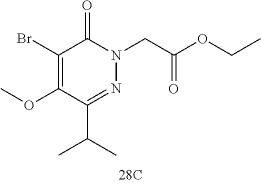 [SMILES: CCOC(=O)Cn1nc(C(C)C)c(OC)c(Br)c1=O] | 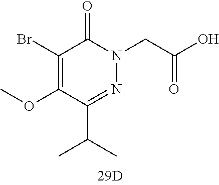 [SMILES: COc1c(C(C)C)nn(CC(=O)O)c(=O)c1Br] |

### Synthesis of 1,3-dioxoisoindolin-2-yl 3-(2-(4-isobutoxy-3-isopropyl-6-oxopyridazin-1(6H)-yl)acetamido)bicyclo[1.1.1]pentane-1-carboxylate

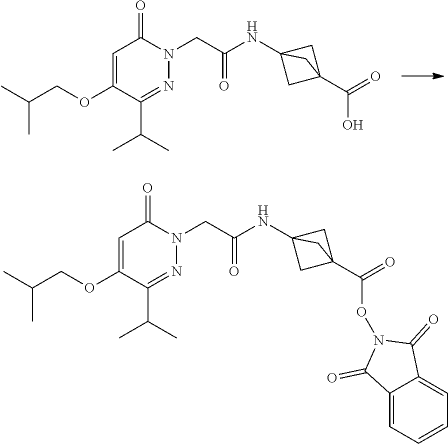

DIC [693-13-0] (200 μL, 0.81 g/mL, 1.28 mmol) was added dropwise to a stirring solution of 3-(2-(4-isobutoxy-3-isopropyl-6-oxopyridazin-1(6H)-yl)acetamido)bicyclo[1.1.1]pentane-1-carboxylic acid 131 (528 mg, 1.27 mmol), N-hydroxyphthalimide [524-38-9] (208 mg, 1.28 mmol) and DMAP [1122-58-3] (15.6 mg, 0.013 mmol) in DCM (2.6 mL). The resulting light yellow reaction mixture was stirred at rt for 72h. The mixture was filtered through celite and the filtrate was removed in vacuo. The crude product was purified by flash column chromatography (silica, EtOAc in Heptane 0/100 to 30/70). The desired fractions were collected and concentrated in vacuo to yield 1,3-dioxoisoindolin-2-yl 3-(2-(4-isobutoxy-3-isopropyl-6-oxopyridazin-1(6H)-yl)acetamido)bicyclo[1.1.1]pentane-1-carboxylate (392 mg, yield 59%) as a white solid and 1,3-dioxoisoindolin-2-yl 3-(2-(4-isobutoxy-3-isopropyl-6-oxopyridazin-1(6H)-yl)acetamido)bicyclo[1.1.1]pentane-1-carboxylate (189.9 mg, yield 29%) as a colorless oil.

### Synthesis of 2-(4-isobutoxy-3-isopropyl-6-oxopyridazin-1(6H)-yl)-N-(3-(propylsulfonyl)bicyclo[1.1.1]pentan-1-yl)acetamide 78

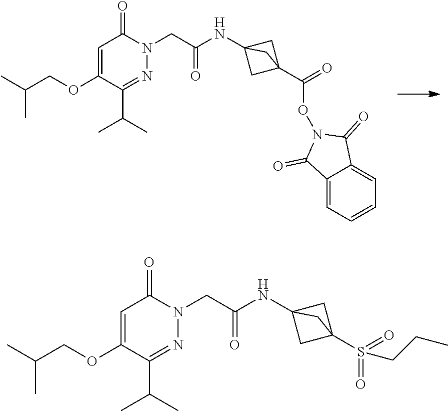 [SMILES: CC(C)COc1cc(=O)n(CC(=O)NC23CC(C(=O)ON4C(=O)c5ccccc5C4=O)(C2)C3)nc1C(C)C ; CCCS(=O)(=O)C12CC(NC(=O)Cn3nc(C(C)C)c(OCC(C)C)cc3=O)(C1)C2]

Dibutyl phosphate [107-66-4] (37.95 μL, 1.06 g/mL, 0.19 mmol), DME (0.56 mL) and ACN (0.56 mL) were added to a mixture of 1,3-dioxoisoindolin-2-yl 3-(2-(4-isobutoxy-3-isopropyl-6-oxopyridazin-1(6H)-yl)acetamido)bicyclo[1.1.1]pentane-1-carboxylate (50 mg, 0.096 mmol), sodium propane-2-sulfinate [4160-19-4] (25.3 mg, 0.19 mmol), 4CZIPN [1416881-52-1] (1.51 mg, 0.0019 mmol) and copper(II) trifluoromethanesulfonate [34946-82-2] (6.92 mg, 0.019 mmol) under N2. The mixture was place in a Penn reactor, Blue LED (100%), 6800 FAN for 12h. Water and DCM were added. Phases were put in a phase separator cartridge eluting with more DCM. The organic phase was concentrated in vacuo and the residue was purified by RP HPLC. Conditions: Stationary phase: C18 XBridge 30×100 mm 10 μm. Mobile phase: NH4HCO3 0.25% solution in Water and CH3CN, yielding 2-(4-isobutoxy-3-isopropyl-6-oxopyridazin-1(6H)-yl)-N-(3-(propylsulfonyl)bicyclo[1.1.1]pentan-1-yl)acetamide 78 (8.2 mg, yield 20%) as a white solid.

Additional analogues were synthesized according to the above procedure, using the appropriate reagents.

| Reagent | Product |
|---|---|
| 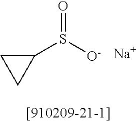 | 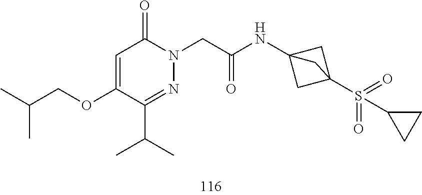 [SMILES: CC(C)COc1cc(=O)n(CC(=O)NC23CC(S(=O)(=O)C4CC4)(C2)C3)nc1C(C)C] |

### Synthesis of methyl 3-(3-(2-(4-isobutoxy-3-isopropyl-6-oxopyridazin-1(6H)-yl)acetamido)bicyclo[1.1.1]pentan-1-yl)-3-oxopropanoate

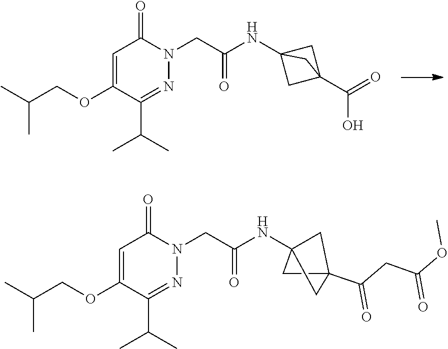 [SMILES: CC(C)COc1cc(=O)n(CC(=O)NC23CC(C(=O)O)(C2)C3)nc1C(C)C ; COC(=O)CC(=O)C12CC(NC(=O)Cn3nc(C(C)C)c(OCC(C)C)cc3=O)(C1)C2]

1,3-Dicyclohexylcarbodiimide [538-75-0] (90.53 mg, 0.44 mmol) was added to a solution of 3-(2-(4-isobutoxy-3-isopropyl-6-oxopyridazin-1(6H)-yl)acetamido)bicyclo[1.1.1]pentane-1-carboxylic acid 131 (150 mg, 0.37 mmol), 2,2-dimethyl-1,3-dioxane-4,6-dione [2033-24-1] (57.97 mg, 0.4 mmol) and 4-dimethylaminopyridine [1122-58-3] (67 mg, 0.55 mmol) in DCM (1.9 mL) and DMF (0.5 mL) at 0° C., then the RM was stirred for 1h and kept at 5° C. overnight (fridge). The precipitate (DCU) was filtered off and the filtrate was washed with HCl 1N and brine, dried over Na2SO4, filtered and the solvent was evaporated till dryness to yield a yellow solid which was dissolved in MeOH (0.75 mL). The mixture was stirred at 70° C. for 16h. The solvent was concentrated in vacuo and the residue dried under vacuo to yield methyl 3-(3-(2-(4-isobutoxy-3-isopropyl-6-oxopyridazin-1(6H)-yl)acetamido)bicyclo[1.1.1]pentan-1-yl)-3-oxopropanoate (120 mg, yield 76%) as a yellow wax, used in the next step without further purification.

### Synthesis of 2-(4-isobutoxy-3-isopropyl-6-oxopyridazin-1(6H)-yl)-N-(3-(1-methyl-5-oxo-4,5-dihydro-1H-pyrazol-3-yl)bicyclo[1.1.1]pentan-1-yl)acetamide 98

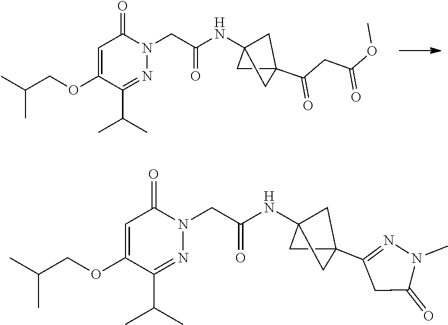

Methylhydrazine (25 μL, 0.88 g/mL, 0.47 mmol) was added dropwise to a solution of methyl 3-(3-(2-(4-isobutoxy-3-isopropyl-6-oxopyridazin-1(6H)-yl)acetamido)bicyclo[1.1.1]pentan-1-yl)-3-oxopropanoate (50 mg, 0.12 mmol) in EtOH (0.5 mL) and acetic acid (0.05 mL). The mixture was stirred at RT for 1h. The solvent was concentrated in vacuo and sent to RP HPLC. Conditions: Stationary phase: C18 XBridge 30×100 mm 10 μm. Mobile phase: NH4HCO3 0.25% solution in Water and CH₃CN, yielding a compound which was dissolved in MeOH and passed through a SCX-2 cartridge eluting with 7N solution of ammonia in MeOH. The solvent was concentrated in vacuo to yield 2-(4-isobutoxy-3-isopropyl-6-oxopyridazin-1(6H)-yl)-N-(3-(1-methyl-5-oxo-4,5-dihydro-1H-pyrazol-3-yl)bicyclo[1.1.1]pentan-1-yl)acetamide 98 (6.3 mg, yield 12%) as a solid.

### Synthesis of 2-(3-cyclopropyl-4-ethoxy-6-oxopyridazin-1(6H)-yl)acetic acid 33C

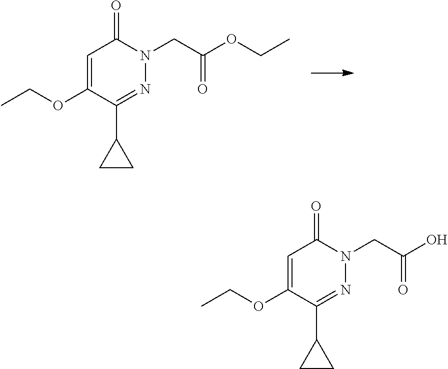

LiOH [1310-65-2] (113 mg, 4.72 mmol) in water (1 mL) was added to a stirred solution of ethyl 2-(3-cyclopropyl-4-ethoxy-6-oxopyridazin-1(6H)-yl)acetate 30B (250 mg, 0.94 mmol) in 1,4-dioxane (1.55 mL). The mixture was stirred at 70° C. for 3 h. The solvent was concentrated in vacuo. The residue was treated with 2 N HCl (1 mL) and extracted with EtOAc (3×5 mL) and THF/EtOAc (3/7) (1×5 mL). The combined organic layers were dried (Na₂SO₄), filtered and concentrated in vacuo. The residue was triturated with Et₂O to yield 2-(3-cyclopropyl-4-ethoxy-6-oxopyridazin-1(6H)-yl)acetic acid 33C (100 mg, yield 44.71%, purity 84%) as a brown solid, used in the next step without further purification.

LCMS (Rₜ: 0.52, Area %: 84, MW: 238.00, BPM1: 239.3, Method 7)

### Synthesis of dimethyl 2-phenoxyfumarate 34A

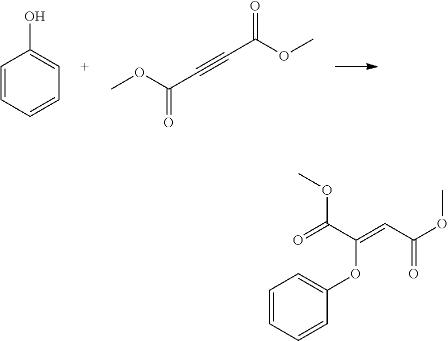

Pyridine [110-86-1] (1.57 mL, 19.52 mmol) was added to a stirred solution of dimethyl acetylenedicarboxylate [762-42-5] (4 mL, 32.54 mmol) and phenol [108-95-2] (3.06 g, 32.54 mmol) in THF (125 mL) under nitrogen. The mixture was stirred at RT for 16 h. The mixture was concentrated in vacuo and the residue was purified by flash column chromatography (silica; EtOAc in Heptane 0/100 10/90). The desired fractions were collected and the solvents evaporated in vacuo to yield dimethyl 2-phenoxyfumarate 34A (7.11 g, 92.5%) as a white solid.

¹H NMR (400 MHz, CDCl₃) δ ppm 3.71 (s, 3H) 3.74 (s, 3H) 6.60 (s, 1H) 6.96 (d, J=8.67 Hz, 2H) 7.08 (t, J=7.37 Hz, 1H) 7.31 (t, J=7.80 Hz, 2H)

### Synthesis of 2-phenoxyfumaric acid 34B

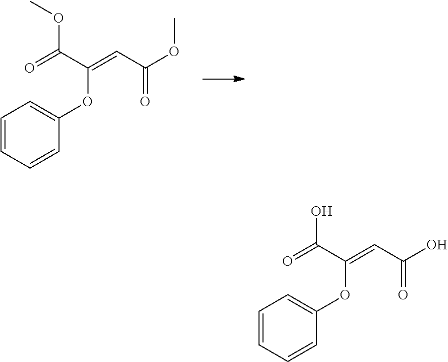 [SMILES: COC(=O)/C=C(\Oc1ccccc1)C(=O)OC ; O=C(O)/C=C(\Oc1ccccc1)C(=O)O]

KOH [1310-58-3] (14 mL, 84.67 mmol) was added to a stirred solution of dimethyl 2-phenoxyfumarate 34A (2.0 g, 8.47 mmol) in MeOH (22.3 mL). The mixture was stirred at RT for 16 h. The mixture was cooled to 0° C. and acidified with ION HCl till pH=2. The aqueous layer was extracted with Et₂O (3×30 ml). The combined organic layers were dried (Na2SO₄), filtered and concentrated in vacuo to yield 2-phenoxyfumaric acid 34B (1760 mg, yield 100%) as a light yellow solid, used in the next step without further purification.

LCMS (Rₜ: 0.17, Area %: 100, MW: 208, BPM2: 207.3, Method 7)

### Synthesis of 3-phenoxyfuran-2,5-dione 34C

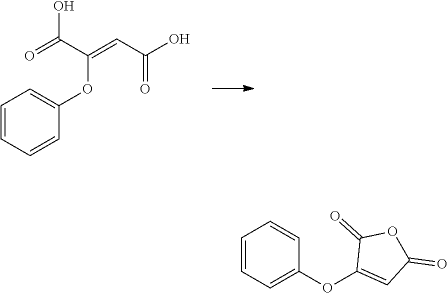 [SMILES: O=C(O)/C=C(\Oc1ccccc1)C(=O)O ; O=C1C=C(Oc2ccccc2)C(=O)O1]

A mixture of 2-phenoxyfumaric acid 34B (1.02 g, 4.9 mmol) and SOCl₂ [7719-09-7](6.44 mL, 1.63 g/mL, 88.2 mmol) was stirred at RT for 1 h and at 80° C. for 24 h. The solvent was concentrated in vacuo. The residue was dissolved in DCM and washed with a sat sol of NaHCO₃. The organic layer was separated, dried (Na₂SO₄), filtered and concentrated in vacuo to 3-phenoxyfuran-2,5-dione 34C (1.07 g, yield quant.) as a light yellow solid, used in the next step without further purification.

¹H NMR (400 MHz, CDCl₃) δ ppm 5.63 (s, 1H) 7.17-7.24 (m, 2H) 7.33-7.44 (m, 1H) 7.45-7.56 (m, 2H)

### Synthesis of ethyl 2-(3,6-dioxo-5-phenoxy-3,6-dihydropyridazin-1(2H)-yl)acetate 34D1 and ethyl 2-(3,6-dioxo-4-phenoxy-3,6-dihydropyridazin-1(2H)-yl)acetate 34D2

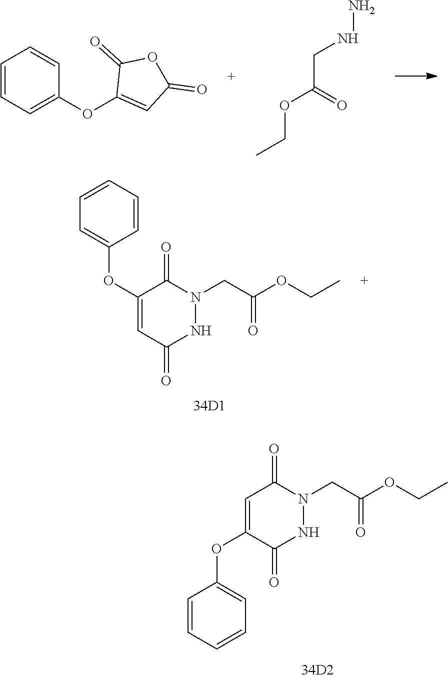

Ethyl hydrazinoacetate hydrochloride [637-80-9] (614 mg, 3.97 mmol) was added to a stirred suspension of 3-phenoxyfuran-2,5-dione 34C (803 mg, 3.97 mmol) in AcOH [64-19-7] (3.60 mL, 63.51 mmol). The mixture was stirred at 70° C. for 16 h. The solvent was concentrated in vacuo and co-evaporated with toluene. The crude (1.07 g) was used without further purification in the next step.

SOCl₂ [7719-09-7] (0.44 mL, 1.64 g/mL, 6.1 mmol) was added dropwise to a stirred solution of the previous crude (1.07 mg, 4.07 mmol) in EtOH [64-17-5] (22.0 mL) at 0° C. Then the mixture was stirred at 70° C. for 16 h. The solvent was concentrated in vacuo to yield ethyl 2-(3,6-dioxo-5-phenoxy-3,6-dihydropyridazin-1(2H)-yl)acetate 34D1 and ethyl 2-(3,6-dioxo-4-phenoxy-3,6-dihydropyridazin-1(2H)-yl)acetate 34D2 (1.18 g, yield 88%, purity 86%, ratio 34D1/34D2: 8/2) as a light yellow solid, used in the next step without further purification.

LCMS (34D1 (Rₜ: 0.53, Area %: 68, MW: 290.00, BPM1: 291.2, BPM2: 289.2, Method 7) 34D2 (Rₜ: 0.39, Area %: 18, MW: 290.00, BPM2: 291.2, BPM2: 289.2, Method 7)).

### Synthesis of ethyl 2-(6-oxo-5-phenoxy-3-(((trifluoromethyl)sulfonyl)oxy)pyridazin-1(6H)-yl)acetate 34E1 and ethyl 2-(6-oxo-4-phenoxy-3-(((trifluoromethyl)sulfonyl)oxy)pyridazin-1(6H)-yl)acetate 34E2

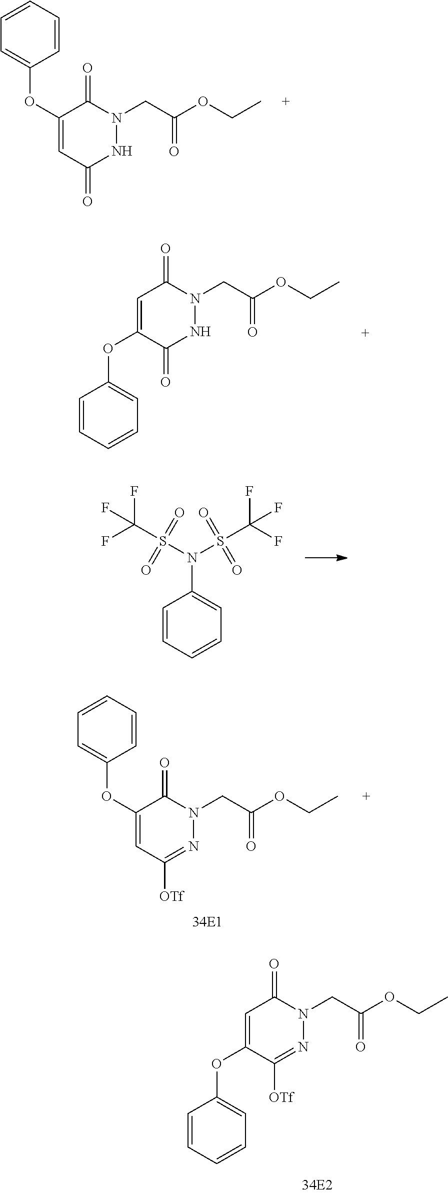 [SMILES: CCOC(=O)Cn1[nH]c(=O)cc(Oc2ccccc2)c1=O ; CCOC(=O)Cn1[nH]c(=O)c(Oc2ccccc2)cc1=O ; O=S(=O)(N(c1ccccc1)S(=O)(=O)C(F)(F)F)C(F)(F)F ; CCOC(=O)Cn1nc(OS(=O)(=O)C(F)(F)F)cc(Oc2ccccc2)c1=O ; CCOC(=O)Cn1nc(OS(=O)(=O)C(F)(F)F)c(Oc2ccccc2)cc1=O]

N-phenyltrifluoromethanesulfonimide [37595-74-7] (1.74 g, 4.88 mmol) was added to a mixture of ethyl 2-(3,6-dioxo-5-phenoxy-3,6-dihydropyridazin-1(2H)-yl)acetate 34D1 and ethyl 2-(3,6-dioxo-4-phenoxy-3,6-dihydropyridazin-1(2H)-yl)acetate 34D2 (1.18 g, 4.07 mmol) and K₂CO₃ [584-08-7] (1.12 g, 8.13 mmol) in THF (16.7 mL). The mixture was heated at 120° C. for 10 min under microwave irradiation. The mixture was diluted with water (50 mL) and extracted with EtOAc (3×20 mL). The combined organic layer was separated, dried (Na₂SO₄), filtered and the solvent evaporated in vacuo. The residue was purified by flash column chromatography (silica; EtOAc in DCM 0/100 to 30/70). The desired fractions were collected and concentrated in vacuo to yield to a mixture of ethyl 2-(6-oxo-5-phenoxy-3-(((trifluoromethyl)sulfonyl)oxy)pyridazin-1(6H)-yl)acetate 34E1 and ethyl 2-(6-oxo-4-phenoxy-3-(((trifluoromethyl)sulfonyl)oxy)pyridazin-1(6H)-yl)acetate 34E2 (1.19 g, yield 60%, purity 86%, ratio 34E1/34E2: 8/2) as yellow oil, used in the next step without further purification.

LCMS (34E1 (Rₜ: 1.51, Area %: 69, MW: 422.00, BPM1: 423.1, Method 7) 34E2 (Rₜ: 1.57, Area %: 17, MW: 422.00, BPM2: 423.1, Method 7))

### Synthesis of ethyl 2-(6-oxo-5-phenoxy-3-(prop-1-en-2-yl)pyridazin-1(6H)-yl)acetate 34F1 and ethyl 2-(6-oxo-4-phenoxy-3-(prop-1-en-2-yl)pyridazin-1(6H)-yl)acetate 34F2

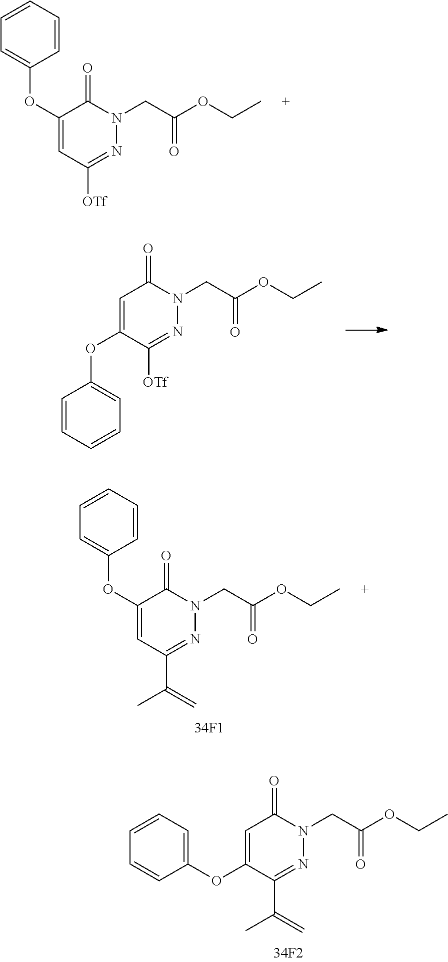

Bis(triphenylphosphine)palladium(II) dichloride [13965-03-2] (119 mg, 0.17 mmol) was added to a stirred mixture of ethyl 2-(6-oxo-5-phenoxy-3-(((trifluoromethyl)sulfonyl)oxy)pyridazin-1(6H)-yl)acetate 34E1 and ethyl 2-(6-oxo-4-phenoxy-3-(((trifluoromethyl)sulfonyl)oxy)pyridazin-1(6H)-yl)acetate 34E2 (1.19 g, 1.24 mmol), 4,4,5,5-tetramethyl-2-(prop-1-en-2-yl)-1,3,2-dioxaborolane [126726-62-3](690 μL, 4.02 mmol) and 2M K₂CO₃ [584-08-7] (2.4 mL, 4.8 mmol) aqueous solution in 1,4-dioxane (21.5 mL). The mixture was stirred at 85° C. for 16 h. Water (30 mL) and EtOAc (50 mL) were added. The organic layer was separated. The aqueous phase was further extracted with EtOAc (30 mL). The combined organic layers were dried (Na2SO₄), filtered and concentrated in vacuo. The residue was purified by flash column chromatography (silica; EtOAc in DCM 0/100 to 50/50). The desired fractions were collected and concentrated in vacuo to yield ethyl 2-(6-oxo-5-phenoxy-3-(prop-1-en-2-yl)pyridazin-1(6H)-yl)acetate 34F1 (432 mg, yield 49%, pure) and a mixture of ethyl 2-(6-oxo-5-phenoxy-3-(prop-1-en-2-yl)pyridazin-1(6H)-yl)acetate 34F1 and ethyl 2-(6-oxo-4-phenoxy-3-(prop-1-en-2-yl)pyridazin-1(6H)-yl)acetate 34F2 (143 mg, yield 16%, purity 98%, ratio: 34F1/34F2: 50/50) as yellow oils.

Analysis of 34F1:

LCMS (Rₜ: 2.52, Area %: 96.24, MW: 314.00, BPM1: 315.2, Method 9)

¹H NMR (500 MHz, CDCl₃) δ ppm 1.31 (t, J=7.2 Hz, 3H) 2.01 (s, 3H) 4.27 (q, J=7.2 Hz, 2H) 4.95 (s, 2H) 5.20 (s, 1H) 5.22 (br q, J=1.4 Hz, 1H) 6.66 (s, 1H) 7.11-7.16 (m, 2H) 7.27-7.32 (m, 1H) 7.41-7.48 (m, 2H)

Analysis of a mixture of 34F1 and 34F2:

LCMS (Two products in the same peak (Rₜ: 2.52, Area %: 98.45, MW: 314.13, BPM1: 315.2, BPM2: 315.2, Method 9))

¹H NMR (500 MHz, CDCl₃) δ ppm 1.29 (t, J=7.2 Hz, 3H) 1.31 (t, J=7.2 Hz, 3H) 2.02 (s, 3H) 2.14-2.17 (m, 3H) 4.19-4.31 (m, 4H) 4.85 (s, 2H) 4.95 (s, 2H) 5.20 (s, 1H) 5.22 (br q, J=1.4 Hz, 1H) 5.50 (quin, J=1.4 Hz, 1H) 5.82-5.85 (m, 1H) 5.94 (s, 1H) 6.66 (s, 1H) 7.07-7.12 (m, 2H) 7.12-7.17 (m, 2H) 7.27-7.33 (m, 2H) 7.41-7.49 (m, 4H)

### Synthesis of dimethyl ethyl 2-(3-isopropyl-6-oxo-5-phenoxypyridazin-1(6H)-yl)acetate 34G

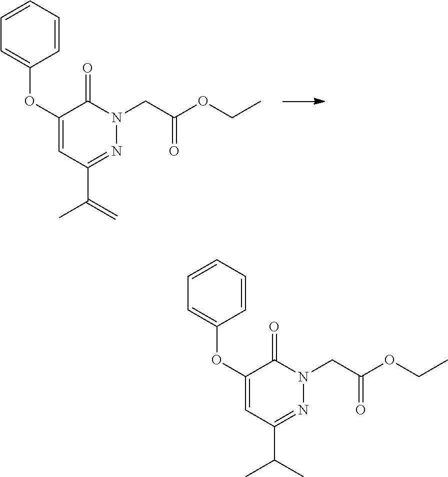 [SMILES: C=C(C)c1cc(Oc2ccccc2)c(=O)n(CC(=O)OCC)n1 ; CCOC(=O)Cn1nc(C(C)C)cc(Oc2ccccc2)c1=O]

A solution of ethyl 2-(6-oxo-5-phenoxy-3-(prop-1-en-2-yl)pyridazin-1(6H)-yl)acetate 34F1 (431 mg, 1.37 mmol) in MeOH (27 mL) and THF (1 mL) was hydrogenated in a H-Cube reactor (1.1 mL/min, 70 mm, 10% Pd/C cartridge, full H₂ mode, at 50° C., 1 cycle). The crude was concentrated in vacuo to yield dimethyl ethyl 2-(3-isopropyl-6-oxo-5-phenoxypyridazin-1(6H)-yl)acetate 34G (403.1 mg, yield 85%, purity 91%) as a colourless oil, used in the next step without further purification.

LCMS (Rₜ: 1.41, Area %: 90.94, MW: 316.00, BPM1: 317.2, Method 7)

¹H NMR (500 MHz, CHLOROFORM-d) δ ppm 1.11 (d, J=7.02 Hz, 6H) 1.30 (t, J=7.10 Hz, 3H) 2.72 (dt, J=13.85, 6.89 Hz, 1H) 4.26 (q, J=7.17 Hz, 2H) 4.91 (s, 2H) 6.24 (s, 1H) 7.12 (d, J=7.63 Hz, 2H) 7.28-7.32 (m, 1H) 7.41-7.48 (m, 2H)

Additional analogues were synthesized according to the above procedure, using the appropriate reagents.

| Intermediate | Product |
|---|---|
| 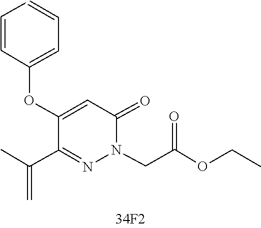 [SMILES: C=C/C=C(\C=C/C)Oc1cc(=O)n(CC(=O)OCC)nc1C(=C)C] | 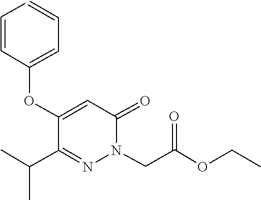 [SMILES: C=C/C=C(\C=C/C)Oc1cc(=O)n(CC(=O)OCC)nc1C(C)C] |

### Synthesis of 2-(2-((tert-butyldimethylsilyl)oxy)ethyl)-5,6-dichloropyridazin-3(2H)-one

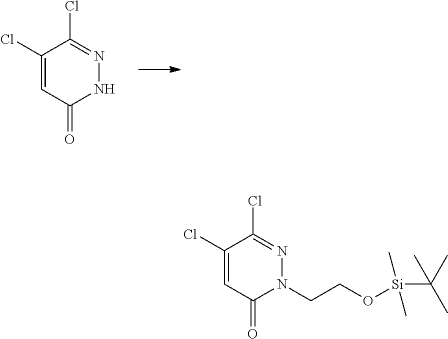 [SMILES: O=c1cc(Cl)c(Cl)n[nH]1 ; CC(C)(C)[Si](C)(C)OCCn1nc(Cl)c(Cl)cc1=O]

Potassium carbonate [584-08-7] (5.03 g, 36.37 mmol) was added to a stirred solution of 5-6-dichloropyridazin-3(2H)-one [17285-36-8] (2 g, 12.12 mmol) and 2-bromoethoxy-tert-butyldimethylsilane [86864-60-0] (3.12 mL, 1.12 g/mL, 14.55 mmol) in DMF (51 mL) at room temperature. The mixture was stirred at room temperature for 16 hours. Then H2O and AcOEt were added, the organic was washed with brine and was separated, dried over MgSO4, filtered and the solvents evaporated in vacuo. The crude product was purified by column chromatography (80 g silica; gradient of heptane/AcOEt 100/0 to 10/90). The desired fractions were collected and concentrated to dryness to afford 2-(2-((tert-butyldimethylsilyl)oxy)ethyl)-5,6-dichloropyridazin-3(2H)-one as a white solid (3.51 g, yield 89%).

LCMS: RT: 1.750, Area %: 99, MH+: 323.0, Method: 13

1H NMR (300 MHz, DMSO-d6) δ ppm 7.56 (s, 1H), 4.13 (t, J=5.4 Hz, 2H), 3.89 (t, J=5.4 Hz, 2H), 0.78 (s, 9H), −0.05 (s, 6H).

Additional analogues were synthesized according to the above procedure, using the appropriate reagents.

| Reagent | Product |
|---|---|
| 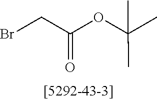 | 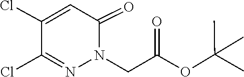 [SMILES: CC(C)(C)OC(=O)Cn1nc(Cl)c(Cl)cc1=O] |

### Synthesis of 2-(3,4-dichloro-6-oxopyridazin-1(6H)-yl)acetic acid

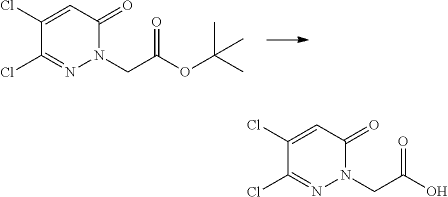 [SMILES: CC(C)(C)OC(=O)Cn1nc(Cl)c(Cl)cc1=O ; O=C(O)Cn1nc(Cl)c(Cl)cc1=O]

TFA [76-05-1] (8.65 mL, 1.54 g/mL, 116.44 mmol) was added to a stirred solution of tert-butyl 2-(3,4-dichloro-6-oxopyridazin-1(6H)-yl)acetate (3.25 g, 11.64 mmol) in DCM at 0° C. The mixture was stirred at rt for 16 hours. The reaction mixture was co-evaporated 4 times with DCM at 40° C. to yield 2-(3,4-dichloro-6-oxopyridazin-1(6H)-yl)acetic acid (2.27 g, yield 87%) as a white solid.

Additional analogues were synthesized according to the above procedure, using the appropriate reagents.

| Reagent | Product |
|---|---|
| 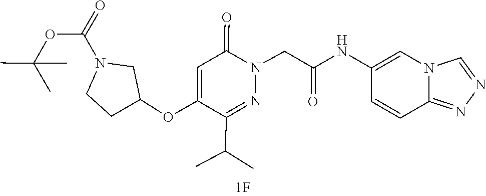 [SMILES: CC(C)c1nn(CC(=O)Nc2ccc3nncn3c2)c(=O)cc1OC1CCN(C(=O)OC(C)(C)C)C1] | 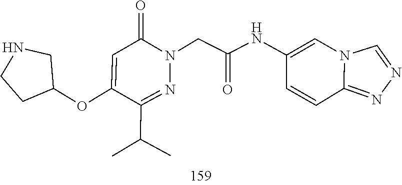 [SMILES: CC(C)c1nn(CC(=O)Nc2ccc3nncn3c2)c(=O)cc1OC1CCNC1] |
| 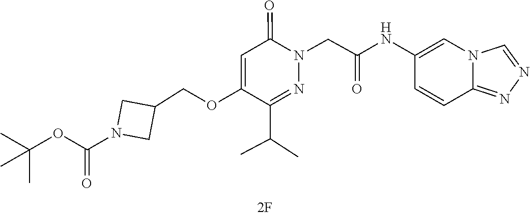 [SMILES: CC(C)c1nn(CC(=O)Nc2ccc3nncn3c2)c(=O)cc1OCC1CN(C(=O)OC(C)(C)C)C1] | 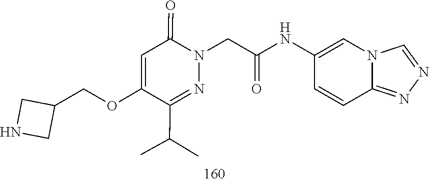 [SMILES: CC(C)c1nn(CC(=O)Nc2ccc3nncn3c2)c(=O)cc1OCC1CNC1] |
| 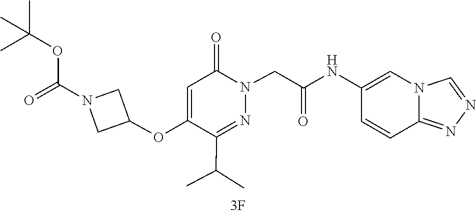 [SMILES: CC(C)c1nn(CC(=O)Nc2ccc3nncn3c2)c(=O)cc1OC1CN(C(=O)OC(C)(C)C)C1] | 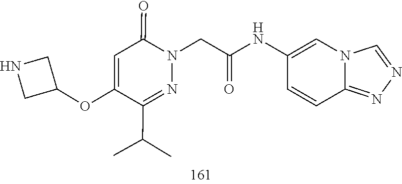 [SMILES: CC(C)c1nn(CC(=O)Nc2ccc3nncn3c2)c(=O)cc1OC1CNC1] |

### Synthesis of 2-(3-chloro-6-oxo-4-(2,2,2-trifluoroethoxy)pyridazin-1(6H)-yl)acetic acid

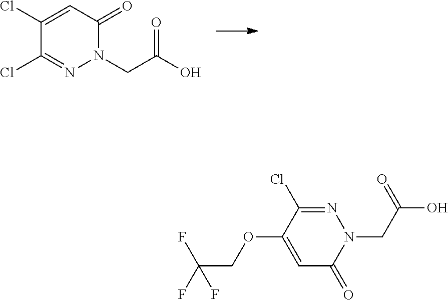 [SMILES: O=C(O)Cn1nc(Cl)c(Cl)cc1=O ; O=C(O)Cn1nc(Cl)c(OCC(F)(F)F)cc1=O]

Solution 1: NaH 60% in mineral oil [7646-69-7] (0.18 g, 4.48 mmol) was added to a stirred solution of trifluoroethanol [75-89-8] (0.32 mL, 1.39 g/mL, 4.48 mmol) in THF dry (14 mL) at 0° C. The reaction mixture was stirred at room temperature for 30 min.

Solution 2: NaH 60% in mineral oil [7646-69-7] (0.18 g, 4.48 mmol) was added to a stirred solution of 2-(3,4-dichloro-6-oxopyridazin-1(6H)-yl)acetic acid (1000 mg, 4.48 mmol) in DMF dry (25 mL) at 0° C. The reaction mixture was stirred at room temperature for 30 min.

Then, solution 1 was added portionwise to solution 2 at 0° C. under nitrogen. The mixture was slowly warmed to rt and stirred at rt for 16h. The reaction mixture was diluted with water and acidified to pH 3 with a N HCl. The organic layer was separated, washed with brine, dried over MgSO4, filtered and concentrated in vacuo to yield 2-(3-chloro-6-oxo-4-(2,2,2-trifluoroethoxy)pyridazin-1(6H)-yl)acetic acid (1200 mg, yield 51%, purity 55%) as a beige solid. The crude product was used without further purification for the next reaction step.

### Synthesis of 2-(6-oxo-3,4-bis(2,2,2-trifluoroethoxy)pyridazin-1(6H)-yl)acetic acid

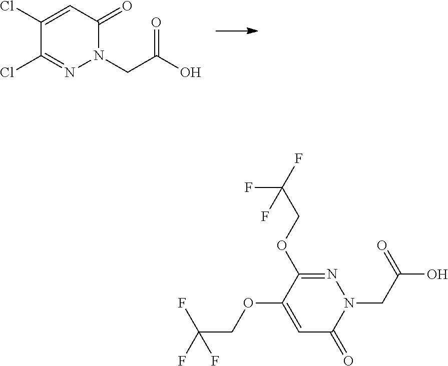 [SMILES: O=C(O)Cn1nc(Cl)c(Cl)cc1=O ; O=C(O)Cn1nc(OCC(F)(F)F)c(OCC(F)(F)F)cc1=O]

Solution 1: NaH 60% in mineral oil [7646-69-7] (280 mg, 6.73 mmol) was added to a stirred solution of trifluoroethanol [75-89-8] (673 mg, 6.73 mmol) in THF dry (14 mL) at 0° C. The reaction mixture was stirred at room temperature for 30 min.

Solution 2: NaH 60% in mineral oil [7646-69-7] (0.18 g, 4.48 mmol) was added to a stirred solution of 2-(3,4-dichloro-6-oxopyridazin-1(6H)-yl)acetic acid (1000 mg, 4.48 mmol) in DMF dry (25 mL) at 0° C. The reaction mixture was stirred at room temperature for 30 min.

Then, solution 1 was added portionwise to solution 2 at 0° C. under nitrogen. The mixture was slowly warmed to rt and stirred at rt for 16h. The reaction mixture was diluted with water and acidified to pH 3 with 1N HCl. The organic layer was separated, washed with brine, dried over MgSO4, filtered and concentrated in vacuo to yield 2-(6-oxo-3,4-bis(2,2,2-trifluoroethoxy)pyridazin-1(6H)-yl)acetic acid (1570 mg, yield 60%, purity 60%) as a beige solid. The crude product was used without further purification for the next reaction step.

### Synthesis of methyl 2-(3,4-diisobutoxy-6-oxopyridazin-1(6H)-yl)acetate

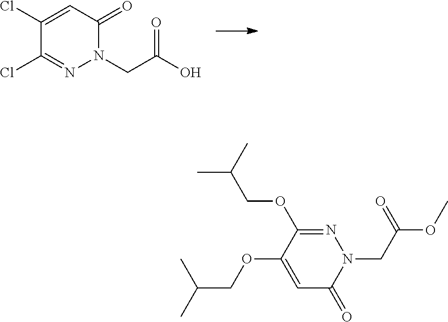 [SMILES: O=C(O)Cn1nc(Cl)c(Cl)cc1=O ; COC(=O)Cn1nc(OCC(C)C)c(OCC(C)C)cc1=O]

NaH [7646-69-7] (0.81 g, 60% dispersion in mineral oil, 20.33 mmol) was added to a stirred solution of 2-methyl-1-propanol [78-83-1] (1.88 mL, 0.8 g/mL, 20.33 mmol) in DMF dry (60 mL) at 0° C. The reaction mixture was stirred at room temperature for 30 min. Then, the mixture was added over 2-(3,4-dichloro-6-oxopyridazin-1(6H)-yl)acetic acid (2.27 g, 10.17 mmol) and the reaction mixture was stirred at 60° C. for 16h. The reaction mixture was diluted with EtOAc and washed twice with a 2% of AcOH solution, followed with brine. Organic layer was dried over MgSO4, filtered and concentrated in vacuo. The crude was dissolved in DMF (25 mL) followed by sequential addition of cesium carbonate [534-17-8] (4.3 g, 13.22 mmol) and iodomethane [74-88-4] (1659 mg, 11.69 mmol). After 2 hours of stirring, the reaction mixture was diluted with EtOAc and washed twice with water, followed with brine. Organic layer was dried over MgSO4, filtered and concentrated in vacuo. The residue was purified by flash column chromatography (SiO2 2 g, MeOH in DCM 0/100 to 10/90 and HCOOH in MeOH in DCM 0/100 to 10/90). The desired fractions were collected and concentrated in vacuo to yield methyl 2-(3,4-diisobutoxy-6-oxopyridazin-1(6H)-yl)acetate (398 mg, yield 12%) as a white solid.

### Synthesis of 2-(2-((tert-butyldimethylsilyl)oxy)ethyl)-6-chloro-5-(cyclopropylmethoxy)pyridazin-3(2H)-one

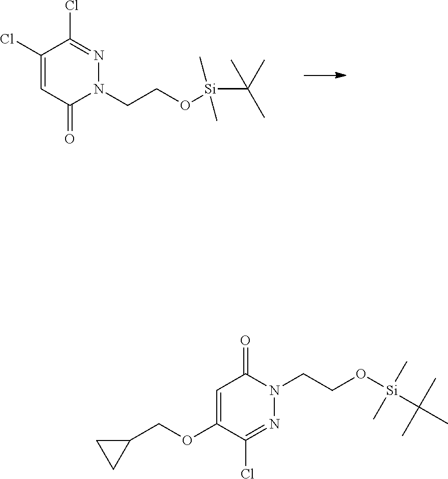 [SMILES: CC(C)(C)[Si](C)(C)OCCn1nc(Cl)c(Cl)cc1=O ; CC(C)(C)[Si](C)(C)OCCn1nc(Cl)c(OCC2CC2)cc1=O]

NaH [7646-69-7] (0.49 g, 60% dispersion in mineral oil, 12.34 mmol) was added to a stirred solution of cyclopropanemethanol [2516-33-8] (1.11 mL, 0.8 g/mL, 12.34 mmol) in THF dry (50 mL) at 0° C. The reaction mixture was stirred at room temperature for 30 min. Then, 2-(2-((tert-butyldimethylsilyl)oxy)ethyl)-5,6-dichloropyridazin-3(2H)-one (2.66 g, 8.23 mmol) was added and the reaction mixture was stirred at 60° C. for 16 h. The reaction mixture was quenched with saturated NH4Cl solution and extracted with AcOEt twice. The organic layers were combined, dried over MgSO4, filtered and concentrated in vacuo. The crude product was purified by flash column chromatography (SiO2 80 g, EtOAc in Heptane 0/100 to 30/70). The desired fractions were collected and concentrated in vacuo to yield 2-(2-((tert-butyldimethylsilyl)oxy)ethyl)-6-chloro-5-(cyclopropylmethoxy)pyridazin-3(2H)-one (1.24 g, yield 42%) as a yellow oil.

Additional analogues were synthesized according to the above procedure, using the appropriate reagents.

| Reagent | Product |
|---|---|
| 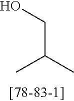 | 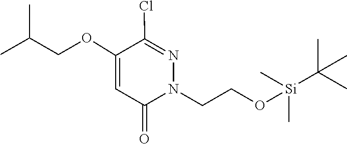 [SMILES: CC(C)COc1cc(=O)n(CCO[Si](C)(C)C(C)(C)C)nc1Cl] |

### Synthesis of 2-(2-((tert-butyldimethylsilyl)oxy)ethyl)-5-(cyclopropylmethoxy)-6-(dimethylamino)pyridazin-3(2H)-one

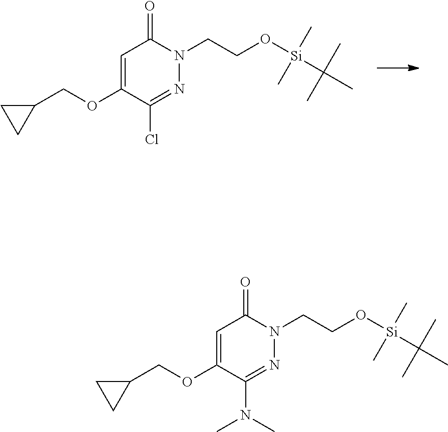 [SMILES: CC(C)(C)[Si](C)(C)OCCn1nc(Cl)c(OCC2CC2)cc1=O ; CN(C)c1nn(CCO[Si](C)(C)C(C)(C)C)c(=O)cc1OCC1CC1]

Pd2(dba)3 [51364-51-3] (314 mg, 0.34 mmol), Xantphos [161265-03-8] (198 mg, 0.34 mmol) and Cs2CO3 [534-17-8] (3.91 g, 11.99 mmol) were added to stirred solution of 2-(2-((tert-butyldimethylsilyl)oxy)ethyl)-6-chloro-5-(cyclopropylmethoxy)pyridazin-3(2H)-one (1.23 g, 3.43 mmol) in DMA (13 mL) at rt under nitrogen atmosphere. Dimethylamine 2M in THF [124-40-3] (3.43 mL, 2 M, 6.85 mmol) was added and the mixture was heated at 90° C. for 6 h. The reaction mixture was diluted with EtOAc and washed twice water and then with brine. Organic layer was dried MgSO4 (anh), filtered and concentrated in vacuo. The crude product was purified by flash column chromatography (silica 25 g; EtOAc in Heptane from 0/100 to 40/60). The desired fractions were collected and concentrated in vacuo to yield 2-(2-((tert-butyldimethylsilyl)oxy)ethyl)-5-(cyclopropylmethoxy)-6-(dimethylamino)pyridazin-3(2H)-one (965 mg, yield 77%) as a brown oil.

Additional analogues were synthesized according to the above procedure, using the appropriate reagents.

| Intermediate | Product |
|---|---|
| 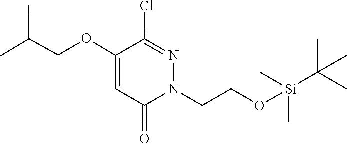 [SMILES: CC(C)COc1cc(=O)n(CCO[Si](C)(C)C(C)(C)C)nc1Cl] | 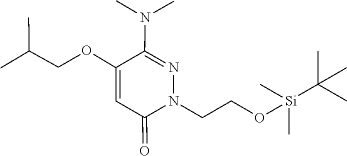 [SMILES: CC(C)COc1cc(=O)n(CCO[Si](C)(C)C(C)(C)C)nc1N(C)C] |

### Synthesis of methyl 2-(3-(dimethylamino)-6-oxo-4-(2,2,2-trifluoroethoxy)pyridazin-1(6H)-yl)acetate

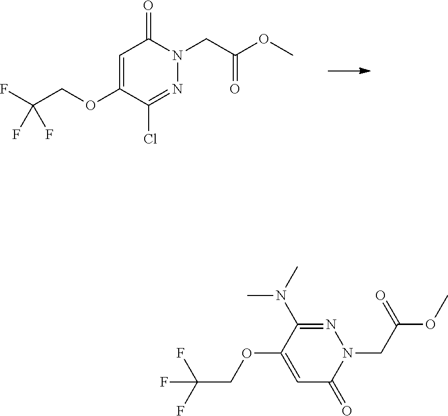 [SMILES: COC(=O)Cn1nc(Cl)c(OCC(F)(F)F)cc1=O ; COC(=O)Cn1nc(N(C)C)c(OCC(F)(F)F)cc1=O]

XPhos [564483-18-7] (0.08 g, 0.14 mmol) and Pd2dba3 [51364-51-3] (0.063 g, 0.069 mmol) were sequentially added to stirred solution of methyl 2-(3-chloro-6-oxo-4-(2,2,2-trifluoroethoxy)pyridazin-1(6H)-yl)acetate (416 mg, 1.38 mmol) and cesium carbonate [534-17-8] (1.35 g, 4.15 mmol) in dry toluene (8 mL) while nitrogen was bubbling. Then dimethylamine 2M in THF [936940-38-4] (1.04 mL, 2 M, 2.08 mmol) was added and the reaction mixture was stirred for 16 hours at 95° C. Water was added and the mixture was extracted with EtOAc (3×). The combined organic layers were dried over MgSO4 and evaporated in vacuo. The crude was purified by flash column chromatography (silica 25 g; EtOAc in heptane from 0/100 to 80/20). The desired fractions were collected and concentrated to yield 2-(3-(dimethylamino)-6-oxo-4-(2,2,2-trifluoroethoxy)pyridazin-1(6H)-yl)acetate (189 mg, yield 44%) as a yellow oil.

### Synthesis of 5-(cyclopropylmethoxy)-6-(dimethylamino)-2-(2-hydroxyethyl)pyridazin-3(2H)-one 157

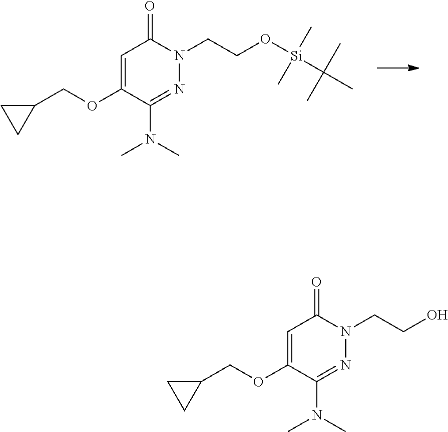 [SMILES: CN(C)c1nn(CCO[Si](C)(C)C(C)(C)C)c(=O)cc1OCC1CC1 ; CN(C)c1nn(CCO)c(=O)cc1OCC1CC1]

TBAF [429-41-4] (3.25 mL, 1 M, 3.15 mmol) was added to a stirred solution of 2-(2-((tert-butyldimethylsilyl)oxy)ethyl)-5-(cyclopropylmethoxy)-6-(dimethylamino)pyridazin-3(2H)-one (965 mg, 2.63 mmol) in THF dry (8 mL) at 0° C. The mixture was stirred at 0° C. to rt for 2 h. The mixture was diluted with a saturated aqueous Na2CO3 solution and extracted with EtOAc. Organic layers were combined and washed with brine, dried over MgSO4, filtered and concentrated in vacuo. The crude product was purified by flash column chromatography (silica 25 g; EtOAc in Heptane from 0/100 to 100/0). The desired fractions were collected and concentrated in vacuo to yield 5-(cyclopropylmethoxy)-6-(dimethylamino)-2-(2-hydroxyethyl)pyridazin-3(2H)-one 157 (645 mg, yield 97%) as an oil.

Additional analogues were synthesized according to the above procedure, using the appropriate reagents.

| Intermediate | Product |
|---|---|
| 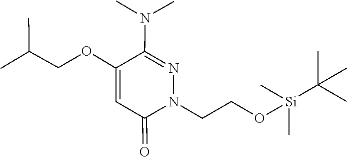 [SMILES: CC(C)COc1cc(=O)n(CCO[Si](C)(C)C(C)(C)C)nc1N(C)C] | 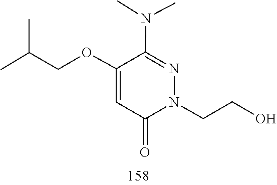 [SMILES: CC(C)COc1cc(=O)n(CCO)nc1N(C)C] |

### Synthesis of 2-(4-(cyclopropylmethoxy)-3-(dimethylamino)-6-oxopyridazin-1(6H)-yl)acetaldehyde

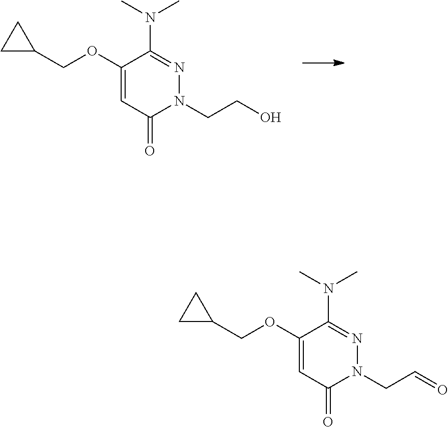 [SMILES: CN(C)c1nn(CCO)c(=O)cc1OCC1CC1 ; CN(C)c1nn(CC=O)c(=O)cc1OCC1CC1]

A solution of DMSO [67-68-5] (0.81 mL, 1.1 g/mL, 11.35 mmol) in DCM dry (3 mL) was added to a solution of oxalyl chloride [79-37-8] (0.46 mL, 1.5 g/mL, 5.34 mmol) in DCM dry (3 mL) at −78° C. for 10 min and the reaction mixture was stirred at the same temperature for 15 min. Then a solution of 5-(cyclopropylmethoxy)-6-(dimethylamino)-2-(2-hydroxyethyl)pyridazin-3(2H)-one 157 (1.2 g, 4.73 mmol) in DCM dry (8 mL) was added to the mixture at −78° C. and the reaction mixture was stirred at the same temperature for 15 min. Then triethylamine [121-44-8] (3.33 mL, 0.73 g/mL, 23.65 mmol) was added and the reaction mixture was stirred and allowed to warm to rt for 16 h. The mixture was diluted with water, a saturated solution of NaHCO3, and brine. The organic phase was dried (MgSO4), filtered and the solvent evaporated in vacuo. The crude product was purified by flash column chromatography (SiO2 25 g; MeOH in DCM 0/100 to 5/95). The desired fractions were collected and concentrated in vacuo to yield 2-(4-(cyclopropylmethoxy)-3-(dimethylamino)-6-oxopyridazin-1(6H)-yl)acetaldehyde (1.12 g, yield 92%) as a white solid.

Additional analogues were synthesized according to the above procedure, using the appropriate reagents.

| Intermediate | Product |
|---|---|
| 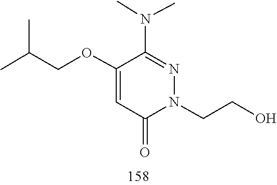 [SMILES: CC(C)COc1cc(=O)n(CCO)nc1N(C)C] | 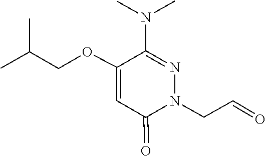 [SMILES: CC(C)COc1cc(=O)n(CC=O)nc1N(C)C] |

### Synthesis of 2-(4-(cyclopropylmethoxy)-3-(dimethylamino)-6-oxopyridazin-1(6H)-yl)acetic acid

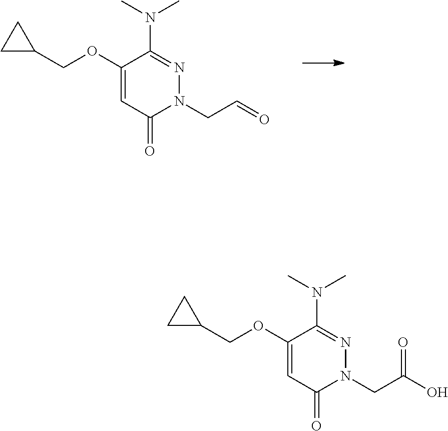 [SMILES: CN(C)c1nn(CC=O)c(=O)cc1OCC1CC1 ; CN(C)c1nn(CC(=O)O)c(=O)cc1OCC1CC1]

2-Methyl-2-butene (2 M in THF) [513-35-9] (10.25 mL, 2 M, 20.5 mmol) was added to a stirred solution of 2-(4-(cyclopropylmethoxy)-3-(dimethylamino)-6-oxopyridazin-1(6H)-yl)acetaldehyde (1.12 g, 4.46 mmol) and sodium phosphate monobasic monohydrate [13472-35-0] (0.93 g, 6.69 mmol) in tert-butanol (45 mL) and water (9 mL). Then sodium chlorite [7758-19-2] (1.51 g, 13.37 mmol) was added portionwise and the mixture was stirred at rt for 2h. The reaction mixture was acidified with 10% aqueous NaHSO3 until pH 3-4 and extracted with DCM-MeOH (4:1). The organic layer was separated, dried (MgSO4), filtered and the solvents evaporated in vacuo. The crude product was purified by flash column chromatography (SiO2 12 g; MeOH in DCM 0/100 to 5/95). The desired fractions were collected and concentrated in vacuo to yield 2-(4-(cyclopropylmethoxy)-3-(dimethylamino)-6-oxopyridazin-1(6H)-yl)acetic acid (0.67 g, yield 52%) as a yellow solid.

Additional analogues were synthesized according to the above procedure, using the appropriate reagents.

| Intermediate | Product |
|---|---|
| 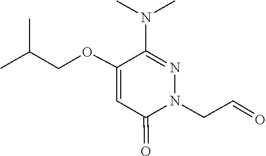 [SMILES: CC(C)COc1cc(=O)n(CC=O)nc1N(C)C] | 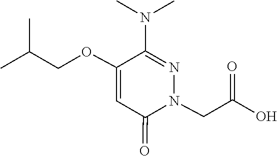 [SMILES: CC(C)COc1cc(=O)n(CC(=O)O)nc1N(C)C] |

### Synthesis of methyl 2-(4-(cyclopropylmethoxy)-3-(dimethylamino)-6-oxopyridazin-1(6H)-yl)acetate

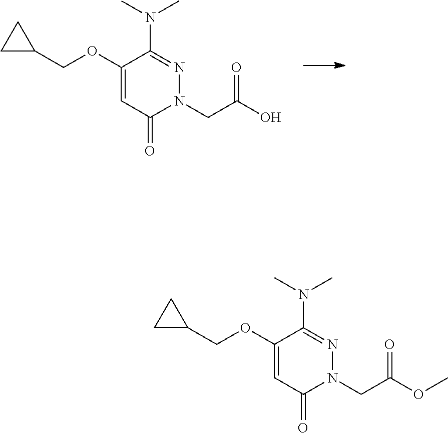 [SMILES: CN(C)c1nn(CC(=O)O)c(=O)cc1OCC1CC1 ; COC(=O)Cn1nc(N(C)C)c(OCC2CC2)cc1=O]

Iodomethane [74-88-4] (0.13 mL, 2.28 g/mL, 2.02 mmol) was added to a stirred solution of 2-(4-(cyclopropylmethoxy)-3-(dimethylamino)-6-oxopyridazin-1(6H)-yl)acetic acid (0.47 g, 1.76 mmol) and cesium carbonate [534-17-8] (0.74 g, 2.29 mmol) in DMF (4.7 mL) at rt. The mixture was stirred at rt for 18h. The mixture was diluted with sat. aqueous NaHCO3 and extracted with AcOEt. The organic layer was separated, dried (MgSO4), filtered and the solvents evaporated in vacuo. The crude was purified by flash column chromatography (silica 25 g; AcOEt in heptane 0/100 to 30/70). The desired fractions were collected and concentrated in vacuo to yield 2-(4-(cyclopropylmethoxy)-3-(dimethylamino)-6-oxopyridazin-1(6H)-yl)acetate (440 mg, yield 85%) as a yellow oil.

Additional analogues were synthesized according to the above procedure, using the appropriate reagents.

| Intermediate | Product |
|---|---|
| 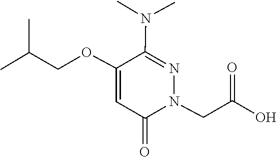 [SMILES: CC(C)COc1cc(=O)n(CC(=O)O)nc1N(C)C] | 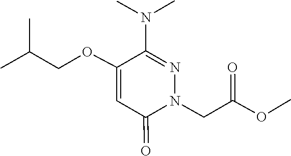 [SMILES: COC(=O)Cn1nc(N(C)C)c(OCC(C)C)cc1=O] |
| 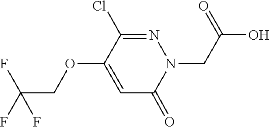 [SMILES: O=C(O)Cn1nc(Cl)c(OCC(F)(F)F)cc1=O] | 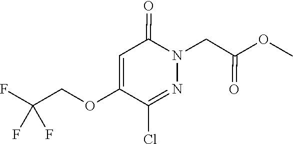 [SMILES: COC(=O)Cn1nc(Cl)c(OCC(F)(F)F)cc1=O] |
| 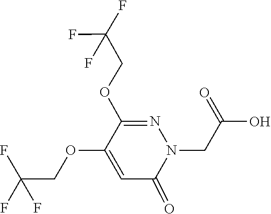 [SMILES: O=C(O)Cn1nc(OCC(F)(F)F)c(OCC(F)(F)F)cc1=O] | 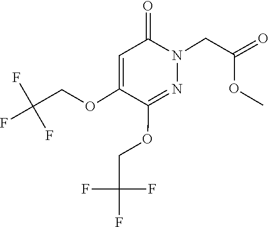 [SMILES: COC(=O)Cn1nc(OCC(F)(F)F)c(OCC(F)(F)F)cc1=O] |

### Synthesis of methyl 3-(hydroxymethyl)bicyclo[1.1.1]pentane-1-carboxylate

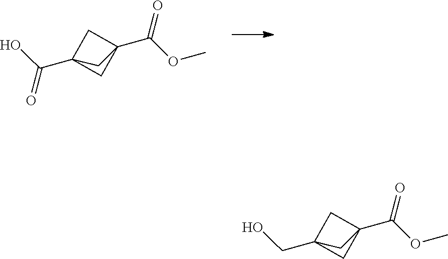

Borane dimethyl sulfide (2 M in THF) [13292-87-0] (11.53 mL, 23.51 mmol) was added dropwise to a solution of bicyclo[1.1.1]pentane-1,3-dicarboxylic acid, 1-methyl ester [83249-10-9] (2 g, 11.75 mmol) in anhydrous THF (50 mL) at 0° C. and the mixture was stirred at rt for 48 h. The mixture was diluted with MeOH and concentrated in vacuo. The residue was dissolved with NaHCO3 (saturated in water) and extracted with EtOAc. The organic layer was dried (MgSO4), filtered and concentrated to yield methyl 3-(hydroxymethyl)bicyclo[1.1.1]pentane-1-carboxylate (1.86 g, yield 91%) as a colourless oil. The crude product was used in the next step without further purification.

### Synthesis of methyl 3-formylbicyclo[1.1.1]pentane-1-carboxylate

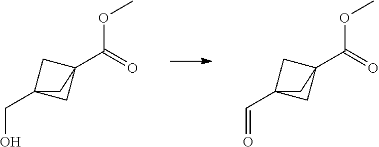

Pyridinium chlorochromate [26299-14-9] (3.513 g, 16.3 mmol) was added to a stirred solution of methyl 3-(hydroxymethyl)bicyclo[1.1.1]pentane-1-carboxylate (2.545 g, 16.3 mmol) in DCM [75-09-2] (64 mL). The mixture was stirred at room temperature for 16 hours. The mixture was filtered over a pad of celite and was washed with DCM. The solvent was removed in vacuo and the crude was purified by flash column chromatography (silica 25 g; EtOAc/Heptane from 0/100 to 50/50). The desired fractions were collected and concentrated in vacuo to yield methyl 3-formylbicyclo[1.1.1]pentane-1-carboxylate (1.140 g, yield 41%) as a colorless oil.

### Synthesis of ethyl (E)-3-(((tert-butylsulfinyl)imino)methyl)bicyclo[1.1.1]pentane-1-carboxylate

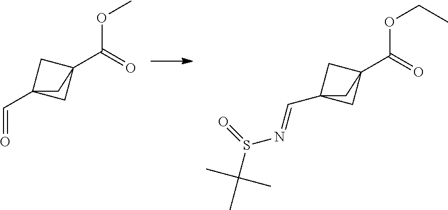 [SMILES: COC(=O)C12CC(C=O)(C1)C2 ; CCOC(=O)C12CC(/C=N/S(=O)C(C)(C)C)(C1)C2]

Titanium(IV) ethoxide [3087-36-3] (3.1 ml, 14.8 mmol) was added to a solution of methyl 3-formylbicyclo[1.1.1]pentane-1-carboxylate (1.140 mg, 7.4 mmol) and 2-methyl-2-propanesulfinamide [146374-27-8] (1.344 g, 11.09 mmol) in tetrahydrofuran (30 ml). The mixture was stirred at 85° C. for 12 h. Water was added to the mixture resulting in formation of a white precipitate. The mixture was diluted with DCM and filtered. The filtrate was washed with brine. The filter cake was washed with DCM. The combined filtrate was concentrated in vacuo. The crude was purified by flash column chromatography (12 g silica; heptane/EtOAc 100/0 to 0/100). The desired fractions were collected and concentrated in vacuo to yield (E)-3-(((tert-butylsulfinyl)imino)methyl)bicyclo[1.1.1]pentane-1-carboxylate (771 mg, yield 38%) as a yellow oil.

### Synthesis of ethyl 3-(1-((tert-butylsulfinyl)amino)-2,2,2-trifluoroethyl)bicyclo[1.1.1]pentane-1-carboxylate

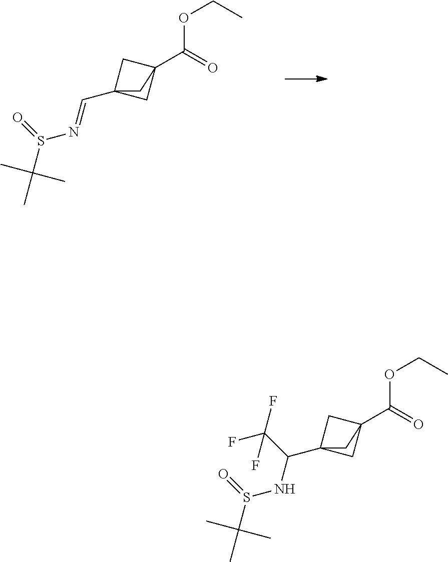 [SMILES: CCOC(=O)C12CC(/C=N/S(=O)C(C)(C)C)(C1)C2 ; CCOC(=O)C12CC(C(NS(=O)C(C)(C)C)C(F)(F)F)(C1)C2]

Trimethyl(trifluoromethyl)silane [81290-20-2] (630 μl, 4.26 mmol) was added dropwise to a mixture of (E)-3-(((tert-butylsulfinyl)imino)methyl)bicyclo[1.1.1]pentane-1-carboxylate (771 mg, 2.84) and tetrabutylammonium fluoride solution [429-41-4] (165 μl, 0.57 mmol) in dry tetrahydrofuran [109-99-9] (37 ml). The reaction mixture was stirred at RT for 18h. Sat. Aq. NH4Cl was added and extracted with EtOAc. The organic layer was separated, dried (MgSO4 anh), filtered and the solvents evaporated in vacuo. The crude was purified by flash column chromatography (12 g silica, EtOAc in Heptane from 100/0 to 50/50). The desired fractions were collected and concentrated in vacuo to yield ethyl 3-(1-((tert-butylsulfinyl)amino)-2,2,2-trifluoroethyl)bicyclo[1.1.1]pentane-1-carboxylate (827 mg, yield 81%) as a yellow oil.

### Synthesis of methyl 3-(methoxymethyl)bicyclo[1.1.1]pentane-1-carboxylate

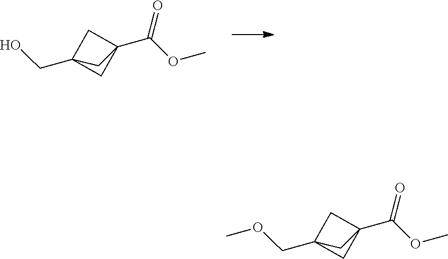

Sodium hydride [7646-69-7] (476.2 mg, 11.91 mmol) was added to a stirred solution of methyl 3-(hydroxymethyl)bicyclo[1.1.1]pentane-1-carboxylate (1.86 g, 11.91 mmol) in anhydrous DMF (12 mL) at 0° C. under nitrogen. The mixture was stirred at rt for 30 min. Then, iodomethane [74-88-4] (2.22 mL, 35.73 mmol) was added dropwise at 0° C. and the mixture stirred at rt for 16 h. The mixture was diluted with water and extracted with diethyl enter. The organic layer was separated, dried (MgSO4), filtered and the solvents evaporated in vacuo to yield methyl 3-(methoxymethyl)bicyclo[1.1.1]pentane-1-carboxylate (1.49 g, yield 66%) as a pale yellow oil. The crude product was used in the next step without further purification.

### Synthesis of 3-(methoxymethyl)bicyclo[1.1.1]pentane-1-carboxylic acid

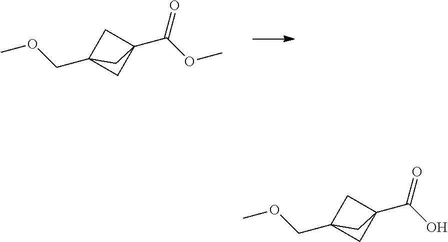

Lithium hydroxide monohydrate [1310-66-3] (551.02 mg, 13.13 mmol) was added to a solution of methyl 3-(methoxymethyl)bicyclo[1.1.1]pentane-1-carboxylate (1.49 g, 8.75 mmol) in THF (89.1 mL), H2O (22.4 mL) and MeOH (22.4 mL) at rt. The reaction mixture was stirred at rt for 16 h. HCl (1M in water) was added until pH=4. The mixture was diluted with water and extracted with EtOAc. The organic layer was separated, dried (MgSO4), filtered and the solvents evaporated in vacuo to yield 3-(methoxymethyl)bicyclo[1.1.1]pentane-1-carboxylic acid (836 mg, yield 55%) as a yellowish oil. The crude product was used in the next step without further purification.

### Synthesis of tert-butyl (3-(methoxymethyl)bicyclo[1.1.1]pentan-1-yl)carbamate

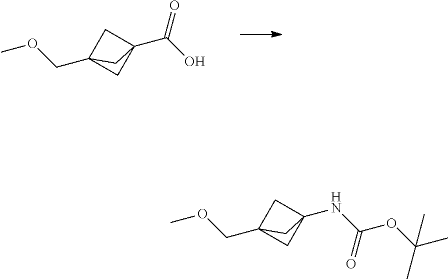 [SMILES: COCC12CC(C(=O)O)(C1)C2 ; COCC12CC(NC(=O)OC(C)(C)C)(C1)C2]

Triethylamine [121-44-8] (2.5 mL, 17.93 mmol) and DPPA [26386-88-9] (1.5 mL, 6.72 mmol) were added to a stirred solution of 3-(methoxymethyl)bicyclo[1.1.1]pentane-1-carboxylic acid (700 mg, 4.48 mmol) in tert-butanol (21 mL) at rt. The mixture was stirred at rt for 1 h and then heated at 80° C. for 18 h. The solvent was removed in vacuo. The residue was dissolved in EtOAc. The organic layer was washed with brine, dried (MgSO4), filtered and the solvents evaporated in vacuo. The crude product was purified by flash column chromatography (silica 25 g; EtOAc in heptane 0/100 to 50/50). The desired fractions were collected and concentrated in vacuo to yield tert-butyl (3-(methoxymethyl)bicyclo[1.1.1]pentan-1-yl)carbamate (109 mg, yield 10%) as a colourless oil.

### Synthesis of 3-(methoxymethyl)bicyclo[1.1.1]pentan-1-amine

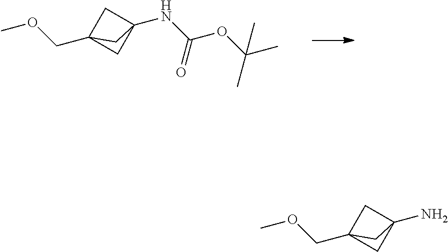

HCl (4N in dioxane) [7647-01-0] (1.9 mL, 7.67 mmol) was added to tert-butyl (3-(methoxymethyl)bicyclo[1.1.1]pentan-1-yl)carbamate (109 mg, 0.48 mmol) and the mixture was stirred at rt for 16 h. The solvent was removed, toluene was added and evaporated twice to yield 3-(methoxymethyl)bicyclo[1.1.1]pentan-1-amine (105 mg, yield 98%) as a white sticky solid. The crude product was used in the next step without further purification.

### Synthesis of methyl 3-(((benzyloxy)carbonyl)amino)bicyclo[1.1.1]pentane-1-carboxylate

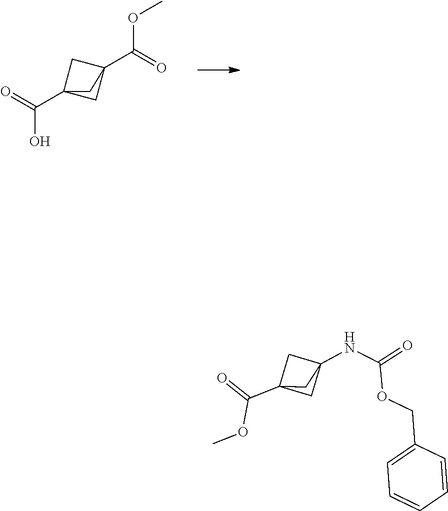 [SMILES: COC(=O)C12CC(C(=O)O)(C1)C2 ; COC(=O)C12CC(NC(=O)OCc3ccccc3)(C1)C2]

Diphenyl phosphoryl azide [26386-88-9] (2.9 mL, 12.93 mmol) was added to a stirred solution of bicyclo[1.1.1]pentane-1,3-dicarboxylic acid, 1-methyl ester [83249-10-9] (2 g, 11.75 mmol) and triethylamine [121-44-8] (4.9 mL, 35.26 mmol) in toluene anhydrous [108-88-3] (58.5 mL) at rt under nitrogen atmosphere. The mixture was stirred at 45° C. for 2 h. Then, benzyl alcohol [100-51-6] (12.2 mL, 117.53 mmol) was added at rt and the mixture was stirred at 80° C. for 16 h. The mixture was cooled down to rt, diluted with sat. NaHCO3 aqueous solution and extracted with EtOAc (×3). The combined organic layers were dried (MgSO4), filtered and solvents evaporated in vacuo. Benzyl alcohol was evaporated in vacuo with a heat gun. The residue was cooled down to rt and the crude product was purified by flash column chromatography (silica 120 g; EtOAc in heptane 0/100 to 13/87). The desired fractions were collected and concentrated in vacuo to yield methyl 3-(((benzyloxy)carbonyl)amino)bicyclo[1.1.1]pentane-1-carboxylate (2 g, yield 61%) as a colorless sticky solid.

Additional analogues were synthesized according to the above procedure, using the appropriate reagent.

| Reagent | Intermediate | Product |
|---|---|---|
| 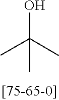 | 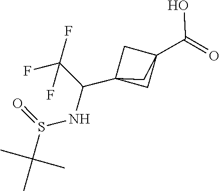 [SMILES: CC(C)(C)S(=O)NC(C(F)(F)F)C12CC(C(=O)O)(C1)C2] | 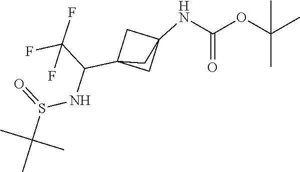 [SMILES: CC(C)(C)OC(=O)NC12CC(C(NS(=O)C(C)(C)C)C(F)(F)F)(C1)C2] |

### Synthesis of 3-(1-amino-2,2,2-trifluoroethyl)bicyclo[1.1.1]pentan-1-amine hydrochloride

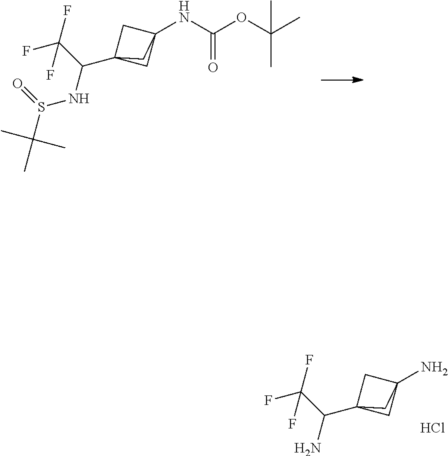 [SMILES: CC(C)(C)OC(=O)NC12CC(C(NS(=O)C(C)(C)C)C(F)(F)F)(C1)C2 ; NC(C(F)(F)F)C12CC(N)(C1)C2 ; Cl]

Tert-butyl (3-(1-((tert-butylsulfinyl)amino)-2,2,2-trifluoroethyl)bicyclo[1.1.1]pentan-1-yl)carbamate (162 mg, 0.42 mmol) was dissolved in methanol [67-56-1] (5.1 ml). The reaction was cooled at 0° C. HCl 4N in dioxane [7647-01-0] (5 ml, 11.8 mmol) was added. The mixture was stirred at rt for 1 h and 30 min. The solvent was evaporated in vacuo to yield 3-(1-amino-2,2,2-trifluoroethyl)bicyclo[1.1.1]pentan-1-amine hydrochloride (114 mg, yield 99%) as a yellow solid.

### Synthesis of benzyl (3-(hydroxymethyl)bicyclo[1.1.1]pentan-1-yl)carbamate

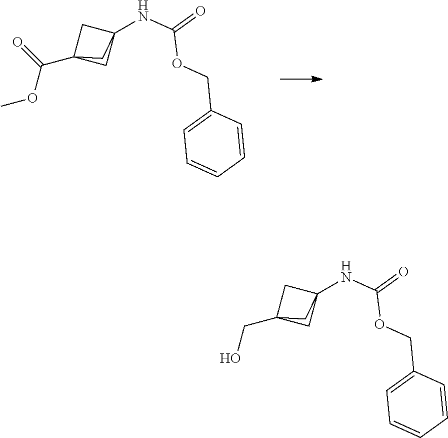 [SMILES: COC(=O)C12CC(NC(=O)OCc3ccccc3)(C1)C2 ; O=C(NC12CC(CO)(C1)C2)OCc1ccccc1]

Sodium borohydride [16940-66-2] (555 mg, 14.53 mmol) was added portionwise to a stirred suspension of calcium chloride [10043-52-4] (814 mg, 7.27 mmol) in anhydrous THF (10 mL) and ethanol absolute [64-17-5] (10 mL) at −20° C. under nitrogen atmosphere and the mixture was stirred for 15 min. Then methyl 3-(((benzyloxy)carbonyl)amino)bicyclo[1.1.1]pentane-1-carboxylate (1 g, 3.63 mmol) diluted in anhydrous THF [109-99-9] (6 mL) and ethanol absolute [64-17-5] (6 mL) was added dropwise to the mixture at −20° C. The reaction mixture was stirred at −20° C. to rt for 16h. The reaction was diluted with water at 0° C. and extracted with EtOAc. The organic layer was dried (MgSO4), filtered and the solvents evaporated in vacuo. The crude product was purified by flash column chromatography (silica 25 g; EtOAc in heptane 0/100 to 50/50). The desired fractions were collected and concentrated in vacuo to afford benzyl (3-(hydroxymethyl)bicyclo[1.1.1]pentan-1-yl)carbamate (880 g, yield 97%) as white solid.

### Synthesis of benzyl (3-(iodomethyl)bicyclo[1.1.1]pentan-1-yl)carbamate

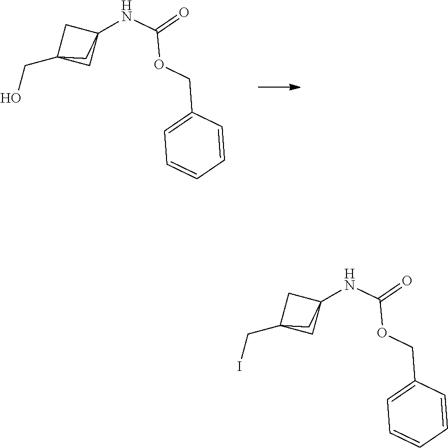 [SMILES: O=C(NC12CC(CO)(C1)C2)OCc1ccccc1 ; O=C(NC12CC(CI)(C1)C2)OCc1ccccc1]

Imidazole [288-32-4] (367 mg, 5.34 mmol) and triphenylphosphine [603-35-0] (1 g, 3.91 mmol) were added to a stirred solution of benzyl (3-(hydroxymethyl)bicyclo[1.1.1]pentan-1-yl)carbamate (880 mg, 3.56 mmol) in THF anhydrous (8 mL) at 0° C. under nitrogen atmosphere. The mixture was stirred 10 min at 0° C. and iodine [7553-56-2] (996 mg, 3.91 mmol) was added portionwise. The mixture was vigorously stirred at rt for 1 h. Then was diluted with 10% w/v Na2S203 aqueous solution and NaHCO3 sat and extracted with EtOAc. The combined organic layers were dried (MgSO4), filtered and solvents evaporated in vacuo. The crude product was purified by flash column chromatography (silica 12 g; EtOAc in heptane 0/100 to 2/98). The desired fractions were collected and concentrated in vacuo to yield benzyl (3-(iodomethyl)bicyclo[1.1.1]pentan-1-yl)carbamate (864 g, yield 67%) as a white solid.

### Synthesis of benzyl (3-((methylsulfonyl)methyl)bicyclo[1.1.1]pentan-1-yl)carbamate

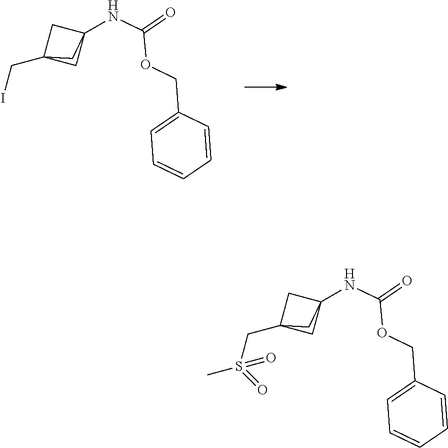 [SMILES: O=C(NC12CC(CI)(C1)C2)OCc1ccccc1 ; CS(=O)(=O)CC12CC(NC(=O)OCc3ccccc3)(C1)C2]

Sodium methanesulfinate [20277-69-4] (63 mg, 0.62 mmol) was added to a solution of benzyl (3-(iodomethyl)bicyclo[1.1.1]pentan-1-yl)carbamate (200 mg, 0.56 mmol) in N,N-dimethylformamide (1.7 ml). The reaction mixture was stirred at 65° C. for 12h. The solvent was evaporated, the residue was taken in water and extracted with EtOAc. The organic layer was separated, dried (MgSO4 anh), filtered and the solvents evaporated in vacuo. The crude was purified by flash column chromatography (12 g silica, EtOAc in Heptane from 100/0 to 50/50) The desired fractions were collected and concentrated in vacuo to yield benzyl (3-((methylsulfonyl)methyl)bicyclo[1.1.1]pentan-1-yl)carbamate (172 mg, yield 98%) as an orange oil.

### Synthesis of 3-((methylsulfonyl)methyl)bicyclo[1.1.1]pentan-1-amine 1,1,1,3,3,3-hexafluoropropan-2-ol salt

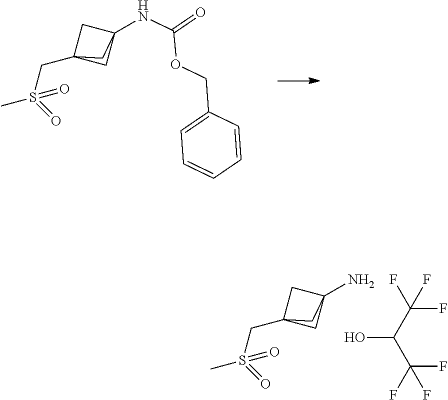

10% Palladium on carbon [7440-05-3] (79 mg, 0.07 mmol) was added to a stirred solution of benzyl (3-((methylsulfonyl)methyl)bicyclo[1.1.1]pentan-1-yl)carbamate (172 mg, 0.56 mmol) in HFIP (4.5 mL) at 0° C. under nitrogen atmosphere. Then, nitrogen atmosphere was replaced by hydrogen (1 atm, balloon) and the reaction mixture was stirred at rt for 6 h. The mixture was filtered off over a thin pad of celite, washed with DCM/MeOH (9:1) and solvents from filtrate were evaporated in vacuo to yield 3-((methylsulfonyl)methyl)bicyclo[1.1.1]pentan-1-amine 1,1,1,3,3,3-hexafluoropropan-2-ol salt (115 mg, yield 59%) as a colorless oil.

### Synthesis of N-([1,2,4]triazolo[4,3-a]pyridin-6-yl)-2-(4-(cyclopropylmethoxy)-3-(dimethylamino)-6-oxopyridazin-1(6H)-yl)acetamide 35

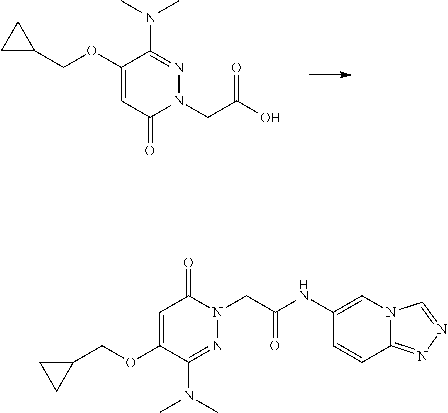 [SMILES: CN(C)c1nn(CC(=O)O)c(=O)cc1OCC1CC1 ; CN(C)c1nn(CC(=O)Nc2ccc3nncn3c2)c(=O)cc1OCC1CC1]

Triethylamine [121-44-8] (0.1 mL, 0.73 g/mL, 0.75 mmol) was added to a stirred solution of 2-(4-(cyclopropylmethoxy)-3-(dimethylamino)-6-oxopyridazin-1(6H)-yl)acetic acid (100 mg, 0.37 mmol) and [1,2,4]-triazolo-[4,3-a]-pyridine-6-amine [1082448-58-5] (75 mg, 0.56 mmol) in DMF anhydrous (1 mL) at rt under nitrogen. The mixture was stirred for 5 min, then propyl phosphonic anhydride solution [68957-94-8] (0.31 mL, 1.07 g/mL, 50% in EtOAc, 0.52 mmol) was added and the mixture was stirred at rt for 18h. The mixture was diluted with saturated aqueous NaHCO3 solution and extracted with EtOAc. Organic layers were combined washed with brine, dried (MgSO4), filtered and concentrated in vacuo. The crude product was purified by flash column chromatography (silica 12 g; MeOH in DCM from 100/0 to 2/98). The desired fractions were collected and concentrated in vacuo. The residue was repurified by reverse phase using as column: Brand Phenomenex; Type Gemini; Product number 00D-4435-EO-AX; I.D. (mm) 100×30; Particle size 5 um (C18) 110A; Installed Gilson 1. Method: MMP4BIC From 81:19 to 45:55 [25 mM NH4HCO3]/[ACN: MeOH (1:1)]. The desired fractions were combined and evaporated in vacuo to yield N-([1,2,4]triazolo[4,3-a]pyridin-6-yl)-2-(4-(cyclopropylmethoxy)-3-(dimethylamino)-6-oxopyridazin-1(6H)-yl)acetamide 35 (60 mg, yield 41%) as a white solid.

Additional analogues were synthesized according to the above procedure, using the appropriate reagents.

| Reagent | Intermediate | Product |
|---|---|---|
| 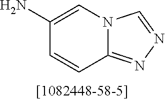 | 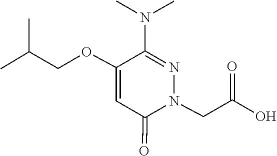 [SMILES: CC(C)COc1cc(=O)n(CC(=O)O)nc1N(C)C] | 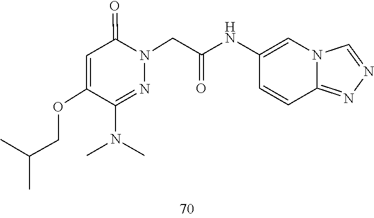 [SMILES: CC(C)COc1cc(=O)n(CC(=O)Nc2ccc3nncn3c2)nc1N(C)C] |
| 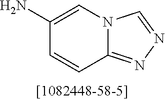 | 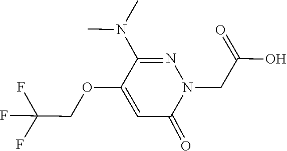 [SMILES: CN(C)c1nn(CC(=O)O)c(=O)cc1OCC(F)(F)F] | 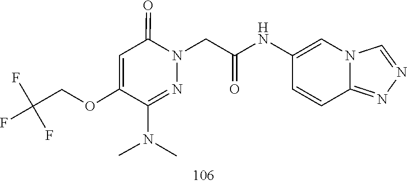 [SMILES: CN(C)c1nn(CC(=O)Nc2ccc3nncn3c2)c(=O)cc1OCC(F)(F)F] |
| 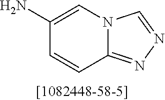 | 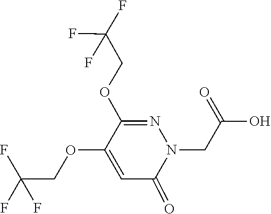 [SMILES: O=C(O)Cn1nc(OCC(F)(F)F)c(OCC(F)(F)F)cc1=O] | 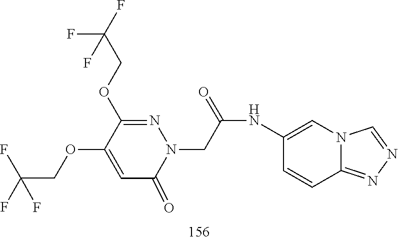 [SMILES: O=C(Cn1nc(OCC(F)(F)F)c(OCC(F)(F)F)cc1=O)Nc1ccc2nncn2c1] |
| 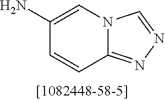 | 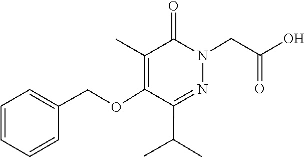 [SMILES: Cc1c(OCc2ccccc2)c(C(C)C)nn(CC(=O)O)c1=O] | 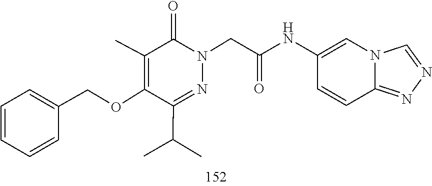 [SMILES: Cc1c(OCc2ccccc2)c(C(C)C)nn(CC(=O)Nc2ccc3nncn3c2)c1=O] |
| 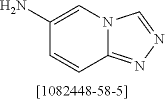 | 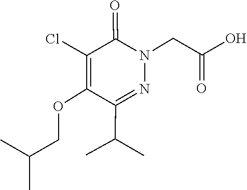 [SMILES: CC(C)COc1c(C(C)C)nn(CC(=O)O)c(=O)c1Cl] | 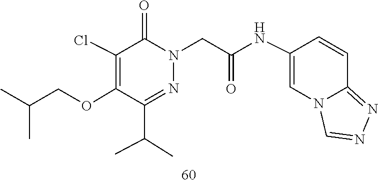 [SMILES: CC(C)COc1c(C(C)C)nn(CC(=O)Nc2ccc3nncn3c2)c(=O)c1Cl] |
| 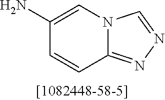 | 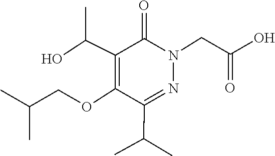 [SMILES: CC(C)COc1c(C(C)C)nn(CC(=O)O)c(=O)c1C(C)O] | 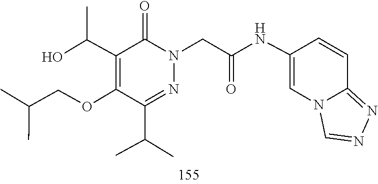 [SMILES: CC(C)COc1c(C(C)C)nn(CC(=O)Nc2ccc3nncn3c2)c(=O)c1C(C)O] |
| 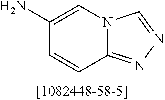 | 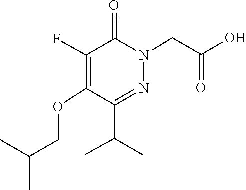 [SMILES: CC(C)COc1c(C(C)C)nn(CC(=O)O)c(=O)c1F] | 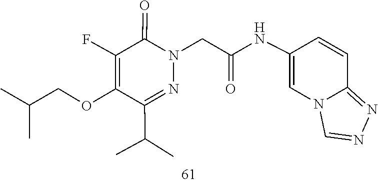 [SMILES: CC(C)COc1c(C(C)C)nn(CC(=O)Nc2ccc3nncn3c2)c(=O)c1F] |
| 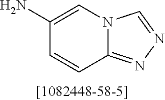 | 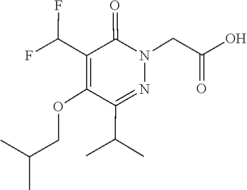 [SMILES: CC(C)COc1c(C(C)C)nn(CC(=O)O)c(=O)c1C(F)F] | 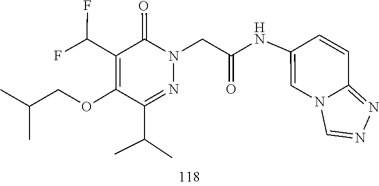 [SMILES: CC(C)COc1c(C(C)C)nn(CC(=O)Nc2ccc3nncn3c2)c(=O)c1C(F)F] |
| 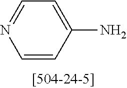 | 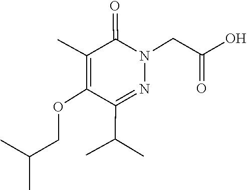 [SMILES: Cc1c(OCC(C)C)c(C(C)C)nn(CC(=O)O)c1=O] | 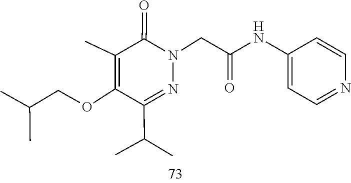 [SMILES: Cc1c(OCC(C)C)c(C(C)C)nn(CC(=O)Nc2ccncc2)c1=O] |
| 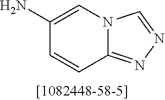 | 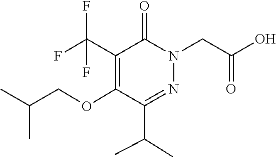 [SMILES: CC(C)COc1c(C(C)C)nn(CC(=O)O)c(=O)c1C(F)(F)F] | 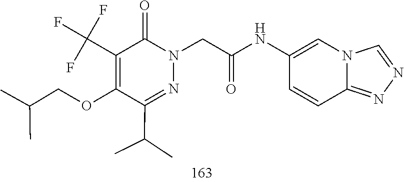 [SMILES: CC(C)COc1c(C(C)C)nn(CC(=O)Nc2ccc3nncn3c2)c(=O)c1C(F)(F)F] |
| 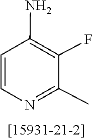 | 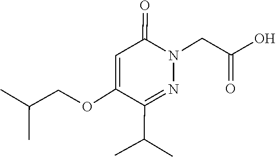 [SMILES: CC(C)COc1cc(=O)n(CC(=O)O)nc1C(C)C] | 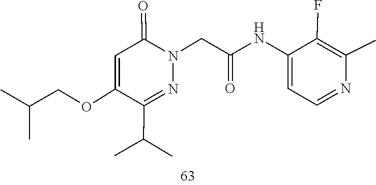 [SMILES: Cc1nccc(NC(=O)Cn2nc(C(C)C)c(OCC(C)C)cc2=O)c1F] |
| 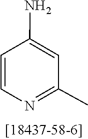 | 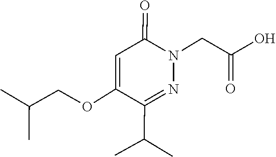 [SMILES: CC(C)COc1cc(=O)n(CC(=O)O)nc1C(C)C] | 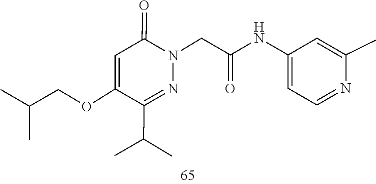 [SMILES: Cc1cc(NC(=O)Cn2nc(C(C)C)c(OCC(C)C)cc2=O)ccn1] |
| 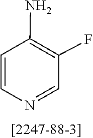 | 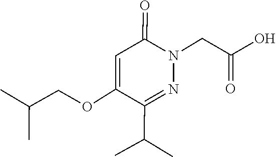 [SMILES: CC(C)COc1cc(=O)n(CC(=O)O)nc1C(C)C] | 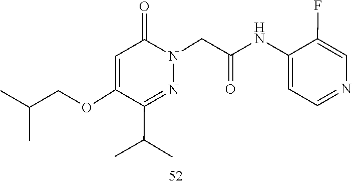 [SMILES: CC(C)COc1cc(=O)n(CC(=O)Nc2ccncc2F)nc1C(C)C] |
| 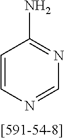 | 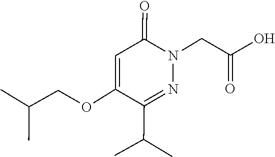 [SMILES: CC(C)COc1cc(=O)n(CC(=O)O)nc1C(C)C] | 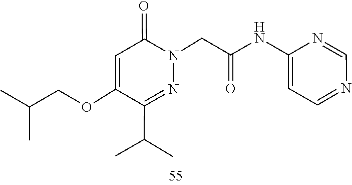 |
| 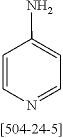 | 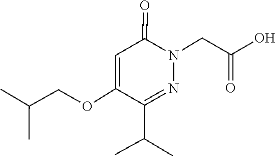 [SMILES: CC(C)COc1cc(=O)n(CC(=O)O)nc1C(C)C] | 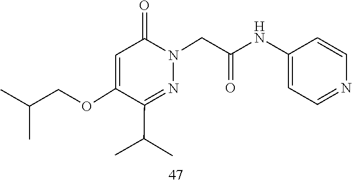 [SMILES: CC(C)COc1cc(=O)n(CC(=O)Nc2ccncc2)nc1C(C)C] |
| 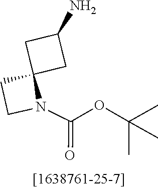 | 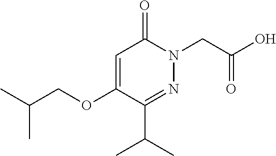 [SMILES: CC(C)COc1cc(=O)n(CC(=O)O)nc1C(C)C] | 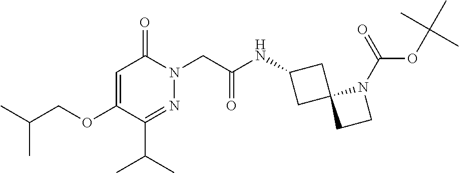 [SMILES: CC(C)COc1cc(=O)n(CC(=O)N[C@H]2C[C@]3(CCN3C(=O)OC(C)(C)C)C2)nc1C(C)C] |
| 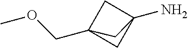 | 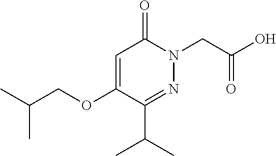 [SMILES: CC(C)COc1cc(=O)n(CC(=O)O)nc1C(C)C] | 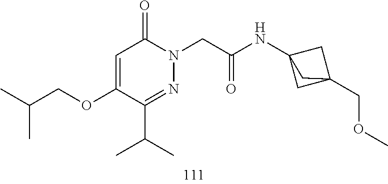 [SMILES: COCC12CC(NC(=O)Cn3nc(C(C)C)c(OCC(C)C)cc3=O)(C1)C2] |
| 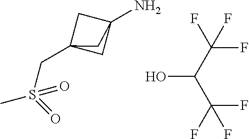 | 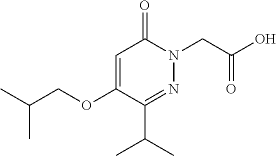 [SMILES: CC(C)COc1cc(=O)n(CC(=O)O)nc1C(C)C] | 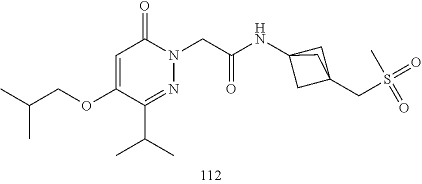 [SMILES: CC(C)COc1cc(=O)n(CC(=O)NC23CC(CS(C)(=O)=O)(C2)C3)nc1C(C)C] |
| 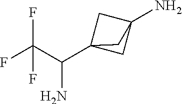 [SMILES: NC(C(F)(F)F)C12CC(N)(C1)C2] | 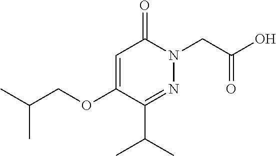 [SMILES: CC(C)COc1cc(=O)n(CC(=O)O)nc1C(C)C] | 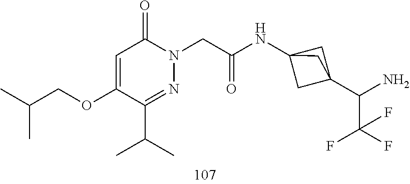 [SMILES: CC(C)COc1cc(=O)n(CC(=O)NC23CC(C(N)C(F)(F)F)(C2)C3)nc1C(C)C] |

### Synthesis of N-([1,2,4]triazolo[4,3-a]pyridin-6-yl)-2-(4-isobutoxy-3-isopropyl-5-methyl-6-oxopyridazin-1(6H)-yl)acetamide 54

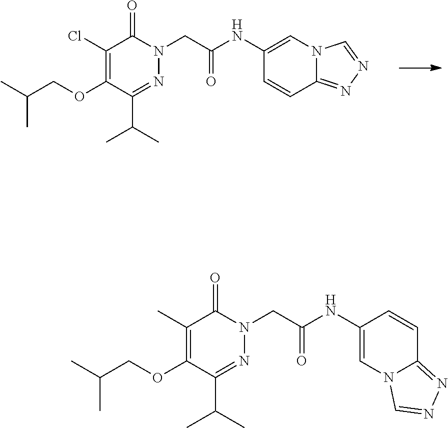 [SMILES: CC(C)COc1c(C(C)C)nn(CC(=O)Nc2ccc3nncn3c2)c(=O)c1Cl ; Cc1c(OCC(C)C)c(C(C)C)nn(CC(=O)Nc2ccc3nncn3c2)c1=O]

N-([1,2,4]triazolo[4,3-a]pyridin-6-yl)-2-(5-chloro-4-isobutoxy-3-isopropyl-6-oxopyridazin-1(6H)-yl)acetamide 60 (47 mg, 0.11 mmol) and bis(tri-tert-butylphosphine)palladium(0) (23 mg, 0.045 mmol, 40 mol %) were placed in a dry 2-mL MW vial. The vial was sealed and placed under nitrogen (3 vacuum/nitrogen cycles) and cooled to 0° C. with an ice-bath. Anhydrous THF (1.2 mL) was added, the mixture was allowed to stir for 2 minutes at 0° C. and MeZnCl (2 M in THF, 168 μL, 0.34 mmol, 3 equiv) was added dropwise over 2 min. The resulting solution was stirred vigorously at r.t. for overnight. The crude mixture was quenched by addition of 0.2M HCl (ca. 5 mL) and extracted twice with DCM, The combined organic layers were dried over Na2SO4, filtered and concentrated in vacuo.

A purification was performed via Prep SFC (Stationary phase: Chiralpak Diacel AD 20×250 mm, Mobile phase: C02, iPrOH+0.4 iPrNH2) followed by a purification via Prep HPLC (Stationary phase: RP XBridge Prep C18 OBD—5 μm, 50×250 mm, Mobile phase: 0.25% NH4HCO3 solution in water, CH3CN) to obtain N-([1,2,4]triazolo[4,3-a]pyridin-6-yl)-2-(4-isobutoxy-3-isopropyl-5-methyl-6-oxopyridazin-1(6H)-yl)acetamide 54 (15 mg, yield 34%).

### Synthesis of N-(5-chlorobenzo[d]oxazol-2-yl)-2-(4-isobutoxy-3-isopropyl-6-oxopyridazin-1(6H)-yl)acetamide 141

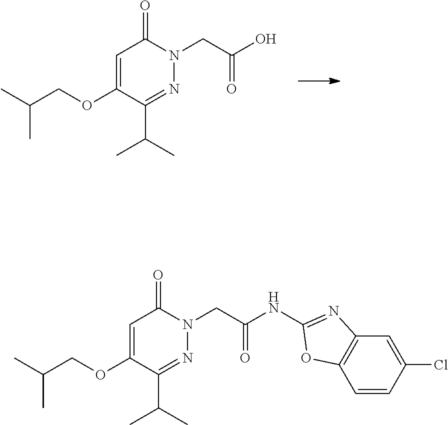 [SMILES: CC(C)COc1cc(=O)n(CC(=O)O)nc1C(C)C ; CC(C)COc1cc(=O)n(CC(=O)Nc2nc3cc(Cl)ccc3o2)nc1C(C)C]

2-(4-Isobutoxy-3-isopropyl-6-oxopyridazin-1(6H)-yl)acetic acid (150 mg, 0.56 mmol), 2-amino-5-chlorobenzoxazole [61-80-3] (113.09 mg, 0.67 mmol) and TCFH [207915-99-9] (313.72 mg, 1.12 mmol) were placed in a MW vial. The solids were suspended in 1-methylimidazole [616-47-7] (222.82 μL, 1.03 g/mL, 2.8 mmol) and ACN (6 mL). The resulting mixture was stirred at r.t. for 16 hours whereupon a thick suspension was formed. RM is partitioned between brine and DCM, organic layer is separated and water layer extracted again with DCM. Combined organic layers are dried, filtered an evaporated under reduced pressure. A purification was performed via Prep HPLC (Stationary phase: RP XBridge Prep C18 OBD-10 μm, 30×150 mm, Mobile phase: 0.25% NH4HCO3 solution in water, CH₃CN), desired fractions are combined and coevaporated twice with MeOH at 55° C. to obtain N-(5-chlorobenzo[d]oxazol-2-yl)-2-(4-isobutoxy-3-isopropyl-6-oxopyridazin-1(6H)-yl)acetamide 141 (40 mg, yield 16% o) as a yellow solid.

Additional analogues were synthesized according to the above procedure, using the appropriate reagents.

| Reagent | Product |
|---|---|
| 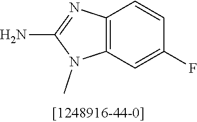 | 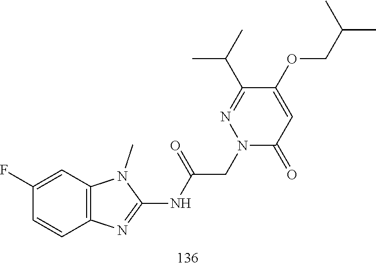 [SMILES: CC(C)COc1cc(=O)n(CC(=O)Nc2nc3ccc(F)cc3n2C)nc1C(C)C] |
| | 136 |
| 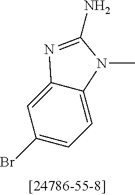 | 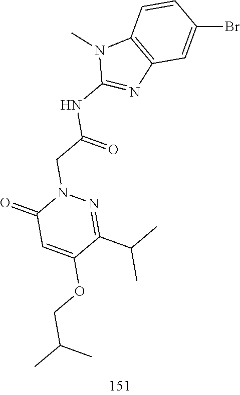 [SMILES: CC(C)COc1cc(=O)n(CC(=O)Nc2nc3cc(Br)ccc3n2C)nc1C(C)C] |
| 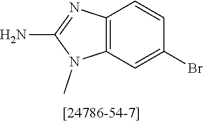 | 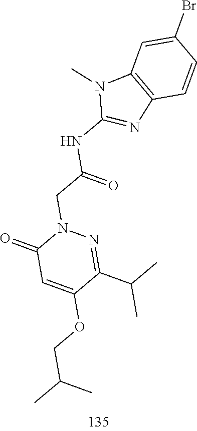 [SMILES: CC(C)COc1cc(=O)n(CC(=O)Nc2nc3ccc(Br)cc3n2C)nc1C(C)C] |
| 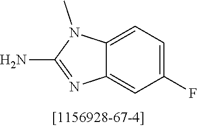 | 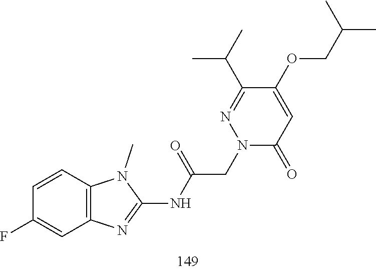 [SMILES: CC(C)COc1cc(=O)n(CC(=O)Nc2nc3cc(F)ccc3n2C)nc1C(C)C] |
| 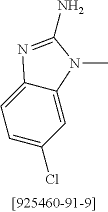 | 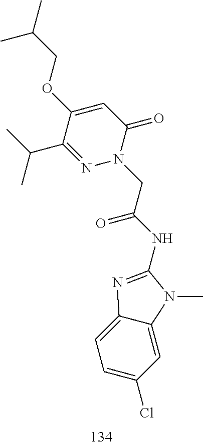 [SMILES: CC(C)COc1cc(=O)n(CC(=O)Nc2nc3ccc(Cl)cc3n2C)nc1C(C)C] |
| 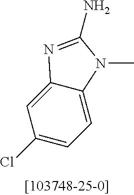 | 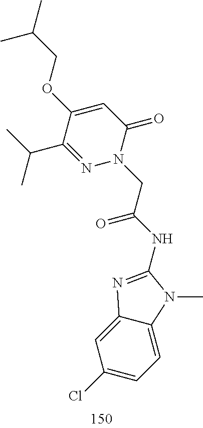 [SMILES: CC(C)COc1cc(=O)n(CC(=O)Nc2nc3cc(Cl)ccc3n2C)nc1C(C)C] |
| 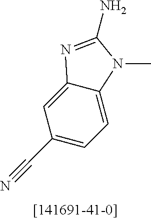 | 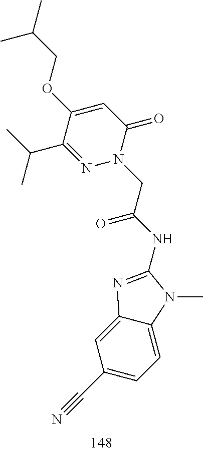 [SMILES: CC(C)COc1cc(=O)n(CC(=O)Nc2nc3cc(C#N)ccc3n2C)nc1C(C)C] |
| 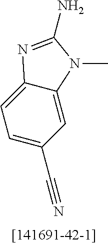 | 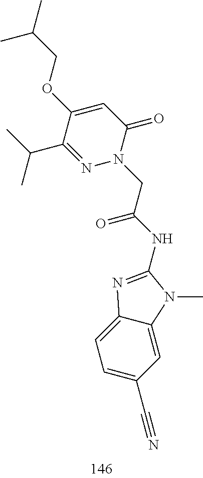 [SMILES: CC(C)COc1cc(=O)n(CC(=O)Nc2nc3ccc(C#N)cc3n2C)nc1C(C)C] |
| 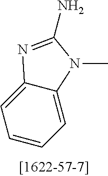 | 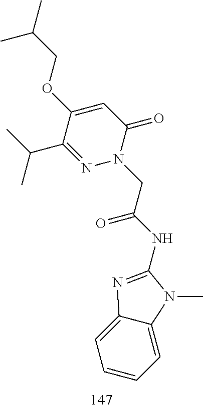 [SMILES: CC(C)COc1cc(=O)n(CC(=O)Nc2nc3ccccc3n2C)nc1C(C)C] |
| 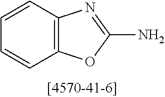 | 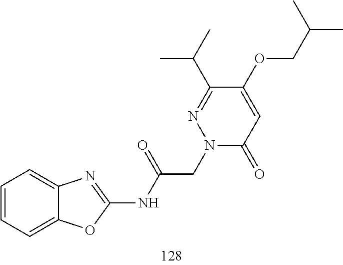 [SMILES: CC(C)COc1cc(=O)n(CC(=O)Nc2nc3ccccc3o2)nc1C(C)C] |
| 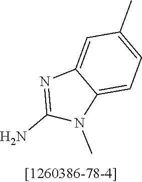 | 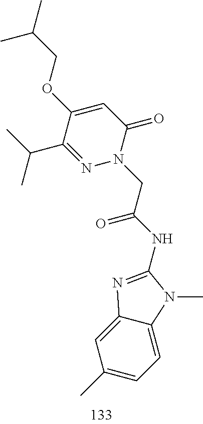 [SMILES: Cc1ccc2c(c1)nc(NC(=O)Cn1nc(C(C)C)c(OCC(C)C)cc1=O)n2C] |
| 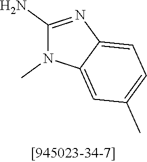 | 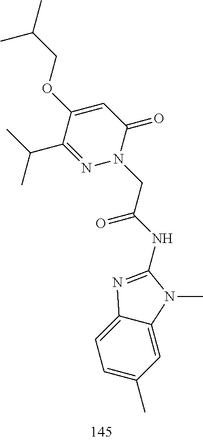 [SMILES: Cc1ccc2nc(NC(=O)Cn3nc(C(C)C)c(OCC(C)C)cc3=O)n(C)c2c1] |
| 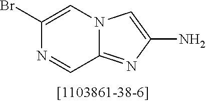 [SMILES: Nc1cn2cc(Br)ncc2n1] | 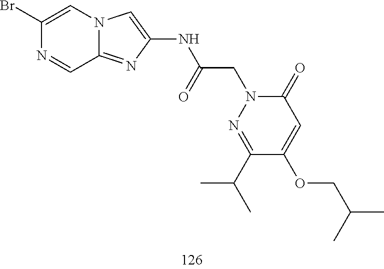 [SMILES: CC(C)COc1cc(=O)n(CC(=O)Nc2cn3cc(Br)ncc3n2)nc1C(C)C] |
| 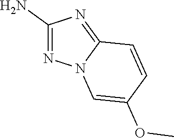 | 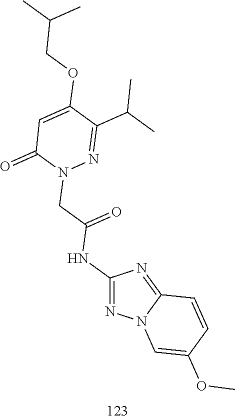 [SMILES: COc1ccc2nc(NC(=O)Cn3nc(C(C)C)c(OCC(C)C)cc3=O)nn2c1] |
| 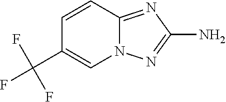 [SMILES: Nc1nc2ccc(C(F)(F)F)cn2n1] | 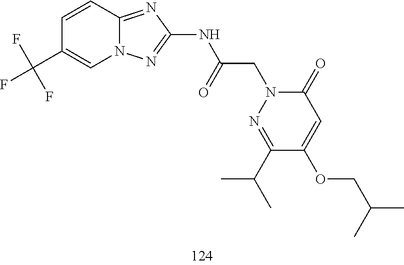 [SMILES: CC(C)COc1cc(=O)n(CC(=O)Nc2nc3ccc(C(F)(F)F)cn3n2)nc1C(C)C] |
| 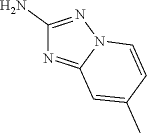 | 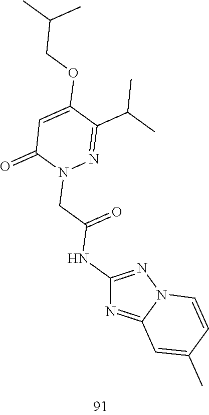 [SMILES: Cc1ccn2nc(NC(=O)Cn3nc(C(C)C)c(OCC(C)C)cc3=O)nc2c1] |
| 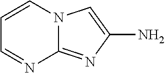 | 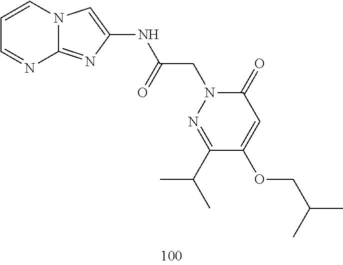 [SMILES: CC(C)COc1cc(=O)n(CC(=O)Nc2cn3cccnc3n2)nc1C(C)C] |
| 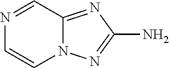 | 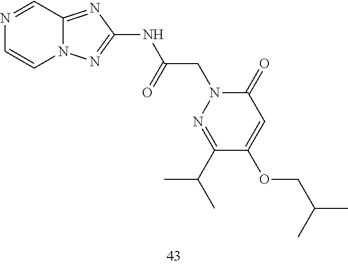 [SMILES: CC(C)COc1cc(=O)n(CC(=O)Nc2nc3cnccn3n2)nc1C(C)C] |
| 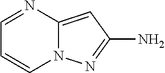 | 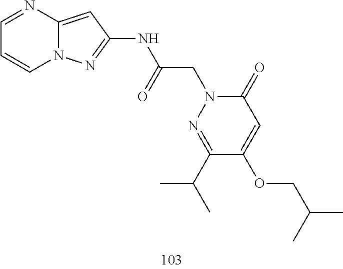 [SMILES: CC(C)COc1cc(=O)n(CC(=O)Nc2cc3ncccn3n2)nc1C(C)C] |
| 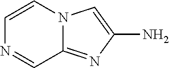 | 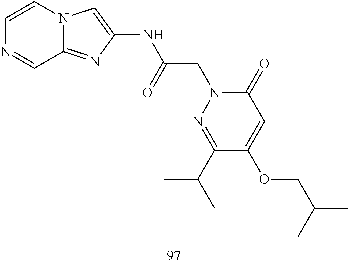 [SMILES: CC(C)COc1cc(=O)n(CC(=O)Nc2cn3ccncc3n2)nc1C(C)C] |
| 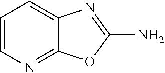 | 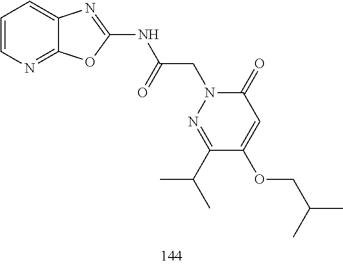 [SMILES: CC(C)COc1cc(=O)n(CC(=O)Nc2nc3cccnc3o2)nc1C(C)C] |
| 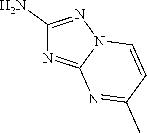 | 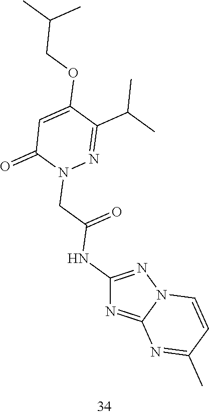 [SMILES: Cc1ccn2nc(NC(=O)Cn3nc(C(C)C)c(OCC(C)C)cc3=O)nc2n1] |
| 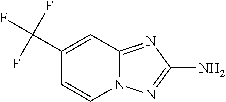 [SMILES: Nc1nc2cc(C(F)(F)F)ccn2n1] | 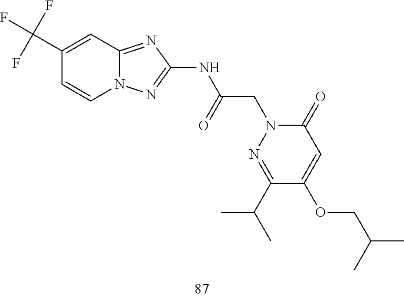 [SMILES: CC(C)COc1cc(=O)n(CC(=O)Nc2nc3cc(C(F)(F)F)ccn3n2)nc1C(C)C] |
| 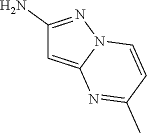 | 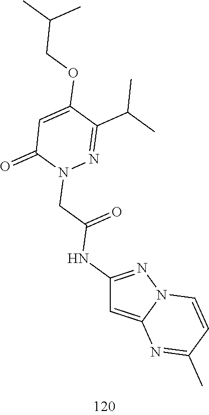 [SMILES: Cc1ccn2nc(NC(=O)Cn3nc(C(C)C)c(OCC(C)C)cc3=O)cc2n1] |
| 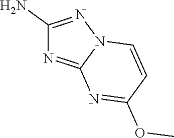 | 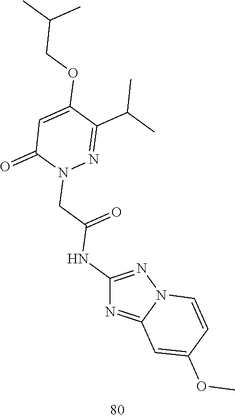 [SMILES: COc1ccn2nc(NC(=O)Cn3nc(C(C)C)c(OCC(C)C)cc3=O)nc2c1] |
| 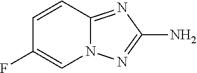 [SMILES: Nc1nc2ccc(F)cn2n1] | 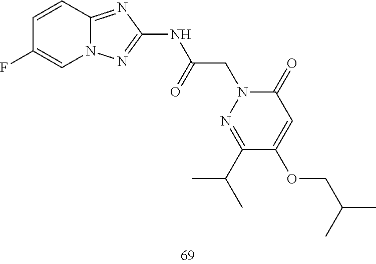 [SMILES: CC(C)COc1cc(=O)n(CC(=O)Nc2nc3ccc(F)cn3n2)nc1C(C)C] |
| 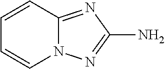 | 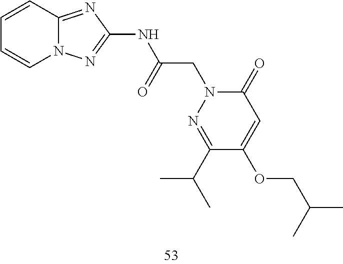 [SMILES: CC(C)COc1cc(=O)n(CC(=O)Nc2nc3ccccn3n2)nc1C(C)C] |
| 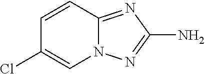 [SMILES: Nc1nc2ccc(Cl)cn2n1] | 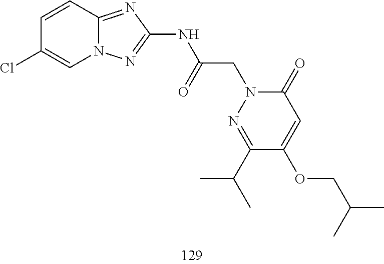 [SMILES: CC(C)COc1cc(=O)n(CC(=O)Nc2nc3ccc(Cl)cn3n2)nc1C(C)C] |
| 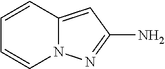 | 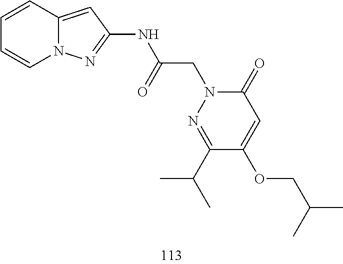 [SMILES: CC(C)COc1cc(=O)n(CC(=O)Nc2cc3ccccn3n2)nc1C(C)C] |
| 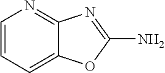 | 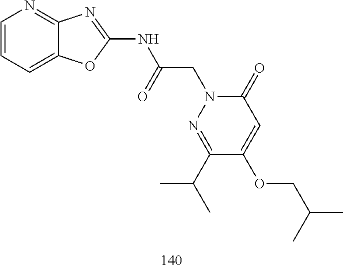 [SMILES: CC(C)COc1cc(=O)n(CC(=O)Nc2nc3ncccc3o2)nc1C(C)C] |
| 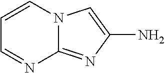 | 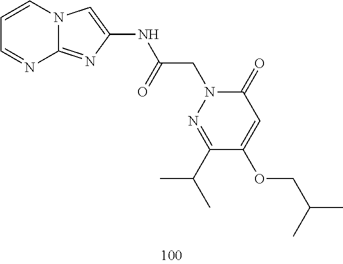 [SMILES: CC(C)COc1cc(=O)n(CC(=O)Nc2cn3cccnc3n2)nc1C(C)C] |
| 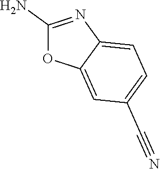 [SMILES: N#Cc1ccc2nc(N)oc2c1] | 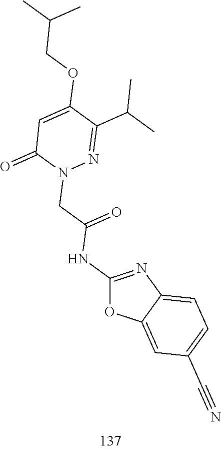 [SMILES: CC(C)COc1cc(=O)n(CC(=O)Nc2nc3ccc(C#N)cc3o2)nc1C(C)C] |

### Synthesis of 2-(4-isobutoxy-3-isopropyl-6-oxopyridazin-1(6H)-yl)-N-(5-methyl-7-(trifluoromethyl)-[1,2,4]triazolo[1,5-a]pyrimidin-2-yl)acetamide 130

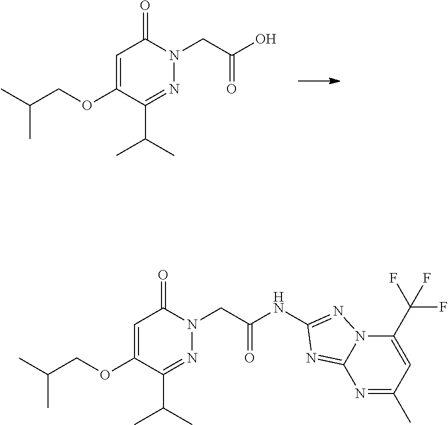 [SMILES: CC(C)COc1cc(=O)n(CC(=O)O)nc1C(C)C ; Cc1cc(C(F)(F)F)n2nc(NC(=O)Cn3nc(C(C)C)c(OCC(C)C)cc3=O)nc2n1]

Oxalyl chloride [79-37-8] (31 μL, 1.5 g/mL, 0.37 mmol) and a drop of DMF [68-12-2] were sequentially added to a stirred suspension of 2-(4-isobutoxy-3-isopropyl-6-oxopyridazin-1(6H)-yl)acetic acid (97.12 mg, 0.36 mmol) in 4 mL of anhydrous DCM. After 1h of stirring at room temperature, the mixture was slowly added to a stirred mixture of 7-methyl-5-(trifluoromethyl)-[1,2,4]triazolo[1,5-A]pyrimidin-2-amine [575496-43-4] (104 mg, 0.48 mmol) in 4 mL of anhydrous pyridine. The resulting yellowish mixture was stirred at room temperature overnight. Water and DCM were added (+brine+bicarbonate). The organic layer was separated and the aqueous layer was back-extracted with DCM (×4). The combined dried (MgSO4) organic layers were evaporated under reduced pressure and a purification was performed via Prep HPLC (Stationary phase: RP XBridge Prep C18 OBD-10 μm, 30×150 mm, Mobile phase: 0.25% NH4HCO3 solution in water, CH3CN) to yield 2-(4-isobutoxy-3-isopropyl-6-oxopyridazin-1(6H)-yl)-N-(5-methyl-7-(trifluoromethyl)-[1,2,4]triazolo[1,5-a]pyrimidin-2-yl)acetamide 130 (15 mg, yield 9%).

### Synthesis of methyl 2-(3-aminobicyclo[1.1.1]pentan-1-yl)acetate

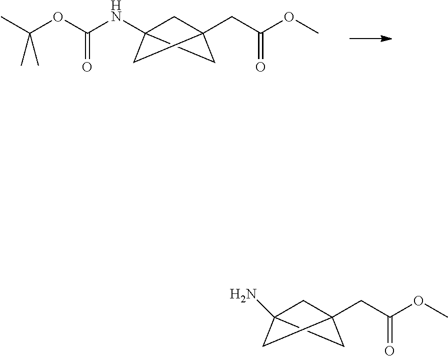 [SMILES: COC(=O)CC12CC(NC(=O)OC(C)(C)C)(C1)C2 ; COC(=O)CC12CC(N)(C1)C2]

The reaction was carried out using methyl 2-(3-((tert-butoxycarbonyl)amino)bicyclo[1.1.1]pentan-1-yl)acetate [1995848-08-2] (100 mg, 0.392 mmol) as starting material and a Synple Boc-deprotection cartridge (Reagent-cartridge Boc deprotection 0.5 mmol) to afford methyl 2-(3-aminobicyclo[1.1.1]pentan-1-yl)acetate (140 mg, assumed quant. yield) as a sticky solid which was used without further purification for the next step.

### Synthesis of methyl 2-(3-(2-(4-isobutoxy-3-isopropyl-6-oxopyridazin-1(6H)-yl)acetamido)bicyclo[1.1.1]pentan-1-yl)acetate 71

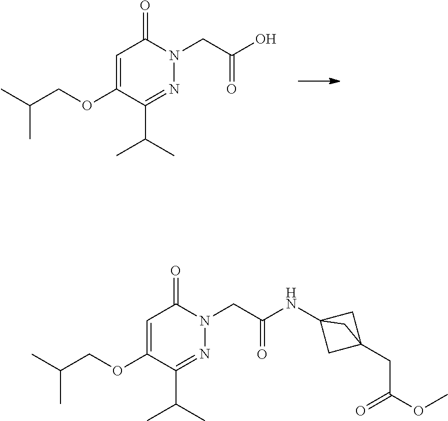 [SMILES: CC(C)COc1cc(=O)n(CC(=O)O)nc1C(C)C ; COC(=O)CC12CC(NC(=O)Cn3nc(C(C)C)c(OCC(C)C)cc3=O)(C1)C2]

2-(4-isobutoxy-3-isopropyl-6-oxopyridazin-1(6H)-yl)acetic acid (80 mg, 0.298 mmol) and methyl 2-(3-aminobicyclo[1.1.1]pentan-1-yl)acetate (120 mg, 0.33 mmol) were dissolved in 2 mL of DCM and 2 mL of EtOH. The resulting solution was used in the Synple system using the amide-bond formation cartridge (3 h). Upon completion, the mixture was evaporated under reduced pressure. A purification was performed via Prep HPLC (Stationary phase: RP XBridge Prep C18 OBD-10 μm, 30×150 mm, Mobile phase: 0.25% NH4HCO3 solution in water, CH3CN). The purest fractions were collected, evaporated under reduced pressure and coevaporated with MeOH to afford methyl 2-(3-(2-(4-isobutoxy-3-isopropyl-6-oxopyridazin-1(6H)-yl)acetamido)bicyclo[1.1.1]pentan-1-yl)acetate 71 (91 mg, yield 75%) as a white solid.

Characterising Data—LC-MS and Melting Point

LCMS: [M+H]⁺ means the protonated mass of the free base of the compound, Rₜ means retention time (in minutes), method refers to the method used for LCMS.

| Number | LCMS |
|---|---|
| 1F | Rt: 1.74, Area %: 98.25, MW: 497.24, BPM1: 498.2, Method 5 |
| 2F | Rt: 1.74, Area %: 98.25, MW: 497.24, BPM1: 498.2, Method 5 |
| 3F | Rt: 1.83, Area %: 100.00, MW: 483.22, BPM1: 484.2, Method 5 |
| 4F | Rt: 1.06, Area %: 100.00, MW: 427.20, BPM1: 428.2, Method 5 |
| 5F | Rt: 1.85, Area %: 100.00, MW: 396.19, BPM1: 397.2, Method 5 |
| 6F | Rt: 0.98, Area %: 97.36, MW: 446.14, BPM1: 447.1, Method 5 |
| 7F | Rt: 1.91, Area %: 100.00, MW: 396.19, BPM1: 397.2, Method 5 |
| 8F | Rt: 1.52, Area %: 100.00, MW: 406.16, BPM1: 407.2, Method 5 |
| 9F | Rt: 1.78, Area %: 100.00, MW: 384.19, BPM1: 385.2, Method 5 |
| 10F | Rt: 1.84, Area %: 100.00, MW: 384.19, BPM1: 385.2, Method 5 |
| 11F | Rt: 1.58, Area %: 98.90, MW: 402.18, BPM1: 403.2, Method 5 |
| 12F | Rt: 0.92, Area %: 100.00, MW: 399.20, BPM1: 400.2, Method 5 |
| 13F | Rt: 1.04, Area %: 100.00, MW: 384.15, BPM1: 385.2, Method 5 |
| 14F | Rt: 1.69, Area %: 100.00, MW: 382.17, BPM1: 383.2, Method 5 |
| 15F | Rt: 1.24, Area %: 100.00, MW: 412.19, BPM1: 413.2, Method 5 |
| 16F | Rt: 1.99, Area %: 100.00, MW: 452.18, BPM1: 453.2, Method 5 |
| 17F | Rt: 1.34, Area %: 98.79, MW: 392.14, BPM1: 393.1, Method 5 |
| 18F | Rt: 1.57, Area %: 95.28, MW: 370.18, BPM1: 371.2, Method 5 |
| 19F | Rt: 1.72, Area %: 100.00, MW: 432.17, BPM1: 433.2, Method 5 |
| 20F | Rt: 1.75, Area %: 100.00, MW: 424.15, BPM1: 425.2, Method 5 |
| 21F | Rt: 1.56, Area %: 100.00, MW: 418.16, BPM1: 419.2, Method 5 |
| 22F | Rt: 1.64, Area %: 100.00, MW: 382.18, BPM1: 383.2, Method 5 |
| 23F | Rt: 1.58, Area %: 100.00, MW: 410.13, BPM1: 411.1, Method 5 |
| 24F | Rt: 1.62, Area %: 99.44, MW: 370.18, BPM1: 371.2, Method 5 |
| 25F | Rt: 1.78, Area %: 100.00, MW: 404.16, BPM1: 405.2, Method 5 |
| 26F | Rt: 1.29, Area %: 100.00, MW: 412.19, BPM1: 413.2, Method 5 |
| 27C | Rt: 1.13, Area %: 100.00, MW: 343.14, BPM1: 344.14, Method 5 |
| 28E | Rt: 1.51, Area %: 90.74, MW: 382.18, BPM1: 383.2, Method 5 |
| 29E | Rt: 1.32, Area %: 97.51, MW: 420.05, BPM1: 421.1, Method 5 |
| 30G | Rt: 1.12, Area %: 100.00, MW: 410.17, BPM1: 411.2, Method 5 |
| 32E | Rt: 1.24, Area %: 95.21, MW: 423.17, BPM1: 424.2, Method 5 |
| 31C | Rt: 1.23, Area %: 100.00, MW: 355.14, BPM1: 356.3, BPM2: 354.4, |
| | Method 8 |
| 33D | Rt: 1.22, Area %: 100.00, MW: 354.14, BPM1: 355.3, BPM2: 353.4, |
| | Method 8 |
| 143 | RT: 1.75, Area %: 90.34, MW: 405.15, BPM1: 406.2, Method: 5 |
| 162 | RT: 1.12, Area %: 100.00, MW: 342.14, BPM1: 343.2, Method: 5 |
| 161 | RT: 0.30, Area %: 97.58, MW: 383.17, BPM1: 384.2, Method: 15 |
| 160 | RT: 0.25, Area %: 97.58, MW: 397.19, BPM1: 398.3, Method: 15 |
| 159 | RT: 0.32, Area %: 100.00, MW: 397.19, BPM1: 398.3, Method: 15 |
| 64 | RT: 1.49, Area %: 98.31, MW: 400.17, BPM1: 401.2, Method: 20 |
| 41 | RT: 1.73, Area %: 100.00, MW: 385.00, BPM1: 386, BPM2: 384, |
| | Method: 4 |
| 49 | RT: 2.07, Area %: 100.00, MW: 384.19, BPM1: 385.19, Method: 5 |
| 158 | RT: 1.080, Area %: 99, MW: 255 BPM1: 256.2, Method: 13 |
| 157 | RT: 2.230, Area %: 99, MW 253 BPM1: 254.1, Method: 1 |
| 39 | RT: 1.87, Area %: 100.00, MW: 419.00, BPM1: 420, BPM2: 420, |
| | Method: 3 |
| 60 | RT: 1.83, Area %: 100.00, MW: 418.00, BPM1: 419, BPM2: 417, |
| | Method: 3 |
| 84 | RT: 1.83, Area %: 94.17, MW: 404.14, BPM1: 405.1, Method: 5 |
| 138 | RT: 0.92, Area %: 94.89, MW: 458.10, BPM1: 459, Method: 16 |
| 114 | RT: 1.76, Area %: 97, MW: 444.09, BPM1: 445.1, Method: 5 |
| 74 | RT: 1.81, Area %: 100.00, MW: 416.14, BPM1: 417.1, Method: 5 |
| 72 | RT: 1.79, Area %: 97.93, MW: 445.09, BPM1: 446.1, Method: 5 |
| 50 | RT: 1.79, Area %: 99.06, MW: 459.10, BPM1: 460.1, Method: 5 |
| 48 | RT: 1.84, Area %: 93.58, MW: 405.13, BPM1: 406.1, Method: 5 |
| 44 | RT: 1.84, Area %: 93.50, MW: 417.13, BPM1: 418.1, Method: 5 |
| 156 | RT: 2.574, Area %: 99, MW: 466, BPM1: 467, Method: 1 |
| 108 | RT: 3.165, Area %: 97, MW 415, BPM1: 416.2, Method: 1 |
| 35 | RT: 2.245, Area %: 99, MW: 383, BPM1: 384.10, Method: 1 |
| 54 | RT: 1.78, Area %: 100.00, MW: 398.2, BPM1: 399.0, BPM2: 397.1, |
| | Method: 3 |
| 70 | RT: 2.460, Area %: 99, MW: 385, BPM1: 386.2, Method: 1 |
| 127 | RT: 1.73, Area %: 100.00, MW: 429.00, BPM1: 430, BPM2: 428, |
| | Method: 4 |
| 155 | RT: 1.73, Area %: 100.00, MW: 428.00, BPM1: 429, BPM2: 427, |
| | Method: 3 |
| 76 | RT: 2.320, Area %: 98, MW: 384, BPM1: 385.2, Method: 1 |
| 142 | RT: 2.652, Area %: 99, MW: 467, BPM1: 468.0, Method: 1 |
| 154 | RT: 2.123, Area %: 99, MW: 399, BPM1: 400.1, Method: 1 |
| 58 | RT: 2.540, Area %: 99, MW: 386, BPM1: 387.2, Method: 1 |
| 61 | RT: 1.75, Area %: 97.69, MW: 402.00, BPM1: 403, BPM2: 401, |
| | Method: 3 |
| 93 | RT: 1.70, Area %: 97.04, MW: 440.12, BPM1: 441.1, Method: 5 |
| 110 | RT: 2.41, Area %: 98.05, MW: 333.21, BPM1: 334.2, Method: 5 |
| 46 | RT: 1.72, Area %: 100.00, MW: 441.11, BPM1: 442.1, Method: 5 |
| 95 | RT: 2.345, MW: 412, BPM1: 413.1, Method: 1 |
| 105 | RT: 1.03, Area %: 100.00, MW: 351.20, BPM1: 352.3, BPM2: 352.2, |
| | Method: 15 |
| 92 | RT: 1.14, Area %: 100.00, MW: 347.22, BPM1: 348.3, BPM2: 346.3, |
| | Method: 15 |
| 66 | RT: 0.96, Area %: 100.00, MW: 358.20, BPM1: 359.2, BPM2: 357.4, |
| | Method: 15 |
| 132 | RT: 1.09, Area %: 100.00, MW: 448.27, BPM1: 449.3, BPM2: 447.4, |
| | Method: 15 |
| 79 | RT: 0.73, Area %: 99.23, MW: 363.22, BPM1: 364.3, BPM2: 362.4, |
| | Method: 15 |
| 83 | RT: 1.08, Area %: 99.36, MW: 383.20, BPM1: 384.3, BPM2: 382.4, |
| | Method: 15 |
| 121 | RT: 1.02, Area %: 99.40, MW: 416.28, BPM1: 417.4, BPM2: 415.4, |
| | Method: 15 |
| 94 | RT: 0.75, Area %: 99.44, MW: 418.26, BPM1: 419.3, BPM2: 417.4, |
| | Method: 15 |
| 122 | RT: 1.19, Area %: 100.00, MW: 476.30, BPM1: 477.4, BPM2: 475.5, |
| | Method: 15 |
| 115 | RT: 1.12, Area %: 100.00, MW: 462.28, BPM1: 463.4, BPM2: 517.4, |
| | Method: 15 |
| 153 | RT: 1.25, Area %: 99.49, MW: 490.32, BPM1: 491.3, BPM2: 489.4, |
| | Method: 15 |
| 36 | RT: 3.368, Area %: 99, MW: 391, BPM1: 392.2, Method: 1 |
| 37 | RT: 0.99, Area %: 99.20, MW: 427.21, BPM1: 428.3, BPM2: 408.4, |
| | Method: 15 |
| 40 | RT: 0.97, Area %: 100.00, MW: 405.23, BPM1: 406.3, BPM2: 460.4, |
| | Method: 15 |
| 38 | RT: 1.02, Area %: 100.00, MW: 419.24, BPM1: 420.3, BPM2: 418.4, |
| | Method: 15 |
| 45 | RT: 1.09, Area %: 100.00, MW: 433.26, BPM1: 434.3, BPM2: 432.4, |
| | Method: 15 |
| 82 | RT: 0.68, Area %: 93.91, MW: 349.20, BPM1: 350.2, BPM2: 348.4, |
| | Method: 15 |
| 88 | RT: 0.74, Area %: 100.00, MW: 391.25, BPM1: 392.3, BPM2: 390.4, |
| | Method: 15 |
| 59 | RT: 0.71, Area %: 99.40, MW: 377.23, BPM1: 378.3, BPM2: 376.4, |
| | Method: 15 |
| 117 | RT: 1.67, Area %: 0.52, MW: 348.22, BPM1: 349.2, Method: 5 |
| 75 | RT: 1.58, Area %: 100.00, MW: 376.25, BPM1: 753.5, Method: 5 |
| 86 | RT: 1.64, Area %: 100.00, MW: 362.23, BPM1: 363, Method: 5 |
| 99 | RT: 1.65, Area %: 100.00, MW: 390.26, BPM1: 391, Method: 5 |
| 96 | RT: 0.80, Area %: 95.06, MW: 391.21, BPM1: 392.2, BPM2: 390.5, |
| | Method: 17 |
| 104 | RT: 1.49, Area %: 100.00, MW: 405.23, BPM1: 406.2, BPM2: 404, |
| | Method: 5 |
| 67 | RT: 1.62, Area %: 100.00, MW: 419.24, BPM1: 420.3, BPM2: 418, |
| | Method: 5 |
| 131 | RT: 1.36, Area %: 94, MW: 377.19, BPM1: 376.2, BPM2: 375, Method: |
| | 18 |
| 125 | RT: 1.41, Area %: 97.44, MW: 413.19, BPM1: 414.2, BPM2: 412, |
| | Method: 5 |
| 62 | RT: 1.89, Area %: 100.00, MW: 435.00, BPM1: 436, BPM2: 434, |
| | Method: 3 |
| 42 | RT: 1.83, Area %: 100.00, MW: 399.00, BPM1: 400, BPM2: 398, |
| | Method: 3 |
| 68 | RT: 1.90, Area %: 100.00, MW: 398.00, BPM1: 399, BPM2: 399, |
| | Method: 4 |
| 118 | RT: 1.87, Area %: 100.00, MW: 434.00, BPM1: 435, BPM2: 433, |
| | Method: 3 |
| 106 | RT: 2.250, Area %: 99, MW: 411, BPM1: 412.1, Method: 1 |
| 73 | RT: 1.90, Area %: 100.00, MW: 358.00, BPM1: 359, BPM2: 357, |
| | Method: 4 |
| 65 | RT: 2.537, Area %: 98, MW: 358, BPM1: 359.200, Method: 1 |
| 52 | RT: 3.162, Area %: 99, MW: 362, BPM1: 363.1000, Method: 1 |
| 55 | RT: 3.212, Area %: 99, MW: 345, BPM1: 346.2000, Method: 1 |
| 47 | RT: 2.459, Area %: 99, MW: 344, BPM1: 345.2000, Method: 1 |
| 63 | RT: 4.086, Area %: 99, MW: 376, BPM1: 377.1, Method: 14 |
| 51 | RT: 1.63, Area %: 100.00, MW: 383.00, BPM1: 384, BPM2: 382, |
| | Method: 3 |
| 101 | RT: 1.96, Area %: 99.38, MW: 411.18, BPM1: 412.2, Method: 5 |
| 152 | RT: 4.188, Area %: 98, MW: 432, BPM1: 433.2000, Method: 1 |
| 90 | RT: 2.28, Area %: 96.77, MW: 415.22, BPM1: 416.2, Method: 5 |
| 139 | RT: 2.35, Area %: 97.33, MW: 414.23, BPM1: 415.2, Method: 5 |
| 119 | RT: 2.05, Area %: 100.00, MW: 415.22, BPM1: 416.2, Method: 5 |
| 56 | RT: 2.683, Area %: 99, MW: 407, BPM1: 408.1000, Method: 1 |
| 98 | RT: 1.79, Area %: 93.31, MW: 429.24, BPM1: 430.2, Method: 5 |
| 85 | RT: 1.06, Area %: 100, MW: 391.00, BPM1: 392, BPM2: 392, Method: |
| | 10 |
| 102 | RT: 3.099, Area %: 99, MW: 425, BPM1: 426.2282, Method: 19 |
| 78 | RT: 2.26, Area %: 90.88, MW: 439.21, BPM1: 440.2, Method: 9 |
| 141 | RT: 1.94, Area %: 95.13, MW: 418.00, BPM1: 251, BPM2: 417, |
| | Method: 4 |
| 116 | RT: 2.10, Area %: 98.38, MW: 437.20, BPM1: 438.2, Method: 5 |
| 57 | RT: 2.707, Area %: 99, MW: 401, BPM1: 402.1000, Method: 1 |
| 136 | RT: 2.12, Area %: 100.00, MW: 415.00, BPM1: 416, BPM2: 414, |
| | Method: 4 |
| 77 | RT: 1.68, Area %: 98.93, MW: 385.00, BPM1: 386, BPM2: 384, |
| | Method: 4 |
| 135 | RT: 2.27, Area %: 100.00, MW: 475.00, BPM1: 478, BPM2: 476, |
| | Method: 12 |
| 151 | RT: 2.24, Area %: 100.00, MW: 475.00, BPM1: 478, BPM2: 476, |
| | Method: 12 |
| 134 | RT: 2.24, Area %: 98.51, MW: 431.00, BPM1: 432, BPM2: 430, |
| | Method: 12 |
| 150 | RT: 2.22, Area %: 100.00, MW: 431.00, BPM1: 432, BPM2: 430, |
| | Method: 12 |
| 149 | RT: 2.07, Area %: 100.00, MW: 415.20, BPM1: 416, BPM2: 414, |
| | Method: 4 |
| 148 | RT: 2.04, Area %: 100.00, MW: 422.00, BPM1: 423, BPM2: 421, |
| | Method: 12 |
| 147 | RT: 2.12, Area %: 100.00, MW: 397.00, BPM1: 398, BPM2: 396, |
| | Method: 12 |
| 128 | RT: 1.94, Area %: 98.94, MW: 384.00, BPM1: 251, BPM2: 251, |
| | Method: 4 |
| 146 | RT: 2.02, Area %: 100.00, MW: 422.00, BPM1: 423, BPM2: 423, |
| | Method: 4 |
| 133 | RT: 2.19, Area %: 100.00, MW: 411.00, BPM1: 412, BPM2: 410, |
| | Method: 4 |
| 145 | RT: 2.19, Area %: 100.00, MW: 411.00, BPM1: 412, BPM2: 410, |
| | Method: 4 |
| 89 | RT: 2.005, Area %: 99, MW: 376, BPM1: 377.2000, Method: 1 |
| 81 | RT: 2.107, Area %: 99, MW: 390, BPM1: 391.2000, Method: 1 |
| 130 | RT: 2.02, Area %: 98.17, MW: 467.00, BPM1: 468, BPM2: 466, |
| | Method: 3 |
| 71 | RT: 1.07, Area %: 100.00, MW: 405.00, BPM1: 406, BPM2: 464, |
| | Method: 11 |
| 111 | RT: 3.194, Area %: 99, MW: 377, BPM1: 378.2, Method: 1 |
| 126 | RT: 1.09, Area %: 100.00, MW: 462.10, BPM1: 251, BPM2: 461, |
| | Method: 11 |
| 123 | RT: 1.00, Area %: 100.00, MW: 414.20, BPM1: 415, BPM2: 413, |
| | Method: 11 |
| 124 | RT: 1.10, Area %: 100.00, MW: 452.18, BPM1: 251, BPM2: 251, |
| | Method: 11 |
| 91 | RT: 1.00, Area %: 100.00, MW: 398.21, BPM1: 399, BPM2: 397, |
| | Method: 11 |
| 100 | RT: 0.97, Area %: 100.00, MW: 384.19, BPM1: 385, BPM2: 385, |
| | Method: 11 |
| 43 | RT: 0.90, Area %: 99.19, MW: 385.19, BPM1: 251, BPM2: 384, |
| | Method: 11 |
| 103 | RT: 0.97, Area %: 100.00, MW: 384.19, BPM1: 251, BPM2: 383, |
| | Method: 11 |
| 97 | RT: 0.97, Area %: 100.00, MW: 384.19, BPM1: 385, BPM2: 383, |
| | Method: 11 |
| 144 | RT: 0.99, Area %: 100.00, MW: 385.18, BPM1: 251, BPM2: 384, |
| | Method: 11 |
| 34 | RT: 0.93, Area %: 100.00, MW: 399.20, BPM1: 400, BPM2: 398, |
| | Method: 11 |
| 163 | RT: 1.91, Area %: 98, MW: 452.1, BPM1: 453, BPM2: 451, Method: 12 |
| 53 | RT: 0.94, Area %: 100.00, MW: 384.19, BPM1: 385, BPM2: 383, |
| | Method: 11 |
| 87 | RT: 1.07, Area %: 100.00, MW: 452.18, BPM1: 453, BPM2: 451, |
| | Method: 11 |
| 120 | RT: 1.00, Area %: 100.00, MW: 398.21, BPM1: 251, BPM2: 397, |
| | Method: 11 |
| 80 | RT: 0.93, Area %: 2.68, MW: 414.20, BPM1: 251, BPM2: 413, Method: |
| | 11 |
| 69 | RT: 0.97, Area %: 100.00, MW: 402.18, BPM1: 403, BPM2: 401, |
| | Method: 11 |
| 129 | RT: 1.02, Area %: 95.03, MW: 418.15, BPM1: 419, BPM2: 417, |
| | Method: 11 |
| 113 | RT: 1.08, Area %: 95.84, MW: 383.20, BPM1: 384, BPM2: 632, |
| | Method: 11 |
| 140 | RT: 0.90, MW: 385.2, BPM1: 384, BPM2: 384, Method: 11 |
| 137 | RT: 1.00, Area %: 100.00, MW: 409.18, BPM1: 251, BPM2: 408, |
| | Method: 11 |
| 112 | RT: 3.144, Area %: 99, MW: 425, BPM1: 426.2000, Method: 1 |
| 107 | RT: 2.547, Area %: 99, MW: 430, BPM1: 431.2000, Method: 1 |
| 109 | RT: 2.650, Area %: 98, MW: 444, BPM1: 445.2000, Method: 1 |

### Characterising Data—Compound+NMR

This is depicted in the following table (it was noted that there was impurity present in Compounds 6F, 15F and 18F):

| Number | NMR |
|---|---|
| 2F | ¹H NMR (400 MHz, DMSO-d₆) δ ppm 1.15 (d, J = 6.70 Hz, 6 H) |
| | 1.38 (s, 9 H) 2.99 (tt, J = 8.29, 5.35 Hz, 1 H) 3.08 (quin, J = 6.88 |
| | Hz, 1 H) 3.74 (br s, 2 H) 3.97 (br s, 2 H) 4.16 (d, J = 5.09 Hz, 2 |
| | H) 4.83 (s, 2 H) 6.33 (s, 1 H) 7.30 (dd, J = 9.83, 1.97 Hz, 1 H) |
| | 7.79 (d, J = 9.71 Hz, 1 H) 9.20 (dd, J = 1.62, 0.92 Hz, 1 H) 9.24 (d, |
| | J = 0.69 Hz, 1 H) 10.55 (br s, 1 H) |
| 3F | ¹H NMR (400 MHz, DMSO-d₆) δ ppm 1.17 (d, J = 6.70 Hz, 6 H) |
| | 1.39 (s, 9 H) 3.15 (spt, J = 6.82 Hz, 1 H) 3.85 (br d, J = 7.17 Hz, 2 |
| | H) 4.34 (br dd, J = 9.13, 6.59 Hz, 2 H) 4.83 (s, 2 H) 5.03-5.11 |
| | (m, 1 H) 6.12 (s, 1 H) 7.29 (dd, J = 9.83, 1.97 Hz, 1 H) 7.79 (d, |
| | J = 9.71 Hz, 1 H) 9.20 (dd, J = 1.62, 0.92 Hz, 1 H) 9.23 (d, J = 0.93 |
| | Hz, 1 H) 10.56 (br s, 1 H) |
| 4F | ¹H NMR (400 MHz, DMSO-d₆) δ ppm 1.17 (d, J = 6.94 Hz, 3 H) |
| | 1.21 (d, J = 6.94 Hz, 3 H) 1.47 (d, J = 6.47 Hz, 3 H) 2.86 (s, 3 H) |
| | 3.08 (s, 3 H) 3.14 (quin, J = 6.82 Hz, 1 H) 4.82 (d, J = 1.16 Hz, 2 |
| | H) 5.42 (q, J = 6.47 Hz, 1 H) 5.99 (s, 1 H) 7.29 (dd, J = 9.71, 1.85 |
| | Hz, 1 H) 7.79 (d, J = 9.71 Hz, 1 H) 9.21 (s, 1 H) 9.23 (d, J = 0.69 |
| | Hz, 1 H) 10.56 (s, 1 H) |
| 5F | ¹H NMR (400 MHz, DMSO-d₆) δ ppm 1.14 (d, J = 6.94 Hz, 6 H) |
| | 1.58-1.83 (m, 6 H) 1.89-2.02 (m, 2 H) 2.99-3.12 (m, 1 H) |
| | 4.81 (s, 2 H) 4.87-4.96 (m, 1 H) 6.26 (s, 1 H) 7.29 (dd, J = 9.71, |
| | 1.85 Hz, 1 H) 7.78 (d, J = 9.71 Hz, 1 H) 9.20 (d, J = 0.69 Hz, 1 H) |
| | 9.23 (d, J = 0.69 Hz, 1 H) 10.38-10.69 (m, 1 H) |
| 6F | ¹H NMR (400 MHz, DMSO-d₆) δ ppm 1.15 (d, J = 6.70 Hz, 6 H) |
| | 2.98-3.09 (m, 1 H) 3.10-3.22 (m, 1 H) 4.05-4.17 (m, 2 H) |
| | 4.22 (d, J = 6.01 Hz, 2 H) 4.29-4.41 (m, 2 H) 4.84 (s, 2 H) 6.34 |
| | (s, 1 H) 7.30 (dd, J = 9.71, 1.85 Hz, 1 H) 7.79 (d, J = 9.94 Hz, 1 H) |
| | 9.19 (s, 1 H) 9.24 (d, J = 0.69 Hz, 1 H) 10.54 (s, 1 H) |
| 7F | ¹H NMR (400 MHz, DMSO-d₆) δ ppm 1.17 (d, J = 6.94 Hz, 6 H) |
| | 1.77-2.01 (m, 4 H) 2.03-2.15 (m, 2 H) 2.70-2.83 (m, 1 H) |
| | 3.10 (quin, J = 6.82 Hz, 1 H) 4.02 (d, J = 6.47 Hz, 2 H) 4.82 (s, 2 |
| | H) 6.29 (s, 1 H) 7.30 (dd, J = 9.83, 1.97 Hz, 1 H) 7.79 (d, J = 9.71 |
| | Hz, 1 H) 9.18-9.21 (m, 1 H) 9.24 (d, J = 0.69 Hz, 1 H) 10.50- |
| | 10.58 (m, 1 H) |
| 8F | ¹H NMR (400 MHz, DMSO-d₆) δ ppm 1.18 (d, J = 6.94 Hz, 6 H) |
| | 1.76 (t, J = 19.30 Hz, 3 H) 3.11 (spt, J = 6.86 Hz, 1 H) 4.42 (t, |
| | J = 12.60 Hz, 2 H) 4.84 (s, 2 H) 6.45 (s, 1 H) 7.30 (dd, J = 9.83, |
| | 1.97 Hz, 1 H) 7.79 (d, J = 9.71 Hz, 1 H) 9.20 (s, 1 H) 9.24 (d, |
| | J = 0.69 Hz, 1 H) 10.57 (br d, J = 4.16 Hz, 1 H) |
| 9F | ¹H NMR (400 MHz, DMSO-d₆) δ ppm 0.94 (t, J = 7.40 Hz, 3 H) |
| | 1.12-1.19 (m, 6 H) 1.27 (d, J = 6.01 Hz, 3 H) 1.59-1.77 (m, 2 |
| | H) 3.09 (dquin, J = 13.70, 6.81, 6.81, 6.81, 6.81 Hz, 1 H) 4.56 |
| | (sxt, J = 5.92 Hz, 1 H) 4.82 (s, 2 H) 6.32 (s, 1 H) 7.31 (dd, |
| | J = 9.71, 1.85 Hz, 1 H) 7.79 (d, J = 9.71 Hz, 1 H) 9.21 (s, 1 H) 9.24 |
| | (d, J = 0.69 Hz, 1 H) 9.71-11.50 (m, 1 H) |
| 10F | ¹H NMR (500 MHz, DMSO-d₆) δ ppm 1.00 (d, J = 6.71 Hz, 6 H) |
| | 1.17 (d, J = 6.87 Hz, 6 H) 2.07 (dquin, J = 13.12, 6.56, 6.56, 6.56, |
| | 6.56 Hz, 1 H) 3.11 (spt, J = 6.84 Hz, 1 H) 3.84 (d, J = 6.10 Hz, 2 H) |
| | 4.82 (s, 2 H) 6.27 (s, 1 H) 7.30 (dd, J = 9.77, 1.98 Hz, 1 H) 7.78 |
| | (d, J = 9.77 Hz, 1 H) 9.20 (s, 1 H) 9.24 (d, J = 0.76 Hz, 1 H) 10.01- |
| | 11.17 (m, 1 H) |
| 11F | ¹H NMR (500 MHz, DMSO-d₆) δ ppm 1.18 (d, J = 6.87 Hz, 6 H) |
| | 1.42 (s, 3 H) 1.47 (s, 3 H) 3.07-3.18 (m, 1 H) 4.03-4.19 (m, 2 |
| | H) 4.84 (s, 2 H) 6.35 (s, 1 H) 7.30 (dd, J = 9.77, 1.83 Hz, 1 H) |
| | 7.79 (d, J = 9.77 Hz, 1 H) 9.20 (s, 1 H) 9.24 (d, J = 0.61 Hz, 1 H) |
| | 10.11-11.03 (m, 1 H) |
| 12F | ¹H NMR (500 MHz, DMSO-d₆) δ ppm 1.15 (d, J = 6.87 Hz, 6 H) |
| | 2.22 (s, 6 H) 2.67 (t, J = 5.42 Hz, 2 H) 3.09 (spt, J = 6.84 Hz, 1 H) |
| | 4.12 (t, J = 5.49 Hz, 2 H) 4.83 (s, 2 H) 6.33 (s, 1 H) 7.30 (dd, |
| | J = 9.69, 1.91 Hz, 1 H) 7.79 (d, J = 9.77 Hz, 1 H) 9.20 (s, 1 H) 9.24 |
| | (d, J = 0.61 Hz, 1 H) 10.37-10.78 (m, 1 H) |
| 13F | ¹H NMR (400 MHz, DMSO-d₆) δ ppm 1.19 (d, J = 6.94 Hz, 6 H) |
| | 3.12-3.23 (m, 1 H) 4.57 (dd, J = 7.86, 4.85 Hz, 2 H) 4.83 (s, 2 |
| | H) 4.96 (t, J = 6.94 Hz, 2 H) 5.31-5.43 (m, 1 H) 6.03 (s, 1 H) |
| | 7.29 (dd, J = 9.71, 1.85 Hz, 1 H) 7.78 (d, J = 9.71 Hz, 1 H) 9.20 (s, |
| | 1 H) 9.23 (s, 1 H) 10.35-10.74 (m, 1 H) |
| 14F | ¹H NMR (400 MHz, DMSO-d₆) δ ppm 1.16 (d, J = 6.94 Hz, 6 H) |
| | 1.59-1.74 (m, 1 H) 1.78-1.90 (m, 1 H) 2.00-2.18 (m, 2 H) |
| | 2.42-2.52 (m, 2 H) 3.03-3.17 (m, 1 H) 4.70-4.92 (m, 3 H) |
| | 6.10 (s, 1 H) 7.30 (dd, J = 9.71, 1.85 Hz, 1 H) 7.78 (d, J = 9.71 Hz, |
| | 1 H) 9.20 (s, 1 H) 9.23 (d, J = 0.92 Hz, 1 H) 10.57 (br s, 1 H) |
| 15F | ¹H NMR (500 MHz, DMSO-d₆) δ ppm 1.17 (d, J = 6.87 Hz, 6 H) |
| | 1.37 (s, 3 H) 3.11 (spt, J = 6.84 Hz, 1 H) 4.12 (s, 2 H) 4.34 (d, |
| | J = 5.95 Hz, 2 H) 4.50 (d, J = 5.95 Hz, 2 H) 4.84 (s, 2 H) 6.36 (s, 1 |
| | H) 7.30 (dd, J = 9.69, 1.91 Hz, 1 H) 7.79 (d, J = 9.77 Hz, 1 H) 9.20 |
| | (dd, J = 1.60, 0.99 Hz, 1 H) 9.24 (d, J = 0.61 Hz, 1 H) 10.55 (br s, |
| | 1 H) |
| 16F | ¹H NMR (500 MHz, DMSO-d₆) δ ppm 1.16 (d, J = 6.87 Hz, 6 H) |
| | 1.25 (s, 6 H) 3.05-3.16 (m, 1 H) 4.08 (s, 2 H) 4.84 (s, 2 H) |
| | 6.41 (s, 1 H) 7.30 (dd, J = 9.77, 1.98 Hz, 1 H) 7.79 (d, J = 9.77 Hz, |
| | 1 H) 9.19 (d, J = 0.61 Hz, 1 H) 9.24 (d, J = 0.61 Hz, 1 H) 10.33- |
| | 10.75 (m, 1 H) |
| 17F | ¹H NMR (500 MHz, DMSO-d₆) δ ppm 1.17 (d, J = 6.87 Hz, 6 H) |
| | 3.04-3.14 (m, 1 H) 4.45 (td, J = 14.69, 3.13 Hz, 2 H) 4.85 (s, 2 |
| | H) 6.26-6.64 (m, 2 H) 7.29 (dd, J = 9.69, 1.91 Hz, 1 H) 7.79 (d, |
| | J = 9.61 Hz, 1 H) 9.20 (dd, J = 1.60, 0.99 Hz, 1 H) 9.24 (d, J = 0.61 |
| | Hz, 1 H) 10.56 (s, 1 H) |
| 18F | ¹H NMR (400 MHz, DMSO-d₆) δ ppm 1.15 (d, J = 6.94 Hz, 6 H) |
| | 1.31 (d, J = 6.01 Hz, 6 H) 3.07 (spt, J = 6.82 Hz, 1 H) 4.72 (spt, |
| | J = 6.01 Hz, 1 H) 4.82 (s, 2 H) 6.31 (s, 1 H) 7.30 (dd, J = 9.94, |
| | 1.85 Hz, 1 H) 7.78 (d, J = 9.71 Hz, 1 H) 9.20 (d, J = 0.69 Hz, 1 H) |
| | 9.23 (d, J = 0.69 Hz, 1 H) 10.53 (s, 1 H) |
| 19F | ¹H NMR (400 MHz, DMSO-d₆) δ ppm 1.16 (d, J = 6.94 Hz, 6 H) |
| | 2.50 (s, 1 H) 2.59-2.83 (m, 4 H) 3.11 (spt, J = 6.74 Hz, 1 H) |
| | 4.12 (d, J = 5.55 Hz, 2 H) 4.83 (s, 2 H) 6.32 (s, 1 H) 7.30 (dd, |
| | J = 9.83, 1.97 Hz, 1 H) 7.79 (d, J = 9.71 Hz, 1 H) 9.19 (dd, J = 1.62, |
| | 0.92 Hz, 1 H) 9.24 (d, J = 0.69 Hz, 1 H) 10.54 (br s, 1 H) |
| 20F | ¹H NMR (400 MHz, DMSO-d₆) δ ppm 1.16 (dd, J = 6.70, 4.62 |
| | Hz, 6 H) 1.47 (d, J = 6.24 Hz, 3 H) 3.06 (spt, J = 6.86 Hz, 1 H) 4.85 |
| | (s, 2 H) 5.49 (spt, J = 6.28 Hz, 1 H) 6.68 (s, 1 H) 7.30 (dd, |
| | J = 9.94, 1.85 Hz, 1 H) 7.79 (d, J = 9.71 Hz, 1 H) 9.20 (s, 1 H) 9.23 |
| | (d, J = 0.69 Hz, 1 H) 10.19-10.93 (m, 1 H) |
| 21F | ¹H NMR (400 MHz, DMSO-d₆) δ ppm 1.17 (dd, J = 6.82, 0.81 |
| | Hz, 6 H) 1.47-1.59 (m, 1 H) 1.79 (tdd, J = 12.02, 12.02, 7.63, |
| | 4.85 Hz, 1 H) 2.23-2.38 (m, 1 H) 3.10 (spt, J = 6.82 Hz, 1 H) |
| | 4.05 (t, J = 9.94 Hz, 1 H) 4.25-4.33 (m, 1 H) 4.83 (s, 2 H) 6.35 |
| | (s, 1 H) 7.30 (dd, J = 9.83, 1.97 Hz, 1 H) 7.79 (d, J = 9.71 Hz, 1 H) |
| | 9.20 (dd, J = 1.62, 0.92 Hz, 1 H) 9.23 (d, J = 0.69 Hz, 1 H) 10.54 |
| | (br s, 1 H) |
| 22F | ¹H NMR (400 MHz, DMSO-d₆) δ ppm 0.32-0.44 (m, 2 H) 0.55- |
| | 0.68 (m, 2 H) 1.18 (d, J = 6.70 Hz, 6 H) 1.23-1.34 (m, 1 H) |
| | 3.12 (spt, J = 6.78 Hz, 1 H) 3.92 (d, J = 6.94 Hz, 2 H) 4.82 (s, 2 H) |
| | 6.25 (s, 1 H) 7.30 (dd, J = 9.71, 1.85 Hz, 1 H) 7.79 (d, J = 9.71 Hz, |
| | 1 H) 9.20 (s, 1 H) 9.24 (d, J = 0.69 Hz, 1 H) 10.54 (br s, 1 H) |
| 23F | ¹H NMR (500 MHz, DMSO-d₆) δ ppm 1.17 (d, J = 6.87 Hz, 6 H) |
| | 3.07 (spt, J = 6.89 Hz, 1 H) 4.86 (s, 2 H) 4.92 (q, J = 8.65 Hz, 2 H) |
| | 6.53 (s, 1 H) 7.30 (dd, J = 9.77, 1.83 Hz, 1 H) 7.79 (d, J = 9.77 Hz, |
| | 1 H) 9.20 (s, 1 H) 9.24 (s, 1 H) 10.58 (br s, 1 H) |
| 24F | ¹H NMR (500 MHz, DMSO-d₆) δ ppm 1.00 (t, J = 7.40 Hz, 3 H) |
| | 1.16 (d, J = 6.87 Hz, 6 H) 1.72-1.82 (m, 2 H) 3.10 (spt, J = 6.84 |
| | Hz, 1 H) 4.01 (t, J = 6.26 Hz, 2 H) 4.83 (s, 2 H) 6.28 (s, 1 H) 7.30 |
| | (dd, J = 9.77, 1.83 Hz, 1 H) 7.79 (d, J = 9.77 Hz, 1 H) 9.20 (d, |
| | J = 0.61 Hz, 1 H) 9.24 (s, 1 H) 10.54 (br s, 1 H) |
| 25F | ¹H NMR (400 MHz, DMSO-d₆) δ ppm 1.28 (d, J = 6.94 Hz, 6 H) |
| | 3.28 (br s, 1 H) 4.87 (s, 2 H) 5.64 (s, 1 H) 7.26-7.35 (m, 3 H) |
| | 7.36-7.44 (m, 1 H) 7.52-7.64 (m, 2 H) 7.79 (d, J = 9.71 Hz, 1 |
| | H) 9.20 (dd, J = 1.73, 1.04 Hz, 1 H) 9.24 (d, J = 0.69 Hz, 1 H) |
| | 10.21-10.95 (m, 1 H) |
| 26F | ¹H NMR (400 MHz, DMSO-d₆) δ ppm 1.11 (d, J = 6.94 Hz, 6 H) |
| | 1.58 (dtd, J = 12.72, 8.32, 8.32, 3.93 Hz, 2 H) 1.87-1.99 (m, 2 |
| | H) 3.00-3.13 (m, 2 H) 3.49 (ddd, J = 11.62, 8.38, 3.12 Hz, 2 H) |
| | 3.69-3.81 (m, 2 H) 4.69 (tt, J = 7.77, 3.79 Hz, 1 H) 4.76 (s, 2 H) |
| | 6.37 (s, 1 H) 7.23 (dd, J = 9.71, 1.85 Hz, 1 H) 7.72 (d, J = 9.71 Hz, |
| | 1 H) 9.10-9.22 (m, 2 H) |
| 31C | 1H NMR (500 MHz, DMSO-d6) δ ppm 0.76-0.85 (m, 2 H) |
| | 0.85-0.94 (m, 2 H) 1.38 (t, J = 7.02 Hz, 3 H) 2.10 (tt, J = 8.24, |
| | 4.96 Hz, 1 H) 4.12 (q, J = 6.97 Hz, 2 H) 4.84 (s, 2 H) 6.26 (s, 1 H) |
| | 7.89 (br d, J = 9.92 Hz, 1 H) 8.25-8.48 (m, 1 H) 9.50 (d, J = 0.61 |
| | Hz, 1 H) 11.38 (br s, 1 H) |
| 64 | 1H NMR (400 MHz, DMSO-d6) d ppm 0.84-0.95 (m, 2 H) |
| | 1.19 (d, J = 6.94 Hz, 8 H) 3.13 (spt, J = 6.82 Hz, 1 H) 4.38 (s, 1 H) |
| | 4.44 (s, 1 H) 4.84 (s, 2 H) 6.34 (s, 1 H) 7.30 (dd, J = 9.83, 1.97 |
| | Hz, 1 H) 7.79 (d, J = 9.71 Hz, 1 H) 9.20 (dd, J = 1.62, 0.92 Hz, 1 |
| | H) 9.24 (d, J = 0.69 Hz, 1 H) 10.55 (s, 1 H) |
| 41 | 1H NMR (500 MHz, DMSO-d6) d ppm 1.00 (d, J = 6.71 Hz, 6 H) |
| | 1.17 (d, J = 6.87 Hz, 6 H) 2.07 (dquin, J = 13.11, 6.49, 6.49, 6.49, |
| | 6.49 Hz, 1 H) 3.11 (spt, J = 6.84 Hz, 1 H) 3.84 (d, J = 6.26 Hz, 2 H) |
| | 4.90 (s, 2 H) 6.27 (s, 1 H) 7.82-7.98 (m, 1 H) 8.34 (dd, |
| | J = 10.07, 0.76 Hz, 1 H) 9.51 (d, J = 0.76 Hz, 1 H) 11.40 (br s, 1 H) |
| 49 | 1H NMR (500 MHz, DMSO-d6) d ppm 1.00 (d, J = 6.87 Hz, 6 H) |
| | 1.17 (d, J = 6.87 Hz, 6 H) 2.07 (dquin, J = 13.13, 6.56, 6.56, 6.56, |
| | 6.56 Hz, 1 H) 3.11 (spt, J = 6.84 Hz, 1 H) 3.84 (d, J = 6.10 Hz, 2 H) |
| | 4.87 (s, 2 H) 6.27 (s, 1 H) 7.72 (d, J = 1.07 Hz, 1 H) 7.82 (br d, |
| | J = 9.31 Hz, 1 H) 8.10 (dd, J = 9.77, 0.61 Hz, 1 H) 8.15 (s, 1 H) |
| | 11.03-11.32 (m, 1 H) |
| 60 | 1H NMR (DMSO-d6, 400 MHz) Shift 10.5-10.7 (m, 1H), 9.24 |
| | (s, 1H), 9.2-9.2 (m, 1H), 7.80 (d, 1H, J = 9.7 Hz), 7.30 (dd, 1H, |
| | J = 1.9, 9.8 Hz), 4.9-5.0 (m, 2H), 4.1-4.2 (m, 2H), 3.1-3.3 (m, |
| | 1H), 2.09 (quind, 1H, J = 6.6, 13.2 Hz), 1.19 (td, 6H, J = 0.9, 6.7 |
| | Hz), 1.0-1.1 (m, 6H) |
| 84 | 1H NMR (500 MHz, CHLOROFORM-d) d ppm 9.98 (s, 1 H) |
| | 9.11 (s, 1 H) 8.68 (d, J = 0.6 Hz, 1 H) 7.61-7.69 (m, 1 H) 7.11 |
| | (dd, J = 9.6, 1.8 Hz, 1 H) 5.04 (s, 2 H) 4.39 (t, J = 6.6 Hz, 2 H) |
| | 3.20 (spt, J = 6.8 Hz, 1 H) 1.81-1.91 (m, 2 H) 1.23 (d, J = 6.9 Hz, |
| | 6 H) 1.07 (t, J = 7.4 Hz, 3 H) |
| 74 | 1H NMR (500 MHz, CHLOROFORM-d) d ppm 9.85 (s, 1 H) |
| | 9.11 (s, 1 H) 8.69 (s, 1 H) 7.65 (d, J = 9.6 Hz, 1 H) 7.08 (dd, |
| | J = 9.8, 1.8 Hz, 1 H) 5.03 (s, 2 H) 4.27 (d, J = 7.3 Hz, 2 H) 3.28 |
| | (spt, J = 6.9 Hz, 1 H) 1.25 (d, J = 6.7 Hz, 7 H) 0.64-0.76 (m, 2 H) |
| | 0.33-0.44 (m, 2 H) |
| 72 | 1H NMR (500 MHz, CHLOROFORM-d) d ppm 9.61 (s, 1 H) |
| | 8.95 (d, J = 0.6 Hz, 1 H) 8.02-8.27 (m, 2 H) 5.10 (s, 2 H) 4.77 |
| | (q, J = 7.9 Hz, 2 H) 3.23 (quin, J = 6.8 Hz, 1 H) 1.25 (d, J = 6.9 Hz, |
| | 6 H) |
| 50 | 1H NMR (500 MHz, CHLOROFORM-d) d ppm 9.95 (br s, 1 H) |
| | 8.94 (d, J = 0.6 Hz, 1 H) 8.00-8.25 (m, 2 H) 5.11 (s, 2 H) 4.64 |
| | (t, J = 6.1 Hz, 2 H) 3.17 (quin, J = 6.8 Hz, 1 H) 2.69 (qt, J = 10.3, |
| | 6.2 Hz, 2 H) 1.23 (d, J = 6.9 Hz, 6 H) |
| 48 | 1H NMR (500 MHz, CHLOROFORM-d) d ppm 9.69 (s, 1 H) |
| | 8.92 (d, J = 0.6 Hz, 1 H) 8.03-8.30 (m, 2 H) 5.05 (s, 2 H) 4.40 |
| | (t, J = 6.6 Hz, 2 H) 3.10-3.34 (m, 1 H) 1.76-1.98 (m, 2 H) 1.24 |
| | (d, J = 6.9 Hz, 6 H) 1.07 (t, J = 7.5 Hz, 3 H) |
| 44 | 1H NMR (500 MHz, CHLOROFORM-d) d ppm 9.71 (br s, 1 H) |
| | 8.92 (d, J = 0.6 Hz, 1 H) 7.90-8.28 (m, 2 H) 5.06 (s, 2 H) 4.27 |
| | (d, J = 7.3 Hz, 2 H) 3.28 (quin, J = 6.8 Hz, 1 H) 1.25 (s, 7 H) 0.65- |
| | 0.73 (m, 2 H) 0.33-0.42 (m, 2 H) |
| 54 | 1H NMR (DMSO-d6, 400 MHz) Shift 10.56 (br s, 1H), 9.23 (d, |
| | 1H, J = 0.7 Hz), 9.21 (d, 1H, J = 1.5 Hz), 7.79 (d, 1H, J = 9.7 Hz), |
| | 7.30 (dd, 1H, J = 1.9, 9.8 Hz), 4.87 (s, 2H), 3.77 (d, 2H, J = 6.2 |
| | Hz), 3.12 (quin, 1H, J = 6.8 Hz), 2.0-2.1 (m, 4H), 1.17 (d, 6H, |
| | J = 6.8 Hz), 1.03 (d, 6H, J = 6.6 Hz) |
| 70 | 1H NMR (400 MHz, DMSO-d6) d ppm 10.45 (s, 1H), 9.24 (s, |
| | 1H), 9.19 (s, 1H), 7.78 (d, J = 9.7 Hz, 1H), 7.30 (d, J = 9.7 Hz, |
| | 1H), 6.25 (s, 1H), 4.73 (s, 2H), 3.84 (d, J = 6.2 Hz, 2H), 2.77 (s, |
| | 6H), 2.14-2.03 (m, 1H), 1.01 (d, J = 6.7 Hz, 6H). |
| 76 | 1H NMR (400 MHz, DMSO-d6) d ppm 11.35 (s, 1H), 9.51 (s, |
| | 1H), 8.35 (d, J = 10.0 Hz, 1H), 7.92 (d, J = 10.0 Hz, 1H), 6.20 (s, |
| | 1H), 4.80 (s, 2H), 3.91 (d, J = 7.2 Hz, 2H), 2.77 (s, 6H), 1.32- |
| | 1.23 (m, 1H), 0.64-0.58 (m, 2H), 0.39-0.34 (m, 2H). |
| 58 | 1H NMR (400 MHz, DMSO-d6) d ppm 11.35 (s, 1H), 9.51 (s, |
| | 1H), 8.35 (d, J = 10.1 Hz, 1H), 7.92 (d, J = 10.0 Hz, 1H), 6.25 (s, |
| | 1H), 4.80 (s, 2H), 3.84 (d, J = 6.2 Hz, 2H), 2.77 (s, 6H), 2.08 |
| | (m, J = 13.2, 6.5 Hz, 1H), 1.00 (d, J = 6.7 Hz, 6H). |
| 61 | 1H NMR (DMSO-d6, 400 MHz) Shift 10.5-10.7 (m, 1H), 9.25 |
| | (d, 1H, J = 0.8 Hz), 9.20 (dd, 1H, J = 1.0, 1.7 Hz), 7.80 (td, 1H, |
| | J = 0.9, 9.8 Hz), 7.30 (dd, 1H, J = 1.9, 9.8 Hz), 4.9-4.9 (m, 2H), |
| | 4.2-4.3 (m, 2H), 3.1-3.3 (m, 1H), 2.05 (quind, 1H, J = 6.6, 13.2 |
| | Hz), 1.1-1.2 (m, 6H), 0.9-1.1 (m, 6H) |
| 93 | 1H NMR (400 MHz, CHLOROFORM-d) d ppm 9.74 (s, 1 H) |
| | 9.13 (s, 1 H) 8.71 (s, 1 H) 7.66 (d, J = 9.7 Hz, 1 H) 7.09 (dd, |
| | J = 9.7, 1.8 Hz, 1 H) 5.05 (s, 2 H) 4.52 (t, J = 11.7 Hz, 2 H) 3.25 |
| | (quin, J = 6.8 Hz, 1 H) 1.79 (t, J = 18.6 Hz, 3 H) 1.25 (d, J = 6.9 Hz, |
| | 6 H) |
| 46 | 1H NMR (400 MHz, CHLOROFORM-d) d ppm 9.64 (s, 1 H) |
| | 8.94 (s, 1 H) 8.03-8.23 (m, 2 H) 5.08 (s, 2 H) 4.52 (t, J = 11.8 |
| | Hz, 2 H) 3.26 (spt, J = 6.8 Hz, 1 H) 1.79 (t, J = 18.7 Hz, 3 H) 1.25 |
| | (d, J = 6.7 Hz, 6 H) |
| 95 | 1H NMR (400 MHz, DMSO) d 11.28 (s, 1H), 9.52 (s, 1H), 8.35 |
| | (d, J = 10.0 Hz, 1H), 7.92 (d, J = 9.9 Hz, 1H), 6.48 (s, 1H), 4.91 |
| | (q, J = 8.7 Hz, 2H), 4.83 (s, 2H), 2.75 (s, 6H). |
| 92 | 1H NMR (400 MHz, CHLOROFORM-d) d ppm 6.75 (br s, 1 H) |
| | 6.09 (s, 1 H) 4.66 (s, 2 H) 3.71 (d, J = 6.2 Hz, 2 H) 3.14 (quin, |
| | J = 6.8 Hz, 1 H) 2.07-2.25 (m, 1 H) 1.92 (s, 6 H) 1.18-1.28 (m, |
| | 9 H) 1.05 (d, J = 6.7 Hz, 6 H) |
| 66 | 1H NMR (500 MHz, CHLOROFORM-d) d ppm 7.30 (br s, 1 H) |
| | 6.09 (s, 1 H) 4.66 (s, 2 H) 3.72 (d, J = 6.3 Hz, 2 H) 3.15 (dt, |
| | J = 13.7, 6.8 Hz, 1 H) 2.53 (s, 6 H) 2.14 (tt, J = 13.1, 6.6 Hz, 1 H) |
| | 1.21 (d, J = 6.9 Hz, 6 H) 1.05 (d, J = 6.9 Hz, 6 H) |
| 79 | 1H NMR (400 MHz, CHLOROFORM-d) d ppm 6.91 (br s, 1 H) |
| | 6.10 (s, 1 H) 4.67 (s, 2 H) 3.61-3.86 (m, 4 H) 3.15 (quin, J = 6.9 |
| | Hz, 1 H) 2.14 (dt, J = 13.2, 6.6 Hz, 1 H) 2.01 (s, 6 H) 1.38 (t, |
| | J = 5.8 Hz, 1 H) 1.21 (d, J = 6.7 Hz, 6 H) 1.05 (d, J = 6.7 Hz, 6 H) |
| 83 | 1H NMR (400 MHz, CHLOROFORM-d) d ppm 7.05 (br s, 1 H) |
| | 6.11 (s, 1 H) 5.57-5.99 (m, 1 H) 4.68 (s, 2 H) 3.72 (d, J = 6.2 |
| | Hz, 2 H) 3.15 (spt, J = 6.9 Hz, 1 H) 2.08-2.26 (m, 7 H) 1.21 (d, |
| | J = 6.9 Hz, 6 H) 1.05 (d, J = 6.7 Hz, 6 H) |
| 94 | 1H NMR (400 MHz, CHLOROFORM-d) d ppm 6.97 (s, 1 H) |
| | 6.10 (s, 1 H) 4.68 (s, 2 H) 3.67-3.76 (m, 6 H) 3.15 (quin, J = 6.8 |
| | Hz, 1 H) 2.39-2.49 (m, 4 H) 2.14 (dt, J = 13.2, 6.6 Hz, 1 H) 2.07 |
| | (s, 6 H) 1.21 (d, J = 6.7 Hz, 6 H) 1.05 (d, J = 6.7 Hz, 6 H) |
| 36 | 1H NMR (500 MHz, CHLOROFORM-d) d ppm 7.04 (br s, 1 H) |
| | 6.10 (s, 1 H) 4.67 (s, 2 H) 3.72 (d, J = 6.3 Hz, 2 H) 3.67 (s, 3 H) |
| | 3.15 (dt, J = 13.7, 6.9 Hz, 1 H) 2.35 (s, 6 H) 2.14 (dt, J = 13.2, 6.6 |
| | Hz, 1 H) 1.21 (d, J = 6.9 Hz, 6 H) 1.04 (d, J = 6.7 Hz, 6 H) |
| 37 | 1H NMR (400 MHz, CHLOROFORM-d) d ppm 7.19 (s, 1 H) |
| | 6.12 (s, 1 H) 4.73 (s, 2 H) 4.13 (s, 6 H) 3.73 (d, J = 6.2 Hz, 2 H) |
| | 3.70 (s, 3 H) 3.16 (quin, J = 6.9 Hz, 1 H) 2.03-2.30 (m, 1 H) |
| | 1.22 (d, J = 6.7 Hz, 6 H) 1.05 (d, J = 6.9 Hz, 6 H) |
| 40 | 1H NMR (500 MHz, CHLOROFORM-d) d ppm 6.95 (s, 1 H) |
| | 6.09 (s, 1 H) 4.68 (s, 2 H) 3.71 (d, J = 6.3 Hz, 2 H) 3.67 (s, 3 H) |
| | 3.15 (dt, J = 13.7, 6.9 Hz, 1 H) 2.24 (br s, 2 H) 2.13 (tt, J = 13.2, |
| | 6.6 Hz, 1 H) 1.90 (s, 4 H) 1.75 (dd, J = 4.0, 2.0 Hz, 2 H) 1.21 (d, |
| | J = 6.9 Hz, 6 H) 1.04 (d, J = 6.9 Hz, 6 H) |
| 38 | 1H NMR (400 MHz, CHLOROFORM-d) d ppm 6.68 (s, 1 H) |
| | 6.10 (s, 1 H) 4.67 (s, 2 H) 3.72 (d, J = 6.2 Hz, 2 H) 3.66 (s, 3 H) |
| | 3.15 (quin, J = 6.8 Hz, 1 H) 1.89-2.21 (m, 7 H) 1.64-1.84 (m, |
| | 4 H) 1.21 (d, J = 6.9 Hz, 6 H) 1.05 (d, J = 6.7 Hz, 6 H) |
| 45 | 1H NMR (500 MHz, CHLOROFORM-d) d ppm 6.15 (s, 1 H) |
| | 6.08 (s, 1 H) 4.61 (s, 2 H) 3.71 (d, J = 6.3 Hz, 2 H) 3.62 (s, 3 H) |
| | 3.14 (dquin, J = 13.7, 6.9, 6.9, 6.9, 6.9 Hz, 1 H) 2.13 (dquin, |
| | J = 13.2, 6.6, 6.6, 6.6, 6.6 Hz, 1 H) 1.80-1.98 (m, 12 H) 1.20 (d, |
| | J = 6.9 Hz, 6 H) 1.04 (d, J = 6.7 Hz, 6 H) |
| 82 | 1H NMR (500 MHz, CHLOROFORM-d) d ppm 6.92 (s, 1 H) |
| | 6.09 (s, 1 H) 4.67 (s, 2 H) 3.71 (d, J = 6.3 Hz, 2 H) 3.14 (dt, |
| | J = 13.7, 6.9 Hz, 1 H) 2.89 (s, 1 H) 2.19-2.27 (m, 6 H) 2.13 (tt, |
| | J = 13.2, 6.6 Hz, 1 H) 1.21 (d, J = 6.9 Hz, 6 H) 1.04 (d, J = 6.7 Hz, 6 H) |
| 88 | 1H NMR (500 MHz, CHLOROFORM-d) d ppm 6.02-6.23 (m, 2 |
| | H) 4.60 (s, 2 H) 3.71 (d, J = 6.3 Hz, 2 H) 3.14 (dt, J = 13.7, 6.9 Hz, |
| | 1 H) 2.13 (tt, J = 13.2, 6.6 Hz, 1 H) 1.93-2.10 (m, 6 H) 1.68- |
| | 1.81 (m, 6 H) 1.20 (d, J = 6.9 Hz, 6 H) 1.04 (d, J = 6.7 Hz, 6 H); 1H |
| | exchangeable |
| 59 | 1H NMR (500 MHz, CHLOROFORM-d) d ppm 6.58 (br s, 1 H) |
| | 6.09 (s, 1 H) 4.65 (s, 2 H) 3.71 (d, J = 6.3 Hz, 2 H) 3.15 (dquin, |
| | J = 13.7, 6.8, 6.8, 6.8, 6.8 Hz, 1 H) 1.99-2.20 (m, 3 H) 1.93 (s, 2 |
| | H) 1.66-1.88 (m, 6 H) 1.21 (d, J = 6.9 Hz, 6 H) 1.04 (d, J = 6.7 |
| | Hz, 6 H); 1H exchangeable |
| 75 | 1H NMR (400 MHz, CHLOROFORM-d) d ppm 6.61 (br s, 1 H) |
| | 6.10 (s, 1 H) 4.66 (s, 2 H) 3.72 (d, J = 6.2 Hz, 2 H) 3.15 (spt, |
| | J = 6.9 Hz, 1 H) 2.14 (dquin, J = 13.2, 6.6, 6.6, 6.6, 6.6 Hz, 1 H) |
| | 1.94-2.05 (m, 2 H) 1.73-1.91 (m, 6 H) 1.53-1.72 (m, 4 H) |
| | 1.21 (d, J = 6.9 Hz, 6 H) 1.05 (d, J = 6.7 Hz, 6 H); 2H |
| | exchangeables |
| 86 | 1H NMR (400 MHz, CHLOROFORM-d) d ppm 6.77 (br s, 1 H) |
| | 6.10 (s, 1 H) 4.67 (s, 2 H) 3.72 (d, J = 6.2 Hz, 2 H) 3.15 (quin, |
| | J = 6.9 Hz, 1 H) 2.14 (dquin, J = 13.2, 6.6, 6.6, 6.6, 6.6 Hz, 1 H) |
| | 1.68-1.94 (m, 8 H) 1.57-1.66 (m, 2 H) 1.21 (d, J = 6.9 Hz, 6 H) |
| | 1.05 (d, J = 6.7 Hz, 6 H) |
| 99 | 1H NMR (400 MHz, CHLOROFORM-d) d ppm 6.08 (s, 2 H) |
| | 4.61 (s, 2 H) 3.71 (d, J = 6.2 Hz, 2 H) 3.15 (spt, J = 6.9 Hz, 1 H) |
| | 2.14 (dquin, J = 13.2, 6.6, 6.6, 6.6, 6.6 Hz, 1 H) 1.85-2.01 (m, |
| | 6 H) 1.54-1.73 (m, 6 H) 1.21 (d, J = 6.7 Hz, 6 H) 1.05 (d, J = 6.9 |
| | Hz, 6 H), 2H exchangeables |
| 96 | 1H NMR (400 MHz, CHLOROFORM-d) d ppm 6.98 (s, 1 H) |
| | 6.17 (s, 1 H) 4.60-4.79 (m, 4 H) 3.73 (d, J = 6.2 Hz, 2 H) 3.16 |
| | (quin, J = 6.9 Hz, 1 H) 2.08-2.33 (m, 3 H) 1.94 (s, 3 H) 1.73- |
| | 1.84 (m, 2 H) 1.21 (d, J = 6.7 Hz, 6 H) 1.05 (d, J = 6.9 Hz, 6 H) |
| 67 | 1H NMR (400 MHz, METHANOL-d4) d ppm 7.55 (s, 1 H) 6.10 |
| | (s, 1 H) 4.53 (s, 2 H) 3.73 (d, J = 6.2 Hz, 2 H) 3.09 (quin, J = 6.9 |
| | Hz, 1 H) 2.04 (dquin, J = 13.2, 6.6, 6.6, 6.6, 6.6 Hz, 1 H) 1.67- |
| | 1.95 (m, 12 H) 1.12 (d, J = 6.9 Hz, 6 H) 0.97 (d, J = 6.7 Hz, 6 H), |
| | 1H exchangeable. |
| 62 | 1H NMR (DMSO-d6, 400 MHz) Shift 11.49 (s, 1H), 9.49 (s, |
| | 1H), 8.32 (d, 1H, J = 10.0 Hz), 7.89 (td, 1H, J = 1.6, 10.0 Hz), 7.04 |
| | (t, 1H, J = 53.3 Hz), 4.9-5.0 (m, 2H), 4.0-4.1 (m, 2H), 3.1-3.3 (m, |
| | 1H), 2.10 (td, 1H, J = 6.6, 13.2 Hz), 1.1-1.2 (m, 6H), 1.0-1.0 (m, 6H) |
| 42 | 1H NMR (DMSO-d6, 400 MHz) Shift 11.3-11.5 (m, 1H), 9.52 |
| | (s, 1H), 8.35 (d, 1H, J = 10.0 Hz), 7.92 (br d, 1H, J = 10.0 Hz), 4.9- |
| | 5.0 (m, 2H), 3.78 (d, 2H, J = 6.3 Hz), 3.1-3.3 (m, 1H), 2.0-2.1 |
| | (m, 4H), 1.1-1.2 (m, 6H), 1.03 (d, 6H, J = 6.7 Hz) |
| 68 | 1H NMR (DMSO-d6, 400 MHz) Shift 11.1-11.2 (m, 1H), 8.15 |
| | (s, 1H), 8.11 (dd, 1H, J = 0.7, 9.8 Hz), 7.82 (br d, 1H, J = 9.7 Hz), |
| | 7.72 (d, 1H, J = 1.3 Hz), 4.9-5.0 (m, 2H), 3.78 (d, 2H, J = 6.3 Hz), |
| | 3.11 (quin, 1H, J = 6.8 Hz), 2.0-2.1 (m, 4H), 1.17 (td, 6H, J = 0.6, |
| | 6.8 Hz), 1.0-1.1 (m, 6H) |
| 73 | 1H NMR (DMSO-d6, 400 MHz) Shift 10.67 (s, 1H), 8.43 (d, 2H, |
| | J = 5.2 Hz), 7.53 (d, 2H, J = 5.2 Hz), 4.85 (s, 2H), 3.7-3.8 (m, 2H), |
| | 3.11 (spt, 1H, J = 6.8 Hz), 2.0-2.1 (m, 4H), 1.16 (d, 6H, J = 6.8 |
| | Hz), 1.03 (d, 6H, J = 6.6 Hz) |
| 65 | 1H NMR (400 MHz, DMSO) d 10.75 (s, 1H), 8.35 (d, J = 5.8 Hz, |
| | 1H), 7.51 (s, 1H), 7.42 (dd, J = 5.7, 1.6 Hz, 1H), 6.28 (s, 1H), |
| | 4.82 (s, 2H), 3.84 (d, J = 6.1 Hz, 2H), 3.11 (dq, J = 13.8, 6.9 Hz, |
| | 1H), 2.45 (s, 3H), 2.14-2.00 (m, 1H), 1.17 (d, J = 6.9 Hz, 6H), |
| | 1.01 (d, J = 6.7 Hz, 6H). |
| 52 | 1H NMR (400 MHz, CDCl3) d 9.60 (s, 1H), 8.40 (d, J = 2.1 Hz, |
| | 1H), 8.35-8.29 (m, 2H), 6.20 (s, 1H), 4.94 (s, 2H), 3.75 (d, J = |
| | 6.2 Hz, 2H), 3.18 (dt, J = 13.7, 6.7 Hz, 1H), 2.15 (dt, J = 13.1, |
| | 6.5 Hz, 1H), 1.23 (d, J = 6.8 Hz, 6H), 1.05 (d, J = 6.7 Hz, 6H). |
| 55 | 1H NMR (400 MHz, CDCl3) d 9.27 (s, 1H), 8.87 (d, J = 1.0 Hz, |
| | 1H), 8.61 (d, J = 5.7 Hz, 1H), 8.12 (dd, J = 5.8, 1.2 Hz, 1H), |
| | 6.17 (s, 1H), 4.93 (s, 2H), 3.74 (d, J = 6.2 Hz, 2H), 3.17 (dt, J = |
| | 13.7, 6.8 Hz, 1H), 2.15 (dp, J = 13.1, 6.6 Hz, 1H), 1.23 (d, J = |
| | 6.9 Hz, 6H), 1.05 (d, J = 6.7 Hz, 6H). |
| 47 | 1H NMR (400 MHz, CDCl3) d 9.80 (s, 1H), 8.37 (d, J = 5.5 Hz, |
| | 2H), 7.37 (s, 2H), 6.17 (s, 1H), 4.93 (d, J = 1.7 Hz, 2H), 3.75 (d, |
| | J = 6.2 Hz, 2H), 3.23-3.09 (m, 1H), 2.15 (tq, J = 13.1, 6.5 Hz, |
| | 1H), 1.23 (d, J = 6.9 Hz, 6H), 1.05 (d, J = 6.7 Hz, 6H). |
| 63 | 1H NMR (400 MHz, CDCl3) d 9.55 (s, 1H), 8.16 (dt, J = 11.0, |
| | 5.5 Hz, 2H), 6.19 (s, 1H), 4.93 (s, 2H), 3.74 (d, J = 6.2 Hz, 2H), |
| | 3.16 (dt, J = 13.7, 6.8 Hz, 1H), 2.48 (d, J = 3.2 Hz, 3H), 2.19- |
| | 2.08 (m, 1H), 1.22 (d, J = 6.9 Hz, 6H), 1.04 (d, J = 6.7 Hz, 6H). |
| 51 | 1H NMR (DMSO-d6, 400 MHz) Shift 11.3-11.5 (m, 1H), 9.51 |
| | (s, 1H), 8.35 (d, 1H, J = 10.0 Hz), 7.92 (br d, 1H, J = 10.0 Hz), |
| | 6.25 (s, 1H), 4.8-5.0 (m, 2H), 3.9-4.0 (m, 2H), 3.1-3.3 (m, 1H), |
| | 1.2-1.3 (m, 1H), 1.1-1.2 (m, 6H), 0.5-0.7 (m, 2H), 0.3-0.4 (m, 2H) |
| 90 | 1H NMR (400 MHz, CHLOROFORM-d) d ppm 7.24 (s, 1 H) |
| | 6.12 (s, 1 H) 4.70 (s, 2 H) 3.73 (d, J = 6.2 Hz, 2 H) 3.16 (spt, |
| | J = 6.9 Hz, 1 H) 2.58 (s, 6 H) 2.37 (s, 3 H) 2.08-2.22 (m, 1 H) |
| | 1.22 (d, J = 6.9 Hz, 6 H) 1.05 (d, J = 6.7 Hz, 6 H) |
| 56 | 1H NMR (400 MHz, CDCl3) d 9.80 (s, 1H), 8.93 (d, J = 0.5 Hz, |
| | 1H), 8.19 (d, J = 10.1 Hz, 1H), 8.09 (d, J = 10.1 Hz, 1H), 6.24 (s, |
| | 1H), 4.98 (s, 2H), 4.11 (t, J = 11.0 Hz, 2H), 3.19 (dt, J = 13.7, |
| | 6.9 Hz, 1H), 1.79 (dd, J = 20.2, 17.0 Hz, 3H), 1.26 (d, J = 6.9 |
| | Hz, 6H). |
| 98 | 1H NMR (400 MHz, CHLOROFORM-d) d ppm 7.12 (s, 1 H) |
| | 6.11 (s, 1 H) 4.68 (s, 2 H) 3.73 (d, J = 6.2 Hz, 2 H) 3.28 (s, 3 H) |
| | 3.04-3.21 (m, 3 H) 2.33 (s, 6 H) 2.08-2.21 (m, 1 H) 1.22 (d, |
| | J = 6.7 Hz, 6 H) 1.05 (d, J = 6.7 Hz, 6 H) |
| 85 | 1H NMR (400 MHz, CHLOROFORM-d) d ppm 1.03-1.07 (m, 6 |
| | H) 1.18 (s, 6 H) 1.21 (d, J = 6.9 Hz, 6 H) 1.70-1.88 (m, 1 H) |
| | 1.97 (s, 6 H) 2.14 (dquin, J = 13.2, 6.6, 6.6, 6.6, 6.6 Hz, 1 H) |
| | 3.10-3.21 (m, 1 H) 3.72 (d, J = 6.2 Hz, 2 H) 4.68 (s, 2 H) 6.10 |
| | (s, 1 H) 6.91 (s, 1 H) |
| 78 | 1H NMR (500 MHz, CHLOROFORM-d) d ppm 7.36 (s, 1 H) |
| | 6.10 (s, 1 H) 4.69 (s, 2 H) 3.73 (d, J = 6.3 Hz, 2 H) 3.15 (quin, |
| | J = 6.9 Hz, 1 H) 2.85-3.01 (m, 2 H) 2.51 (s, 6 H) 2.14 (dt, |
| | J = 13.1, 6.4 Hz, 1 H) 1.81-1.97 (m, 2 H) 1.21 (d, J = 6.9 Hz, 6 H) |
| | 1.01-1.12 (m, 9 H) |
| 57 | 1H NMR (400 MHz, CDCl3) d 9.92 (s, 1H), 8.92 (s, 1H), 8.20 |
| | (d, J = 10.1 Hz, 1H), 8.09 (d, J = 10.1 Hz, 1H), 6.23 (s, 1H), 4.97 |
| | (s, 2H), 4.23 (d, J = 20.6 Hz, 2H), 3.24 (hept, J = 6.8 Hz, 1H), |
| | 1.31 (t, J = 7.4 Hz, 2H), 1.26 (d, J = 6.8 Hz, 6H), 0.85 (q, J = 7.6 |
| | Hz, 2H). |
| 77 | 1H NMR (400 MHz, DMSO-d6, 27° C.) d ppm 0.98-1.04 (m, 6 |
| | H), 1.13-1.21 (m, 6 H), 2.08 (dt, J = 13.1, 6.6 Hz, 1 H), 3.11 |
| | (quin, J = 6.8 Hz, 1 H), 3.17 (d, J = 5.2 Hz, 1 H), 3.84 (d, J = 6.2 Hz, |
| | 2 H), 4.86-5.05 (m, 2 H), 6.27 (s, 1 H), 7.30 (dd, J = 6.7, 4.4 Hz, |
| | 1 H), 8.79 (dd, J = 4.4, 1.9 Hz, 1 H), 9.30 (dd, J = 6.7, 1.9 Hz, 1 |
| | H), 11.25-11.37 (m, 1 H) |
| 89 | 1H NMR (400 MHz, CDCl3) d 6.67 (d, J = 7.4 Hz, 1H), 6.11 (s, |
| | 1H), 4.70 (s, 2H), 4.11-3.99 (m, 1H), 3.73 (d, J = 6.2 Hz, 2H), |
| | 3.16 (dd, J = 13.7, 6.9 Hz, 1H), 3.10 (t, J = 6.8 Hz, 2H), 2.40 |
| | (ddd, J = 9.9, 6.5, 2.9 Hz, 2H), 2.19 (s, 3H), 2.14 (dd, J = 13.2, |
| | 6.5 Hz, 3H), 1.98 (ddd, J = 12.1, 7.6, 3.0 Hz, 2H), 1.21 (d, J = |
| | 6.9 Hz, 6H), 1.05 (d, J = 6.7 Hz, 6H). |
| 81 | 1H NMR (400 MHz, CDCl3) d 6.68 (d, J = 7.1 Hz, 1H), 6.11 (s, |
| | 1H), 4.69 (s, 2H), 4.11-3.97 (m, 1H), 3.72 (d, J = 6.2 Hz, 2H), |
| | 3.16 (dq, J = 13.7, 6.9 Hz, 1H), 3.08 (dd, J = 12.7, 5.9 Hz, 2H), |
| | 2.48-2.37 (m, 4H), 2.14 (dd, J = 12.9, 6.1 Hz, 3H), 2.07- |
| | 1.98 (m, 2H), 1.21 (d, J = 6.9 Hz, 6H), 1.05 (d, J = 6.7 Hz, 6H), |
| | 0.98 (t, J = 7.2 Hz, 3H). |
| 71 | 1H NMR (400 MHz, CHLOROFORM-d) d ppm 1.05 (d, J = 6.7 |
| | Hz, 6 H) 1.21 (d, J = 6.9 Hz, 6 H) 2.06 (s, 6 H) 2.09-2.20 (m, 1 |
| | H) 2.58 (s, 2 H) 3.15 (spt, J = 6.8 Hz, 1 H) 3.66 (s, 3 H) 3.72 (d, |
| | J = 6.2 Hz, 2 H) 4.67 (s, 2 H) 6.10 (s, 1 H) 6.86 (br s, 1 H) |
| 43 | 1H NMR (400 MHz, DMSO-d6, 27° C.) d ppm 1.00 (br d, J = 6.5 |
| | Hz, 6 H) 1.17 (br d, J = 6.6 Hz, 6 H) 2.07 (dt, J = 12.7, 6.5 Hz, 1 H) |
| | 2.99-3.20 (m, 1 H) 3.84 (br d, J = 5.7 Hz, 2 H) 4.95 (br s, 2 H) |
| | 6.27 (s, 1 H) 8.23 (br d, J = 4.1 Hz, 1 H) 9.00 (br d, J = 3.6 Hz, 1 |
| | H) 9.24 (s, 1 H) 11.43 (br s, 1 H) |
| 53 | 1H NMR (400 MHz, DMSO-d6, 27° C.) d ppm 1.00 (br d, J = 6.7 |
| | Hz, 6 H) 1.17 (br d, J = 6.8 Hz, 6 H) 2.07 (tt, J = 13.0, 6.7 Hz, 1 H) |
| | 3.03-3.18 (m, 1 H) 3.84 (br d, J = 6.0 Hz, 2 H) 4.68-5.15 (m, 2 |
| | H) 6.27 (s, 1 H) 7.14 (br t, J = 6.7 Hz, 1 H) 7.60-7.67 (m, 1 H) |
| | 7.68-7.74 (m, 1 H) 8.84 (br d, J = 6.7 Hz, 1 H) 10.94-11.24 |
| | (m, 1 H) |
| 39 | ¹H NMR (400 MHz, DMSO-d₆, 27° C.) δ ppm 1.02 (d, J = 6.8 Hz, |
| | 4 H), 1.10-1.20 (m, 5 H), 2.09 (dt, J = 13.2, 6.6 Hz, 1 H), 3.17 |
| | (dt, J = 13.6, 6.8 Hz, 1 H), 4.11-4.15 (m, 2 H), 4.98-5.05 (m, 2 |
| | H), 7.86-7.94 (m, 1 H), 8.35 (dd, J = 10.0, 0.8 Hz, 1 H), 9.52 (d, |
| | J = 0.8 Hz, 1 H), 11.42-11.50 (m, 1 H) |

### Example B—Pharmaceutical Compositions

A compound of the invention (for instance, a compound of the examples) is brought into association with a pharmaceutically acceptable carrier, thereby providing a pharmaceutical composition comprising such active compound. A therapeutically effective amount of a compound of the invention (e.g. a compound of the examples) is intimately mixed with a pharmaceutically acceptable carrier, in a process for preparing a pharmaceutical composition.

### Example C—Biological Examples

The activity of a compound according to the present invention can be assessed by in vitro methods. A compound the invention exhibits valuable pharmacological properties, e.g. properties susceptible to inhibit NLRP3 activity, for instance as indicated the following test, and are therefore indicated for therapy related to NLRP3 inflammasome activity.

PBMC Assay

Peripheral venous blood was collected from healthy individuals and human peripheral blood mononuclear cells (PBMCs) were isolated from blood by Ficoll-Histopaque (Sigma-Aldrich, A0561) density gradient centrifugation. After isolation, PBMCs were stored in liquid nitrogen for later use. Upon thawing, PBMC cell viability was determined in growth medium (RPMI media supplemented with 10% fetal bovine serum, 1% Pen-Strep and 1% L-glutamine). Compounds were spotted in a 1:3 serial dilution in DMSO and diluted to the final concentration in 30 μl medium in 96 well plates (Falcon, 353072). PBMCs were added at a density of 7.5×10⁴ cells per well and incubated for 30 min in a 5% CO2 incubator at 37° C. LPS stimulation was performed by addition of 100 ng/ml LPS (final concentration, Invivogen, tlrl-smlps) for 6 hrs followed by collection of cellular supernatant and the analysis of IL-1β (μM) and TNF cytokines levels (μM) via MSD technology according to manufacturers' guidelines (MSD, K151A0H).

IC₅₀ and AC₅₀ values (for IL-10) and EC₅₀ and AC₅₀ values (TNF) were obtained on compounds of the invention/examples, and the AC₅₀ values are depicted in the following table:

| | IL1β | TNF |
|---|---|---|
| Compound | AC₅₀ (μM) | AC₅₀ (μM) |
| 1F | >15 | >10 |
| 2F | >15 | >10 |
| 3F | >15 | >10 |
| 4F | >15 | >10 |
| 5F | 0.47 | >10 |
| 6F | >15 | >10 |
| 7F | 0.21 | >10 |
| 8F | 0.19 | >10 |
| 9F | 0.56 | >10 |
| 10F | 0.06 | >10 |
| 11F | 0.32 | >10 |
| 12F | >15 | >10 |
| 13F | >15 | >10 |
| 14F | 0.68 | >10 |
| 15F | 2.30 | >10 |
| 16F | 1.56 | >10 |
| 17F | 0.69 | >10 |
| 18F | 0.66 | >10 |
| 19F | 5.33 | >10 |
| 20F | 1.29 | >10 |
| 21F | 0.65 | >10 |
| 22F | 0.11 | >10 |
| 23F | 0.16 | >10 |
| 24F | 0.19 | >10 |
| 25F | >15 | >10 |
| 26F | 13.6 | >10 |
| 27C | 13.3 | >10 |
| 28E | >15 | >10 |
| 29E | >15 | >10 |
| 30G | 14.6 | >10 |
| 32E | 14.6 | >10 |
| 31C | 0.92 | >10 |
| 33D | 1.25 | >10 |
| 34 | NA | NA |
| 35 | NA | NA |
| 36 | 0.01 | 3.81 |
| 37 | 0.02 | 2.73 |
| 38 | 0.02 | 12.20 |
| 39 | 0.02 | 0.60 |
| 40 | 0.02 | 8.85 |
| 41 | 0.04 | >20 |
| 42 | 0.05 | 9.41 |
| 43 | 0.07 | >20 |
| 44 | 0.07 | 3.53 |
| 45 | 0.07 | >20 |
| 46 | 0.08 | 8.17 |
| 47 | 0.08 | >20 |
| 48 | 0.09 | 4.98 |
| 49 | 0.09 | >20 |
| 50 | 0.09 | 1.62 |
| 51 | 0.09 | >20 |
| 52 | 0.09 | >20 |
| 53 | 0.10 | >20 |
| 54 | 0.10 | 15.55 |
| 55 | 0.11 | >20 |
| 56 | 0.13 | >20 |
| 57 | 0.14 | >20 |
| 58 | 0.15 | >10 |
| 59 | 0.16 | >20 |
| 60 | 0.16 | >20 |
| 61 | 0.16 | >20 |
| 62 | 0.18 | 3.75 |
| 63 | 0.19 | >20 |
| 64 | 0.20 | >20 |
| 65 | 0.21 | >20 |
| 66 | 0.22 | >20 |
| 67 | 0.24 | >20 |
| 68 | 0.24 | >20 |
| 69 | 0.25 | >20 |
| 70 | 0.26 | >20 |
| 71 | 0.26 | >20 |
| 72 | 0.27 | 16.62 |
| 73 | 0.33 | >20 |
| 74 | 0.36 | >20 |
| 75 | 0.36 | >20 |
| 76 | 0.37 | >20 |
| 77 | 0.40 | >20 |
| 78 | 0.42 | >20 |
| 79 | 0.43 | >20 |
| 80 | 0.45 | >20 |
| 81 | 0.48 | >20 |
| 82 | 0.50 | >20 |
| 83 | 0.51 | >20 |
| 84 | 0.63 | 19.45 |
| 85 | 0.65 | >20 |
| 86 | 0.66 | >20 |
| 87 | 0.66 | >20 |
| 88 | 0.66 | >20 |
| 89 | 0.67 | >20 |
| 90 | 0.68 | >20 |
| 91 | 0.69 | >20 |
| 92 | 0.71 | >20 |
| 93 | 0.77 | 8.92 |
| 94 | 0.78 | >20 |
| 95 | 0.79 | 18.16 |
| 96 | 0.87 | >20 |
| 97 | 0.95 | 5.01 |
| 98 | 0.96 | >20 |
| 99 | 0.97 | >20 |
| 100 | 1.00 | 4.81 |
| 101 | 1.02 | >20 |
| 102 | 1.06 | >20 |
| 103 | 1.10 | >20 |
| 104 | 1.12 | >20 |
| 105 | 1.21 | >20 |
| 106 | 1.21 | >20 |
| 107 | 1.31 | >20 |
| 108 | 1.33 | >20 |
| 109 | 1.35 | >20 |
| 110 | 1.44 | >20 |
| 111 | 1.47 | >20 |
| 112 | 1.55 | >20 |
| 113 | 1.79 | >20 |
| 114 | 1.85 | >20 |
| 115 | 1.88 | >20 |
| 116 | 1.91 | >20 |
| 117 | 2.04 | >20 |
| 118 | 2.30 | >20 |
| 119 | 2.52 | >20 |
| 120 | 2.66 | >20 |
| 121 | 2.71 | >20 |
| 122 | 2.81 | >20 |
| 123 | 2.91 | >20 |
| 124 | 3.40 | >20 |
| 125 | 3.41 | >20 |
| 126 | 3.56 | >20 |
| 127 | 3.88 | 19.39 |
| 128 | 4.28 | >20 |
| 129 | 4.70 | >20 |
| 130 | 8.73 | >20 |
| 131 | 9.63 | >20 |
| 132 | 9.79 | >20 |
| 133 | >10 | >10 |
| 134 | >10 | >10 |
| 135 | >10 | >10 |
| 136 | >10 | >10 |
| 137 | 10.19 | >20 |
| 138 | 10.68 | >20 |
| 139 | 10.80 | >20 |
| 140 | 11.79 | >20 |
| 141 | 12.16 | >20 |
| 142 | 15.70 | >20 |
| 143 | 18.01 | >20 |
| 144 | >20 | >20 |
| 145 | >20 | >20 |
| 146 | >20 | >20 |
| 147 | >20 | >20 |
| 148 | >20 | >20 |
| 149 | >20 | >20 |
| 150 | >20 | >20 |
| 151 | >20 | >20 |
| 152 | >20 | >20 |
| 153 | >20 | >20 |
| 154 | >20 | >20 |
| 155 | >20 | >20 |
| 156 | >20 | >20 |
| 157 | >20 | >20 |
| 158 | >20 | >20 |
| 159 | >20 | >20 |
| 160 | >20 | >20 |
| 161 | >20 | >20 |
| 162 | >20 | >20 |
| 163 | >20 | >20 |

### Example D—Further Testing

One or more compound(s) of the invention (including compounds of the final examples) is/are tested in a number of other methods to evaluate, amongst other properties, permeability, stability (including metabolic stability and blood stability) and solubility.

### Permeability Test

The in vitro passive permeability and the ability to be a transported substrate of P-glycoprotein (P-gp) is tested using MDCK cells stably transduced with MDR1 (this may be performed at a commercial organization offering ADME, PK services, e.g. Cyprotex). Permeability experiments are conducted in duplicate at a single concentration (5 μM) in a transwell system with an incubation of 120 min. The apical to basolateral (AtoB) transport in the presence and absence of the P-gp inhibitor GF120918 and the basolateral to apical (BtoA) transport in the absence of the P-gp inhibitor is measured and permeation rates (Apparent Permeability) of the test compounds (Pₐₚₚ×10⁻⁶ cm/sec) are calculated.

### Metabolic Stability Test in Liver Microsomes

The metabolic stability of a test compound is tested (this may be performed at a commercial organization offering ADME, PK services, e.g. Cyprotex) by using liver microsomes (0.5 mg/ml protein) from human and preclinical species incubated up to 60 minutes at 37° C. with 1 μM test compound.

The in vitro metabolic half-life (t_{1/2}) is calculated using the slope of the log-linear regression from the percentage parent compound remaining versus time relationship (κ),

t_{1/2}=−ln(2)/κ.

The in vitro intrinsic clearance (Clᵢₙₜ) (ml/min/mg microsomal protein) is calculated using the following formula:

$${Cl}_{int} = \frac{0.693}{{t}_{1 / 2}} \times \frac{{V}_{i n c}}{{W}_{mic prot , inc}}$$

Where: V_{inc}=incubation volume,

W_{mic prot,inc}=weight of microsomal protein in the incubation.

### Metabolic Stability Test in Liver Hepatocytes

The metabolic stability of a test compound is tested using liver hepatocytes (1 milj cells) from human and preclinical species incubated up to 120 minutes at 37° C. with 1 μM test compound.

The in vitro metabolic half-life (t_{1/2}) is calculated using the slope of the log-linear regression from the percentage parent compound remaining versus time relationship (κ),

t_{1/2}=−ln(2)/κ.

The in vitro intrinsic clearance (Clᵢₙₜ) (μl/min/million cells) is calculated using the following formula:

$${Cl}_{int} = \frac{0.693}{{t}_{1 / 2}} \times \frac{{V}_{i n c}}{ {cells}_{inc}} \times 1000$$

Where: V_{inc}=incubation volume,

#cells_{inc}=number of cells (×10⁶) in the incubation

### Solubility Test

The test/assay is run in triplicate and is semi-automated using the Tecan Fluent for all liquid handling with the following general steps:
20 μl of 10 mM stock solution is dispensed in a 500 μl 96 well plate
DMSO is evaporated (Genevac)
a stir bar and 400p of buffer/biorelevant media is added
the solution is stirred for 72h (pH2 and pH7) or 24h (FaSSIF and FeSSIF)
the solution is filtered
the filtrate is quantified by UPLC/UV using a three-points calibration curve

The LC conditions are:
Waters Acquity UPLC
Mobile phase A: 0.1% formic acid in H2O, B: 0.1% formic acid in CH3CN
Column: Waters HSS T3 1.8 μm 2.1×50 mm
Column temp.: 55° C.
Inj. vol.: 2 μl
Flow: 0.6 ml/min
Wavelength UV: 250_350 nm
Gradient: 0 min: 0% B, 0.3 min: 5% B, 1.8 min: 95% B, 2.6 min: 95% B
   Blood Stability assay

The compound of the invention/examples is spiked at a certain concentration in plasma or blood from the agreed preclinical species; then after incubating to predetermined times and conditions (37° C., 0° C. (ice) or room temperature) the concentration of the test compound in the blood or plasma matrix with LCMS/MS can then be determined.

1. A compound of formula (I), 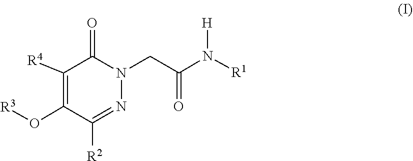
or a pharmaceutically acceptable salt thereof, wherein:
R¹ represents:
(i) C₃₋₈ cycloalkyl optionally substituted with one or more substituents independently selected from halo; cyano; C₁₋₃ alkyl; haloC₁₋₃ alkyl; —OH; —O—C₁₋₃ alkyl; —O—C₃₋₆ cycloalkyl; —NH₂; —NH-t.Boc; —NHC₁₋₃ alkyl; —N(C₁₋₃ alkyl)₂; piperidine; morpholine; hydroxyC₁₋₃ alkyl; C₁₋₃ alkyl substituted with —NH₂, —NH—C₁₋₃ alkyl, —O—C₁₋₃ alkyl or —SO₂—C₁₋₃ alkyl; —COOH, —COOC₁₋₃ alkyl, —CO—NH—NH₂, —CONH₂, —CONHC₁₋₃ alkyl, —CONHC₃alkynyl, —CON(C₁₋₃ alkyl)₂, —SO₂—C₁₋₃ alkyl, —SO₂—C₃₋₆cycloalkyl, heteroaryl or heterocyclyl;
(ii) aryl or heteroaryl, each of which is optionally substituted with 1 to 3 substituents independently selected from halo, —OH, —O—C₁₋₃ alkyl, —C₁₋₃ alkyl, haloC₁₋₃ alkyl, hydroxyC₁₋₃ alkyl, hydroxyC₁₋₃ alkoxy, haloC₁₋₃alkoxy; or
(iii) heterocyclyl, optionally substituted with 1 to 3 substituents independently selected from C₁₋₃ alkyl and C₃₋₆ cycloalkyl;
R² represents:
(i) C₁₋₃ alkyl optionally substituted with one or more substituents independently selected from halo, —OH and —OC₁₋₃ alkyl;
(ii) C₃₋₆ cycloalkyl; or
(iii) C₂₋₄ alkenyl optionally substituted with —OC₁₋₃ alkyl;
(iv) —N(R^{2a})R^{2b};
R^{2a} and R^{2b} each represent hydrogen or C₁₋₄ alkyl, or R^{2a} and R^{2b} may be linked together to form a 3- to 4-membered ring optionally substituted by one or more fluoro atoms;
R³ represents:
(i) C₁₋₆ alkyl optionally substituted with one or more substituents independently selected from halo, —OH, —OC₁₋₃ alkyl, —NH₂, —N(H)C₁₋₃ alkyl, —N(C₁₋₃ alkyl)₂ and —C(O)N(C₁₋₃ alkyl)₂;
(ii) C₂₋₆ alkenyl optionally substituted with one or more substituents independently selected from halo, —OH, —OC₁₋₃ alkyl, —NH₂, —N(H)C₁₋₃ alkyl, —N(C₁₋₃ alkyl)₂ and —C(O)N(C₁₋₃ alkyl)₂;
(iii) aryl or heteroaryl, each of which is optionally substituted with 1 to 3 substituents independently selected from halo, —OH, —O—C₁₋₃ alkyl, —C₁₋₃ alkyl, haloC₁₋₃ alkyl, hydroxyC₁₋₃ alkyl, hydroxyC₁₋₃ alkoxy, haloC₁₋₃alkoxy;
(iv) —X^{1a}—Y¹a, in which Y^{1a} represents C₃₋₆ cycloalkyl optionally substituted with one or more substituents independently selected from halo, —OH and —C₁₋₃ alkyl; or
(v) —X^{1b}—Y^{1b}, in which Y^{1b} represents heterocyclyl, optionally substituted with 1 to 3 substituents independently selected from halo, ═O, C₁₋₃ alkyl and —C(O)—C₁₋₆ alkyl;
X^{1a} and X^{1b} independently represent a —CH₂— linker group or a direct bond (i.e. is not present);
R⁴ represents:
(i) hydrogen;
(ii) halo;
(iii) C₁₋₄ alkyl optionally substituted with one or more substituents independently selected from halo, —OH and —OC₁₋₃ alkyl;
(iv) C₃₋₆ cycloalkyl; or
(v) —OC₁₋₃ alkyl.

2. The compound of claim 1, wherein R¹ represents C₃₋₆ cycloalkyl optionally substituted by one or two substituents selected from C₁₋₃ alkyl and —OH.

3. The compound of claim 2, wherein R¹ represents: 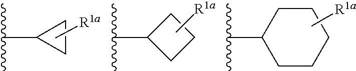
where each R^{1a} represents one or two optional substituents selected from —OH and C₁₋₃ alkyl.

4. The compound of claim 1, wherein R¹ represents: (i) phenyl; (ii) a 6-membered mono-cyclic heteroaryl group; or (iii) a 9- or 10-membered bicyclic heteroaryl group, all of which are optionally substituted with one or two substituent(s) selected from halo, —OH, C₁₋₃ alkyl and —OC₁₋₃ alkyl.

5. The compound of claim 4, wherein R¹ represents phenyl or a mono-cyclic 6-membered heteroaryl group: 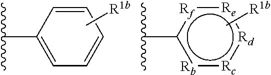
wherein R^{1b} represents one or two optional substituents selected from halo, —CH₃, —OH and —OCH₃, and, either one or two of R_{b}, R_{c}, R_{d}, Rₑ and R_{f} represent(s) a nitrogen heteroatom (and the others represent a CH).

6. The compound of claim 4, wherein R¹ represents a 9- or 10-membered bicyclic heteroaryl group, for instance: 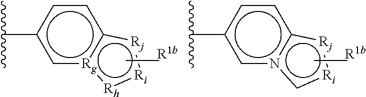
wherein R^{1b} represents one or two optional substituent selected from halo, —OH and —OCH₃, each ring of the bicyclic system is aromatic, R_{g} represents a N or C atom and any one or two of Rₕ, Rᵢ and Rⱼ represents N and the other(s) represent(s) C.

7. The compound of claim 1, wherein R² represents: (i) C₁₋₃ alkyl optionally substituted with one or more substituents independently selected from halo, —OH and —OC₁₋₂ alkyl; (ii) C₃₋₆ cycloalkyl; or (iii) C₂₋₄ alkenyl optionally substituted with —OC₁₋₂ alkyl.

8. The compound of claim 7, wherein R² represents unsubstituted C₁₋₃ alkyl.

9. The compound of claim 1, wherein R³ represents: (i) C₁₋₆ alkyl optionally substituted by one or more substituents independently selected from fluoro, —N(C₁₋₃ alkyl)₂ and —C(O)N(CH₃)₂; (ii) aryl optionally substituted by one or more substituents selected from halo, —OC₁₋₃ alkyl, —C₁₋₃ alkyl and haloC₁₋₃ alkyl; (iii) —X^{1a}—Y¹a, in which X^{1a} represents —CH₂— or a direct bond, and Y^{1a} represents C₃₋₆ cycloalkyl optionally substituted by one or more halo atoms; (iv) —X^{1b}—Y^{1b}, in which X^{1b} represents —CH₂— or a direct bond, and Y^{1b} represents heterocyclyl, for instance a 4-6 membered heterocyclyl group, optionally bridged, and containing one heteroatom selected from nitrogen, oxygen and sulfur, and which heterocyclyl group is optionally substituted by one or more substituents selected from halo, ═O, C₁₋₃ alkyl and —C(O)C₁₋₄ alkyl.

10. The compound of claim 1, wherein R⁴ represents H, halo, C₁₋₃ alkyl or C₃₋₆ cycloalkyl.

11. A pharmaceutical composition comprising a therapeutically effective amount of a compound as defined in claim 1 and a pharmaceutically acceptable carrier.

12. A process for preparing a pharmaceutical composition, comprising intimately mixing a pharmaceutically acceptable carrier with a therapeutically effective amount of a compound as defined in claim 1.

13. (canceled)

14. A combination comprising: (a) a compound according to claim 1; and (b) one or more other therapeutic agents.

15. (canceled)

16. A method of treating a disease or disorder associated with inhibition of NLRP3 inflammasome activity in a subject in need thereof, comprising administering to said subject a therapeutically effective amount of the compound according to claim 1.

17. The method according to claim 16 wherein the disease or disorder associated with inhibition of NLRP3 inflammasome activity is selected from inflammasome related diseases and disorders, immune diseases, inflammatory diseases, auto-immune diseases, auto-inflammatory fever syndromes, cryopyrin-associated periodic syndrome, chronic liver disease, viral hepatitis, non-alcoholic steatohepatitis, alcoholic steatohepatitis, alcoholic liver disease, inflammatory arthritis related disorders, gout, chondrocalcinosis, osteoarthritis, rheumatoid arthritis, chronic arthropathy, acute arthropathy, kidney related disease, hyperoxaluria, lupus nephritis, Type I and Type II diabetes, nephropathy, retinopathy, hypertensive nephropathy, hemodialysis related inflammation, neuroinflammation-related diseases, multiple sclerosis, brain infection, acute injury, neurodegenerative diseases, Alzheimer's disease, cardiovascular diseases, metabolic diseases, cardiovascular risk reduction, hypertension, atherosclerosis, peripheral artery disease, acute heart failure, inflammatory skin diseases, acne, wound healing and scar formation, asthma, sarcoidosis, age-related macular degeneration, colon cancer, lung cancer, myeloproliferative neoplasms, leukemias, myelodysplastic syndromes and myelofibrosis.

18. A process for the preparation of the compound of formula (I) as claimed in claim 1, comprising:
(i) reacting a compound of formula (II), 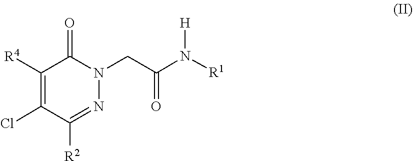
or a derivative thereof, wherein R¹ and R² are as defined in claim 1, R⁴ is hydrogen, with a compound of formula (III),
HO—R³ (111)
or a derivative thereof, wherein R³ is as defined in claim 1;
(ii) reacting a compound of formula (IV), 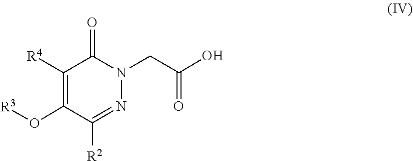
or a derivative thereof, wherein R², R³ and R⁴ are as hereinbefore defined, with a compound of formula (V),
H₂N—R¹ (V)
or a derivative thereof, wherein R¹ is as hereinbefore defined, under amide-forming reaction conditions;
(iii) reacting a compound of formula (VI), 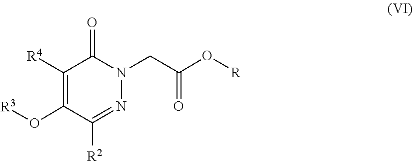
or a derivative thereof, wherein R is C₁₋₄ alkyl and R², R³ and R⁴ are as hereinbefore defined, with a compound of formula (V) as defined above, under amide-forming reaction conditions,
(iv).

19. The process of claim 18, wherein the compound of formula (II) or the compound of formula (IV) is: 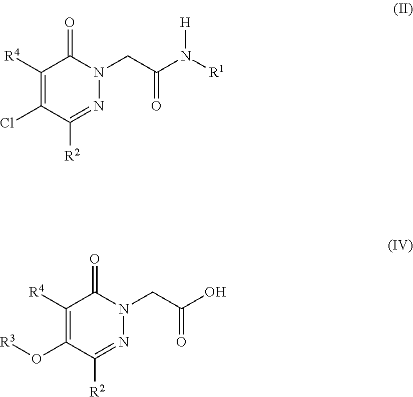
wherein
R¹ represents:
(iv)C₃₋₈ cycloalkyl optionally substituted with one or more substituents independently selected from halo: cyano; C₁₋₃ alkyl; haloC₁₋₃ alkyl; —OH; —O—C₁₋₃ alkyl; —O—C₃₋₆ cycloalkyl: —NH₂: —NH-t.Boc: —NHC₁₋₃ alkyl; —N(C₁₋₃ alkyl)₂: piperidine; morpholine; hydroxyC₁₋₃ alkyl: C₁₋₃ alkyl substituted with —NH₂, —NH— C₁₋₃ alkyl, —O—C₁₋₃ alkyl or —SO₂—C₁₋₃ alkyl: —COOH, —COOC₁₋₃ alkyl, —CO—NH—NH₂, —CONH₂, —CONHC₁₋₃ alkyl, —CONHC₃alkynyl, —CON(C₁₋₃ alkyl)₂, —SO₂—C₁₋₃ alkyl, —SO₂—C₃₋₆cycloalkyl, heteroaryl or heterocyclyl;
(v) aryl or heteroaryl, each of which is optionally substituted with 1 to 3 substituents independently selected from halo, —OH, —O—C₁₋₃ alkyl, —C₁₋₃ alkyl, haloC₁₋₃ alkyl, hydroxyC₁₋₃ alkyl, hydroxyC₁₋₃ alkoxy, haloC₁₋₃alkoxy: or
(vi) heterocyclyl, optionally substituted with 1 to 3 substituents independently selected from C₁₋₃ alkyl and C₃₋₆ cycloalkyl;
R² represents:
(v) C₁₋₃ alkyl optionally substituted with one or more substituents independently selected from halo, —OH and —OC₁₋₃ alkyl;
(vi)C₃₋₆ cycloalkyl; or
(vii) C₂₋₄ alkenyl optionally substituted with —OC₁₋₃ alkyl;
(viii) —N(R^{2a})R^{2b};
R^{2a} and R^{2b} each represent hydrogen or C₁₋₄ alkyl, or R^{2a} and R^{2b} may be linked together to form a 3- to 4-membered ring optionally substituted by one or more fluoro atoms;
R³ represents:
(vi)C₁₋₆ alkyl optionally substituted with one or more substituents independently selected from halo, —OH, —OC₁₋₃ alkyl, —NH₂, —N(H)C₁₋₃ alkyl, —N(C₁₋₃ alkyl)₂ and —C(O)N(C₁₋₃ alkyl)₂;
(vii) C₂₋₆ alkenyl optionally substituted with one or more substituents independently selected from halo, —OH, —OC₁₋₃ alkyl, —NH₂, —N(H)C₁₋₃ alkyl, —N(C₁₋₃ alkyl)₂ and —C(O)N(C₁₋₃ alkyl)₂;
(viii) aryl or heteroaryl, each of which is optionally substituted with 1 to 3 substituents independently selected from halo, —OH, —O—C₁₋₃ alkyl, —C₁₋₃ alkyl, haloC₁₋₃ alkyl, hydroxyC₁₋₃ alkyl, hydroxyC₁₋₃ alkoxy, haloC₁₋₃alkoxy;
(ix) —X^{1a}—Y^{1a}, in which Y^{1a} represents C₃₋₆ cycloalkyl optionally substituted with one or more substituents independently selected from halo, —OH and —C₁₋₃ alkyl; or
(x) —X^{1b}—Y^{1b}, in which Y^{1b} represents heterocyclyl, optionally substituted with 1 to 3 substituents independently selected from halo, ═O, C₁₋₃ alkyl and —C(O)—C₁₋₆ alkyl;
X^{1a} and X^{1b} independently represent a —CH₂— linker group or a direct bond (i.e. is not present);
R⁴ represents:
(vi) hydrogen;
(vii) halo;
(viii) C₁₋₄ alkyl optionally substituted with one or more substituents independently selected from halo, —OH and —OC₁₋₃ alkyl;
(ix) C₃₋₆ cycloalkyl; or
(x) —OC₁₋₃ alkyl.
